(19) United States
(12) Patent Application Publication
(10) Pub. No.: US 2021/0038577 A1
(43) Pub. Date: Feb. 11, 2021

(54) THIOREDOXIN REDUCTASE INHIBITORS FOR USE IN THE TREATMENT OF CANCER

(71) Applicant: Cinda Pharma AB, Gothenburg (SE)

(72) Inventors: Owe Orwar, Hovas (SE); Max Davidson, Gothenburg (SE)

(21) Appl. No.: 16/969,165

(22) PCT Filed: Feb. 12, 2019
(86) PCT No.: PCT/EP2019/053444

§ 371 Date: Aug. 11, 2020

(30) Foreign Application Priority Data
Feb. 12, 2018 (GB) 1802264.0
May 4, 2018 (GB) 1807415.3

(51) Int. Cl. A61K 31/444; A61P 35/00; A61K 9/00; A61K 31/55; C07K 16/18

(52) U.S. Cl. CPC A61K 31/444; A61P 35/00; A61K 45/06; A61K 31/55; C07K 16/18; A61K 9/0019

(57) ABSTRACT

The present invention provides inhibitors of thioredoxin reductase, in particular selenium compromised thioredoxin reductase-derived apoptotic protein (SecTRAP) forming agents, for use in treating an immune cell infiltrated cancer (e.g. a T-cell infiltrated cancer) in a subject, wherein said agents stimulate an anti-cancer immune response. The present invention also provides combinations comprising a SecTRAP forming agent and other therapeutic agents for use in treating cancer.

The present invention relates generally to Thioredoxin reductase (TrxR) inhibitors, particularly SecTRAP forming agents. More particularly, the invention relates to such agents for use in the treatment of cancer.

Cancer treatment is still one of the biggest unmet medical needs. While there have been advances in cancer therapy during the last decades, cancer remains a leading cause of death. Thus, the demand for new cancer therapies is ever increasing.

One of the hallmarks of cancer is to try to evade an anti-tumor immune response.

What are needed in the art are new cancer therapies which are able to make use of the immune system of the subject being treated, for example work synergistically with the immune system of the subject being treated, in order for there to be, or to enhance, a clinically beneficial anti-cancer immune response.

Mammalian thioredoxin reductases (TrxR, E.C. 1.8.1.9) are selenoproteins, i.e. they belong to the unique family of proteins that contain a selenocysteine (Sec, U in one-letter code) residue. TrxR has, together with the principle substrate thioredoxin (Trx), a wide range of functions in cells as a major reducing system for DNA synthesis, redox regulatory functions and antioxidant defense. TrxR family enzymes are pyridine nucleotide oxidoreductases. Three mammalian isoenzymes of TrxR have been identified, namely the most abundant predominantly cytosolic TrxR1, mitochondrial TrxR2 and TGR (thioredoxin glutathione reductase), the latter mainly expressed in testis. It should be noted that TrxR proteins of other organisms such as bacteria, archaea, plants or insects, are typically not selenoproteins. There is also a lack of consensus for nomenclature of TrxR, sometimes abbreviated as TR or TXNRD, with additional abbreviations occurring, e.g. mitochondrial TrxR2 is the same enzyme as TR3 and TGR has also been called TR2.

SecTRAPs (selenium compromised thioredoxin reductase-derived apoptotic proteins may be described as derivatives of thioredoxin reductase (TrxR) that have (i) a compromised Sec residue (selenocysteine residue), (ii) reduced or inhibited (or abolished) thioredoxin reducing ability and (ill) a capacity to induce cell death by gain of function (Anestål et al. PLOS One, (2008) Vol:3 (4), e1846). SecTRAPs can be considered to be pro-oxidant killers of cells, which trigger mechanisms beyond those of a mere loss of thioredoxin reductase activity (Anestål et al., supra).

Anestal et al. (supra) describe how TrxR with a compromised Sec residue show cytotoxic properties as SecTRAPs and that the cell death observed has apoptotic and necrotic properties. Anestal et al. describe a change of function of TrxR to a prooxidant enzyme upon its conversion to a SecTRAP, which it is stated may be done using electrophilic compounds that target TrxR.

Thus, classically, anti-cancer cell activity of SecTRAP forming agents is thought to occur by directly inducing cell death by apoptosis and/or necrosis. The present inventors have surprisingly found that SecTRAP forming agents additionally exert anti-cancer activity in a different, and indirect, way that is therapeutically beneficial. In this regard, the present inventors have found that SecTRAP forming agents can elicit a therapeutically beneficial anti-cancer immune response. Thus, the present inventors have surprisingly found that SecTRAP forming agents have a dual mode of action in the context of cancer treatments, a direct cell death effect caused by direct cytotoxic/cytolytic action, and an indirect effect that harnesses the patient's immune system to target the cancer. Thus, the SecTRAP formers have been found to be immunoactivating, and/or have additive or synergistic action with the immune system in fighting cancers. This surprising finding has opened up the prospect for improved cancer treatments. Additionally, with this invention it is also possible to identify responders and stratify patients in large patient populations to yield maximum therapeutic benefit from said treatment.

Thus, in one aspect, the present invention provides a selenium compromised thioredoxin reductase-derived apoptotic protein (SecTRAP) forming agent for use in treating a T-cell infiltrated cancer in a subject, wherein said agent has immunostimulatory activity thereby causing said subject to raise an immune response against said cancer.

The agent has immunostimulatory activity via the formation of the SecTRAP. SecTRAPs are described elsewhere herein.

Alternatively viewed, the present invention provides a selenium compromised thioredoxin reductase-derived apoptotic protein (SecTRAP) forming agent for use in treating a T-cell infiltrated cancer in a subject, wherein said agent stimulates (or causes or elicits or enhances) an anti-cancer immune response.

A SecTRAP forming agent (or TrxR inhibitor) for use in accordance with the present invention may be characterized by three main characteristics, (i), the compound (or agent) binds to TrxR at C-terminal active site Sec-residue (the C-terminal active site is characterised by a surface exposed selenocysteine (Sec) residue); (ii) the compound inhibits (e.g. significantly inhibits or partially inhibits or fully inhibits) the ability of TrxR to reduce Trx (a normal cellular substrate for the C-terminal active site) or other substrates (e.g. DTNB) at the C-terminal active site (e.g. in an NADPH dependent manner, in the presence of NADPH); (ill) TrxR is still able to have activity (or retain or maintain activity) (e.g. juglone reducing activity) at the intact N-terminal redox active site.

Upon treatment with a SecTRAP forming agent, TrxR typically retains oxidative capacity and becomes a free radical generator. Upon treatment with a SecTRAP forming agent, TrxR can still have redox activity (oxidoreductase) (e.g. at the N-terminal active site), albeit TrxR has reduced or abolished ability to reduce Trx (due to the inhibition at the C-terminal active site). Treatment with a SecTRAP forming agent can be considered to convert TrxR into a prooxidant enzyme.

The enzyme TrxR works by using reducing equivalents from NADPH to perform direct antioxidant activity as well as transfer reducing equivalents to other redox enzymes within the cell. TrxR thus provides reducing equivalents to proteins involved in antioxidant activity, cell death regulation, DNA synthesis, and more.

Thioredoxin reductase (TrxR) proteins are members of the pyridine nucleotide disulfide oxidoreductase family. Different from most proteins, they contain an additional amino acid to the common 20 amino acids found in proteins of all organisms. This amino acid is called selenocysteine (Sec), and has been coined the 21st amino acid.

TrxR proteins support multiple cellular signaling processes and directly perform antioxidant activities. TrxR is reduced by NADPH (via electron donation), then TrxR reduces many cellular substrates, the main substrate being thioredoxin (Trx). Trx in reduced form then exerts antioxidant activity, cell death, and proliferative roles in the cell via several pathways.

Located at the penultimate residue in the protein sequence in TrxR, the Sec amino acid forms a selenothiol bond with a neighboring Cys and serves as the main catalytic residue when the enzyme is reduced. The process of reducing TrxR (e.g. TrxR1) occurs through an electron flow from the redox cofactor Flavin adenine dinucleotide (FAD), to the N-terminus of one subunit in the dimer, then finally to the C-terminus of the other subunit. More specifically, reduction of TrxR begins with NADPH binding to one of the dimer subunits and transferring electrons to the FAD. The electrons from the FADH2 are then transferred to a dithiol motif in the N-terminus of the same TrxR subunit. This reduced moiety then reduces the selenothiol motif in the C-terminus of the other subunit within the dimer, fully activating the enzyme complex for catalysis.

A molecule-specific effect of TrxR inhibition is the ability for the enzyme to form a selenium compromised thioredoxin reductase-derived apoptotic protein (SecTRAP). In order for a SecTRAP to be formed, a small molecule inhibitor has to bind to the reduced C-terminal redox motif of TrxR where the Sec amino acid is located, while leaving the other redox active moieties (FAD and N-terminus dithiol motif) of the enzyme intact.

When small molecules inhibit the Sec residue and not other parts of the enzyme, the enzyme can still react with NADPH and become reduced at the N-terminus. In this form, TrxR is unable to react with Trx and other typical substrates of the enzyme, leading to the formation or production of oxidized Trx and PDI (protein disulphide isomerase). This will typically also lead to buildup of downstream oxidized substrates such as peroxiredoxins etc. The enzyme remains redox active at the N-terminal active site, sustaining NADPH consumption, resulting in a SecTRAP that actively produces oxidative stress. It is believed that the sole function of the N-terminal active site in the non-inhibited enzyme is to transfer electrons from the FAD moiety to the C-terminal selenol-thiol active motif.

Under normal conditions (e.g. in the absence of a SecTRAP forming agent), the pro-oxidant activity of TrxR is absent because full electron transfer from TrxR to Trx can occur, resulting in a net antioxidant effect. Therefore, uninhibited TrxR promotes cell survival and proliferation. Inhibition of TrxR theoretically results in a net increase in cellular oxidation as its inability to activate Trx, and its antioxidant properties, are lost. However, SecTRAP formation results in an active production of pro-oxidant units beyond the mere loss of TrxR-specific antioxidant activities. As a SecTRAP, TrxR cannot reduce Trx, but its NADPH oxidase activity remains intact.

SecTRAPs essentially display sustained NADPH oxidase activity although it is prevented from donating electrons to its natural substrates. In the absence of its typical electron-accepting substrates (e.g. oxidized Trx), SecTRAP formation results in an increased production of H₂O₂ within cancer cells. This active increase in H₂O₂, in a cellular context, shows how TrxR is converted from an antioxidant enzyme into a pro-oxidant enzyme. The active increase in the oxidative tension on cells through SecTRAP formation results in both passive and active initiation of cell death.

Whether or not a given agent (or compound) has SecTRAP forming activity (i.e. is able to generate a SecTRAP) may be determined by any appropriate means and the skilled person is familiar with appropriate methods and assays to use.

For example, whether or not a given agent (or compound) is a SecTRAP forming agent can be determined in vitro using the following methods:

C-terminal activity (C-terminal active site activity) of TrxR may be determined according to the following procedure (assay):
1) TrxR is reduced with NADPH
2) Reduced TrxR is mixed with the compound under investigation allowing the compound to bind
3) After incubation, the mixture is desalted using a spin column to remove unbound compound
4) a model substrate is added to the mixture
5) C-terminal reducing activity is measured by determining reduction of the model substrate.

A particularly preferred C-terminal activity assay is described in the Example section herein. A typical model substrate is DTNB (5,5′-dithio-bis(2-dinitrobenzoic acid). Reduction of DTNB is followed by monitoring TNB-production using a spectrophotometer. To confirm that the compound binds irreversibly, the enzyme compound mixture is passed over a spin column (step 3) to remove unbound compound before adding the model substrate. If model substrate reduction is blocked after compound removal, the binding event was irreversible. Reduction of substrate should only occur if TrxR was first reduced by NADPH.

N-terminal activity (N-terminal active site activity) is determined using, e.g., a juglone reduction assay in which
1) NADPH-reduced, compound-treated TrxR is mixed with juglone
2) Reduction of juglone is measured indirectly based on NADPH consumption.

A particularly preferred N-terminal activity assay is described in the Example section herein. The choice of juglone (5-Hydroxy-1,4-naphthoquinone) is based on the finding that juglone is mainly reduced by the N-terminal active site dithiol motif, unlike other substrates that are reduced mainly or solely by the C-terminal selenolthiol motif.

Using the above two methods (i.e. assessing C-terminal activity and N-terminal activity) whether or not a given compound (agent) is a SecTRAP forming agent can be determined. A given compound (agent) would typically be classified as a SecTRAP forming agent if C-terminal activity is inhibited (or diminished or abolished) e.g. as assessed by the above type of C-terminal activity assay, but N-terminal activity is not significantly inhibited (or not fully or completely inhibited) e.g. as assessed in the above type of N-terminal activity assay.

Preferred SecTRAP forming agents are those that exhibit (or maintain or retain) at least 10%, at least 20%, at least 30%, more preferably at least 40%, preferably at least 50% or at least 60%, more preferably at least 70%, at least 80%, at least 90% or at least 100% of juglone reducing activity e.g. in the above-mentioned N-terminal activity assay when the concentration of the SecTRAP forming agent (or TrxR inhibitor) used in the assay is a concentration (preferably the minimal concentration) that causes (or achieves) 100% inhibition (or close to 100% inhibition) in the C-terminal activity assay as described above. The % activities mentioned above are the % of juglone reducing activity as compared to the juglone reducing activity observed in an assay performed in the absence of a SecTRAP forming agent (or TrxR inhibitor). Put another way, in the absence of any compound (SecTRAP forming agent or TrxR inhibitor) the juglone reducing activity would represent the “100%” value (or “100%” control value). The juglone reducing activity level may be measured (or quantified) in absence of compound, then the activity in the presence of the SecTRAP forming agent or TrxR inhibitor) is measured (or quantified) and a normalisation to a % is done. By way of example, exhibiting (or retaining or maintaining) at least 50% juglone reducing activity means exhibiting (or retaining or maintaining) at least 50% of the activity exhibited (or observed) in the absence of a SecTRAP forming agent (or TrxR inhibitor).

Preferably, the TrxR used in the C-terminal activity assay and the N-terminal activity assay is a recombinant TrxR, preferably recombinant human TrxR or recombinant rat TrxR. In some embodiments, the TrxR used in the C-terminal activity assay and the N-terminal activity assay is recombinant human TrxR. In some embodiments, the TrxR used in the C-terminal activity assay and the N-terminal activity assay is recombinant rat TrxR.

Thus, further alternatively viewed, the present invention provides an inhibitor of the enzyme thioredoxin reductase (TrxR e.g. TrxR1) which inhibits (or reduces or diminishes or abolishes) the reductase activity at the C-terminal active site (which may be characterized by the presence of a Sec residue) but does not inhibit or diminish (or do not significantly inhibit or diminish) the redox activity at the N-terminal active site (or N-terminal redox active site or N-terminal active site dithiol motif) for use in treating a T-cell infiltrated cancer in a subject, wherein said inhibitor has immunostimulatory activity thereby causing said subject to raise an immune response against said cancer. Alternatively viewed, such Trx inhibitors are for use treating a T-cell infiltrated cancer in a subject, wherein said inhibitor stimulates (or causes or elicits) an anti-cancer immune response. In some embodiments, such TrxR inhibitors may be characterized by an ability to inhibit (or block or abolish or reduce), preferably significantly inhibit, the ability of TrxR (e.g. TrxR1) to reduce thioredoxin (Trx, e.g. Trx1)) or other substrates, e.g. DTNB (substrates that are usually or normally reduced or reducible at the TrxR C-terminal active site) but not to inhibit (or not to significantly inhibit, reduce or abolish) the reduction of e.g. juglone (5-Hydroxy-1,4-naphthoquinone) (a substrate that is usually or normally reduced or reducable at the TrxR N-terminal active site).

In some embodiments, the IC₅₀ (concentration at which 50% of Thioredoxin Reductase (TrxR) activity is inhibited) is 1×10⁻⁴ M to 1×10⁻¹¹M (e.g. as assessed in a C-terminal activity assay as described herein). In some embodiments, the IC₅₀ is less than 1×10⁻⁴ M, less than 1×10⁻⁵M, less than 1×10⁻⁶M, less than 1×10⁻⁷M, less than 1×10⁻⁸M, less than 1×10⁻⁹M, less than 1×10⁻¹⁰ M (e.g. as low as 1×10⁻¹¹M). Exemplary IC₅₀ values are given in the Example section herein.

Typically, SecTRAP forming agents used in accordance with the invention cause (or elicit) a reduction in the level (or concentration) of intracellular thioredoxin (Trx) in cells (e.g. in cancer cells or in cancer cell lines e.g. the cell line MDA-MB-231). Thioredoxin (also referred to herein as Trx) is a usual (or normal) cellular substrate for the C-terminal active site of TrxR (e.g. TrxR1). The C-terminal active site of TrxR has reductase activity towards thioredoxin, i.e. it reduces thioredoxin. In some preferred embodiments, SecTRAP forming agents used in accordance with the invention cause (or elicit) a reduction (e.g. as described above) in the level (or concentration) of intracellular reduced thioredoxin (Trx) in cells (i.e. a reduction in the reduced form of Trx as opposed to the oxidised form). The oxidation state of Trx, e.g. the percentage of reduced and oxidized forms of the total amount, in extracts and cells can be determined by a combination of polyacrylamide gel electrophoresis, immunoelectrophoresis and enzyme activity (e.g. as described by Holmgren, A. & Fagerstedt, M., 1982. The Journal of biological chemistry, 257(12), pp. 6926-30. 1982).

In some embodiments SecTRAP forming agents (or TrxR inhibitors) used in accordance with the invention may reduce the level of intracellular thioredoxin (Trx) in cells (e.g. in cancer cells or in cancer cell lines e.g. a breast cancer cell line such as the cell line MDA-MB-231) by at least 10%, at least 20%, at least 30%, at least 40%, at least 50%, at least 60%, at least 70%, at least 80%, or at least 90%. Such % reductions are in relation to level of intracellular thioredoxin (Trx) in untreated control cells. In some preferred embodiments, SecTRAP forming agents used in accordance with the invention cause (or elicit) a reduction (e.g. as described above) in the level (or concentration) of intracellular reduced thioredoxin (Trx) in cells (i.e. a reduction in the reduced form of Trx as opposed to the oxidised form). By way of example, in some embodiments SecTRAP forming agents (or TrxR inhibitors) used in accordance with the invention may reduce the level of intracellular thioredoxin (Trx) levels (e.g. reduced form of Trx) in MDA-MB-231 cells (a breast cancer cell line) by at least 10%, at least 20%, at least 30%, at least 40%, at least 50%, at least 60%, at least 70%, at least 80%, or at least 90% as compared with untreated control cells, wherein intracellular levels of Trx are measured (e.g. in ng/ml) 96 hours after the start of treatment with the agent and wherein the agent is used at 10 μM. In some other embodiments, the SecTRAP forming agents kill TrxR/Trx-rich cancer cells and therefore reduce the total amount of TrxR and Trx (and/or the total amount of additional substrates such as protein disulphide isomerase (PDI), and peroxiredoxins), in the cancer tissue.

Intracellular levels of Trx may be measured using any suitable method and the skilled person will be familiar with such methods. In some embodiments, an ELISA is used. For example, in some embodiments the intracellular level of Trx in a cancer cell line (e.g. MDA-MB-231 cells) is assessed and, at a given sampling time (e.g. 96 h after the start of treatment), the cell supernatant is removed from the cells, the cells are washed and lysed, and the total amount of Trx from all cells in the cell lysates (or Trx concentration) is determined using ELISA. A particularly preferred method for measuring the intracellular level (or concentration) of Trx is provided in the Example section herein.

Trx is a natural substrate for the C-terminal active site of the enzyme TrxR. A role of Trx is to reduce (or facilitate the reduction of) other proteins in the cell.

Typically, inhibition of TrxR (and formation of a SecTRAP) results in oxidative stress, and results in decreases in Trx levels intracellularly.

Typically, SecTRAP forming agents used in accordance with the invention may be thought of as causing (or eliciting) a change in function of the TrxR enzyme, converting it from an anti-oxidant to a pro-oxidant enzyme. Typically this conversion is non-reversible.

Without wishing to be bound by theory, it is thought that inhibition of thioredoxin reductase is obtained by the utilization of strong electrophilicity of small molecule inhibitors in combination with a pronounced inherent nucleophilicity of NADPH-reduced, but not oxidized, thioredoxin reductase (TrxR), resulting in selective and potent inhibition of said enzyme without major targeting of other cellular pathways or enzymes.

In some embodiments, the SecTRAP forming agent (or TrxR inhibitor) for use in the invention is a compound of formula I, II, III, IV, V, VI, VII, VIII, IX, X or XI as described herein.

Compounds of formula I

In some embodiments, the SecTRAP forming agent is a compound of formula I

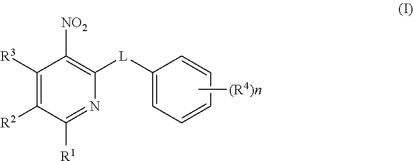

or a pharmaceutically acceptable salt thereof, wherein:
L represents —S(O)₂—;
n represents 0 to 5;
R¹, R² and R³ each independently represent H, halo, R^{a1}, —CN, -A^{a1}-C(Q^{a1})R^{b1}, -A^{b1}-C(Q^{b1})N(R^{c1})R^{d1}, -A^{c1}-C(Q^{c1})OR^{e1}, -A^{d1}-S(O)ₚR^{f1}, -A^{e1}-S(O)ₚN(R^{g1})R^{h1}, -A^{f1}-S(O)ₚORⁱ¹, —N₃, —N(R^{j1})R^{k1}, —N(H)CN, —NO₂, —ONO₂, —OR^{l1} or —SR^{m1};
each A^{a1} to A^{f1} independently represents a single bond, —N(R^{p1})— or —O—;
each Q^{a1} to Q^{c1} independently represents ═O, ═S, ═NRⁿ¹ or ═N(OR^{o1});
each R^{a1} and R^{f1} independently represents C₁₋₆ alkyl optionally substituted by one or more groups independently selected from G^{1a}, or heterocycloalkyl optionally substituted by one or more groups independently selected from G^{1b};
each R^{p1} independently represents H or C₁₋₆ alkyl optionally substituted by one or more fluoro;
each R^{b1}, R^{c1}, R^{d1}, R^{e1}, R^{g1}, R^{h1}, Rⁱ¹, R^{j1}, R^{k1}, R^{l1}, R^{m1}, Rⁿ¹ and R^{o1} independently represents H, C₁₋₆ alkyl optionally substituted by one or more groups independently selected from G^{1a} or heterocycloalkyl optionally substituted by one or more groups independently selected from G^{1b}; or
any of R^{c1} and R^{d1}, R^{g1} and R^{h1} and/or R^{j1} and R^{k1} are linked together to form, together with the nitrogen atom to which they are attached, a 3- to 6-membered ring, which ring optionally contains one further heteroatom and which ring optionally is substituted by one or more groups independently selected from halo, C₁₋₃ alkyl optionally substituted by one or more halo, and ═O;
each R⁴ independently represents halo, R^{a2}, —CN, -A^{a2}-C(Q^{a2})R^{b2}, -A^{b2}-C(Q^{b2})N(R^{c2})R^{d2}, -A^{c2}-C(Q^{c2})OR^{e2}, -A^{d2}-S(O)_{q}R^{f2}, -A^{e2}-S(O)_{q}N(R^{g2})R^{h2}, -A^{f2}-S(O)_{q}ORⁱ², —N₃, —N(R^{j2})R^{k2}, —N(H)CN, —NO₂, —ONO₂, —OR^{l2} or —SR^{m2};
each Q^{a2} to Q^{c2} independently represents ═O, ═S, ═NRⁿ² or ═N(OR^{o2});
each A^{a2} to A^{f2} independently represents a single bond, —N(R^{p2})— or —O—;
each R^{a2} and R^{f2} independently represents C₁₋₆ alkyl optionally substituted by one or more groups independently selected from G^{2a} or heterocycloalkyl optionally substituted by one or more groups independently selected from G^{2b};
each R^{p2} independently represents H or C₁₋₆ alkyl optionally substituted by one or more fluoro;
each R^{b2}, R^{c2}, R^{d2}, R^{e2}, R^{g2}, R^{h2}, Rⁱ², R^{j2}, R^{k2}, R^{l2}, R^{m2}, Rⁿ² and R^{o2} independently represents H, C₁₋₆ alkyl optionally substituted by one or more groups independently selected from G^{2a}, or heterocycloalkyl optionally substituted by one or more groups independently selected from G^{2b}; or
any two R^{c2} and R^{d2}, R^{g2} and R^{h2} and/or R^{j2} and R^{k2} are linked together to form, along with the nitrogen atom to which they are attached, a 3- to 6-membered ring, which ring optionally contains one further heteroatom and which ring optionally is substituted by one or more groups independently selected from halogen, C₁₋₃alkyl optionally substituted by one or more halogens, and ═O;
each G^{1a}, G^{1b}, G^{2a} and G^{2b} independently represents halo, —CN, —N(R^{a3})R^{b3}, —OR^{c3}, —SR^{d3} or ═O;
each R^{a3}, R^{b3}, R^{c3} and R^{d3} independently represents H or C₁₋₆ alkyl optionally substituted by one or more fluoro;
or R^{a3} and R^{b3} are linked together to form, along with the nitrogen atom to which they are attached, a 3- to 6-membered ring, which ring optionally contains one further heteroatom and which ring optionally is substituted by one or more groups independently selected from fluoro, C₁₋₃alkyl optionally substituted by one or more fluoro, and ═O; and
each p and q independently represents 1 or 2.

In some preferred embodiments, the SecTRAP forming agent is a compound of formula I, wherein R₃ and/or R₂ (preferably R₃ and R₂) represent H.

In some preferred embodiments, the SecTRAP forming agent is a compound of formula I, wherein R₁ is OR^{l1}, preferably R^{l1} represents C₁₋₆ alkyl, more preferably C₁ alkyl.

In some preferred embodiments the SecTRAP forming agent is a compound of formula I, wherein R₄ represents halo, preferably chloro, and preferably n represents 1.

A particularly preferred SecTRAP forming agent is a compound of formula I having the structure:

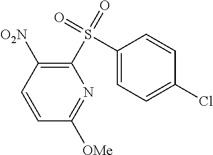 [SMILES: COc1ccc([N+](=O)[O-])c(S(=O)(=O)c2ccc(Cl)cc2)n1]

This compound is also referred to herein as 2-((4-Chlorophenyl)sulfonyl)-6-methoxy-3-nitropyridine and OBT-1000.

In some embodiments, the compound of formula I is not a compound selected from the list consisting of compounds:
(1) 6-methoxy-3-nitro-2-(phenylsulphonyl)pyridine;
(2) 6-methoxy-3-nitro-2-tosylpyridine;
(3) 5-methyl-3-nitro-2-(phenylsulphonyl)pyridine;
(4) 3-nitro-2-tosylpyridine;
(5) 2-((4-chlorophenyl)sulphonyl)-6-methoxy-3-nitropyridine;
(6) 3-nitro-2-(phenylsulphonyl)pyridine;
(7) 2-methyl-3,5-dinitro-6-(phenylsulphonyl)pyridine; and
(8) N-(2-((5-chloro-3-nitropyridin-2-yl)sulphonyl)phenyl)acetamide.

### Compounds of Formula II

In some embodiments, the SecTRAP forming agent is a compound of formula II

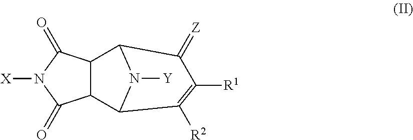

or a pharmaceutically acceptable salt thereof, wherein:
X represents C₁₋₁₂ alkyl optionally substituted by one or more groups independently selected from G^{1a}, heterocycloalkyl optionally substituted by one or more groups independently selected from G^{1b}, aryl optionally substituted by one or more groups independently selected from G^{1c}, or heteroaryl optionally substituted by one or more groups independently selected from G^{1d};
Y represents C₁₋₁₂ alkyl optionally substituted by one or more groups independently selected from G^{2a}; heterocycloalkyl optionally substituted by one or more groups independently selected from G^{2b}, aryl optionally substituted by one or more groups independently selected from G^{2c}, or heteroaryl optionally substituted by one or more groups independently selected from G^{2d};
Z represents O, S, NR^{a} or N(OR^{b});
R¹ and R² independently represents H or C₁₋₆ alkyl, the latter group being optionally substituted by one or more groups independently selected from halo and —OC₁₋₆ alkyl optionally substituted by one or more halo;
each G^{1a}, G^{1b}, G^{1c} and G^{1d} independently represents halo, R^{a1}, —CN, -A^{a1}-C(Q^{a1})R^{b1}, -A^{b1}-C(Q^{b1})N(R^{c1})R^{d1}, -A^{c1}-C(Q^{c1})OR^{e1}, -A^{d1}-S(O)ₙR^{f1}, -A^{e1}-S(O)ₙC(O)R^{g1}, -A^{f1}-S(O)ₙN(R^{h1})Rⁱ¹, -A^{g1}-S(O)ₙOR^{j1}, —N₃, —N(R^{k1})R^{l1}, —N(H)CN, —NO₂, —OR^{m1}, —SRⁿ¹ or =Q^{d1};
each A^{a1} to A^{g1} independently represents a single bond, —N(R^{o1})—, —C(Q^{e1})N(R^{p1})— or —O—;
each Q^{a1} to Q^{e1} independently represents ═O, ═S, ═NR^{q1} or ═N(OR^{r1});
R^{a} and R^{b} each independently represent H or C₁₋₆ alkyl, the latter group being optionally substituted by one or more groups independently selected from halo and —OC₁₋₆ alkyl optionally substituted by one or more halo;
each R^{a1} and R^{f1} independently represents C₁₋₆ alkyl optionally substituted by one or more groups independently selected from G^{3a}, heterocycloalkyl optionally substituted by one or more groups independently selected from G^{3b}, aryl optionally substituted by one or more groups independently selected from G^{3c}, or heteroaryl optionally substituted by one or more groups independently selected from G^{3d};
each R^{b1}, R^{c1}, R^{d1}, R^{e1}, R^{g1}, R^{h1}, Rⁱ¹, R^{j1}, R^{k1}, R^{l1}, R^{m1}, Rⁿ¹, R^{q1} and R^{r1} independently represents H, C₁₋₆ alkyl optionally substituted by one or more groups independently selected from G^{3a}, heterocycloalkyl optionally substituted by one or more groups independently selected from G^{3b}, aryl optionally substituted by one or more groups independently selected from G^{3c}, or heteroaryl optionally substituted by one or more groups independently selected from G^{3d};
or any two R^{c1} and R^{d1}, R^{h1} and Rⁱ¹ and/or R^{k1} and R^{l1} are linked together to form, along with the nitrogen atom to which they are attached, a 3- to 6-membered ring, which ring optionally contains one further heteroatom and which ring optionally is substituted by one or more groups independently selected from halo, C₁₋₃ alkyl optionally substituted by one or more halo, and ═O;
each R^{o1} and R^{p1} independently represents H or C₁₋₆ alkyl optionally substituted by one or more halo;
each G^{2a}, G^{2b}, G^{2c} and G^{2d} independently represents halo, R^{a2}, —CN, -A^{a2}-C(Q^{a2})R^{b2}, -A^{b2}-C(Q^{b2})N(R^{c2})R^{d2}, -A^{c2}-C(Q^{c2})OR^{e}, -A^{d2}-S(O)ₚR^{f2}, -A^{e2}-S(O)ₚC(O)R^{g2}, -A^{f2}-S(O)ₚN(R^{h2})Rⁱ², -A^{g2}-S(O)ₚOR^{j2}, —N₃, —N(R^{k2})R^{l2}, —N(H)CN, —NO₂, —OR^{m2}, —SRⁿ² or =Q^{d2};
each A^{a2} to A⁹² independently represents a single bond, —N(R^{o2})—, —C(Q^{e2})N(R^{p2})— or —O—;
each Q^{a2} to Q^{e3} independently represents ═O, ═S, ═NR^{q2} or ═N(OR^{r2});
each R^{a2} independently represents heterocycloalkyl optionally substituted by one or more groups independently selected from G^{4b}, aryl optionally substituted by one or more groups independently selected from G^{4c}, or heteroaryl optionally substituted by one or more groups independently selected from G^{4d};
each R^{f2} independently represents C₁₋₆ alkyl optionally substituted by one or more groups independently selected from G^{4a}, heterocycloalkyl optionally substituted by one or more groups independently selected from G^{4b}, aryl optionally substituted by one or more groups independently selected from G^{4c}, or heteroaryl optionally substituted by one or more groups independently selected from G^{4d};
each R^{b2}, R^{c2}, R^{d2}, R^{e2}, R^{g2}, R^{h2}, Rⁱ², R^{j2}, R^{k2}, R^{l2}, R^{m2}, Rⁿ², R^{q2} and R^{r2} independently represents H, C₁₋₆ alkyl optionally substituted by one or more groups independently selected from G^{4a}, heterocycloalkyl optionally substituted by one or more groups independently selected from G^{4b}, aryl optionally substituted by one or more groups independently selected from G^{4c}, or heteroaryl optionally substituted by one or more groups independently selected from G^{4d};
or any two R^{c2} and R^{d2}, R^{h2} and Rⁱ² and/or R^{k2} and R^{l2} are linked together to form, along with the nitrogen atom to which they are attached, a 3- to 6-membered ring, which ring optionally contains one further heteroatom and which ring optionally is substituted by one or more groups independently selected from halo, C₁₋₃ alkyl optionally substituted by one or more halo, and ═O;
each R^{o2} and R^{p2} independently represents H or C₁₋₆ alkyl optionally substituted by one or more halo;
each G^{3a} independently represents halo, —CN, -A^{a3}-C(Q^{a3})R^{b3}, -A^{b3}-C(Q^{b3})N(R^{c3})R^{d3}, -A^{c3}-C(Q^{c3})OR^{e3}, -A^{d3}-S(O)_{q}R^{f3}, -A^{e3}-S(O)_{q}C(O)R^{g3}, -A^{f3}-S(O)_{q}N(R^{h3})Rⁱ³, -A^{g3}-S(O)_{q}OR^{j3}, —N₃,
—N(R^{k3})R^{l3}, —N(H)CN, —NO₂, —OR^{m3}, —SRⁿ³ or =Q^{d3};
each G^{3b}, G^{3c} and G^{3d} independently represents halo, R^{a3}, —CN, -A³-C(Q^{a3})R^{b3}, -A^{b3}-C(Q^{b3})N(R^{c3})R^{d3}, -A^{c3}-C(Q^{c3})OR^{e3}, -A^{d3}-S(O)_{q}R^{f3}, -A^{e3}-S(O)_{q}C(O)R^{g3}, -A^{f3}-S(O)_{q}N(R^{h3})Rⁱ³, -A^{g3}-S(O)_{q}OR^{j3}, —N₃, —N(R^{k3})R^{l3}, —N(H)CN, —NO₂, —OR^{m3}, —SRⁿ³ or =Q^{d3};
each A^{a3} to A⁹³ independently represents a single bond, —N(R^{o3})—, —C(Q^{e3})N(R^{p3})— or —O—;
each Q^{a3} to Q^{e3} independently represents ═O, ═S, ═NR^{q3} or ═N(OR^{r3});
each R^{a3} and R^{f3} independently represents C₁₋₆ alkyl optionally substituted by one or more groups independently selected from G^{5a}, or heterocycloalkyl optionally substituted by one or more groups independently selected from G^{5b};
each R^{b3}, R^{c3}, R^{d3}, R^{e3}, R^{g3}, R^{h3}, Rⁱ³, R^{j3}, R^{k3}, R^{l3}, R^{m3}, Rⁿ³, R^{q3} and R^{r3} independently represents H, C₁₋₆ alkyl optionally substituted by one or more groups independently selected from G^{5a}, or heterocycloalkyl optionally substituted by one or more groups independently selected from G^{5b};
or any two R^{c3} and R^{d3}, R^{h3} and Rⁱ³ and/or R^{k3} and R^{l3} are linked together to form, along with the nitrogen atom to which they are attached, a 3- to 6-membered ring, which ring optionally contains one further heteroatom and which ring optionally is substituted by one or more groups independently selected from halo, C₁₋₃ alkyl optionally substituted by one or more halo, and ═O;
each R^{o3} and R^{p3} independently represents H or C₁₋₆ alkyl optionally substituted by one or more halo;
each G^{4a} independently represents halogen, —CN, -A^{a4}-C(Q^{a4})R^{b4}, -A^{b4}-C(Q^{b4})N(R^{c4})R^{d4},
-A^{c4}-C(Q^{c4})OR^{e4}, -A^{d4}-S(O)ᵣR^{f4}, -A^{e4}-S(O)ᵣC(O)R^{g4}, -A^{f4}-S(O)ᵣN(R^{h4})Rⁱ⁴, -A^{g4}-S(O)ᵣOR^{j4}, —N₃, —N(R^{k4})R^{l4}, —N(H)CN, —NO₂, —OR^{m4}, —SRⁿ⁴ or =Q^{d4};
each G^{4b}, G^{4c} and G^{4d} independently represents halo, R^{a4}, —CN, -A^{a4}-C(Q^{a4})R^{b4}, -A^{b4}-C(Q^{b4})N(R^{c4})R^{d4}, -A^{c4}-C(Q^{c4})OR^{e4}, -A^{d4}-S(O)ᵣR^{f4}, -A^{e4}-S(O)ᵣC(O)R^{g4}, -A^{f4}-S(O)ᵣN(R^{h4})Rⁱ⁴, -A^{g4}-S(O)ᵣOR^{j4}, —N₃, —N(R^{k4})R^{l4}, —N(H)CN, —NO₂, —OR^{m4}, —SRⁿ⁴ or =Q^{d4};
each A^{a4} to A⁹⁴ independently represents a single bond, —N(R^{o4})—, —C(Q^{e4})N(R^{p4})— or —O—;
each Q^{a4} to Q^{e4} independently represents ═O, ═S, ═NR^{q4} or ═N(OR^{r4});
each R^{a4} and R^{f4} independently represents C₁₋₆ alkyl optionally substituted by one or more groups independently selected from G^{6a}, heterocycloalkyl optionally substituted by one or more groups independently selected from G^{6b}, or aryl optionally substituted by one or more groups independently selected from G^{6c};
each R^{b4}, R^{o4}, R^{d4}, R^{e4}, R^{g4}, R^{h4}, Rⁱ⁴, R^{j4}, R^{k4}, R^{l4}, R^{m4}, Rⁿ⁴, R^{q4} and R^{r4} independently represents H, C₁₋₆ alkyl optionally substituted by one or more groups independently selected from G^{6a}, or heterocycloalkyl optionally substituted by one or more groups independently selected from G^{6b};
or any two R^{o4} and R^{d4}, R^{h4} and Rⁱ⁴ and/or R^{k4} and R^{l4} are linked together to form, along with the nitrogen atom to which they are attached, a 3- to 6-membered ring, which ring optionally contains one further heteroatom and which ring optionally is substituted by one or more groups independently selected from halo, C₁₋₃ alkyl optionally substituted by one or more halo, and ═O;
each R^{o4} and R^{p4} independently represents H or C₁₋₆ alkyl optionally substituted by one or more halo;
each G^{5a} and G^{6a} independently represents halo or —OC₁₋₆ alkyl optionally substituted by one or more halo;
each G^{5b}, G^{6b} and G^{6c} represents halo, C₁₋₆ alkyl optionally substituted by one or more halogens, or —OC₁₋₆ alkyl optionally substituted by one or more halo;
each n independently represents 1 or 2;
each p independently represents 1 or 2;
each q independently represents 1 or 2; and
each r independently represents 1 or 2.

In some preferred embodiments, the SecTRAP forming agent is a compound of formula II, wherein X is C₁₋₁₂ alkyl (preferably C₁ alkyl), substituted by G^{1a}, preferably G^{1a} is R^{a1} and preferably R^{a1} is aryl (preferably phenyl).

In some preferred embodiments, the SecTRAP forming agent is a compound of formula II, wherein R₁ and/or R₂ (preferably R₁ and R₂) represent H. In some preferred embodiments, the SecTRAP forming agent is a compound of formula II, wherein Z represents O.

In some preferred embodiments, the SecTRAP forming agent is a compound of formula II, wherein Y is C₁₋₁₂ alkyl (preferably C₁ alkyl), substituted by G^{2a}, preferably G^{2a} is R^{a2} and preferably R^{a2} is aryl (preferably phenyl).

A particularly preferred SecTRAP forming agent is a compound of formula II having the structure:

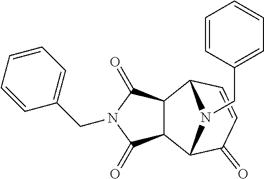 [SMILES: O=C1C=C[C@H]2[C@@H]3C(=O)N(Cc4ccccc4)C(=O)[C@@H]3[C@@H]1N2Cc1ccccc1]

This compound is also referred to herein as (exo-4,11-Dibenzyl-4,11-diazatricyclo[5.3.1.0^{2,6}]undec-9-ene-3,5,8-trione) and OBT-2056.

In some embodiments, the compound of formula II is not a compound selected from the list consisting of compounds:
exo-11-methyl-4-phenyl-4,11-diazatricyclo[5.3.1.0^{2,6}]undec-9-ene-3,5,8-trione;
11-methyl-4-phenyl-4,11-diazatricyclo[5.3.1.0^{2,6}]undec-9-ene-3,5,8-trione;
endo-methyl 3,5,10-trioxo-4-phenyl-4,11-diazatricyclo[5.3.1.0^{2,6}]undec-8-en-11-carboxylate;
exo-methyl 3,5,10-trioxo-4-phenyl-4,11-diazatricyclo[5.3.1.0^{2,6}]undec-8-en-11-carboxylate;
exo-4,11-diphenyl-4,11-diazatricyclo[5.3.1.0^{2,6}]undec-9-ene-3,5,8-trione;
endo-4,11-diphenyl-4,11-diazatricyclo[5.3.1.0^{2,6}]undec-9-ene-3,5,8-trione;
diphenyl-4,11-diazatricyclo[5.3.1.0^{2,6}]undec-9-ene-3,5,8-trione;
11-(4-bromobenzyl)-4-methyl-4,11-diazatricyclo[5.3.1.0^{2,6}]undec-9-ene-3,5,8-trione;
endo-4-phenyl-11-(4-pyridyl)-4,11-diazatricyclo[5.3.1.0^{2,6}]undec-9-ene-3,5,8-trione;
endo-4-phenyl-11-(2-pyridyl)-4,11-diazatricyclo[5.3.1.0^{2,6}]undec-9-ene-3,5,8-trione;
exo-11-(2-iodobenzyl)-4-methyl-4,11-diazatricyclo[5.3.1.0^{2,6}]undec-9-ene-3,5,8-trione;
endo-11-(2-iodobenzyl)-4-methyl-4,11-diazatricyclo[5.3.1.0^{2,6}]undec-9-ene-3,5,8-trione;
exo-11-benzyl-4-phenyl-4,11-diazatricyclo[5.3.1.0^{2,6}]undec-9-ene-3,5,8-trione;
11-benzyl-4-phenyl-4,11-diazatricyclo[5.3.1.0^{2,6}]undec-9-ene-3,5,8-trione;
exo-4-methyl-11-(2-vinylphenyl)-4,11-diazatricyclo[5.3.1.0^{2,6}]undec-9-ene-3,5,8-trione;
endo-4-methyl-11-(2-vinylphenyl)-4,11-diazatricyclo[5.3.1.0^{2,6}]undec-9-ene-3,5,8-trione;
exo-4-phenyl-11-(2-pyridyl)-4,11-diazatricyclo[5.3.1.0^{2,6}]undec-9-ene-3,5,8-trione;
endo-11-(3-oxocyclohex-1-en-1-yl)-4-phenyl-4,11-diazatricyclo[5.3.1.0^{2,6}]undec-9-ene-3,5,8-trione;
endo-4-(3,5,10-trioxo-4-phenyl-4,11-diazatricyclo[5.3.1.0^{2,6}]undec-8-en-11-yl)pyridine-1-oxide;
exo-4-phenyl-11-styryl-4,11-diazatricyclo[5.3.1.0^{2,6}]undec-9-ene-3,5,8-trione;
endo-11-(6,6-dimethyl-3-oxocyclohex-1-en-1-yl)-4-phenyl-4,11-diazatricyclo[5.3.1.0^{2,6}]undec-9-ene-3,5,8-trione;
11-(4-ted-butylbenzyl)-4-phenyl-4,11-diazatricyclo[5.3.1.0^{2,6}]undec-9-ene-3,5,8-trione;
exo-11-(2-iodobenzyl)-4-phenyl-4,11-diazatricyclo[5.3.1.0^{2,6}]undec-9-ene-3,5,8-trione;
endo-11-(4,6-dimethylpyrimidin-2-yl)-4-phenyl-4,11-diazatricyclo[5.3.1.0^{2,6}]undec-9-ene-3,5,8-trione;
exo-11-(4,6-dimethylpyrimidin-2-yl)-4-phenyl-4,11-diazatricyclo[5.3.1.0^{2,6}]undec-9-ene-3,5,8-trione;
endo-11-(4,4-dimethyl-3-oxopent-1-ene-1-yl)-4-phenyl-4,11-diazatricyclo[5.3.1.0^{2,6}]-undec-9-ene-3,5,8-trione;
exo-4-(4-ethylphenyl)-11-(2-iodobenzyl)-4,11-diazatricyclo[5.3.1.0^{2,6}]undec-9-ene-3,5,8-trione;
endo-11-(6-chloropyridazin-3-yl)-4-phenyl-4,11-diazatricyclo[5.3.1.0^{2,6}]undec-9-ene-3,5,8-trione;
exo-11-(6-chloropyridazin-3-yl)-4-phenyl-4,11-diazatricyclo[5.3.1.0^{2,6}]undec-9-ene-3,5,8-trione;
exo-11-(4,6-dimethoxy-1,3,5-triazin-2-yl)-4-phenyl-4,11-diazatricyclo[5.3.1.0^{2,6}]undec-9-ene-3,5,8-trione;
exo-11-(2-pyridylmethyl)-4-phenyl-4,11-diazatricyclo[5.3.1.0²′⁶]undec-9-ene-3,5,8-trione;
endo-11-(2,4-dinitrophenyl)-4-phenyl-4,11-diazatricyclo[5.3.1.0^{2,6}]undec-9-ene-3,5,8-trione;
endo-4-phenyl-11-(6-phenylpyridazin-3-yl)-4,11-diazatricyclo[5.3.1.0^{2,6}]undec-9-ene-3,5,8-trione;
exo-11-(4,6-diphenyl-1,3,5-triazin-2-yl)-4-phenyl-4,11-diazatricyclo[5.3.1.0^{2,6}]undec-9-ene-3,5,8-trione;
exo-4-(2,6-diisopropylphenyl)-11-(2-iodobenzyl)-4,11-diazatricyclo[5.3.1.0^{2,6}]undec-9-ene-3,5,8-trione;
endo-4-(2,6-diisopropylphenyl)-11-(2-iodobenzyl)-4,11-diazatricyclo[5.3.1.0^{2,6}]undec-9-ene-3,5,8-trione;
exo-11-((E)-3-(4-bromophenyl)-3-oxoprop-1-en-1-yl)-4-phenyl-4,11-diazatricyclo[5.3.1.0^{2,6}]undec-9-ene-3,5,8-trione;
exo-11-((E)-3-(4-chlorophenyl)-3-oxoprop-1-en-1-yl)-4-phenyl-4,11-diazatricyclo-[5.3.1.0^{2,6}]undec-9-ene-3,5,8-trione;
exo-11-((E)-3-(2,4-dinitrophenyl)-3-oxoprop-1-en-1-yl)-4-phenyl-4,11-diazatricyclo-[5.3.1.0^{2,6}]undec-9-ene-3,5,8-trione;
exo,exo-1,2-bis-(4-phenyl-4,11-diazatricyclo[5.3.1.0^{2,6}]undec-9-ene-3,5,8-trione-11-yl)-ethane;
endo,exo-1,2-bis-(4-phenyl-4,11-diazatricyclo[5.3.1.0^{2,6}]undec-9-ene-3,5,8-trione-11-yl)ethane;
exo,exo-1,3-bis-(4-phenyl-4,11-diazatricyclo[5.3.1.0^{2,6}]undec-9-ene-3,5,8-trione-11-yl)-propane;
endo,exo-1,3-bis-(4-phenyl-4,11-diazatricyclo[5.3.1.0^{2,6}]undec-9-ene-3,5,8-trione-11-yl)propane;
4-phenyl-11-(3-phenyl-1,2,4-thiadiazol-5-yl)-4,11-diazatricyclo[5.3.1.0^{2,6}]undec-9-ene-3,5,8-trione;
exo-11-(5,6-diphenyl-1,2,4-triazin-3-yl)-4-phenyl-4,11-diazatricyclo[5.3.1.0^{2,6}]undec-9-ene-3,5,8-trione;
endo-11-(5,6-diphenyl-1,2,4-triazin-3-yl)-4-phenyl-4,11-diazatricyclo[5.3.1.0^{2,6}]undec-9-ene-3,5,8-trione; and
endo-11-(1,2-bis(3-nitrophenyl)vinyl)-4-phenyl-4,11-diazatricyclo[5.3.1.0^{2,6}]undec-9-ene-3,5,8-trione.

### Compounds of Formula III

In some embodiments, the SecTRAP forming agent is a compound of formula III

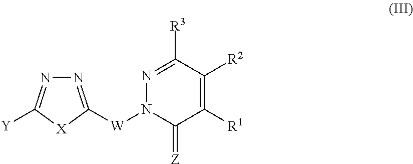

or a pharmaceutically acceptable salt thereof, wherein:
W represents C₁ alkylene optionally substituted by one or more groups independently selected from R⁴;
X represents O or S;
Y represents C₁₋₆ alkyl optionally substituted by one or more groups independently selected from G^{1a}, heterocycloalkyl optionally substituted by one or more groups independently selected from G^{1b}, aryl optionally substituted by one or more groups independently selected from G^{1c}, or heteroaryl optionally substituted by one or more groups independently selected from G^{1d};
Z represents O, S or NR⁵;
R¹ represents H, halo, R^{a1}, —CN, —C(Q^{a1})R^{b1}, —C(Q^{b1})N(R^{c1})R^{d1}, —C(Q^{c1})OR^{e1}, —S(O)ₙR^{f1}, —S(O)ₚN(R^{g1})R^{h1}, —S(O)ₚORⁱ¹ or —NO₂;
R² represents H, halo, —CN or —N₃;
R³ represents H, halo or R^{j1};
R⁴ represents halo or C₁₋₆ alkyl optionally substituted by one or more groups independently selected from G^{1e};
R⁵ represents H, R^{k1}, —OR^{l1} or —N(R^{m1})Rⁿ¹;
Q^{a1} to Q^{c1} each independently represents ═O, ═S, ═NR^{o1} or ═N(OR^{p1});
each R^{a1}, R^{f1}, R^{j1} and R^{k1} independently represents C₁₋₆ alkyl optionally substituted by one or more groups independently selected from G^{2a}, or heterocycloalkyl optionally substituted by one or more groups independently selected from G^{2b};
each R^{b1}, R^{c1}, R^{d1}, R^{e1}, R^{g1}, R^{h1}, Rⁱ¹, R^{l1}, R^{m1}, Rⁿ¹, R^{o1} and R^{p1} independently represents H, C₁₋₆ alkyl optionally substituted by one or more groups independently selected from G^{2a}, or heterocycloalkyl optionally substituted by one or more groups independently selected from G^{2b};
or any two R^{c1} and R^{d1}, R^{g1} and R^{h1} and/or R^{m1} and Rⁿ¹ are linked together to form, along with the nitrogen atom to which they are attached, a 3- to 6-membered ring, which ring optionally contains one further heteroatom and which ring optionally is substituted by one or more groups independently selected from halogen, C₁₋₃ alkyl optionally substituted by one or more halogens, and ═O;
each G^{1a}, G^{1b}, G^{1c} and G^{1d} represent halogen, R^{a2}, —CN, -A^{a1}-C(Q^{a2})R^{b2}, -A^{b1}-C(Q^{b2})N(R^{c2})R^{d2}, -A^{c1}-C(Q^{c2})OR^{e2}, -A^{d1}-S(O)_{q}R^{f2}, -A^{e1}-S(O)_{q}C(O)R^{g2}, -A^{f1}-S(O)_{q}N(R^{h2})Rⁱ², -A^{g1}-S(O)_{q}OR^{j2}, —N₃, —N(R^{k2})R^{l2}, —N(H)CN, —NO₂, —OR^{m2}, —SRⁿ² or =Q^{d2};
A^{a1} to A^{g1} each independently represents a single bond, —N(R⁶)—, —C(Q^{e2})N(R⁷)— or —O—;
Q^{a2} to Q^{e2} each independently represents ═O, ═S, ═NR^{o2} or ═N(OR^{p2});
each R⁶ and R⁷ independently represents H or C₁₋₆ alkyl optionally substituted by one or more F;
each R^{a2} and R^{f2} independently represents C₁₋₆ alkyl optionally substituted by one or more groups independently selected from G^{3a} or heterocycloalkyl optionally substituted by one or more groups independently selected from G^{3b};
each R^{b2}, R^{c2}, R^{d2}, R^{e2}, R^{g2}, R^{h2}, Rⁱ², R^{j2}, R^{k2}, R^{l2}, R^{m2}, Rⁿ², R^{o2} and R^{p2} independently represents H, C₁₋₆ alkyl optionally substituted by one or more groups independently selected from G^{3a} or heterocycloalkyl optionally substituted by one or more groups independently selected from G^{3b}; or
any two R^{c2} and R^{d2}, R^{h2} and Rⁱ² and/or R^{k2} and R^{l2} are linked together to form, along with the nitrogen atom to which they are attached, a 3- to 6-membered ring, which ring optionally contains one further heteroatom and which ring optionally is substituted by one or more groups independently selected from halogen, C₁₋₃ alkyl optionally substituted by one or more halogens, and ═O;
each G^{1e} independently represents halo, R^{a2}, —CN, —N(R^{a3})R^{b3}, —OR^{c3} or —SR^{d3};
R^{a3}, R^{b3}, R^{c3} and R^{d3} each independently represents H or C₁₋₆ alkyl optionally substituted by one or more F;
or R^{a3} and R^{b3} are linked together to form, along with the nitrogen atom to which they are attached, a 3- to 6-membered ring, which ring optionally contains one further heteroatom and which ring optionally is substituted by one or more groups independently selected from fluoro, C₁₋₃ alkyl optionally substituted by one or more fluoro, and ═O;
each G^{2a} and G^{2b} independently represents halo, —CN, —N(R^{a4})R^{b4}, —OR^{o4}, —SR^{d4} or ═O;
each R^{a4}, R^{b4}, R^{o4} and R^{d4} independently represents H or C₁₋₆ alkyl optionally substituted by one or more F;
or R^{a4} and R^{b4} are linked together to form, along with the nitrogen atom to which they are attached, a 3- to 6-membered ring, which ring optionally contains one further heteroatom and which ring optionally is substituted by one or more groups independently selected from fluoro, C₁₋₃ alkyl optionally substituted by one or more fluoro, and ═O;
each G^{3a} and G^{3b} independently represents halo, —CN, —N(R^{a5})R^{b5}, —OR^{c5}, —SR^{d5} or ═O;
each R^{a5}, R^{b5}, R^{c5} and R^{d5} independently represents H or C₁₋₆ alkyl optionally substituted by one or more fluoro;
or R^{a5} and R^{b5} are linked together to form, along with the nitrogen atom to which they are attached, a 3- to 6-membered ring, which ring optionally contains one further heteroatom and which ring optionally is substituted by one or more groups independently selected from fluoro, C₁₋₃ alkyl optionally substituted by one or more fluoro, and ═O;
each n independently represents 0, 1 or 2,
each p independently represents 1 or 2,
each q independently represents 1 or 2.

In some embodiments, the compound of formula III is not a compound selected from the list consisting of compounds:
4,5-dichloro-2-((5-(4-chlorophenyl)-1,3,4-oxadiazol-2-yl)methyl)pyridazin-3(2H)-one,
4,5-dichloro-2-((5-phenyl-1,3,4-oxadiazol-2-yl)methyl)pyridazin-3(2H)-one,
4,5-dichloro-2-((5-(p-tolyl)-1,3,4-oxadiazol-2-yl)methyl)pyridazin-3(2H)-one,
4,5-dichloro-2-(1-(5-(4-chlorophenyl)-1,3,4-oxadiazol-2-yl)propyl)pyridazin-3(2H)-one,
4,5-dichloro-2-(1-(5-phenyl-1,3,4-oxadiazol-2-yl)propyl)pyridazin-3(2H)-one,
4,5-dichloro-2-(1-(5-(p-tolyl)-1,3,4-oxadiazol-2-yl)propyl)pyridazin-3(2H)-one, and
4,5-dichloro-2-(1-(5-(4-methoxyphenyl)-1,3,4-oxadiazol-2-yl)propyl)pyridazin-3(2H)-one;
2-((5-(thiophen-2-yl)-1,3,4-oxadiazol-2-yl)methyl)-6-(trifluoromethyl)pyridazin-3(2H)-one,
4,5-dibromo-2-((5-methyl-1,3,4-oxadiazol-2-yl)methyl)pyridazin-3(2H)-one,
5-iodo-2-((5-methyl-1,3,4-oxadiazol-2-yl)methyl)pyridazin-3(2H)-one,
2-((5-ethyl-1,3,4-oxadiazol-2-yl)methyl)-5-iodopyridazin-3(2H)-one,
4,5-dichloro-2-((5-ethyl-1,3,4-oxadiazol-2-yl)methyl)pyridazin-3(2H)-one,
4,5-dibromo-2-((5-ethyl-1,3,4-oxadiazol-2-yl)methyl)pyridazin-3(2H)-one,
4,5-dichloro-2-((5-methyl-1,3,4-oxadiazol-2-yl)methyl)pyridazin-3(2H)-one,
2-((5-(tert-butyl)-1,3,4-oxadiazol-2-yl)methyl)-4,5-dichloropyridazin-3(2H)-one,
2-((5-isopropyl-1,3,4-oxadiazol-2-yl)methyl)-6-(trifluoromethyl)pyridazin-3(2H)-one,
6-(tert-butyl)-2-((5-(4-chlorophenyl)-1,3,4-oxadiazol-2-yl)methyl)pyridazin-3(2H)-one,
2-((5-(3-methoxyphenyl)-1,3,4-oxadiazol-2-yl)methyl)-6-methylpyridazin-3(2H)-one,
6-methyl-2-((5-(4-nitrophenyl)-1,3,4-oxadiazol-2-yl)methyl)pyridazin-3(2H)-one,
5,6-diethyl-2-((5-isopropyl-1,3,4-oxadiazol-2-yl)methyl)-3-oxo-2,3-dihydropyridazine-4-carbonitrile,
2-((5-(4-bromophenyl)-1,3,4-oxadiazol-2-yl)methyl)-5,6-diethyl-3-oxo-2,3-dihydropyridazine-4-carbonitrile,
4,5-dichloro-2-((5-(4-nitrophenyl)-1,3,4-oxadiazol-2-yl)methyl)pyridazin-3(2H)-one,
4,5-dichloro-2-((5-(3,4,5-trimethoxyphenyl)-1,3,4-oxadiazol-2-yl)methyl)pyridazin-3(2H)-one,
2-((5-cyclopropyl-1,3,4-oxadiazol-2-yl)methyl)-5,6-diethyl-3-oxo-2,3-dihydropyridazine-4-carbonitrile,
6-cyclopropyl-2-((5-ethyl-1,3,4-oxadiazol-2-yl)methyl)-4-(trifluoromethyl)pyridazin-3(2H)-one,
2-((5-(tert-butyl)-1,3,4-oxadiazol-2-yl)methyl)-6-cyclopropyl-4-(trifluoromethyl)pyridazin-3(2H)-one,
6-cyclopropyl-2-((5-methyl-1,3,4-oxadiazol-2-yl)methyl)-4-(trifluoromethyl)pyridazin-3(2H)-one,
6-cyclopropyl-2-((5-cyclopropyl-1,3,4-oxadiazol-2-yl)methyl)-4-(trifluoromethyl)pyridazin-3(2H)-one,
5,6-dimethyl-2-((5-(4-nitrophenyl)-1,3,4-oxadiazol-2-yl)methyl)-3-oxo-2,3-dihydropyridazine-4-carbonitrile,
5,6-dimethyl-3-oxo-2-((5-(3,4,5-trimethoxyphenyl)-1,3,4-oxadiazol-2-yl)methyl)-2,3-dihydropyridazine-4-carbonitrile,
2-((5-(4-bromophenyl)-1,3,4-oxadiazol-2-yl)methyl)-6-cyclopropyl-4-(trifluoromethyl)pyridazin-3(2H)-one,
2-((5-(3-bromophenyl)-1,3,4-oxadiazol-2-yl)methyl)-6-cyclopropyl-4-(trifluoromethyl)pyridazin-3(2H)-one,
6-cyclopropyl-4-(trifluoromethyl)-2-((5-(3,4,5-trimethoxyphenyl)-1,3,4-oxadiazol-2-yl)methyl)pyridazin-3(2H)-one,
2-((5-(3-bromophenyl)-1,3,4-oxadiazol-2-yl)methyl)-4,5-dichloropyridazin-3(2H)-one,
4,5-dichloro-2-((5-(4-methoxyphenyl)-1,3,4-oxadiazol-2-yl)methyl)pyridazin-3(2H)-one,
2-((5-(5-bromofuran-2-yl)-1,3,4-oxadiazol-2-yl)methyl)-4,5-dichloropyridazin-3(2H)-one,
5,6-dimethyl-3-oxo-2-((5-phenyl-1,3,4-oxadiazol-2-yl)methyl)-2,3-dihydropyridazine-4-carbonitrile,
2-((5-(4-methoxyphenyl)-1,3,4-oxadiazol-2-yl)methyl)-5,6-dimethyl-3-oxo-2,3-dihydropyridazine-4-carbonitrile,
2-((5-(4-bromophenyl)-1,3,4-oxadiazol-2-yl)methyl)-4,5-dichloropyridazin-3(2H)-one,
4,5-dichloro-2-((5-(thiophen-2-yl)-1,3,4-oxadiazol-2-yl)methyl)pyridazin-3(2H)-one,
2-((5-(2-bromophenyl)-1,3,4-oxadiazol-2-yl)methyl)-4,5-dichloropyridazin-3(2H)-one.

### Compounds of Formula IV

In some embodiments, the SecTRAP forming agent is a compound of formula IV

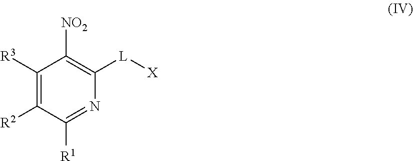

or a pharmaceutically acceptable salt thereof, wherein:
L represents —S(O)₂—;
X represents a heteroaryl group, attached to L via a carbon atom, optionally substituted by one or more groups independently selected from Y;
R¹, R² and R³ each independently represent H, halo, R^{a1}, —CN, -A^{a1}-C(Q^{a1})R^{b1}, -A^{b1}-C(Q^{b1})N(R^{c1})R^{d1}, -A^{c1}-C(Q^{c1})OR^{e1}, -A^{d1}-S(O)ₚR^{f1}, -A^{e1}-S(O)ₚN(R^{g1})R^{h1}, -A^{f1}-S(O)ₚORⁱ¹, —N₃, —N(R^{j1})R^{k1}, —N(H)CN, —NO₂, —ONO₂, —OR^{l1} or —SR^{m1};
each A^{a1} to A^{f1} independently represents a single bond, —N(R^{p1})— or —O—;
each Q^{a1} to Q^{c1} independently represents ═O, ═S, ═NRⁿ¹ or ═N(OR^{o1});
each R^{a1} and R^{f1} independently represents C₁₋₆ alkyl, C₂₋₆ alkenyl or C₂₋₆ alkynyl each optionally substituted by one or more groups independently selected from G^{1a}, or heterocyclyl optionally substituted by one or more groups independently selected from G^{1b};
each R^{b1}, R^{c1}, R^{d1}, R^{e1}, R^{g1}, R^{h1}, Rⁱ¹, R^{j1}, R^{k1}, R^{l1}, R^{m1}, Rⁿ¹, R^{o1} and R^{p1} independently represents H, C₁₋₆ alkyl, C₂₋₆ alkenyl or C₂₋₆ alkynyl each optionally substituted by one or more groups independently selected from G^{1a} or heterocyclyl optionally substituted by one or more groups independently selected from G^{1b}; or
any of R^{c1} and R^{d1}, R^{g1} and R^{h1} and/or R^{j1} and R^{k1} are linked together to form, together with the nitrogen atom to which they are attached, a 3- to 6-membered ring, which ring optionally contains one further heteroatom and which ring optionally is substituted by one or more groups independently selected from halo, C₁₋₃ alkyl, C₂₋₃ alkenyl or C₂₋₃ alkynyl each optionally substituted by one or more halo, and ═O;
each G^{1a} and G^{1b} independently represents halo, —CN, —N(R^{a2})R^{b2}, —OR^{c2}, —SR^{d2} or ═O;
each R^{a2}, R^{b2}, R^{c2} and R^{d2} independently represents H or C₁₋₆ alkyl, C₂₋₆ alkenyl or C₂₋₆ alkynyl each optionally substituted by one or more fluoro; or
R^{a2} and R^{b2} are linked together to form, along with the nitrogen atom to which they are attached, a 3- to 6-membered ring, which ring optionally contains one further heteroatom and which ring optionally is substituted by one or more groups independently selected from fluoro and C₁₋₃ alkyl, C₂₋₃ alkenyl or C₂₋₃ alkynyl each optionally substituted by one or more fluoro;
each Y independently represents halo, R^{a3}, —CN, -A^{a2}-C(Q^{a2})R^{b3}, -A^{b2}-C(Q^{b2})N(R^{c3})R^{d3}, -A^{c2}-C(Q^{c2})OR^{e3}, -A^{d2}-S(O)_{q}R^{f3}, -A^{e2}-S(O)_{q}N(R^{g3})R^{h3}, -A^{f2}-S(O)_{q}ORⁱ³, —N₃, —N(R^{j3})R^{k3}, —N(H)CN, —NO₂, —ONO₂, —OR^{l3} or —SR^{m3};
each Q^{a2} to Q^{c2} independently represents ═O, ═S, ═NRⁿ³ or ═N(OR^{o3});
each A^{a2} to A^{f2} independently represents a single bond, —N(R^{p3})— or —O—;
each R^{a3} and R^{f3} independently represents C₁₋₆ alkyl, C₂₋₆ alkenyl or C₂₋₆ alkynyl each optionally substituted by one or more groups independently selected from G^{2a}, heterocyclyl optionally substituted by one or more groups independently selected from G^{2b}, aryl optionally substituted by one or more groups independently selected from G^{2c}, or heteroaryl optionally substituted by one or more groups independently selected from G^{2d};
each R^{b3}, R^{c3}, R^{d3}, R^{e3}, R^{g3}, R^{h3}, Rⁱ³, R^{j3}, R^{k3}, R^{l3}, R^{m3}, Rⁿ³, R^{o3} and R^{p3} independently represents H, C₁₋₆ alkyl, C₂₋₆ alkenyl or C₂₋₆ alkynyl each optionally substituted by one or more groups independently selected from G^{2a}, heterocyclyl optionally substituted by one or more groups independently selected from G^{2b}, aryl optionally substituted by one or more groups independently selected from G^{2c}, or heteroaryl optionally substituted by one or more groups independently selected from G^{2d}; or
any two R^{c3} and R^{d3}, R^{g3} and R^{h3} and/or R^{j3} and R^{k3} are linked together to form, along with the nitrogen atom to which they are attached, a 3- to 6-membered ring, which ring optionally contains one further heteroatom and which ring optionally is substituted by one or more groups independently selected from heterocyclyl optionally substituted by one or more groups independently selected from G^{2b}, aryl optionally substituted by one or more groups independently selected from G^{2c}, or heteroaryl optionally substituted by one or more groups independently selected from G^{2d}, and ═O;
each G^{2a} independently represents halo, —CN, —N(R^{j4})R^{k4}, —OR^{l4}, —SR^{m4} or ═O;
each G^{2b} independently represents halo, R^{a4}, —CN, —N(R^{j4})R^{k4}, —OR^{l4}, —SR^{m4} or ═O;
each G^{2c} and G^{2d} independently represents halo, R^{a4}, —CN, -A^{a3}-C(Q^{a4})R^{b4}, -A^{b3}-C(Q^{b3})N(R^{c4})R^{d4}, -A^{c3}-C(Q^{c3})OR^{e4}, -A^{d3}-S(O)_{q}R^{f4}, -A^{e3}-S(O)_{q}N(R^{g4})R^{h4}, -A^{f3}-S(O)_{q}ORⁱ⁴, —N₃, —N(R^{j4})R^{k4}, —N(H)CN, —NO₂, —ONO₂, —OR^{l4} or —SR^{m4};
each Q^{a3} to Q^{c3} independently represents ═O, ═S, ═NRⁿ⁴ or ═N(OR^{o4});
each A^{a3} to A^{f3} independently represents a single bond, —N(R^{p4})— or —O—;
each R^{a4} and R^{f4} independently represents C₁₋₆ alkyl, C₂₋₆ alkenyl or C₂₋₆ alkynyl each optionally substituted by one or more groups independently selected from G^{3a}, heterocyclyl optionally substituted by one or more groups independently selected from G^{3b}, aryl optionally substituted by one or more groups independently selected from G^{3c}, or heteroaryl optionally substituted by one or more groups independently selected from G^{3d};
each R^{b4}, R^{c4}, R^{d4}, R^{e4}, R^{g4}, R^{h4}, Rⁱ⁴, R^{j4}, R^{k4}, R^{l4}, R^{m4}, Rⁿ⁴, R^{o4} and R^{p4} independently represents H, C₁₋₆ alkyl, C₂₋₆ alkenyl or C₂₋₆ alkynyl each optionally substituted by one or more groups independently selected from G^{3a} or heterocyclyl optionally substituted by one or more groups independently selected from G^{3b}, aryl optionally substituted by one or more groups independently selected from G^{3c}, or heteroaryl optionally substituted by one or more groups independently selected from G^{3d}; or
any of R^{o4} and R^{d4}, R^{g4} and R^{h4} and/or R^{j4} and R^{k4} are linked together to form, together with the nitrogen atom to which they are attached, a 3- to 6-membered ring, which ring optionally contains one further heteroatom and which ring optionally is substituted by one or more groups independently selected G^{3b};
each G^{3a} and G^{3b} independently represents halo, R^{a5}, —CN, —N(R^{b5})R^{c5}, —OR^{d5}, —SR^{e5} or ═O;
each G^{3c} and G^{3d} independently representing halo, R^{a5}, —CN, -A^{a4}-C(Q^{a4})R^{b5}, -A^{b4}-C(Q^{b4})N(R^{c5})R^{d5}, -A^{c4}-C(Q^{c4})OR^{e5}, -A^{d5}-S(O)_{q}R^{f5}, -A^{e4}-S(O)_{q}N(R^{g5})R^{h5}, -A^{f4}-S(O)_{q}ORⁱ⁵, —N₃, —N(R^{j5})R^{k5}, —N(H)CN, —NO₂, —ONO₂, —OR^{l5} or —SR^{m5},
each Q^{a4} to Q^{c4} independently represents ═O, ═S, ═NRⁿ⁵ or ═N(OR^{o5});
each A^{a4} to A^{f4} independently represents a single bond, —N(R^{p5})— or —O—;
with each R^{f5} to R^{p5} independently representing H, or C₁₋₆ alkyl, C₂₋₆ alkenyl or C₂₋₆ alkynyl each optionally substituted by one or more groups independently selected from G⁴, or with each R^{g5} and R^{h5}, and R^{j5} and R^{k5} being linked together to form, together with the nitrogen atom to which they are attached, a 3- to 6-membered ring, which ring optionally contains one further heteroatom and which ring optionally is substituted by one or more groups independently selected from G⁴;
each R^{a5} independently represents C₁₋₆ alkyl, C₂₋₆ alkenyl or C₂₋₆ alkynyl each optionally substituted by one or more groups independently selected from G⁴;
each R^{b5}, R^{c5}, R^{d5} and R^{e5} independently represents H, or C₁₋₆ alkyl, C₂₋₆ alkenyl or C₂₋₆ alkynyl each optionally substituted by one or more groups independently selected from G⁴; or
each R^{b5} and R^{c5} are linked together to form, together with the nitrogen atom to which they are attached, a 3- to 6-membered ring, which ring optionally contains one further heteroatom and which ring optionally is substituted by one or more groups independently selected from G⁴;
each G⁴ independently represents halo, R^{a6}, —CN, —N(R^{b6})R^{c6}, —OR^{d6} or ═O;
each R^{a6} independently represents C₁₋₆ alkyl, C₂₋₆ alkenyl or C₂₋₆ alkynyl each optionally substituted by one or more fluoro;
each R^{b6}, R^{c6} and R^{d6} independently represents H, or C₁₋₆ alkyl, C₂₋₆ alkenyl or C₂₋₆ alkynyl each optionally substituted by one or more fluoro; and
each p and q independently represents 1 or 2.

In some preferred embodiments, the SecTRAP forming agent is a compound of formula IV, wherein R₃ and/or R₂ (preferably R₃ and R₂) represent H.

In some preferred embodiments, the SecTRAP forming agent is a compound of formula IV, wherein R₁ is OR^{l1}, preferably R^{l1} represents C₁₋₆ alkyl, more preferably C₁ alkyl.

In some preferred embodiments, the SecTRAP forming agent is a compound of formula IV, wherein X is a pyridine ring, preferably an unsubstituted pyridine ring.

A particularly preferred SecTRAP forming agent is a compound of formula IV having the structure:

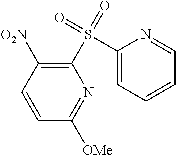 [SMILES: COc1ccc([N+](=O)[O-])c(S(=O)(=O)c2ccccn2)n1]

This compound is also referred to herein as (6-Methoxy-3-nitro-2-(pyridin-2-ylsulfonyl)pyridine) and OT-1012.

In some embodiments, the compound of formula IV is not a compound selected from the list consisting of compounds:
3-nitro-2-(pyridin-2-ylsulfonyl)pyridine,
2-((3-nitropyridin-2-yl)sulfonyl)pyrimidine, or
N-(6-chloro-2-((5-chloro-3-nitropyridin-2-yl)sulfonyl)pyridin-3-yl)acetamide.

### Compounds of Formula V

In some embodiments, the SecTRAP forming agent is a compound of formula V

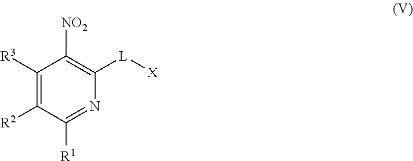

or a pharmaceutically acceptable salt thereof, wherein:
L represents —S(O)₂—;
X represents C₁₋₁₂ alkyl, C₂₋₁₂ alkenyl or C₂₋₁₂ alkynyl each optionally substituted by one or more groups independently selected from Y;
R¹, R² and R³ each independently represent H, halo, R^{a1}, —CN, -A^{a1}-C(Q^{a1})R^{b1}, -A^{b1}-C(Q^{b1})N(R^{c1})R^{d1}, -A^{c1}-C(Q^{c1})OR^{e1}, -A^{d1}-S(O)ₚR^{f1}, -A^{e1}-S(O)ₚN(R^{g1})R^{h1}, -A^{f1}-S(O)ₚORⁱ¹, —N₃, —N(R^{j1})R^{k1}, —N(H)CN, —NO₂, —ONO₂, —OR^{l1} or —SR^{m1};
each A^{a1} to A^{f1} independently represents a single bond, —N(R^{p1})— or —O—;
each Q^{a1} to Q^{c1} independently represents ═O, ═S, ═NRⁿ¹ or ═N(OR^{o1});
each R^{a1} and R^{f1} independently represents C₁₋₆ alkyl, C₂₋₆ alkenyl or C₂₋₆ alkynyl each optionally substituted by one or more groups independently selected from G^{1a}, or heterocyclyl optionally substituted by one or more groups independently selected from G^{1b};
each R^{b1}, R^{c1}, R^{d1}, R^{e1}, R^{g1}, R^{h1}, Rⁱ¹, R^{j1}, R^{k1}, R^{l1}, R^{m1}, Rⁿ¹, R^{o1} and R^{p1} independently represents H, C₁₋₆ alkyl, C₂₋₆ alkenyl or C₂₋₆ alkynyl each optionally substituted by one or more groups independently selected from G^{1a}, or heterocyclyl optionally substituted by one or more groups independently selected from G^{1b}; or
any of R^{c1} and R^{d1}, R^{g1} and R^{h1} and/or R^{j1} and R^{k1} are linked together to form, together with the nitrogen atom to which they are attached, a 3- to 6-membered ring, which ring optionally contains one further heteroatom and which ring optionally is substituted by one or more groups independently selected from G^{1b}, C₁₋₃ alkyl, C₂₋₃ alkenyl or C₂₋₃ alkynyl each optionally substituted by one or more G^{1a}, and ═O;
each G^{1a} and G^{1b} independently represents halo, —CN, —N(R^{a2})R^{b2}, —OR^{c2}, —SR^{d2} or ═O;
each R^{a2}, R^{b2}, R^{c2} and R^{d2} independently represents H, or C₁₋₆ alkyl, C₂₋₆ alkenyl or C₂₋₆ alkynyl each optionally substituted by one or more fluoro; or
R^{a2} and R^{b2} are linked together to form, along with the nitrogen atom to which they are attached, a 3- to 6-membered ring, which ring optionally contains one further heteroatom and which ring optionally is substituted by one or more groups independently selected from fluoro and C₁₋₃ alkyl, C₂₋₃ alkenyl or C₂₋₃ alkynyl each optionally substituted by one or more fluoro;
each Y independently represents halo, R^{a3}, —CN, -A^{a2}-C(Q^{a2})R^{b3}, -A^{b2}-C(Q^{b2})N(R^{c3})R^{d3}, -A^{c2}-C(Q^{c2})OR^{e3}, -A^{d2}-S(O)_{q}R^{f3}, -A^{e2}-S(O)_{q}N(R^{g3})R^{h3}, -A^{f2}-S(O)_{q}ORⁱ³, —N₃, —N(R^{j3})R^{k3}, —N(H)CN, —NO₂, —ONO₂, —OR^{l3}, —SR^{m3} or ═O;
each Q^{a2} to Q^{c2} independently represents ═O, ═S, ═NRⁿ³ or ═N(OR^{o3});
each A^{a2} to A^{f2} independently represents a single bond, —N(R^{p3})— or —O—;
each R^{a3} independently represents heterocyclyl optionally substituted by one or more groups independently selected from G^{2b}, aryl optionally substituted by one or more groups independently selected from G^{2c}, or heteroaryl optionally substituted by one or more groups independently selected from G^{2d};
each R^{f3} independently represents C₁₋₆ alkyl optionally substituted by one or more groups independently selected from G^{2a}, heterocyclyl optionally substituted by one or more groups independently selected from G^{2b}, aryl optionally substituted by one or more groups independently selected from G^{2c}, or heteroaryl optionally substituted by one or more groups independently selected from G^{2d};
each R^{b3}, R^{c3}, R^{d3}, R^{e3}, R^{g3}, R^{h3}, Rⁱ³, R^{j3}, R^{k3}, R^{l3}, R^{m3}, Rⁿ³, R^{o3} and R^{p3} independently represents H, C₁₋₆ alkyl optionally substituted by one or more groups independently selected from G^{2a}, heterocyclyl optionally substituted by one or more groups independently selected from G^{2b}, aryl optionally substituted by one or more groups independently selected from G^{2c}, or heteroaryl optionally substituted by one or more groups independently selected from G^{2d}; or
any two R^{c3} and R^{d3}, R^{g3} and R^{h3} and/or R^{j3} and R^{k3} are linked together to form, along with the nitrogen atom to which they are attached, a 3- to 6-membered ring, which ring optionally contains one further heteroatom and which ring optionally is substituted by one or more groups independently selected from heterocyclyl optionally substituted by one or more groups independently selected from G^{2b}, aryl optionally substituted by one or more groups independently selected from G^{2c}, or heteroaryl optionally substituted by one or more groups independently selected from G^{2d}, and ═O;
each G^{2a} independently represents halo, —CN, —N(R^{j4})R^{k4}, —OR^{l4}, —SR^{m4} or ═O;
each G^{2b} independently represents halo, R^{a4}, —CN, —N(R^{j4})R^{k4}, —OR^{l4}, —SR^{m4} or ═O;
each G^{2c} and G^{2d} independently represents halo, R^{a4}, —CN, -A^{a3}-C(Q^{a4})R^{b4}, -A^{b3}-C(Q^{b3})N(R^{c4})R^{d4}, -A^{c3}-C(Q^{c3})OR^{e4}, -A^{d3}-S(O)_{q}R^{f4}, -A^{e3}-S(O)_{q}N(R^{g4})R^{h4}, -A^{f3}-S(O)_{q}ORⁱ⁴, —N₃, —N(R^{j4})R^{k4}, —N(H)CN, —NO₂, —ONO₂, —OR^{l4} or —SR^{m4};
each Q^{a3} to Q^{c3} independently represents ═O, ═S, ═NRⁿ⁴ or ═N(OR^{o4});
each A^{a3} to A^{f3} independently represents a single bond, —N(R^{p4})— or —O—;
each R^{a4} and R^{f4} independently represents C₁₋₆ alkyl optionally substituted by one or more groups independently selected from G^{3a}, heterocyclyl optionally substituted by one or more groups independently selected from G^{3b}, aryl optionally substituted by one or more groups independently selected from G^{3c}, or heteroaryl optionally substituted by one or more groups independently selected from G^{3d};
each R^{b4}, R^{c4}, R^{d4}, R^{e4}, R^{g4}, R^{h4}, Rⁱ⁴, R^{j4}, R^{k4}, R^{l4}, R^{m4}, Rⁿ⁴, R^{o4} and R^{p4} independently represents H, C₁₋₆ alkyl, C₂₋₆ alkenyl or C₂₋₆ alkynyl each optionally substituted by one or more groups independently selected from G^{3a} or heterocyclyl optionally substituted by one or more groups independently selected from G^{3b}, aryl optionally substituted by one or more groups independently selected from G^{3c}, or heteroaryl optionally substituted by one or more groups independently selected from G^{3d}; or
any of R^{o4} and R^{d4}, R^{g4} and R^{h4} and/or R^{j4} and R^{k4} are linked together to form, together with the nitrogen atom to which they are attached, a 3- to 6-membered ring, which ring optionally contains one further heteroatom and which ring optionally is substituted by one or more groups independently selected G^{3b};
each G^{3a} and G^{3b} independently represents halo, R^{a5}, —CN, —N(R^{b5})R^{c5}, —OR^{d5}, —SR^{e5} or ═O;
each G^{3c} and G^{3d} independently representing halo, R^{a5}, —CN, -A^{a4}-C(Q^{a4})R^{b5}, -A^{b4}-C(Q^{b4})N(R^{c5})R^{d5}, -A^{c4}-C(Q^{c4})OR^{e5}, -A^{d5}-S(O)_{q}R^{f5}, -A^{e4}-S(O)_{q}N(R^{g5})R^{h5}, -A^{f4}-S(O)_{q}ORⁱ⁵, —N₃, —N(R^{j5})R^{k5}, —N(H)CN, —NO₂, —ONO₂, —OR^{l5} or —SR^{m5},
each Q^{a4} to Q^{c4} independently represents ═O, ═S, ═NRⁿ⁵ or ═N(OR^{o5});
each A^{a4} to A^{f4} independently represents a single bond, —N(R^{p5})— or —O—;
with each R^{f5} to R^{p5} independently representing H, or C₁₋₆ alkyl, C₂₋₆ alkenyl or C₂₋₆ alkynyl each optionally substituted by one or more groups independently selected from G⁴, or with each R^{g5} and R^{h5}, and R^{j5} and R^{k5} being linked together to form, together with the nitrogen atom to which they are attached, a 3- to 6-membered ring, which ring optionally contains one further heteroatom and which ring optionally is substituted by one or more groups independently selected from G⁴;
each R^{a5} independently represents C₁₋₆ alkyl, C₂₋₆ alkenyl or C₂₋₆ alkynyl each optionally substituted by one or more groups independently selected from G⁴;
each R^{b5}, R^{c5}, R^{d5} and R^{e5} independently represents H, or C₁₋₆ alkyl, C₂₋₆ alkenyl or C₂₋₆ alkynyl each optionally substituted by one or more groups independently selected from G⁴; or
each R^{b5} and R^{c5} are linked together to form, together with the nitrogen atom to which they are attached, a 3- to 6-membered ring, which ring optionally contains one further heteroatom and which ring optionally is substituted by one or more groups independently selected from G⁴;
each G⁴ independently represents halo, R^{a6}, —CN, —N(R^{b6})R^{c6}, —OR^{d6} or ═O;
each R^{a6} independently represents C₁₋₆ alkyl, C₂₋₆ alkenyl or C₂₋₆ alkynyl each optionally substituted by one or more fluoro;
each R^{b6}, R^{c6} and R^{d6} independently represents H, or C₁₋₆ alkyl, C₂₋₆ alkenyl or C₂₋₆ alkynyl each optionally substituted by one or more fluoro; and
each p and q independently represents 1 or 2.

In some preferred embodiments, the SecTRAP forming agent is a compound of formula V, wherein R₃ and/or R₂ (preferably R₃ and R₂) represent H.

In some preferred embodiments, the SecTRAP forming agent is a compound of formula V, wherein R¹ is OR^{l1}, preferably R^{l1} represents C₁₋₆ alkyl, more preferably C₁ alkyl.

In some preferred embodiments, the SecTRAP forming agent is a compound of formula V, wherein R₁ is halo (e.g. chloro), or OR^{l1} (preferably R^{l1} represents C₁₋₆ alkyl, more preferably C₁ alkyl) or N(R^{j1})R^{k1} (preferably R^{j1} and R^{k1} are H or N(R^{j1})R^{k1} is N(CH₃)₂) or SR^{m1}.

In some preferred embodiments, the SecTRAP forming agent is a compound of formula V, wherein X is C₁₋₁₂ alkyl.

In some preferred embodiments, the SecTRAP forming agent is a compound of formula V, wherein X is C₁₋₁₂ alkyl (preferably C₂ alkyl), or X is C₁₋₁₂ alkyl (preferably C₁ alkyl) substituted by Y, wherein Y is R^{a3} and R^{a3} is aryl (preferably phenyl), or X is C₁₋₁₂ alkyl (preferably C₂ alkyl) substituted by Y, wherein Y is -A^{c2}-C(Q^{c2})OR^{e3} (preferably A^{c2} is a single bond, Q^{c2} is ═O and R^{e3} is C₁₋₆ alkyl (preferably C₄ alkyl), or X is C₁₋₁₂alkyl, preferably C₁₋₁₀ alkyl or C₁₋₈ alkyl (preferably C₈ alkyl).

In some preferred embodiments, the SecTRAP forming agent is a compound of formula V, wherein X is C₁₋₁₂ alkyl (preferably C₂ alkyl).

In some preferred embodiments, the SecTRAP forming agent is a compound of formula V, wherein X is C₁₋₁₂ alkyl (preferably C₁ alkyl) substituted by Y, wherein Y is R^{a3} and R^{a3} is aryl (preferably phenyl).

In some preferred embodiments, the SecTRAP forming agent is a compound of formula V, wherein X is C₁₋₁₂ alkyl (preferably C₂ alkyl) substituted by Y, wherein Y is -A^{c2}-C(Q^{c2})OR^{e3} (preferably A^{c2} is a single bond, Q^{o2} is ═O and R^{e3} is C₁₋₆ alkyl (preferably C₁ alkyl).

In some embodiments, the SecTRAP forming agent is a compound of formula V, wherein X is C₁₋₁₂ alkyl, preferably C₁₋₁₀ alkyl or C₁₋₈ alkyl. In some embodiments, the SecTRAP forming agent is a compound of formula V, wherein X is C₈ alkyl. In some embodiments, such alkyl groups are unsubstituted and are preferably unbranched.

A particularly preferred SecTRAP forming agent is a compound of formula V having the structure:

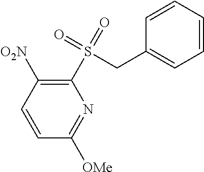 [SMILES: COc1ccc([N+](=O)[O-])c(S(=O)(=O)Cc2ccccc2)n1]

This compound is also referred to herein as (2-Benzylsulfonyl-6-methoxy-3-nitropyridine) and OT-1011.

Another particularly preferred SecTRAP forming agent is a compound of formula V having the structure:

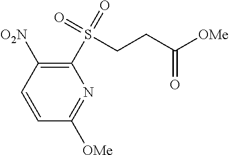 [SMILES: COC(=O)CCS(=O)(=O)c1nc(OC)ccc1[N+](=O)[O-]]

This compound is also referred to herein as (methyl 3-((6-methoxy-3-nitropyridin-2-yl)sulfonyl)propanoate) and OT-1113.

Another particularly preferred SecTRAP forming agent is a compound of formula V having the structure:

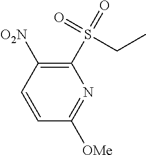 [SMILES: CCS(=O)(=O)c1nc(OC)ccc1[N+](=O)[O-]]

This compound is also referred to herein as (2-(ethylsulfonyl)-6-methoxy-3-nitropyridine) and OT-1129.

Another particularly preferred SecTRAP forming agent is a compound of formula V having the structure:

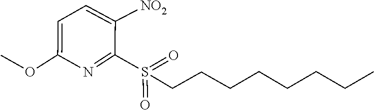 [SMILES: CCCCCCCCS(=O)(=O)c1nc(OC)ccc1[N+](=O)[O-]]

This compound is also referred to herein as (6-methoxy-3-nitro-2-(octylsulfonyl)pyridine) and OT-1096.

Thus in some embodiments the SecTRAP forming agent is a compound of formula V selected from the group consisting of (or comprising) OT-1011, OT-1113, OT-1129, and OT-1096.

### Compounds of Formula VI

In some embodiments, the SecTRAP forming agent is a compound of formula VI

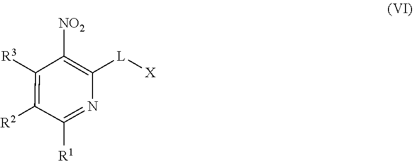

or a pharmaceutically acceptable salt thereof, wherein:
L represents —S(O)₂—;
X represents heterocyclyl, connected to L via a carbon atom, and optionally substituted by one or more groups independently selected from Y;
R¹, R² and R³ each independently represent H, halo, R^{a1}, —CN, -A^{a1}-C(Q^{a1})R^{b1}, -A^{b1}-C(Q^{b1})N(R^{c1})R^{d1}, -A^{c1}-C(Q^{c1})OR^{e1}, -A^{d1}-S(O)ₚR^{f1}, -A^{e1}-S(O)ₚN(R^{g1})R^{h1}, -A^{f1}-S(O)ₚORⁱ¹, —N₃, —N(R^{j1})R^{k1}, —N(H)CN, —NO₂, —ONO₂, —OR^{l1} or —SR^{m1};
each A^{a1} to A^{f1} independently represents a single bond, —N(R^{p1})— or —O—;
each Q^{a1} to Q^{c1} independently represents ═O, ═S, ═NRⁿ¹ or ═N(OR^{o1});
each R^{a1} and R^{f1} independently represents C₁₋₆ alkyl, C₂₋₆ alkenyl or C₂₋₆ alkynyl each optionally substituted by one or more groups independently selected from G^{1a}, or heterocyclyl optionally substituted by one or more groups independently selected from G^{1b};
each R^{b1}, R^{c1}, R^{d1}, R^{e1}, R^{g1}, R^{h1}, Rⁱ¹, R^{j1}, R^{k1}, R^{l1}, R^{m1}, Rⁿ¹, R^{o1} and R^{p1} independently represents H, C₁₋₆ alkyl, C₂₋₆ alkenyl or C₂₋₆ alkynyl each optionally substituted by one or more groups independently selected from G^{1a}, or heterocyclyl optionally substituted by one or more groups independently selected from G^{1b}; or
any of R^{c1} and R^{d1}, R^{g1} and R^{h1} and/or R^{j1} and R^{k1} are linked together to form, together with the nitrogen atom to which they are attached, a 3- to 6-membered ring, which ring optionally contains one further heteroatom and which ring optionally is substituted by one or more groups independently selected from G^{1b}, C₁₋₃ alkyl, C₂₋₃ alkenyl or C₂₋₃ alkynyl each optionally substituted by one or more G^{1a}, and ═O;
each G^{1a} and G^{1b} independently represents halo, —CN, —N(R^{a2})R^{b2}, —OR^{c2}, —SR^{d2} or ═O;
each R^{a2}, R^{b2}, R^{c2} and R^{d2} independently represents H or C₁₋₆ alkyl, C₂₋₆ alkenyl or C₂₋₆ alkynyl each optionally substituted by one or more fluoro; or
R^{a2} and R^{b2} are linked together to form, along with the nitrogen atom to which they are attached, a 3- to 6-membered ring, which ring optionally contains one further heteroatom and which ring optionally is substituted by one or more groups independently selected from fluoro and C₁₋₃ alkyl, C₂₋₃ alkenyl or C₂₋₃ alkynyl each optionally substituted by one or more fluoro;
each Y independently represents halo, R^{a3}, —CN, -A^{a2}-C(Q^{a2})R^{b3}, -A^{b2}-C(Q^{b2})N(R^{c3})R^{d3}, -A^{c2}-C(Q^{c2})OR^{e3}, -A^{d2}-S(O)_{q}R^{f3}, -A^{e2}-S(O)_{q}N(R^{g3})R^{h3}, -A^{f2}-S(O)_{q}ORⁱ³, —N₃, —N(R^{j3})R^{k3}, —N(H)CN, —NO₂, —ONO₂, —OR^{l3}, —SR^{m3} or ═O;
each Q^{a2} to Q^{c2} independently represents ═O, ═S, ═NRⁿ³ or ═N(OR^{o3});
each A^{a2} to A^{f2} independently represents a single bond, —N(R^{p3})— or —O—;
each R^{a3} and R^{f3} independently represents C₁₋₆ alkyl, C₂₋₆ alkenyl or C₂₋₆ alkynyl each optionally substituted by one or more groups independently selected from G^{2a}, heterocyclyl optionally substituted by one or more groups independently selected from G^{2b}, aryl optionally substituted by one or more groups independently selected from G^{2c}, or heteroaryl optionally substituted by one or more groups independently selected from G^{2d};
each R^{b3}, R^{c3}, R^{d3}, R^{e3}, R^{g3}, R^{h3}, Rⁱ³, R^{j3}, R^{k3}, R^{l3}, R^{m3}, Rⁿ³, R^{o3} and R^{p3} independently represents H, C₁₋₆ alkyl, C₂₋₆ alkenyl or C₂₋₆ alkynyl each optionally substituted by one or more groups independently selected from G^{2a}, heterocyclyl optionally substituted by one or more groups independently selected from G^{2b}, aryl optionally substituted by one or more groups independently selected from G^{2c}, or heteroaryl optionally substituted by one or more groups independently selected from G^{2d}; or
any two R^{c3} and R^{d3}, R^{g3} and R^{h3} and/or R^{j3} and R^{k3} are linked together to form, along with the nitrogen atom to which they are attached, a 3- to 6-membered ring, which ring optionally contains one further heteroatom and which ring optionally is substituted by one or more groups independently selected from heterocyclyl optionally substituted by one or more groups independently selected from G^{2b}, aryl optionally substituted by one or more groups independently selected from G^{2c}, or heteroaryl optionally substituted by one or more groups independently selected from G^{2d}, and ═O;
each G^{2a} independently represents halo, —CN, —N(R^{j4})R^{k4}, —OR^{l4}, —SR^{m4} or ═O;
each G^{2b} independently represents halo, R^{a4}, —CN, —N(R^{j4})R^{k4}, —OR^{l4}, —SR^{m4} or ═O;
each G^{2c} and G^{2d} independently represents halo, R^{a4}, —CN, -A^{a3}-C(Q^{a4})R^{b4}, -A^{b3}-C(Q^{b3})N(R^{c4})R^{d4}, -A^{c3}-C(Q^{c3})OR^{e4}, -A^{d3}-S(O)_{q}R^{f4}, -A^{e3}-S(O)_{q}N(R^{g4})R^{h4}, -A^{f3}-S(O)_{q}ORⁱ⁴, —N₃, —N(R^{j4})R^{k4}, —N(H)CN, —NO₂, —ONO₂, —OR^{l4} or —SR^{m4};
each Q^{a3} to Q^{c3} independently represents ═O, ═S, ═NRⁿ⁴ or ═N(OR^{o4});
each A^{a3} to A^{f3} independently represents a single bond, —N(R^{p4})— or —O—;
each R^{a4} and R^{f4} independently represents C₁₋₆ alkyl, C₂₋₆ alkenyl or C₂₋₆ alkynyl each optionally substituted by one or more groups independently selected from G^{3a}, or heterocyclyl optionally substituted by one or more groups independently selected from G^{3b};
each R^{b4}, R^{c4}, R^{d4}, R^{e4}, R^{g4}, R^{h4}, Rⁱ⁴, R^{j4}, R^{k4}, R^{l4}, R^{m4}, Rⁿ⁴, R^{o4} and R^{p4} independently represents H, C₁₋₆ alkyl, C₂₋₆ alkenyl or C₂₋₆ alkynyl each optionally substituted by one or more groups independently selected from G^{3a} or heterocyclyl optionally substituted by one or more groups independently selected from G^{3b}; or
any of R^{c4} and R^{d4}, R^{g4} and R^{h4} and/or R^{j4} and R^{k4} are linked together to form, together with the nitrogen atom to which they are attached, a 3- to 6-membered ring, which ring optionally contains one further heteroatom and which ring optionally is substituted by one or more groups independently selected G^{3b};
each G^{3a} and G^{3b} independently represents halo, R^{a5}, —CN, —N(R^{b5})R^{c5}, —OR^{d5}, —SR^{e5} or ═O;
each R^{a5} independently represents C₁₋₆ alkyl, C₂₋₆ alkenyl or C₂₋₆ alkynyl each optionally substituted by one or more groups independently selected from G⁴;
each R^{b5}, R^{c5}, R^{d5} and R^{e5} independently represents H, C₁₋₆ alkyl, C₂₋₆ alkenyl or C₂₋₆ alkynyl each optionally substituted by one or more groups independently selected from G⁴; or
each R^{b5} and R^{c5} are linked together to form, together with the nitrogen atom to which they are attached, a 3- to 6-membered ring, which ring optionally contains one further heteroatom and which ring optionally is substituted by one or more groups independently selected from G⁴;
each G⁴ independently represents halo, R^{a6}, —CN, —N(R^{b6})R^{c6}, —OR^{d6} or ═O;
each R^{a6} independently represents C₁₋₆ alkyl, C₂₋₆ alkenyl or C₂₋₆ alkynyl each optionally substituted by one or more fluoro;
each R^{b6}, R^{c6} and R^{d6} independently represents H, or C₁₋₆ alkyl, C₂₋₆ alkenyl or C₂₋₆ alkynyl each optionally substituted by one or more fluoro; and
each p and q independently represents 1 or 2

### Compounds of Formula VII

In some embodiments, the SecTRAP forming agent is a compound of formula VII

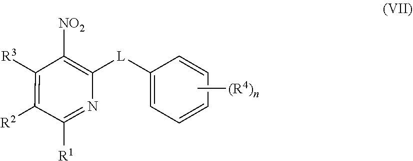

or a pharmaceutically acceptable salt thereof, wherein:
L represents —S(O)—;
n represents 0 to 5;
R¹, R² and R³ each independently represent H, halo, R^{a1}, —CN, -A^{a1}-C(Q^{a1})R^{b1}, -A^{b1}-C(Q^{b1})N(R^{c1})R^{d1}, -A^{c1}-C(Q^{c1})OR^{e1}, -A^{d1}-S(O)ₚR^{f1}, -A^{e1}-S(O)ₚN(R^{g1})R^{h1}, -A^{f1}-S(O)ₚORⁱ¹, —N₃, —N(R^{j1})R^{k1}, —N(H)CN, —NO₂, —ONO₂, —OR^{l1} or —SR^{m1};
each A^{a1} to A^{f1} independently represents a single bond, —N(R^{p1})— or —O—;
each Q^{a1} to Q^{c1} independently represents ═O, ═S, ═NRⁿ¹ or ═N(OR^{o1});
each R^{a1} and R^{f1} independently represents C₁₋₆ alkyl, C₂₋₆ alkenyl or C₂₋₆ alkynyl each optionally substituted by one or more groups independently selected from G^{1a}, heterocyclyl optionally substituted by one or more groups independently selected from G^{1b}, aryl optionally substituted by one or more groups independently selected from G^{1c}, or heteroaryl optionally substituted by one or more groups independently selected from G^{1d};
each R^{b1}, R^{c1}, R^{d1}, R^{e1}, R^{g1}, R^{h1}, Rⁱ¹, R^{j1}, R^{k1}, R^{l1}, R^{m1}, Rⁿ¹, R^{o1} and R^{p1} independently represents H, C₁₋₆ alkyl, C₂₋₆ alkenyl or C₂₋₆ alkynyl each optionally substituted by one or more groups independently selected from G^{1a}, heterocyclyl optionally substituted by one or more groups independently selected from G^{1b}, aryl optionally substituted by one or more groups independently selected from G^{1c}, or heteroaryl optionally substituted by one or more groups independently selected from G^{1d}; or
any of R^{c1} and R^{d1}, R^{g1} and R^{h1} and/or R^{j1} and R^{k1} are linked together to form, together with the nitrogen atom to which they are attached, a 3- to 6-membered ring, which ring optionally contains one further heteroatom and which ring optionally is substituted by one or more groups independently selected from halo, C₁₋₃ alkyl, C₂₋₃ alkenyl or C₂₋₃ alkynyl each optionally substituted by one or more halo, and ═O;
each R⁴ independently represents halo, R^{a2}, —CN, -A^{a2}-C(Q^{a2})R^{b2}, -A^{b2}-C(Q^{b2})N(R^{c2})R^{d2}, -A^{c2}-C(Q^{c2})OR^{e2}, -A^{d2}-S(O)_{q}R^{f2}, -A^{e2}-S(O)_{q}N(R^{g2})R^{h2}, -A^{f2}-S(O)_{q}ORⁱ², —N₃, —N(R^{j2})R^{k2}, —N(H)CN, —NO₂, —ONO₂, —OR^{l2} or —SR^{m2};
each Q^{a2} to Q^{c2} independently represents ═O, ═S, ═NRⁿ² or ═N(OR^{o2});
each A^{a2} to A^{f2} independently represents a single bond, —N(R^{p2})— or —O—;
each R^{a2} and R^{f2} independently represents C₁₋₆ alkyl, C₂₋₆ alkenyl or C₂₋₆ alkynyl each optionally substituted by one or more groups independently selected from G^{2a}, heterocyclyl optionally substituted by one or more groups independently selected from G^{2b}, aryl optionally substituted by one or more groups independently selected from G^{2c}, or heteroaryl optionally substituted by one or more groups independently selected from G^{2d};
each R^{b2}, R^{c2}, R^{d2}, R^{e2}, R^{g2}, R^{h2}, Rⁱ², R^{j2}, R^{k2}, R^{l2}, R^{m2}, Rⁿ², R^{o2} and R^{p2} independently represents H, C₁₋₆ alkyl, C₂₋₆ alkenyl or C₂₋₆ alkynyl each optionally substituted by one or more groups independently selected from G^{2a}, heterocyclyl optionally substituted by one or more groups independently selected from G^{2b}, aryl optionally substituted by one or more groups independently selected from G^{2c}, or heteroaryl optionally substituted by one or more groups independently selected from G^{2d}; or
any two R^{c2} and R^{d2}, R^{g2} and R^{h2} and/or R^{j2} and R^{k2} are linked together to form, along with the nitrogen atom to which they are attached, a 3- to 6-membered ring, which ring optionally contains one further heteroatom and which ring optionally is substituted by one or more groups independently selected from halogen, C₁₋₃ alkyl, C₂₋₃ alkenyl or C₂₋₃ alkynyl each optionally substituted by one or more halogens, and ═O;
each G^{1a}, G^{1b}, G^{1c}, G^{1d}, G^{2a}, G^{2b}, G^{2c} and G^{2d} independently represents halo, —CN, —N(R^{a3})R^{b3}, —OR^{c3}, —SR^{d3} or ═O;
each R^{a3}, R^{b3}, R^{c3} and R^{d3} independently represents H, or C₁₋₆ alkyl, C₂₋₆ alkenyl or C₂₋₆ alkynyl each optionally substituted by one or more fluoro;
or R^{a3} and R^{b3} are linked together to form, along with the nitrogen atom to which they are attached, a 3- to 6-membered ring, which ring optionally contains one further heteroatom and which ring optionally is substituted by one or more groups independently selected from fluoro, C₁₋₃ alkyl optionally substituted by one or more fluoro, and ═O; and
each p and q independently represents 1 or 2.

In some embodiments, the compound of formula VII is not a compound selected from the list consisting of compounds:
3-nitro-2-(phenylsulfinyl)pyridine,
3-nitro-2-(p-tolylsulfinyl)pyridine,
2-((4-bromophenyl)sulfinyl)-3-nitropyridine,
2-((3-chlorophenyl)sulfinyl)-3-nitropyridine, or
3-nitro-2-((3-(trifluoromethyl)phenyl)-sulfinyl)pyridine.

### Compounds of Formula VIII

In some embodiments, the SecTRAP forming agent is a compound of formula VIII

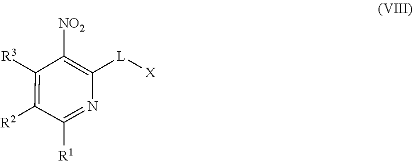

or a pharmaceutically acceptable salt thereof, wherein:
L represents —S(O)ₙ—;
n represents 2 or 1;
X represents a heteroaryl group, attached to L via a carbon atom, optionally substituted by one or more groups independently selected from Y;
R¹, R² and R³ each independently represent H, halo, R^{a1}, —CN, -A^{a1}-C(Q^{a1})R^{b1}, -A^{b1}-C(Q^{b1})N(R^{c1})R^{d1}, -A^{c1}-C(Q^{c1})OR^{e1}, -A^{d1}-S(O)ₚR^{f1}, -A^{e1}-S(O)ₚN(R^{g1})R^{h1}, -A^{f1}-S(O)ₚORⁱ¹, —N₃, —N(R^{j1})R^{k1}, —N(H)CN, —NO₂, —ONO₂, —OR^{l1} or —SR^{m1};
each A^{a1} to A^{f1} independently represents a single bond, —N(R^{p1})— or —O—;
each Q^{a1} to Q^{c1} independently represents ═O, ═S, ═NRⁿ¹ or ═N(OR^{o1});
each R^{a1} and R^{f1} independently represents C₁₋₆ alkyl, C₂₋₆ alkenyl or C₂₋₆ alkynyl each optionally substituted by one or more groups independently selected from G^{1a}, heterocyclyl optionally substituted by one or more groups independently selected from G^{1b}, aryl optionally substituted by one or more groups independently selected from G^{1c}, or heteroaryl optionally substituted by one or more groups independently selected from G^{1d};
each R^{b1}, R^{c1}, R^{d1}, R^{e1}, R^{g1}, R^{h1}, Rⁱ¹, R^{j1}, R^{k1}, R^{l1}, R^{m1}, Rⁿ¹, R^{o1} and R^{p1} independently represents H, C₁₋₆ alkyl, C₂₋₆ alkenyl or C₂₋₆ alkynyl each optionally substituted by one or more groups independently selected from G^{1a}, heterocyclyl optionally substituted by one or more groups independently selected from G^{1b}, aryl optionally substituted by one or more groups independently selected from G^{1c}, or heteroaryl optionally substituted by one or more groups independently selected from G^{1d};
any of R^{c1} and R^{d1}, R^{g1} and R^{h1} and/or R^{j1} and R^{k1} are linked together to form, together with the nitrogen atom to which they are attached, a 3- to 6-membered ring, which ring optionally contains one further heteroatom and which ring optionally is substituted by one or more groups independently selected from halo, and C₁₋₃ alkyl, C₂₋₃ alkenyl or C₂₋₃ alkynyl each optionally substituted by one or more halo, and ═O;
each G^{1a} and G^{1b} independently represents halo, —CN, —N(R^{a2})R^{b2}, —OR^{c2}, —SR^{d2} or ═O;
each G^{1c} and G^{1d} independently represents halo, —CN, —N(R^{a2})R^{b2}, —OR^{c2} or —SR^{d2};
each R^{a2}, R^{b2}, R^{c2} and R^{d2} independently represents H or C₁₋₆ alkyl, C₂₋₆ alkenyl or C₂₋₆ alkynyl each optionally substituted by one or more fluoro; or
R^{a2} and R^{b2} are linked together to form, along with the nitrogen atom to which they are attached, a 3- to 6-membered ring, which ring optionally contains one further heteroatom and which ring optionally is substituted by one or more groups independently selected from fluoro, C₁₋₃ alkyl, C₂₋₃ alkenyl or C₂₋₃ alkynyl each optionally substituted by one or more fluoro;
each Y independently represents halo, R^{a3}, —CN, -A^{a2}-C(Q^{a2})R^{b3}, -A^{b2}-C(Q^{b2})N(R^{c3})R^{d3}, -A^{c2}-C(Q^{c2})OR^{e3}, -A^{d2}-S(O)_{q}R^{f3}, -A^{e2}-S(O)_{q}N(R^{g3})R^{h3}, -A^{f2}-S(O)_{q}ORⁱ³, —N₃, —N(R^{j3})R^{k3}, —N(H)CN, —NO₂, —ONO₂, —OR^{l3} or —SR^{m3};
each Q^{a2} to Q^{c2} independently represents ═O, ═S, ═NRⁿ³ or ═N(OR^{o3});
each A^{a2} to A^{f2} independently represents a single bond, —N(R^{p3})— or —O—;
each R^{a3} and R^{f3} independently represents C₁₋₆ alkyl, C₂₋₆ alkenyl or C₂₋₆ alkynyl each optionally substituted by one or more groups independently selected from G^{2a}, heterocyclyl optionally substituted by one or more groups independently selected from G^{2b}, aryl optionally substituted by one or more groups independently selected from G^{2c}, or heteroaryl optionally substituted by one or more groups independently selected from G^{2d};
each R^{b3}, R^{c3}, R^{d3}, R^{e3}, R^{g3}, R^{h3}, Rⁱ³, R^{j3}, R^{k3}, R^{l3}, R^{m3}, Rⁿ³, R^{o3} and R^{p3} independently represents H, C₁₋₆ alkyl, C₂₋₆ alkenyl or C₂₋₆ alkynyl each optionally substituted by one or more groups independently selected from G^{2a}, heterocyclyl optionally substituted by one or more groups independently selected from G^{2b}, aryl optionally substituted by one or more groups independently selected from G^{2c}, or heteroaryl optionally substituted by one or more groups independently selected from G^{2d}; or
any two R^{c3} and R^{d3}, R^{g3} and R^{h3} and/or R^{j3} and R^{k3} are linked together to form, along with the nitrogen atom to which they are attached, a 3- to 6-membered ring, which ring optionally contains one further heteroatom and which ring optionally is substituted by one or more groups independently selected from heterocyclyl optionally substituted by one or more groups independently selected from G^{2b}, aryl optionally substituted by one or more groups independently selected from G^{2c}, or heteroaryl optionally substituted by one or more groups independently selected from G^{2d}, and ═O;
each G^{2a} independently represents halo, —CN, —N(R^{j4})R^{k4}, —OR^{l4}, —SR^{m4} or ═O;
each G^{2b} independently represents halo, R^{a4}, —CN, —N(R^{j4})R^{k4}, —OR^{l4}, —SR^{m4} or ═O;
each G^{2c} and G^{2d} independently represents halo, R^{a4}, —CN, -A^{a3}-C(Q^{a4})R^{b4}, -A^{b3}-C(Q^{b3})N(R^{c4})R^{d4}, -A^{c3}-C(Q^{c3})OR^{e4}, -A^{d3}-S(O)_{q}R^{f4}, -A^{e3}-S(O)_{q}N(R^{g4})R^{h4}, -A^{f3}-S(O)_{q}ORⁱ⁴, —N₃, —N(R^{j4})R^{k4}, —N(H)CN, —NO₂, —ONO₂, —OR^{l4} or —SR^{m4};
each Q^{a3} to Q^{c3} independently represents ═O, ═S, ═NRⁿ⁴ or ═N(OR^{o4});
each A^{a3} to A^{f3} independently represents a single bond, —N(R^{p4})— or —O—;
each R^{a4} and R^{f4} independently represents C₁₋₆ alkyl, C₂₋₆ alkenyl or C₂₋₆ alkynyl each optionally substituted by one or more groups independently selected from G^{3a}, heterocyclyl optionally substituted by one or more groups independently selected from G^{3b}, aryl optionally substituted by one or more groups independently selected from G^{3c}, or heteroaryl optionally substituted by one or more groups independently selected from G^{3d};
each R^{b4}, R^{c4}, R^{d4}, R^{e4}, R^{g4}, R^{h4}, Rⁱ⁴, R^{j4}, R^{k4}, R^{l4}, R^{m4}, Rⁿ⁴, R^{o4} and R^{p4} independently represents H, C₁₋₆ alkyl, C₂₋₆ alkenyl or C₂₋₆ alkynyl each optionally substituted by one or more groups independently selected from G^{3a} or heterocyclyl optionally substituted by one or more groups independently selected from G^{3b}, aryl optionally substituted by one or more groups independently selected from G^{3c}, or heteroaryl optionally substituted by one or more groups independently selected from G^{3d}; or
any of R^{c4} and R^{d4}, R^{g4} and R^{h4} and/or R^{j4} and R^{k4} are linked together to form, together with the nitrogen atom to which they are attached, a 3- to 6-membered ring, which ring optionally contains one further heteroatom and which ring optionally is substituted by one or more groups independently selected G^{3b};
each G^{3a} and G^{3b} independently represents halo, R^{a5}, —CN, —N(R^{b5})R^{c5}, —OR^{d5}, —SR^{e5} or ═O;
each G^{3c} and G^{3d} independently representing halo, R^{a5}, —CN, -A^{a4}-C(Q^{a4})R^{b5}, -A^{b4}-C(Q^{b4})N(R^{c5})R^{d5}, -A^{c4}-C(Q^{c4})OR^{e5}, -A^{d5}-S(O)_{q}R^{f5}, -A^{e4}-S(O)_{q}N(R^{g5})R^{h5}, -A^{f4}-S(O)_{q}ORⁱ⁵, —N₃, —N(R^{j5})R^{k5}, —N(H)CN, —NO₂, —ONO₂, —OR^{l5} or —SR^{m5},
each Q^{a4} to Q^{c4} independently represents ═O, ═S, ═NRⁿ⁵ or ═N(OR^{o5});
each A^{a4} to A^{f4} independently represents a single bond, —N(R^{p5})— or —O—;
with each R^{f5} to R^{p5} independently representing H, or C₁₋₆ alkyl, C₂₋₆ alkenyl or C₂₋₆ alkynyl each optionally substituted by one or more groups independently selected from G⁴, or with each R^{g5} and R^{h5}, and R^{j5} and R^{k5} being linked together to form, together with the nitrogen atom to which they are attached, a 3- to 6-membered ring, which ring optionally contains one further heteroatom and which ring optionally is substituted by one or more groups independently selected from G⁴;
each R^{a5} independently represents C₁₋₆ alkyl, C₂₋₆ alkenyl or C₂₋₆ alkynyl each optionally substituted by one or more groups independently selected from G⁴;
each R^{b5}, R^{c5}, R^{d5} and R^{e5} independently represents H, or C₁₋₆ alkyl, C₂₋₆ alkenyl or C₂₋₆ alkynyl each optionally substituted by one or more groups independently selected from G⁴; or
each R^{b5} and R^{c5} are linked together to form, together with the nitrogen atom to which they are attached, a 3- to 6-membered ring, which ring optionally contains one further heteroatom and which ring optionally is substituted by one or more groups independently selected from G⁴;
each G⁴ independently represents halo, R^{a6}, —CN, —N(R^{b6})R^{c6}, —OR^{d6} or ═O;
each R^{a6} independently represents C₁₋₆ alkyl, C₂₋₆ alkenyl or C₂₋₆ alkynyl each optionally substituted by one or more fluoro;
each R^{b6}, R^{c6} and R^{d6} independently represents H, or C₁₋₆ alkyl, C₂₋₆ alkenyl or C₂₋₆ alkynyl each optionally substituted by one or more fluoro; and
each p and q independently represents 1 or 2.

In some embodiments, the SecTRAP forming agent is a compound of formula VIII, wherein n=1.

In some embodiments, the compound of formula VIII is not a compound selected from the list consisting of compounds:
3-nitro-2-(pyridin-2-ylsulfonyl)pyridine,
2-((3-nitropyridin-2-yl)sulfonyl)pyrimidine,
N-(6-chloro-2-((5-chloro-3-nitropyridin-2-yl)sulfonyl)pyridin-3-yl)acetamide, or
3-nitro-2-[(3-nitro-2-thienyl)sulfinyl]-pyridine.

### Compounds of Formula IX

In some embodiments, the SecTRAP forming agent is a compound of formula IX

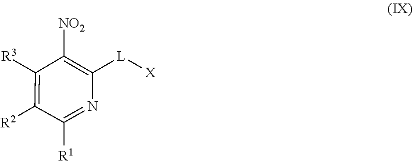

or a pharmaceutically acceptable salt thereof, wherein:
L represents —S(O)ₙ—;
n represents 2 or 1;
X represents C₁₋₁₂ alkyl, C₂₋₁₂ alkenyl or C₂₋₁₂ alkynyl each optionally substituted by one or more groups independently selected from Y;
R¹ represents halo, —N(R^{j1})R^{k1}, —OR^{l1} or —SR^{m1};
R² and R³ each independently represent H, halo, R^{a1}, —CN, -A^{a1}-C(Q^{a1})R^{b1}, -A^{b1}-C(Q^{b1})N(R^{c1})R^{d1}, -A^{c1}-C(Q^{c1})OR^{e1}, -A^{d1}-S(O)ₚR^{f1}, -A^{e1}-S(O)ₚN(R^{g1})R^{h1}, -A^{f1}-S(O)ₚORⁱ¹, —N₃, —N(R^{j1})R^{k1}, —N(H)CN, —NO₂, —ONO₂, —OR^{l1} or —SR^{m1};
each A^{a1} to A^{f1} independently represents a single bond, —N(R^{p1})— or —O—;
each Q^{a1} to Q^{c1} independently represents ═O, ═S, ═NRⁿ¹ or ═N(OR^{o1});
each R^{a1} and R^{f1} independently represents C₁₋₆ alkyl, C₂₋₆ alkenyl or C₂₋₆ alkynyl each optionally substituted by one or more groups independently selected from G^{1a}, heterocyclyl optionally substituted by one or more groups independently selected from G^{1b}, aryl optionally substituted by one or more groups independently selected from G^{1c}, or heteroaryl optionally substituted by one or more groups independently selected from G^{1d};
each R^{b1}, R^{c1}, R^{d1}, R^{e1}, R^{g1}, R^{h1}, Rⁱ¹, R^{j1}, R^{k1}, R^{l1}, R^{m1}, Rⁿ¹, R^{o1} and R^{p1} independently represents H, C₁₋₆ alkyl, C₂₋₆ alkenyl or C₂₋₆ alkynyl each optionally substituted by one or more groups independently selected from G^{1a}, heterocyclyl optionally substituted by one or more groups independently selected from G^{1b}, aryl optionally substituted by one or more groups independently selected from G^{1c}, or heteroaryl optionally substituted by one or more groups independently selected from G^{1d};
any of R^{c1} and R^{d1}, R^{g1} and R^{h1} and/or R^{j1} and R^{k1} are linked together to form, together with the nitrogen atom to which they are attached, a 3- to 6-membered ring, which ring optionally contains one further heteroatom and which ring optionally is substituted by one or more groups independently selected from G^{1b}, C₁₋₃ alkyl, C₂₋₃ alkenyl or C₂₋₃ alkynyl each optionally substituted by one or more G^{1a}, and ═O;
each G^{1a} and G^{1b} independently represents halo, —CN, —N(R^{a2})R^{b2}, —OR^{c2}, —SR^{d2} or ═O;
each R^{a2}, R^{b2}, R^{c2} and R^{d2} independently represents H, or C₁₋₆ alkyl, C₂₋₆ alkenyl or C₂₋₆ alkynyl each optionally substituted by one or more fluoro; or
R^{a2} and R^{b2} are linked together to form, along with the nitrogen atom to which they are attached, a 3- to 6-membered ring, which ring optionally contains one further heteroatom and which ring optionally is substituted by one or more groups independently selected from fluoro and C₁₋₃ alkyl, C₂₋₃ alkenyl or C₂₋₃ alkynyl each optionally substituted by one or more fluoro;
each Y independently represents halo, R^{a3}, —CN, -A^{a2}-C(Q^{a2})R^{b3}, -A^{b2}-C(Q^{b2})N(R^{c3})R^{d3}, -A^{c2}-C(Q^{c2})OR^{e3}, -A^{d2}-S(O)_{q}R^{f3}, -A^{e2}-S(O)_{q}N(R^{g3})R^{h3}, -A^{f2}-S(O)_{q}ORⁱ³, —N₃, —N(R^{j3})R^{k3}, —N(H)CN, —NO₂, —ONO₂, —OR^{l3}, —SR^{m3} or ═O;
each Q^{a2} to Q^{c2} independently represents ═O, ═S, ═NRⁿ³ or ═N(OR^{o3});
each A^{a2} to A^{f2} independently represents a single bond, —N(R^{p3})— or —O—;
each R^{a3} independently represents heterocyclyl optionally substituted by one or more groups independently selected from G^{2b}, aryl optionally substituted by one or more groups independently selected from G^{2c}, or heteroaryl optionally substituted by one or more groups independently selected from G^{2d};
each R^{f3} independently represents C₁₋₆ alkyl optionally substituted by one or more groups independently selected from G^{2a}, heterocyclyl optionally substituted by one or more groups independently selected from G^{2b}, aryl optionally substituted by one or more groups independently selected from G^{2c}, or heteroaryl optionally substituted by one or more groups independently selected from G^{2d};
each R^{b3}, R^{c3}, R^{d3}, R^{e3}, R^{g3}, R^{h3}, Rⁱ³, R^{j3}, R^{k3}, R^{l3}, R^{m3}, Rⁿ³, R^{o3} and R^{p3} independently represents H, C₁₋₆ alkyl optionally substituted by one or more groups independently selected from G^{2a}, heterocyclyl optionally substituted by one or more groups independently selected from G^{2b}, aryl optionally substituted by one or more groups independently selected from G^{2c}, or heteroaryl optionally substituted by one or more groups independently selected from G^{2d}; or
any two R^{c3} and R^{d3}, R^{g3} and R^{h3} and/or R^{j3} and R^{k3} are linked together to form, along with the nitrogen atom to which they are attached, a 3- to 6-membered ring, which ring optionally contains one further heteroatom and which ring optionally is substituted by one or more groups independently selected from heterocyclyl optionally substituted by one or more groups independently selected from G^{2b}, aryl optionally substituted by one or more groups independently selected from G^{2c}, or heteroaryl optionally substituted by one or more groups independently selected from G^{2d}, and ═O;
each G^{2a} independently represents halo, —CN, —N(R^{j4})R^{k4}, —OR^{l4}, —SR^{m4} or ═O;
each G^{2b} independently represents halo, R^{a4}, —CN, —N(R^{j4})R^{k4}, —OR^{l4}, —SR^{m4} or ═O;
each G^{2c} and G^{2d} independently represents halo, R^{a4}, —CN, -A^{a3}-C(Q^{a4})R^{b4}, -A^{b3}-C(Q^{b3})N(R^{c4})R^{d4}, -A^{c3}-C(Q^{c3})OR^{e4}, -A^{d3}-S(O)_{q}R^{f4}, -A^{e3}-S(O)_{q}N(R^{g4})R^{h4}, -A^{f3}-S(O)_{q}ORⁱ⁴, —N₃, —N(R^{j4})R^{k4}, —N(H)CN, —NO₂, —ONO₂, —OR^{l4} or —SR^{m4};
each Q^{a3} to Q^{c3} independently represents ═O, ═S, ═NRⁿ⁴ or ═N(OR^{o4});
each A^{a3} to A^{f3} independently represents a single bond, —N(R^{p4})— or —O—;
each R^{a4} and R^{f4} independently represents C₁₋₆ alkyl optionally substituted by one or more groups independently selected from G^{3a}, heterocyclyl optionally substituted by one or more groups independently selected from G^{3b}, aryl optionally substituted by one or more groups independently selected from G^{3c}, or heteroaryl optionally substituted by one or more groups independently selected from G^{3d};
each R^{b4}, R^{c4}, R^{d4}, R^{e4}, R^{g4}, R^{h4}, Rⁱ⁴, R^{j4}, R^{k4}, R^{l4}, R^{m4}, Rⁿ⁴, R^{o4} and R^{p4} independently represents H, C₁₋₆ alkyl, C₂₋₆ alkenyl or C₂₋₆ alkynyl each optionally substituted by one or more groups independently selected from G^{3a} or heterocyclyl optionally substituted by one or more groups independently selected from G^{3b}, aryl optionally substituted by one or more groups independently selected from G^{3c}, or heteroaryl optionally substituted by one or more groups independently selected from G^{3d}; or
any of R^{c4} and R^{d4}, R^{g4} and R^{h4} and/or R^{j4} and R^{k4} are linked together to form, together with the nitrogen atom to which they are attached, a 3- to 6-membered ring, which ring optionally contains one further heteroatom and which ring optionally is substituted by one or more groups independently selected G^{3b};
each G^{3a} and G^{3b} independently represents halo, R^{a5}, —CN, —N(R^{b5})R^{c5}, —OR^{d5}, —SR^{e5} or ═O;
each G^{3c} and G^{3d} independently representing halo, R^{a5}, —CN, -A^{a4}-C(Q^{a4})R^{b5}, -A^{b4}-C(Q^{b4})N(R^{c5})R^{d5}, -A^{c4}-C(Q^{c4})OR^{e5}, -A^{d5}-S(O)_{q}R^{f5}, -A^{e4}-S(O)_{q}N(R^{g5})R^{h5}, -A^{f4}-S(O)_{q}ORⁱ⁵, —N₃, —N(R^{j5})R^{k5}, —N(H)CN, —NO₂, —ONO₂, —OR^{l5} or —SR^{m5},
each Q^{a4} to Q^{c4} independently represents ═O, ═S, ═NRⁿ⁵ or ═N(OR^{o5});
each A^{a4} to A^{f4} independently represents a single bond, —N(R^{p5})— or —O—;
with each R^{f5} to R^{p5} independently representing H, or C₁₋₆ alkyl, C₂₋₆ alkenyl or C₂₋₆ alkynyl each optionally substituted by one or more groups independently selected from G⁴, or with each R^{g5} and R^{h5}, and R^{j5} and R^{k5} being linked together to form, together with the nitrogen atom to which they are attached, a 3- to 6-membered ring, which ring optionally contains one further heteroatom and which ring optionally is substituted by one or more groups independently selected from G⁴;
each R^{a5} independently represents C₁₋₆ alkyl, C₂₋₆ alkenyl or C₂₋₆ alkynyl each optionally substituted by one or more groups independently selected from G⁴;
each R^{b5}, R^{c5}, R^{d5} and R^{e5} independently represents H, or C₁₋₆ alkyl, C₂₋₆ alkenyl or C₂₋₆ alkynyl each optionally substituted by one or more groups independently selected from G⁴; or
each R^{b5} and R^{c5} are linked together to form, together with the nitrogen atom to which they are attached, a 3- to 6-membered ring, which ring optionally contains one further heteroatom and which ring optionally is substituted by one or more groups independently selected from G⁴;
each G⁴ independently represents halo, R^{a6}, —CN, —N(R^{b6})R^{c6}, —OR^{d6} or ═O;
each R^{a6} independently represents C₁₋₆ alkyl, C₂₋₆ alkenyl or C₂₋₆ alkynyl each optionally substituted by one or more fluoro;
each R^{b6}, R^{c6} and R^{d6} independently represents H, or C₁₋₆ alkyl, C₂₋₆ alkenyl or C₂₋₆ alkynyl each optionally substituted by one or more fluoro; and
each p and q independently represents 1 or 2.

In some embodiments, the compound of formula IX is not a compound selected from the list consisting of compounds:
2-((1-chloropropan-2-yl)sulfonyl)-6-methoxy-3-nitropyridine,
2-((6-methoxy-3-nitropyridin-2-yl)sulfonyl)ethane-1-sulfonamide,
2-((2-chloroethyl)sulfonyl)-6-methoxy-3-nitropyridine,
2-((4-chlorobutan-2-yl)sulfonyl)-6-methoxy-3-nitropyridine,
2-((6-methoxy-3-nitropyridin-2-yl)sulfonyl)ethane-1-sulfonyl chloride,
2-((3-chloro-2-methylpropyl)sulfonyl)-6-methoxy-3-nitropyridine,
2-((3-chloropropyl)sulfonyl)-6-methoxy-3-nitropyridine,
6-methoxy-3-nitro-2-(vinylsulfonyl)pyridine,
6-methoxy-2-(methylsulfonyl)-3-nitropyridine,
6-(2,6-dichloro-4-(trifluoromethyl)phenoxy)-2-(methylsulfonyl)-3-nitropyridine,
6-(2,6-dichloro-4-(trifluoromethoxy)phenoxy)-2-(methylsulfonyl)-3-nitropyridine,
6-(2,6-dichloro-4-(trifluoromethyl)phenoxy)-2-(ethylsulfonyl)-3-nitropyridine, or
2-(butylsulfinyl)-3-nitro-pyridine,
3-[(3-nitro-2-pyridinyl)sulfinyl]-2-propenoic acid methyl ester,
3-[(3-nitro-2-pyridinyl)sulfinyl]-2-propenoic acid ethyl ester,
6-[(2-methylpropyl)sulfinyl]-5-nitro-2-methanesulfonate-2-pyridinol,
3-chloro-2-[(6-chloro-3-nitro-2-pyridinyl)sulfinyl]-benzoic acid ethyl ester,
3-nitro-2-[(4-piperidinylmethyl)sulfinyl]-pyridine,
3-nitro-2-[(3-pyrrolidinylmethyl)sulfinyl]-pyridine,
3-nitro-2-[(3-piperidinylmethyl)sulfinyl]-pyridine,
3-nitro-2-[(2-pyrrolidinylmethyl)sulfinyl]-pyridine,
3-nitro-2-[(2-piperidinylmethyl)sulfinyl]-pyridine,
4-[[(3-nitro-2-pyridinyl)sulfinyl]methyl]-1-piperidinecarboxylic acid 1,1-dimethylethyl ester,
3-[[(3-nitro-2-pyridinyl)sulfinyl]methyl]-1-piperidinecarboxylic acid 1,1-dimethylethyl ester,
3-[[(3-nitro-2-pyridinyl)sulfinyl]methyl]-1-pyrrolidinecarboxylic acid, 1,1-dimethylethyl ester,
2-[[(3-nitro-2-pyridinyl)sulfinyl]methyl]-1-pyrrolidinecarboxylic acid 1,1-dimethylethyl ester,
2-[[(3-nitro-2-pyridinyl)sulfinyl]methyl]-1-piperidinecarboxylic acid 1,1-dimethylethyl ester,
6-[2,6-dichloro-4-(trifluoromethoxy)phenoxy]-2-(methylsulfinyl)-3-nitro-pyridine, or
6-[2,6-dichloro-4-(trifluoromethyl)phenoxy]-2-(ethylsulfinyl)-3-nitro-pyridine.

In some preferred embodiments, the SecTRAP forming agent is a compound of formula IX, wherein R₃ and/or R₂ (preferably R₃ and R₂) represent H.

In some preferred embodiments, the SecTRAP forming agent is a compound of formula IX, wherein n represents 1.

In some preferred embodiments, the SecTRAP forming agent is a compound of formula IX, wherein R₁ is OR^{l1}, preferably R^{l1} represents C₁₋₆ alkyl, more preferably C₁ alkyl.

In some preferred embodiments, the SecTRAP forming agent is a compound of formula IX, wherein X is C₁₋₁₂ alkyl, preferably C₂ alkyl.

A preferred SecTRAP forming agent is a compound of formula IX having the structure:

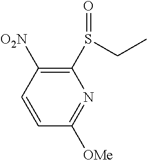

This compound is also referred to herein as (2-(ethylsulfinyl)-6-methoxy-3-nitropyridine) and OT-1131.

### Compounds of Formula X

In some embodiments, the SecTRAP forming agent is a compound of formula X

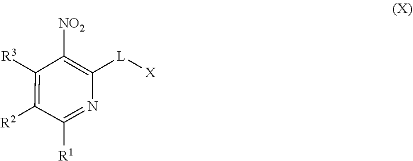

or a pharmaceutically acceptable salt thereof, wherein:
L represents —S(O)ₙ—;
n represents 2 or 1;
X represents heterocyclyl, connected to L via a carbon atom, and optionally substituted by one or more groups independently selected from Y;
R¹, R² and R³ each independently represent H, halo, R^{a1}, —CN, -A^{a1}-C(Q^{a1})R^{b1}, -A^{b1}-C(Q^{b1})N(R^{c1})R^{d1}, -A^{c1}-C(Q^{c1})OR^{e1}, -A^{d1}-S(O)ₚR^{f1}, -A^{e1}-S(O)ₚN(R^{g1})R^{h1}, -A^{f1}-S(O)ₚORⁱ¹, —N₃, —N(R^{j1})R^{k1}, —N(H)CN, —NO₂, —ONO₂, —OR^{l1} or —SR^{m1};
each A^{a1} to A^{f1} independently represents a single bond, —N(R^{p1})— or —O—;
each Q^{a1} to Q^{c1} independently represents ═O, ═S, ═NRⁿ¹ or ═N(OR^{o1});
each R^{a1} and R^{f1} independently represents C₁₋₆ alkyl, C₂₋₆ alkenyl or C₂₋₆ alkynyl each optionally substituted by one or more groups independently selected from G^{1a}, heterocyclyl optionally substituted by one or more groups independently selected from G^{1b}, aryl optionally substituted by one or more groups independently selected from G^{1c}, or heteroaryl optionally substituted by one or more groups independently selected from G^{1d};
each R^{b1}, R^{c1}, R^{d1}, R^{e1}, R^{g1}, R^{h1}, Rⁱ¹, R^{j1}, R^{k1}, R^{l1}, R^{m1}, Rⁿ¹, R^{o1} and R^{p1} independently represents H, C₁₋₆ alkyl, C₂₋₆ alkenyl or C₂₋₆ alkynyl each optionally substituted by one or more groups independently selected from G^{1a}, heterocyclyl optionally substituted by one or more groups independently selected from G^{1b}, aryl optionally substituted by one or more groups independently selected from G^{1c}, or heteroaryl optionally substituted by one or more groups independently selected from G^{1d}; or
any of R^{c1} and R^{d1}, R^{g1} and R^{h1} and/or R^{j1} and R^{k1} are linked together to form, together with the nitrogen atom to which they are attached, a 3- to 6-membered ring, which ring optionally contains one further heteroatom and which ring optionally is substituted by one or more groups independently selected from G^{1b}, C₁₋₃ alkyl, C₂₋₃ alkenyl or C₂₋₃ alkynyl each optionally substituted by one or more G^{1a}, and ═O;
each G^{1a} and G^{1b} independently represents halo, —CN, —N(R^{a2})R^{b2}, —OR^{c2}, —SR^{d2} or ═O;
each R^{a2}, R^{b2}, R^{c2} and R^{d2} independently represents H or C₁₋₆ alkyl, C₂₋₆ alkenyl or C₂₋₆ alkynyl each optionally substituted by one or more fluoro; or R^{a2} and R^{b2} are linked together to form, along with the nitrogen atom to which they are attached, a 3- to 6-membered ring, which ring optionally contains one further heteroatom and which ring optionally is substituted by one or more groups independently selected from fluoro and C₁₋₃ alkyl, C₂₋₃ alkenyl or C₂₋₃ alkynyl each optionally substituted by one or more fluoro;
each Y independently represents halo, R^{a3}, —CN, -A^{a2}-C(Q^{a2})R^{b3}, -A^{b2}-C(Q^{b2})N(R^{c3})R^{d3}, -A^{c2}-C(Q^{c2})OR^{e3}, -A^{d2}-S(O)_{q}R^{f3}, -A^{e2}-S(O)_{q}N(R^{g3})R^{h3}, -A^{f2}-S(O)_{q}ORⁱ³, —N₃, —N(R^{j3})R^{k3}, —N(H)CN, —NO₂, —ONO₂, —OR^{l3}, —SR^{m3} or ═O;
each Q^{a2} to Q^{c2} independently represents ═O, ═S, ═NRⁿ³ or ═N(OR^{o3});
each A^{a2} to A^{f2} independently represents a single bond, —N(R^{p3})— or —O—;
each R^{a3} and R^{f3} independently represents C₁₋₆ alkyl, C₂₋₆ alkenyl or C₂₋₆ alkynyl each optionally substituted by one or more groups independently selected from G^{2a}, heterocyclyl optionally substituted by one or more groups independently selected from G^{2b}, aryl optionally substituted by one or more groups independently selected from G^{2c}, or heteroaryl optionally substituted by one or more groups independently selected from G^{2d};
each R^{b3}, R^{c3}, R^{d3}, R^{e3}, R^{g3}, R^{h3}, Rⁱ³, R^{j3}, R^{k3}, R^{l3}, R^{m3}, Rⁿ³, R^{o3} and R^{p3} independently represents H, C₁₋₆ alkyl, C₂₋₆ alkenyl or C₂₋₆ alkynyl each optionally substituted by one or more groups independently selected from G^{2a}, heterocyclyl optionally substituted by one or more groups independently selected from G^{2b}, aryl optionally substituted by one or more groups independently selected from G^{2c}, or heteroaryl optionally substituted by one or more groups independently selected from G^{2d}; or
any two R^{c3} and R^{d3}, R^{g3} and R^{h3} and/or R^{j3} and R^{k3} are linked together to form, along with the nitrogen atom to which they are attached, a 3- to 6-membered ring, which ring optionally contains one further heteroatom and which ring optionally is substituted by one or more groups independently selected from heterocyclyl optionally substituted by one or more groups independently selected from G^{2b}, aryl optionally substituted by one or more groups independently selected from G^{2c}, or heteroaryl optionally substituted by one or more groups independently selected from G^{2d}, and ═O;
each G^{2a} independently represents halo, —CN, —N(R^{j4})R^{k4}, —OR^{l4}, —SR^{m4} or ═O;
each G^{2b} independently represents halo, R^{a4}, —CN, —N(R^{j4})R^{k4}, —OR^{l4}, —SR^{m4} or ═O;
each G^{2c} and G^{2d} independently represents halo, R^{a4}, —CN, -A^{a3}-C(Q^{a4})R^{b4}, -A^{b3}-C(Q^{b3})N(R^{c4})R^{d4}, -A^{c3}-C(Q^{c3})OR^{e4}, -A^{d3}-S(O)_{q}R^{f4}, -A^{e3}-S(O)_{q}N(R^{g4})R^{h4}, -A^{f3}-S(O)_{q}ORⁱ⁴, —N₃, —N(R^{j4})R^{k4}, —N(H)CN, —NO₂, —ONO₂, —OR^{l4} or —SR^{m4};
each Q^{a3} to Q^{c3} independently represents ═O, ═S, ═NRⁿ⁴ or ═N(OR^{o4});
each A^{a3} to A^{f3} independently represents a single bond, —N(R^{p4})— or —O—;
each R^{a4} and R^{f4} independently represents C₁₋₆ alkyl, C₂₋₆ alkenyl or C₂₋₆ alkynyl each optionally substituted by one or more groups independently selected from G^{3a}, or heterocyclyl optionally substituted by one or more groups independently selected from G^{3b};
each R^{b4}, R^{c4}, R^{d4}, R^{e4}, R^{g4}, R^{h4}, Rⁱ⁴, R^{j4}, R^{k4}, R^{l4}, R^{m4}, Rⁿ⁴, R^{o4} and R^{p4} independently represents H, C₁₋₆ alkyl, C₂₋₆ alkenyl or C₂₋₆ alkynyl each optionally substituted by one or more groups independently selected from G^{3a} or heterocyclyl optionally substituted by one or more groups independently selected from G^{3b}; or
any of R^{c4} and R^{d4}, R^{g4} and R^{h4} and/or R^{j4} and R^{k4} are linked together to form, together with the nitrogen atom to which they are attached, a 3- to 6-membered ring, which ring optionally contains one further heteroatom and which ring optionally is substituted by one or more groups independently selected G^{3b};
each G^{3a} and G^{3b} independently represents halo, R^{a5}, —CN, —N(R^{b5})R^{c5}, —OR^{d5}, —SR^{e5} or ═O;
each R^{a5} independently represents C₁₋₆ alkyl, C₂₋₆ alkenyl or C₂₋₆ alkynyl each optionally substituted by one or more groups independently selected from G⁴;
each R^{b5}, R^{c5}, R^{d5} and R^{e5} independently represents H, C₁₋₆ alkyl, C₂₋₆ alkenyl or C₂₋₆ alkynyl each optionally substituted by one or more groups independently selected from G⁴; or
each R^{b5} and R^{c5} are linked together to form, together with the nitrogen atom to which they are attached, a 3- to 6-membered ring, which ring optionally contains one further heteroatom and which ring optionally is substituted by one or more groups independently selected from G⁴;
each G⁴ independently represents halo, R^{a6}, —CN, —N(R^{b6})R^{c6}, —OR^{d6} or ═O;
each R^{a6} independently represents C₁₋₆ alkyl, C₂₋₆ alkenyl or C₂₋₆ alkynyl each optionally substituted by one or more fluoro;
each R^{b6}, R^{c6} and R^{d6} independently represents H, or C₁₋₆ alkyl, C₂₋₆ alkenyl or C₂₋₆ alkynyl each optionally substituted by one or more fluoro; and
each p and q independently represents 1 or 2.

In some embodiments, the SecTRAP forming agent is a compound of formula X, wherein n=1.

In some embodiments, the compound of formula X is not a compound selected from the list consisting of compounds:
3-nitro-2-(piperidin-4-ylsulfonyl)pyridine,
tert-butyl 3-((3-nitropyridin-2-yl)sulfonyl)pyrrolidine-1-carboxylate,
tert-butyl (R)-3-((3-nitropyridin-2-yl)sulfonyl)pyrrolidine-1-carboxylate,
tert-butyl (S)-3-((3-nitropyridin-2-yl)sulfonyl)pyrrolidine-1-carboxylate,
tert-butyl 3-((3-nitropyridin-2-yl)sulfonyl)piperidine-1-carboxylate,
tert-butyl 4-((3-nitropyridin-2-yl)sulfonyl)piperidine-1-carboxylate,
3-nitro-2-(4-piperidinylsulfinyl)-pyridine,
3-nitro-2-(3-pyrrolidinylsulfinyl)-pyridine,
3-nitro-2-(3-piperidinylsulfinyl)-pyridine,
4-[(3-nitro-2-pyridinyl)sulfinyl]-1-piperidinecarboxylic acid 1,1-dimethylethyl ester,
3-[(3-nitro-2-pyridinyl)sulfinyl]-1-pyrrolidinecarboxylic acid 1,1-dimethylethyl ester,
3-[(3-nitro-2-pyridinyl)sulfinyl]-1-piperidinecarboxylic acid 1,1-dimethylethyl ester, or
3-[(3-nitro-2-pyridinyl)sulfinyl]-7-oxabicyclo[2.2.1]hept-5-ene-2-carboxylic acid ethyl ester.

### Compounds of Formula XI

In some embodiments, the SecTRAP forming agent is a compound of formula XI

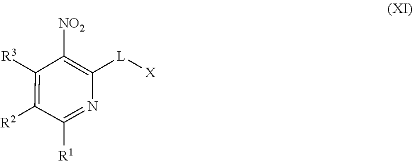

or a pharmaceutically acceptable salt thereof, wherein:
L represents —S(O)₂— or —S(O)—
X represents a heteroaryl group or heterocyclyl, connected to L via a carbon atom, or C₁₋₁₂ alkyl, C₂₋₁₂ alkenyl, C₂₋₁₂ alkynyl, or phenyl, each optionally substituted by one or more groups independently selected from Y;
R¹, R² and R³ each independently represent H, halo, R^{a1}, —CN, -A^{a1}-C(Q^{a1})R^{b1}, -A^{b1}-C(Q^{b1})N(R^{c1})R^{d1}, -A^{c1}-C(Q^{c1})OR^{e1}, -A^{d1}-S(O)ₚR^{f1}, -A^{e1}-S(O)ₚN(R^{g1})R^{h1}, -A^{f1}-S(O)ₚORⁱ¹, —N₃, —N(R^{j1})R^{k1}, —N(H)CN, —NO₂, —ONO₂, —OR^{l1} or —SR^{m1};
each A^{a1} to A^{f1} independently represents a single bond, —N(R^{p1})— or —O—;
each Q^{a1} to Q^{c1} independently represents ═O, ═S, ═NRⁿ¹ or ═N(OR^{o1});
each R^{a1} and R^{f1} independently represents C₁₋₆ alkyl, C₂₋₆ alkenyl or C₂₋₆ alkynyl each optionally substituted by one or more groups independently selected from G^{1a}, or heterocyclyl optionally substituted by one or more groups independently selected from G^{1b};
each R^{b1}, R^{c1}, R^{d1}, R^{e1}, R^{g1}, R^{h1}, Rⁱ¹, R^{j1}, R^{k1}, R^{l1}, R^{m1}, Rⁿ¹, R^{o1} and R^{p1} independently represents H, C₁₋₆ alkyl, C₂₋₆ alkenyl or C₂₋₆ alkynyl each optionally substituted by one or more groups independently selected from G^{1a}, or heterocyclyl optionally substituted by one or more groups independently selected from G^{1b}; or
any of R^{c1} and R^{d1}, R^{g1} and R^{h1} and/or R^{j1} and R^{k1} are linked together to form, together with the nitrogen atom to which they are attached, a 3- to 6-membered ring, which ring optionally contains one further heteroatom and which ring optionally is substituted by one or more groups independently selected from G^{1b}, C₁₋₃ alkyl, C₂₋₃ alkenyl or C₂₋₃ alkynyl each optionally substituted by one or more G^{1a}, and ═O;
each G^{1a} and G^{1b} independently represents halo, —CN, —N(R^{a2})R^{b2}, —OR^{c2}, —SR^{d2} or ═O;
each R^{a2}, R^{b2}, R^{c2} and R^{d2} independently represents H or C₁₋₆ alkyl, C₂₋₆ alkenyl or C₂₋₆ alkynyl each optionally substituted by one or more fluoro; or
R^{a2} and R^{b2} are linked together to form, along with the nitrogen atom to which they are attached, a 3- to 6-membered ring, which ring optionally contains one further heteroatom and which ring optionally is substituted by one or more groups independently selected from fluoro and C₁₋₃ alkyl, C₂₋₃ alkenyl or C₂₋₃ alkynyl each optionally substituted by one or more fluoro;
each Y independently represents halo, R^{a3}, —CN, -A^{a2}-C(Q^{a2})R^{b3}, -A^{b2}-C(Q^{b2})N(R^{c3})R^{d3}, -A^{c2}-C(Q^{c2})OR^{e3}, -A^{d2}-S(O)_{q}R^{f3}, -A^{e2}-S(O)_{q}N(R^{g3})R^{h3}, -A^{f2}-S(O)_{q}ORⁱ³, —N₃, —N(R^{j3})R^{k3}, —N(H)CN, —NO₂, —ONO₂, —OR^{l3}, —SR^{m3} or ═O
each Q^{a2} to Q^{c2} independently represents ═O, ═S, ═NRⁿ³ or ═N(OR^{o3});
each A^{a2} to A^{f2} independently represents a single bond, —N(R^{p3})— or —O—;
each R^{a3} and R^{f3} independently represents C₁₋₆ alkyl, C₂₋₆ alkenyl or C₂₋₆ alkynyl each optionally substituted by one or more groups independently selected from G^{2a}, heterocyclyl optionally substituted by one or more groups independently selected from G^{2b}, aryl optionally substituted by one or more groups independently selected from G^{2c}, or heteroaryl optionally substituted by one or more groups independently selected from G^{2d};
each R^{b3}, R^{c3}, R^{d3}, R^{e3}, R^{g3}, R^{h3}, Rⁱ³, R^{j3}, R^{k3}, R^{l3}, R^{m3}, Rⁿ³, R^{o3} and R^{p3} independently represents H, C₁₋₆ alkyl, C₂₋₆ alkenyl or C₂₋₆ alkynyl each optionally substituted by one or more groups independently selected from G^{2a}, heterocyclyl optionally substituted by one or more groups independently selected from G^{2b}, aryl optionally substituted by one or more groups independently selected from G^{2c}, or heteroaryl optionally substituted by one or more groups independently selected from G^{2d}; or
any two R^{c3} and R^{d3}, R^{g3} and R^{h3} and/or R^{j3} and R^{k3} are linked together to form, along with the nitrogen atom to which they are attached, a 3- to 6-membered ring, which ring optionally contains one further heteroatom and which ring optionally is substituted by one or more groups independently selected from halogen, C₁₋₃ alkyl optionally substituted by one or more halogens, ═O, heterocyclyl optionally substituted by one or more groups independently selected from G^{2b}, aryl optionally substituted by one or more groups independently selected from G^{2c}, or heteroaryl optionally substituted by one or more groups independently selected from G^{2d};
each G^{2a} independently represents halo, —CN, —N(R^{j4})R^{k4}, —OR^{l4}, —SR^{m4} or ═O;
each G^{2b} independently represents halo, R^{a4}, —CN, —N(R^{j4})R^{k4}, —OR^{l4}, —SR^{m4} or ═O;
each G^{2c} and G^{2d} independently represents halo, R^{a4}, —CN, -A^{a3}-C(Q^{a4})R^{b4}, -A^{b3}-C(Q^{b3})N(R^{c4})R^{d4}, -A^{c3}-C(Q^{c3})OR^{e4}, -A^{d3}-S(O)_{q}R^{f4}, -A^{e3}-S(O)_{q}N(R^{g4})R^{h4}, -A^{f3}-S(O)_{q}ORⁱ⁴, —N₃, —N(R^{j4})R^{k4}, —N(H)CN, —NO₂, —ONO₂, —OR^{l4} or —SR^{m4};
each Q^{a3} to Q^{c3} independently represents ═O, ═S, ═NRⁿ⁴ or ═N(OR^{o4});
each A^{a3} to A^{f3} independently represents a single bond, —N(R^{p4})— or —O—;
each R^{a4} and R^{f4} independently represents C₁₋₆ alkyl, C₂₋₆ alkenyl or C₂₋₆ alkynyl each optionally substituted by one or more groups independently selected from G^{3a}, heterocyclyl optionally substituted by one or more groups independently selected from G^{3b}, aryl optionally substituted by one or more groups independently selected from G^{3c}, or heteroaryl optionally substituted by one or more groups independently selected from G^{3d};
each R^{b4}, R^{o4}, R^{d4}, R^{e4}, R^{g4}, R^{h4}, Rⁱ⁴, R^{j4}, R^{k4}, R^{l4}, R^{m4}, Rⁿ⁴, R^{o4} and R^{p4} independently represents H, C₁₋₆ alkyl, C₂₋₆ alkenyl or C₂₋₆ alkynyl each optionally substituted by one or more groups independently selected from G^{3a} or heterocyclyl optionally substituted by one or more groups independently selected from G^{3b}, aryl optionally substituted by one or more groups independently selected from G^{3c}, or heteroaryl optionally substituted by one or more groups independently selected from G^{3d}; or
any of R^{c4} and R^{d4}, R^{g4} and R^{h4} and/or R^{j4} and R^{k4} are linked together to form, together with the nitrogen atom to which they are attached, a 3- to 6-membered ring, which ring optionally contains one further heteroatom and which ring optionally is substituted by one or more groups independently selected G^{3b};
each G^{3a} and G^{3b} independently represents halo, R^{a5}, —CN, —N(R^{b5})R^{c5}, —OR^{d5}, —SR^{e5} or ═O;
each G^{3c} and G^{3d} independently representing halo, R^{a5}, —CN, -A^{a4}-C(Q^{a4})R^{b5}, -A^{b4}-C(Q^{b4})N(R^{c5})R^{d5}, -A^{c4}-C(Q^{c4})OR^{e5}, -A^{d5}-S(O)_{q}R^{f5}, -A^{e4}-S(O)_{q}N(R^{g5})R^{h5}, -A^{f4}-S(O)_{q}ORⁱ⁵, —N₃, —N(R^{j5})R^{k5}, —N(H)CN, —NO₂, —ONO₂, —OR^{l5} or —SR^{m5},
each Q^{a4} to Q^{c4} independently represents ═O, ═S, ═NRⁿ⁵ or ═N(OR^{o5});
each A^{a4} to A^{f4} independently represents a single bond, —N(R^{p5})— or —O—;
with each R^{f5} to R^{p5} independently representing H, or C₁₋₆ alkyl, C₂₋₆ alkenyl or C₂₋₆ alkynyl each optionally substituted by one or more groups independently selected from G⁴, or with each R^{g5} and R^{h5}, and R^{j5} and R^{k5} being linked together to form, together with the nitrogen atom to which they are attached, a 3- to 6-membered ring, which ring optionally contains one further heteroatom and which ring optionally is substituted by one or more groups independently selected from G⁴;
each R^{a5} independently represents C₁₋₆ alkyl, C₂₋₆ alkenyl or C₂₋₆ alkynyl each optionally substituted by one or more groups independently selected from G⁴;
each R^{b5}, R^{c5}, R^{d5} and R^{e5} independently represents H, or C₁₋₆ alkyl, C₂₋₆ alkenyl or C₂₋₆ alkynyl each optionally substituted by one or more groups independently selected from G⁴; or
each R^{b5} and R^{c5} are linked together to form, together with the nitrogen atom to which they are attached, a 3- to 6-membered ring, which ring optionally contains one further heteroatom and which ring optionally is substituted by one or more groups independently selected from G⁴;
each G⁴ independently represents halo, R^{a6}, —CN, —N(R^{b6})R^{c6}, —OR^{d6} or ═O;
each R^{a6} independently represents C₁₋₆ alkyl, C₂₋₆ alkenyl or C₂₋₆ alkynyl each optionally substituted by one or more fluoro;
each R^{b6}, R^{c6} and R^{d6} independently represents H, or C₁₋₆ alkyl, C₂₋₆ alkenyl or C₂₋₆ alkynyl each optionally substituted by one or more fluoro; and
each p and q independently represents 1 or 2.

Formula XI includes compounds of formulae I, IV, V and VI. Methods for preparing such compounds are described elsewhere herein.

Preferred compounds of formula XI may be selected from the group consisting of (or comprising): OT-1000, OT-1011, OT-1012, OT-1096, OT-1113, OT-1129 and OT-1131.

In some preferred embodiments, the SecTRAP forming agent is selected from the group consisting of (or comprising) the compounds OT-1000, OT-1011, OT-1012, OT-1096, OT-1113, OT-1129, OT-1131 and OT-2056.

In preferred embodiments, the SecTRAP forming agent is OT-1000, OT-1129, OT-1096 or OT-2056.

In one preferred embodiment, the SecTRAP forming agent is OT-1000.

In one preferred embodiment, the SecTRAP forming agent is OT-1129.

In one preferred embodiment, the SecTRAP forming agent is OT-1096.

In one preferred embodiment, the SecTRAP forming agent is OT-2056.

In one embodiment, the SecTRAP forming agent is not a compound of formula I.
In one embodiment, the SecTRAP forming agent is not a compound of formula II.
In one embodiment, the SecTRAP forming agent is not a compound of formula III.
In one embodiment, the SecTRAP forming agent is not a compound of formula I, II or III.
In one embodiment, the SecTRAP forming agent is not the compound OT-1000.
In one embodiment, the SecTRAP forming agent is not the compound OT-2056.
In one embodiment, the SecTRAP forming agent is not the compound OT-1000 or OT-2056.
In one embodiment, the SecTRAP forming agent is not a compound of formula IV.
In one embodiment, the SecTRAP forming agent is not a compound of formula V.
In one embodiment, the SecTRAP forming agent is not a compound of formula IV or V.
In one embodiment, the SecTRAP forming agent is not the compound OT-1012.
In one embodiment, the SecTRAP forming agent is not the compound OT-1011.
In one embodiment, the SecTRAP forming agent is not the compound OT-1113.
In one embodiment, the SecTRAP forming agent is not the compound OT-1129.
In one embodiment, the SecTRAP forming agent is not the compound OT-1096.
In one embodiment, the SecTRAP forming agent is not the compound OT-1012, OT-1011, OT-1113, OT-1129 or OT-1096.
In one embodiment, the SecTRAP forming agent is not a compound of formula VIII.
In one embodiment, the SecTRAP forming agent is not a compound of formula IX.
In one embodiment, the SecTRAP forming agent is not the compound OT-1131.
In one embodiment, the SecTRAP forming agent is not a compound of formula VIII or IX.
In one embodiment, the SecTRAP forming agent is not a compound of formula IV or V or VIII or IX.
In one embodiment, the SecTRAP forming agent is not a compound of formula VI.
In one embodiment, the SecTRAP forming agent is not a compound of formula VII.
In one embodiment, the SecTRAP forming agent is not a compound of formula X.
In one embodiment, the SecTRAP forming agent is a compound selected from the group consisting of:

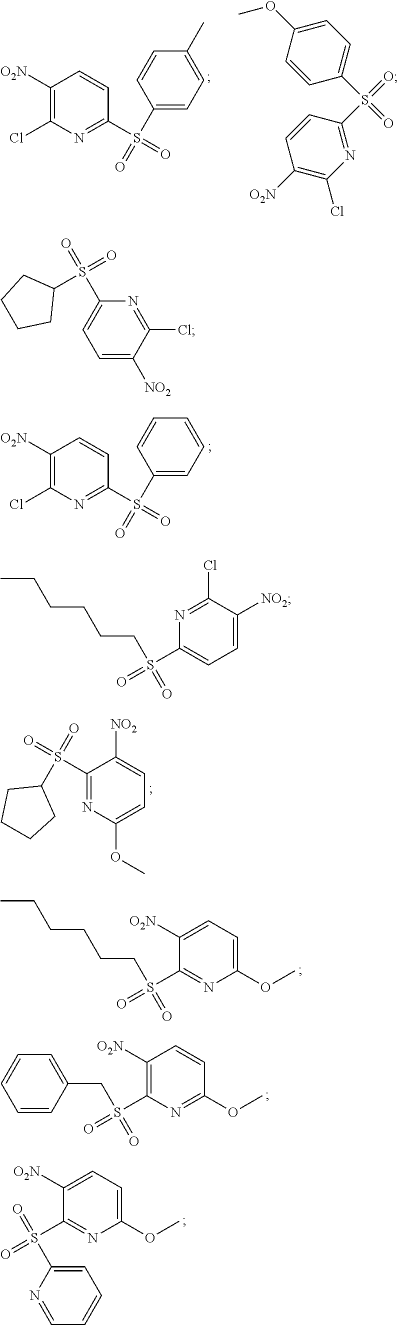

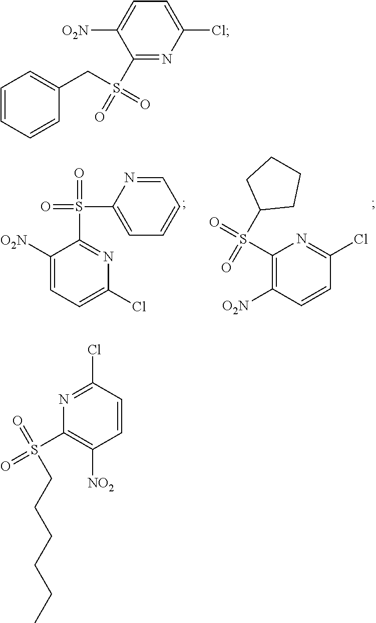

In one embodiment, the SecTRAP forming agent is not a compound selected from the group consisting of the above 13 compounds.

Pharmaceutically-acceptable salts include acid addition salts and base addition salts. Such salts may be formed by conventional means, for example by reaction of a free acid or a free base form of a compound (SecTRAP forming agent) for use in the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound for use in the invention in the form of a salt with another counter-ion, for example using a suitable ion exchange resin.

Particular acid addition salts that may be mentioned include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, hydroxyethanesulphonate, 1- or 2-naphthalene-sulphonate or 1,5-naphthalenedisulphonate salts) or sulphate, pyrosulphate, bisulphate, sulphite, bisulphite, phosphate, monohydrogenphosphate, dihydrogenphosphate, metaphosphate, pyrophosphate or nitrate salts, and the like.

Particular base addition salts that may be mentioned include salts formed with alkali metals (such as Na and K salts), alkaline earth metals (such as Mg and Ca salts), organic bases (such as ethanolamine, diethanolamine, triethanolamine, tromethamine and lysine) and inorganic bases (such as ammonia and aluminium hydroxide). More particularly, base addition salts that may be mentioned include Mg, Ca and, most particularly, K and Na salts.

For the avoidance of doubt, compounds for use in the invention may exist as solids, and thus the scope of the invention includes all amorphous, crystalline and part crystalline forms thereof, and may also exist as oils. Where compounds for use in the invention exist in crystalline and part crystalline forms, such forms may include solvates, which are included in the scope of the invention. Compounds for use in the invention may also exist in solution.

Compounds for use in the invention may contain double bonds and may thus exist as E (entgegen) and Z (zusammen) geometric isomers about each individual double bond. All such isomers and mixtures thereof are included within the scope of the invention.

Compounds for use in the invention may also exhibit tautomerism. All tautomeric forms and mixtures thereof are included within the scope of the invention.

Compounds for use in the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution); for example, with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

As used herein, references to halo and/or halogen will independently refer to fluoro, chloro, bromo and iodo (for example, fluoro and chloro).

Unless otherwise specified, C_{1-z} alkyl groups (where z is the upper limit of the range) defined herein may be straight-chain or, when there is a sufficient number (i.e. a minimum of two or three, as appropriate) of carbon atoms, be branched-chain, and/or cyclic (so forming a C_{3-z}-cycloalkyl group). When there is a sufficient number (i.e. a minimum of four) of carbon atoms, such groups may also be part cyclic. Part cyclic alkyl groups that may be mentioned include cyclopropylmethyl and cyclohexylethyl. When there is a sufficient number of carbon atoms, such groups may also be multicyclic (e.g. bicyclic or tricyclic) or spirocyclic. Such alkyl groups may also be saturated or, when there is a sufficient number (i.e. a minimum of two) of carbon atoms, be unsaturated (forming, for example, a C_{2-z} alkenyl or a C_{2-z} alkynyl group).

Unless otherwise specified, C_{1-z} alkylene groups (where z is the upper limit of the range) defined herein may (in a similar manner to the definition of C_{1-z} alkyl) be straight-chain or, when there is a sufficient number (i.e. a minimum of two or three, as appropriate) of carbon atoms, be branched-chain, and/or cyclic (so forming a C₃₋ₜ-cycloalkylene group). When there is a sufficient number (i.e. a minimum of four) of carbon atoms, such groups may also be part cyclic. When there is a sufficient number of carbon atoms, such groups may also be multicyclic (e.g. bicyclic or tricyclic) or spirocyclic. Such alkylene groups may also be saturated or, when there is a sufficient number (i.e. a minimum of two) of carbon atoms, be unsaturated (forming, for example, a C_{2-z} alkenylene or a C_{2-z} alkynylene group). Particular alkylene groups that may be mentioned include those that are straight-chained or cyclic and saturated.

Unless otherwise specified, C_{2-z} alkynyl groups (where z is the upper limit of the range) defined herein may be straight-chain or, when there is a sufficient number (i.e. a minimum of four) of carbon atoms, be branched-chain.

For the avoidance of doubt, the skilled person will understand that the term alkyl will refer to saturated hydrocarbon moieties, whereas the term alkenyl will refer to unsaturated hydrocarbon moieties containing at least one carbon-carbon double bond and the term alkynyl will refer to unsaturated hydrocarbon moieties containing at least one carbon-carbon triple bond, which alkyl, alkenyl and alkynyl groups may be referred to collectively as hydrocarbyl groups. Further, such unsaturated hydrocarbon moieties will be referred to by reference to the highest degree of unsaturation comprised therein (e.g. a hydrocarbon moiety comprising at least one carbon-carbon double bond and at least one carbon-carbon triple bond will be referred to as alkynyl, although such moieties may also be referred to using terms such as “alkenyl alkynyl” and the like).

As used herein, the term heterocycloalkyl may refer to non-aromatic monocyclic and bicyclic heterocycloalkyl groups (which groups may further be bridged) in which at least one (e.g. one to four) of the atoms in the ring system is other than carbon (i.e. a heteroatom), and in which the total number of atoms in the ring system is between three and twelve (e.g. between five and ten and, most preferably, between three and eight, e.g. a 5- or 6-membered heterocycloalkyl group). Further, such heterocycloalkyl groups may be saturated or unsaturated containing one or more double and/or triple bonds, forming for example a C_{2-z} (e.g. C_{4-z}) heterocycloalkenyl (where z is the upper limit of the range) or a C_{7-z} heterocycloalkynyl group. C_{2-z} heterocycloalkyl groups that may be mentioned include 7-azabicyclo-[2.2.1]heptanyl, 6-azabicyclo[3.1.1]heptanyl, 6-azabicyclo[3.2.1]-octanyl, 8-azabicyclo[3.2.1]octanyl, aziridinyl, azetidinyl, 2,3-dihydroisothiazolyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, isothiazolidinyl, morpholinyl, 7-oxabicyclo[2.2.1]heptanyl, 6-oxabicyclo[3.2.1]-octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulpholanyl, 3-sulpholenyl, tetrahydropyranyl, tetrahydrofuryl, tetrahydropyridyl (such as 1,2,3,4-tetrahydropyridyl and 1,2,3,6-tetrahydropyridyl), thietanyl, thiiranyl, thiolanyl, tetrahydrothiopyranyl, thiomorpholinyl, trithianyl (including 1,3,5-trithianyl), tropanyl and the like. Substituents on heterocycloalkyl groups may, where appropriate, be located on any atom in the ring system including a heteroatom. Further, in the case where the substituent is another cyclic compound, then the cyclic compound may be attached through a single atom on the heterocycloalkyl group, forming a so-called “spiro”-compound. The point of attachment of heterocycloalkyl groups may be via any atom in the ring system including (where appropriate) a further heteroatom (such as a nitrogen atom), or an atom on any fused carbocyclic ring that may be present as part of the ring system. Heterocycloalkyl groups may also be in the N- or S-oxidised form.

At each occurrence when mentioned herein, particular heterocycloalkyl groups that may be mentioned include 3- to 8-membered heterocycloalkyl groups (e.g. a 4- to 6-membered heterocycloalkyl group).

As may be used herein, the term aryl includes references to C₆₋₁₄ (e.g. C₆₋₁₀) aromatic groups. Such groups may be monocyclic or bicyclic and, when bicyclic, be either wholly or partly aromatic. C₆₋₁₀ aryl groups that may be mentioned include phenyl, naphthyl, 1,2,3,4-tetrahydronaphthyl, indanyl, and the like (e.g. phenyl, naphthyl and the like, such as phenyl). For the avoidance of doubt, the point of attachment of substituents on aryl groups may be via any carbon atom of the ring system.

As may be used herein, the term heteroaryl (or heteroaromatic) includes references to 5- to 14- (e.g. 5- to 10-) membered heteroaromatic groups containing one or more heteroatoms selected from oxygen, nitrogen and/or sulphur. Such heteroaryl groups may comprise one, two, or three rings, of which at least one is aromatic. Substituents on heteroaryl/heteroaromatic groups may, where appropriate, be located on any atom in the ring system including a heteroatom. The point of attachment of heteroaryl/heteroaromatic groups may be via any atom in the ring system including (where appropriate) a heteroatom. Bicyclic heteroaryl/heteroaromatic groups may comprise a benzene ring fused to one or more further aromatic or non-aromatic heterocyclic rings, in which instances, the point of attachment of the polycyclic heteroaryl/heteroaromatic group may be via any ring including the benzene ring or the heteroaryl/heteroaromatic or heterocycloalkyl ring. Examples of heteroaryl/heteroaromatic groups that may be mentioned include pyridinyl, pyrrolyl, furanyl, thiophenyl, oxadiazolyl, thiadiazolyl, thiazolyl, oxazolyl, pyrazolyl, triazolyl, tetrazolyl, isoxazolyl, isothiazolyl, imidazolyl, imidazopyrimidinyl, imidazothiazolyl, thienothiophenyl, pyrimidinyl, furopyridinyl, indolyl, azaindolyl, pyrazinyl, pyrazolopyrimidinyl, indazolyl, pyrimidinyl, quinolinyl, isoquinolinyl, quinazolinyl, benzofuranyl, benzothiophenyl, benzoimidazolyl, benzoxazolyl, benzothiazolyl, benzotriazolyl and purinyl. The oxides of heteroaryl/heteroaromatic groups are also embraced within the scope of the invention (e.g. the N-oxide). As stated above, heteroaryl includes polycyclic (e.g. bicyclic) groups in which one ring is aromatic (and the other may or may not be aromatic). Hence, other heteroaryl groups that may be mentioned include e.g. benzo[1,3]dioxolyl, benzo[1,4]dioxinyl, dihydrobenzo[c/]isothiazole, 3,4-dihydrobenz[1,4]oxazinyl, dihydrobenzothiophenyl, indolinyl, 5H,6H,7H-pyrrolo[1,2-b]pyrimidinyl, 1,2,3,4-tetrahydroquinolinyl, thiochromanyl and the like.

For the avoidance of doubt, as used herein, references to heteroatoms will take their normal meaning as understood by one skilled in the art. Particular heteroatoms that may be mentioned include phosphorus, selenium, tellurium, silicon, boron, oxygen, nitrogen and sulphur (e.g. oxygen, nitrogen and sulphur).

For the avoidance of doubt, references to polycyclic (e.g. bicyclic) groups (e.g. when employed in the context of heterocycloalkyl groups) will refer to ring systems wherein more than two scissions would be required to convert such rings into a straight chain, with the minimum number of such scissions corresponding to the number of rings defined (e.g. the term bicyclic may indicate that a minimum of two scissions would be required to convert the rings into a straight chain). For the avoidance of doubt, the term bicyclic (e.g. when employed in the context of heterocycloalkyl groups) may refer to groups in which the second ring of a two-ring system is formed between two adjacent atoms of the first ring, and may also refer to groups in which two non-adjacent atoms are linked by either an alkylene or heteroalkylene chain (as appropriate), which later groups may be referred to as bridged.

For the avoidance of doubt, when an aryl or an heteroaryl group is substituted with a group via a double bond, such as ═O, it is understood that the aryl or heteroaryl group is partly aromatic, i.e. the aryl or heteroaryl group consists of at least two rings where at least one ring is not aromatic.

As used herein, the term heterocyclyl may refer to non-aromatic monocyclic and bicyclic heterocyclyl groups (which groups may further be bridged) in which at least one (e.g. one to four) of the atoms in the ring system is other than carbon (i.e. a heteroatom), and in which the total number of atoms in the ring system is between three and twelve (e.g. between five and ten and, most preferably, between three and eight, e.g. a 5- or 6-membered heterocyclyl group). Further, such heterocyclyl groups may be saturated, forming a heterocycloalkyl, or unsaturated containing one or more carbon-carbon or, where possible, carbon-heteroatom or heteroatom-heteroatom double and/or triple bonds, forming for example a C_{2-z} (e.g. C_{4-z}) heterocycloalkenyl (where z is the upper limit of the range) or a C_{7-z} heterocycloalkynyl group. C_{2-z} heterocyclyl groups that may be mentioned include 7-azabicyclo-[2.2.1]heptanyl, 6-azabicyclo[3.1.1]heptanyl, 6-azabicyclo[3.2.1]-octanyl, 8-azabicyclo[3.2.1]octanyl, aziridinyl, azetidinyl, 2,3-dihydroisothiazolyl, dihydropyranyl, dihydropyridinyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, isothiazolidinyl, morpholinyl, 7-oxabicyclo[2.2.1]heptanyl, 6-oxabicyclo[3.2.1]-octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuryl, tetrahydropyridinyl (such as 1,2,3,4-tetrahydropyridinyl and 1,2,3,6-tetrahydropyridinyl), thietanyl, thiiranyl, thiolanyl, tetrahydrothiopyranyl, thiomorpholinyl, trithianyl (including 1,3,5-trithianyl), tropanyl and the like. Substituents on heterocyclyl groups may, where appropriate, be located on any atom in the ring system including a heteroatom. Further, in the case where the substituent is another cyclic compound, then the cyclic compound may be attached through a single atom on the heterocyclyl group, forming a so-called “spiro”-compound. The point of attachment of heterocyclyl groups may be via any atom in the ring system including (where appropriate) a further heteroatom (such as a nitrogen atom), or an atom on any fused carbocyclic ring that may be present as part of the ring system. Heterocyclyl groups may also be in the N- or S-oxidised form.

At each occurrence when mentioned herein, particular heterocyclyl groups that may be mentioned include 3- to 8-membered heterocyclyl groups (e.g. a 4- to 6-membered heterocyclyl group).

The present invention also embraces isotopically-labelled compounds for use in the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Hence, the compounds of the invention also include deuterated compounds, i.e. in which one or more hydrogen atoms are replaced by the hydrogen isotope deuterium.

For the avoidance of doubt, in cases in which the identity of two or more substituents in a compound of the invention may be the same, the actual identities of the respective substituents are not in any way interdependent. For example, in the situation in which two or more R⁴ groups are present, those R⁴ groups may be the same or different. Similarly, where two or more R⁴ groups are present and each represent R^{a2}, the R^{2a} groups in question may be the same or different. Likewise, when more than one R^{a1} is present and each independently represents C₁₋₆ alkyl substituted by one or more G^{1a} group, the identities of each G^{1a} are in no way interdependent.

For the avoidance of doubt, when a term such as “A^{a1} to A^{f1}” is employed herein, this will be understood by the skilled person to mean A^{a1}, A^{b1}, A^{c1}, A^{d1}, A^{e1} and A^{f1} inclusively. Unless otherwise stated, the same reasoning will apply to other such terms used herein.

Other agents that may be used as SecTRAP forming agents in accordance with the present invention include those in the following Table:

| | |
|---|---|
| Auranofin | (Eriksson 2011), |
| | (Cheng et al. 2010), |
| | (Sachweh et al. 2015) |
| MJ25 ((2-{[2-(1,3-benzothiazol-2- | (Sachweh et al. 2015) |
| ylsulfonyl)ethyl] thio}-1,3-benzoxazole)) | |
| Certain Indolin-2-one compounds | (Kaminska et al. 2016) |
| | (compounds 4 and 5) |
| Iniparib (4-Iodo-3-nitrobenzamide) | |
| DNCB (1-chloro-2,4-dinitrobenzene) | (Arnér et al. 1995), |
| | (Nordberg et al. 1998) |
| Juglone (5-hydroxy-1,4-naphthoquinone) | (Cenas et al. 2004), |
| | (Cheng et al. 2010) |
| DNFB (1-fluoro-2,4-dinitrobenzene) | (Nordberg et al. 1998) |
| Curcumin (Diferuloylmethane) | (Fang et al. 2005) |
| Mechlorethamine (analog of 1,4- | (Jan etal. 2014) |
| naphthoquinone) | |
| Cisplatin | (Anestål & Arnér 2003), |
| | (Witte et al. 2005) |
| Shikonin (5,8-dihydroxy-2-(1-hydroxy-4- | (Duan et al. 2014) |
| methylpent-3-enyl)naphthalene-1,4-dione) | |
| Parthenolide | (Duan et al. 2016) |

Other agents that may be used as SecTRAP forming agents in accordance with the present invention may include those in the following Table:

| | |
|---|---|
| ATO (arsenic trioxide)* | (Anestål et al. 2008), |
| | (Lu et al. 2007) |
| Cyclophosphamide | (Wang et al. 2007) |
| Oxaliplatin | (Witte et al. 2005) |
| Protoporphyrin IX | (Stafford 2015) |
| b-AP15 | (Stafford 2015) |
| HNE (4-hydroxy-2-nonenal) | (Arnér 2009) |
| Benzenesulfonyl-6F-indole-substituted quinol | (Chew et al. 2008) |
| Mitomycin C | (Paz et al. 2012) |
| lodoacetamide (IAA) | (Nordberg et al. 1998) |
| 4-VP (4-vinylpyridine) | (Nordberg et al. 1998) |
| Gold thioglucose | (Anestål et al. 2008), |
| | (Anestål & Arnér 2003) |
| BCNU (1,3-bis-(2-chloro-ethyl)-1-nitrosourea) | (Saccoccia et al. 2014) |
| 2,4-DHBA (2,4-Dihydroxybenzylamine) | (Saccoccia et al. 2014) |
| Carmustine (N,N′-Bis(2-chloroethyl)-N- | (Witte et al. 2005) |
| nitrosourea) | |
| Chlorambucil (N,N-Di-2-chloroethyl-gamma-P- | (Witte et al. 2005) |
| aminophenylbutyric acid) | |
| Melphalan (4-(Bis(2-chloroethyl)amino)-L- | (Witte et al. 2005) |
| phenylalanine) | |
| DNFB (1-fluoro-2,4-dinitrobenzen) | (Arnér 2009) |
| DNBB (1-bromo-2,4-dinitrobenzene) | (Arnér 2009) |
| [Au(d2pype)2]Cl | (Rackham et al. 2011) |
| RITA (NSC 652287, 2,5-bis(5-hydroxymethyl- | (Hedström et al. 2009) |
| 2-thienyl)furan) | |
| Isofosfamide | (Arnér 2009) |
| Acrolein (1-Propen-3-one) | (Arnér 2009) |
| Myricetin (3,5,7-trihydroxy-2-(3,4,5- | (Arnér 2009) |
| trihydroxyphenyl)-4H-1-benzopyran-4-one) | |
| Quercetin (3,3′,4′,5,7-pentahydroxyflavone) | (Arnér 2009) |
| 4,6-dinitrobenzofuroxan | (Arnér 2009) |
| Monomethylarsonous acid | (Arnér 2009) |
| Gambogic acid | (Kaminska et al. 2016) |
| PMX464 (4-(1,3-benzothiazol-2-yl)-4- | (Kaminska et al. 2016) |
| hydroxycyclohexa-2,5-dien-1-one) | |
| BCA (2-benzoyloxycinnamaldehyde) | (Kaminska et al. 2016) |
| methylene quinuclidinone (MQ). MQ may also | (Peng et al. 2013) |
| be referred to as 2-methylene-3- | |
| quinuclidinone. | |
| MQ may be provided in the form of the | |
| prodrug APR-246 (PubChem ID: 52918385) | |
| (2-hydroxymethyl-2- | |
| methoxymethylazabicyclo(2.2.2)octan-3-one) | |

In some embodiments, the SecTRAP forming agent for use in accordance with the invention is methylene quinuclidinone (MQ). In some embodiments, MQ may be provided in the form of its prodrug, APR-246. MQ is a conversion product of APR-246. APR-246 is available from Aprea AB (Stockholm, Sweden). In some embodiments, heat treated APR-246 may be used (e.g. heat treated at 90° C. for 15 mins) as heat treatment of APR-246 can generate MQ.

In some preferred embodiments, the compound for use in the present invention is selected from the group consisting of (or comprising) OT-1000, QT-1011, OT-1012, OT-1096, OT-1113, OT-1129, OT-1131, OT-2056, Auranofin and Iniparib.

In some preferred embodiments, the compound for use in the present invention is selected from the group consisting of (or comprising) OT-1000, OT-1011, OT-1012, OT-1096, OT-1113, OT-1129, OT-1131 and OT-2056.

In some preferred embodiments, the compound for use in the present invention is selected from the group consisting of (or comprising) OT-1011, OT-1012, OT-1096, OT-1113, OT-1129 and OT-1131.

In some preferred embodiments, the compound for use in the present invention is selected from the group consisting of OT-1000, OT-1129, Auranofin or Iniparib.

In some preferred embodiments, the compound for use in the present invention is selected from the group consisting of (or comprising) OT-1000 and OT-1129.

In some particularly preferred embodiments, the compound for use in the present invention is OT-1096.

In some preferred embodiments, the compound for use in the present invention is selected from the group consisting of Auranofin or Iniparib.

In some preferred embodiments, the compound for use in the present invention is Auranofin.

In some preferred embodiments, the compound for use in the present invention is Iniparib.

In some preferred embodiments, the compound for use in the present invention is not Iniparib.

In some preferred embodiments, the compound for use in the present invention is not cisplatin.

In some preferred embodiments, the compound for use in the present invention is not arsenic trioxide (also referred to as ATO).

In some preferred embodiments, the compound for use in the present invention is not Auranofin.

In some preferred embodiments, the compound for use in the present invention is not cisplatin, arsenic trioxide, Auranofin or Iniparib.

In some embodiments, a single (i.e. one) SecTRAP forming agent is used for the treatment of cancer (e.g. a T-cell infiltrated cancer). However, in some embodiments, more than one SecTRAP forming agents (e.g. 2, 3, 4 or 5 different SecTRAP forming agents) are used for the treatment of cancer (e.g. a T-cell infiltrated cancer). In some embodiments, two different SecTRAP forming agents are used. In some embodiments where more than one SecTRAP forming agents are used, preferably at least one of the SecTRAP forming agents is a compound of Formula I, II, III, IV, V, VI, VII, VIII, IX, X or XI. In some embodiments, where more than one SecTRAP forming agents are used, preferably two or more of the SecTRAP forming agents are compounds of Formula I, II, III, IV, V, VI, VII, VIII, IX, X or XI. In some embodiments, where more than one SecTRAP forming agents are used, preferably at least one of the SecTRAP forming agents is a compound of Formula I, II III, IV, V, VI, VII, VIII, IX, X or XI. In some embodiments where more than one SecTRAP forming agent is used, one of the SecTRAP forming agents is OT-1096. In some embodiments, where more than one SecTRAP forming agent is used, one of the SecTRAP forming agents is Iniparib. In some embodiments, where more than one SecTRAP forming agents are used, at least one of the SecTRAP forming agents is a compound of Formula I, II, III, IV, V, VI, VII, VIII, IX, X or XI and one of the compounds is Iniparib. In some embodiments, where more than one SecTRAP forming agents are used, one of the SecTRAP forming agents is OT-1096 and one of the SecTRAP forming agents is Iniparib. In some embodiments, where two SecTRAP forming agents are used, one of the SecTRAP forming agents is OT-1096 and the other is Iniparib. Where either a single SecTRAP forming agent or multiple different (e.g. 2, 3, 4 or 5) SecTRAP forming agents are used, the treatment of cancer (or therapeutic regimen) may, in some embodiments, additionally comprise the use (or administration) of one or more further, non-SecTRAP forming, agents.

The skilled person will appreciate that compounds of the invention that are the subject of this invention include those that are stable. That is, compounds of the invention include those that are sufficiently robust to survive isolation, e.g. from a reaction mixture, to a useful degree of purity.

Compounds for use in the invention as described herein (e.g. compounds of formulae I to XI) may be prepared in accordance with techniques that are well known to those skilled in the art, such as those described hereinafter.

### Preparation of Compounds of Formula I

A suitable process for the preparation of a compound of formula I as hereinbefore defined may comprise:

(i) reaction of a compound of formula IA

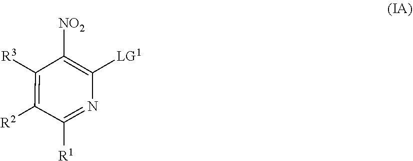

wherein R¹, R² and R³ are as defined herein in formula I (or any particular feature or embodiment thereof) and LG¹ represents a suitable leaving group (such as halo, e.g. chloro), with a compound of formula IB

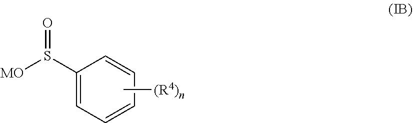

wherein R⁴ and n are as defined herein in formula I (or any particular feature or embodiments thereof) and M represents an alkali metal ion (such as a Na ion), in the presence of a suitable acid (such as a concentrated acid, e.g. a concentrated mineral acid, for example concentrated HCl, e.g. concentrated aqueous HCl) and in the presence of a suitable solvent (such as a polar organic solvent, e.g. N,N′-dimethylacetamide, N,N′-dimethylformamide or tetrahydrofuran), and optionally in the presence of a suitable phase transfer catalyst (such as a quaternary ammonium salt, e.g. tetra-butyl ammonium chloride);
(ii) reaction of a compound of formula IC (particularly where at least one R⁴ is present and represents an electron-withdrawing group, such as —NO₂)

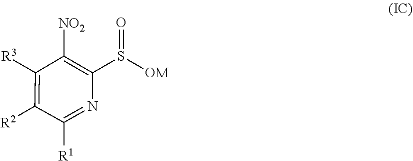

wherein R¹, R² and R³ are as defined herein in formula I (or any particular feature or embodiment thereof) and M represents an alkali metal ion (such as a Na ion), with a compound of formula ID

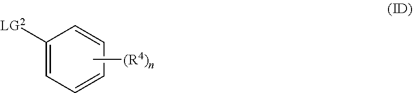

wherein R⁴ and n are as defined herein in formula I (or any particular feature or embodiments thereof) and LG² represents a suitable leaving group (such as halo, e.g. chloro), in the presence of a suitable acid (such as a concentrated acid, e.g. a concentrated mineral acid, for example concentrated HCl, e.g. concentrated aqueous HCl) and in the presence of a suitable solvent (such as a polar organic solvent, e.g. N,N′-dimethylacetamide, N,N′-dimethylformamide or tetrahydrofuran), and optionally in the presence of a suitable phase transfer catalyst (such as a quaternary ammonium salt, e.g. tetra-butyl ammonium chloride);
(iii) reaction of a compound of formula IA as hereinbefore defined with a compound of formula IB as hereinbefore defined, in the presence of a suitable metal halide (such as a suitable metal iodide, e.g. CuI, or a suitable metal bromide, e.g. CuBr; which metal halide may be present in excess, such as in amount corresponding to at least 2 molar equivalents of the compound of formula IA and/or the compound of formula IB) and in the presence of a suitable solvent (such as a polar organic solvent, e.g. N,N′-dimethylacetamide, N,N′-dimethylformamide, tetrahydrofuran or 3-dimethyl-2-imidazolidinone), under conditions known to those skilled in the art;
(iv) reaction of a compound of formula IC as hereinbefore defined (particularly where at least one R⁴ is present and represents an electron-withdrawing group, such as —NO₂) with a compound of formula ID as hereinbefore defined, in the presence of a suitable metal halide (such as a suitable metal iodide, e.g. CuI, or a suitable metal bromide, e.g. CuBr; which metal halide may be present in excess, such as in amount corresponding to at least 2 molar equivalents of the compound of formula IC and/or the compound of formula ID) and in the presence of a suitable solvent (such as a polar organic solvent, e.g. N,N′-dimethylacetamide, N,N′-dimethylformamide, tetrahydrofuran or 3-dimethyl-2-imidazolidinone), under conditions known to those skilled in the art;
(v) reaction of a compound of formula IE

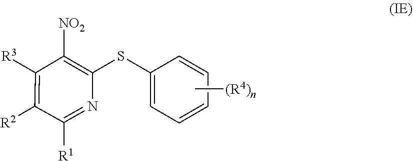

wherein R¹ to R⁴ and n are as defined herein in formula I (or any particular feature or embodiments thereof), with a suitable oxidising agent (such as a hypochlorite salt, e.g. sodium hypochlorite, a peroxymonosuIphate salt, e.g. potassium peroxymonosuIphate (Oxone), a percarboxylic acid, e.g. meta-chloroperoxybenzoic acid (mCPBA), or potassium permanganate) in the presence of a suitable solvent (such as a polar organic solvent, e.g. N,N′-dimethylacetamide, N,N′-dimethylformamide or terahydrofuran), and optionally in the presence of water, under conditions known to those skilled in the art;
(vi) reaction of a compound of formula IF

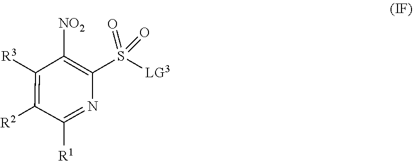

wherein R¹, R² and R³ are as defined herein in formula I (or any particular feature or embodiment thereof) and LG³ represents a suitable leaving group (such as halo, e.g. chloro) with a compound of formula IG

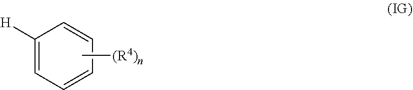

wherein R⁴ and n are as defined herein in formula I (or any particular feature or embodiments thereof; particularly where one or more R⁴ is present and represents an electron donating group, such as an alkyl group), in the presence of a suitable Lewis acid (such as AlCl₃) and in the presence of a suitable solvent (such as an organic solvent, e.g. dichloromethane or dichloroethane);
(vii) reaction of a compound of formula IF as defined herein with a compound of formula IG as defined herein (for example, where one or more R⁴ is present in the ortho position and represents suitable directing group), in the presence of a suitable catalyst (such as palladium(II) acetate) and a suitable base (such as a alkali metal carbonate, e.g. potassium carbonate), and in the presence of a suitable solvent (such as an organic solvent, e.g. dichloromethane);
(viii) reaction of a compound of formula IF as defined herein with a compound of formula IH

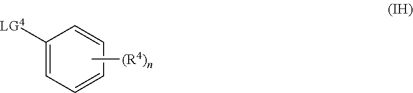

wherein R⁴ and n are as defined herein in formula I (or any particular feature or embodiments thereof) and LG⁴ represents a suitable leaving group (such as a boronic acid), in the presence of a suitable catalyst (such as a suitable metal halide, e.g. CuBr, or phenanthroline) and in the presence of a suitable solvent (such as an organic solvent, e.g. dichloromethane or dichloroethane);
(ix) reaction of a compound of formula IC as defined herein with (a) a compound of formula IG as defined herein having at least one R⁴ group, or (b) a compound of formula IG as defined herein but having a group that may be converted to an R⁴ group, wherein the R⁴ group or group that may be converted to an R⁴ group is present ortho to the essential H substituent and represents a suitable directing group (such as a suitable amide, e.g. —C(O)N(H)C(CH₃)₂-2-pyridinyl), in the presence of a suitable catalyst and/or oxidant (such as copper(II) acetate and/or silver carbonate), and in the presence of a suitable solvent (such as an organic solvent, e.g. dichloroethane), which step may further comprise conversion of the group that may be converted to an R⁴ group to the required R⁴ group, under conditions known to those skilled in the art.

Compounds of formulae IA, IC, IB, ID, IE, IF, IG and IH are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.

In particular, compounds of formula IE may be prepared by reaction of a compound of formula IJ

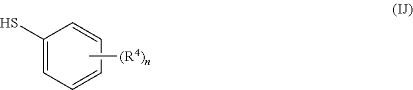

wherein R⁴ and n are as defined herein in formula I (or any particular feature or embodiments thereof), with a compound of formula IA as herein before defined, under conditions known to those skilled in the art, such as in the presence of a suitable base (such as a metal carbonate, e.g. potassium carbonate, a metal hydroxide, e.g. sodium hydroxide, or an amine base, e.g. triethyl amine), and in the presence of a suitable solvent (such as a polar organic solvent, e.g. N,N′-dimethylacetamide, N,N′-dimethylformamide or tetrahydrofuran, or a mixture of a polar organic solvent and water), under conditions known to those skilled in the art.

Similarly, compounds of formula IE (particularly where at least one R⁴ is present and represents an electron-withdrawing group, such as —NO₂) may be prepared by reaction of a compound of formula IK

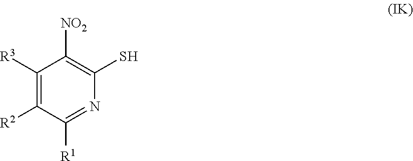

wherein R¹, R² and R³ are as defined herein in formula I (or any particular feature or embodiments thereof), with a compound of formula ID as described herein, under conditions known to those skilled in the art (for example, where the R⁴ groups present in the compound of formula ID are not sufficiently electron withdrawing, the reaction may be performed in the presence of a suitable catalyst, such as palladium(II) acetate or copper oxide, in which case the suitable base may be an alkali metal tert-butoxide, such as Kt-OBu).

Similarly, compounds of formulae IJ and IK are either commercially available, are known in the literature, or may be obtained either by analogy with the processes described herein, or by conventional synthetic procedures, in accordance with standard techniques, from available starting materials using appropriate reagents and reaction conditions.

The substituents R¹ to R⁴, as hereinbefore defined, may be modified one or more times, after or during the processes described above for preparation of compounds of formula I by way of methods that are well known to those skilled in the art. Examples of such methods include substitutions, reductions, oxidations, dehydrogenations, alkylations, dealkylations, acylations, hydrolyses, esterifications, etherifications, halogenations and nitrations. The precursor groups can be changed to a different such group, or to the groups defined in formula I, at any time during the reaction sequence. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.

Compounds of the invention may be isolated from their reaction mixtures and, if necessary, purified using conventional techniques as known to those skilled in the art. Thus, processes for preparation of compounds of the invention as described herein may include, as a final step, isolation and optionally purification of the compound of the invention (e.g. isolation and optionally purification of the compound of formula I).

It will be appreciated by those skilled in the art that, in the processes described above and hereinafter, the functional groups of intermediate compounds may need to be protected by protecting groups. The protection and deprotection of functional groups may take place before or after a reaction in the above-mentioned schemes.

Protecting groups may be applied and removed in accordance with techniques that are well known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3rd edition, T. W. Greene & P. G. M. Wutz, Wiley-Interscience (1999).

### Preparation of Compounds of Formula II

A suitable process for the preparation of a compound of formula II as hereinbefore defined may comprise:

(i) reaction of a compound of formula IIA

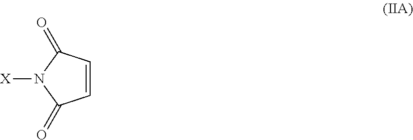

wherein X is as defined herein in formula II (or any particular feature or embodiments thereof), with a compound of formula IIB

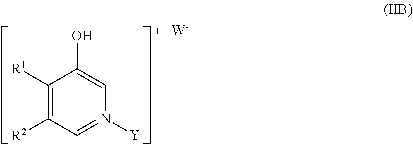

wherein R¹, R² and Y are as defined herein in formula II (or any particular feature or embodiments thereof), in the presence of a suitable solvent (such as an organic solvent, e.g. tetrahydrofuran or toluene) and (in certain instances, optionally) in the presence of a suitable base (e.g. triethylamine or K₂CO₃) (W⁻ represents a counterion in the form of an anion);
(ii) reaction of a compound of formula IIA as defined herein with a compound of formula IIC

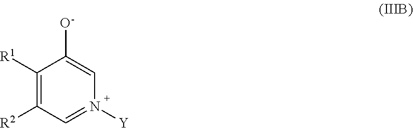

wherein R¹, R² and Y are as defined herein in formula II (or any particular feature or embodiments thereof), in the presence of a suitable solvent (such as an organic solvent, e.g. tetrahydrofuran or toluene);
(iii) reaction of a compound of formula IIA as defined herein with a compound of formula IID

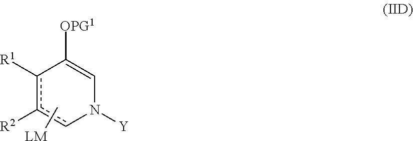

wherein R¹, R² and Y are as defined herein in formula II (or any particular feature or embodiments thereof; particularly where R¹ and R² are H), PG¹ is a suitable protecting group (such as a C₁₋₆ alkyl, e.g. methyl) and LM is a suitable metal complex (such as molybdenum(hydridotris(1-pyrazolyl)borate)(CO)₂), in the presence of a suitable catalyst (such as a Lewis acid catalyst, e.g. EtAlCl₂) and a suitable solvent (such as an organic solvent, e.g. dichloromethane, tetrahydrofuran or toluene), followed by treatment with a suitable oxidizing agent (such as ceric ammonium nitrate) in a suitable solvent (e.g. a mixture of an organic solvent (e.g. tetrahydrofuran) and water);
(iv) where Z represents NR^{a}, reaction of a compound of formula II wherein Z represents O with a compound of formula IIE

HN—R^{a} (IIE)

where R^{a} is as defined herein in formula I (or any particular feature or embodiments thereof), in the presence of a suitable solvent (such as an organic solvent, e.g. toluene) and optionally under conditions suitable for the removal of water (such as in the presence of molecular sieves (e.g. 4 Å molecular sieves) or using Dean-Stark apparatus);
(v) where Z represents NOR^{b}, reaction of a compound of formula II wherein Z represents O with a compound of formula IIF

HN—OR^{b} (IIF)

or a suitable salt thereof (e.g. a HCl or H₂SO₄ salt), where R^{b} is as defined herein in formula II (or any particular feature or embodiments thereof), in the presence of a suitable solvent (such as an organic solvent, e.g. toluene) and in the presence of a suitable base (such as sodium hydroxide or sodium acetate);
(vi) where Z represents S, reaction of a compound of formula II wherein Z represents O, with a suitable reagent (i.e. a reagent suitable for forming a thiocarbonyl, such as Lawesson's reagent) and in the presence of a suitable solvent (such as an organic solvent, e.g. toluene or pyridine); or
(vii) reaction of a compound corresponding to a compound of formula II but wherein Y represents H with a compound of formula IIG

Y-LG² (IIG)

wherein Y is as defined herein in formula I (or any particular feature or embodiments thereof) and LG² is a suitable leaving group (for example, when Y is alkyl, a chloro or bromo, or when Y is aromatic, a bromo or, particularly, an iodo or a boronic acid or ester), in the presence of a suitable solvent (such as an organic solvent, e.g. tetrahydrofuran or dichloromethane) and (in certain instances, optionally) a suitable base (and, in certain instances, optionally in the presence of a suitable catalyst (such as Cu(OAC)₂)), under conditions known to those skilled in the art.

Compounds of formulae IIA, IIB, IIC, IID, IIE, IIF and IIG are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.

For example, compounds of formula IIA may be prepared by:
(a) reaction of a compound of formula IIH

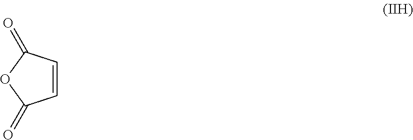

with a compound of formula IIJ

X—NH₂ (IIJ)

wherein X is as defined herein in formula II (or any particular feature or embodiments thereof), in an appropriate solvent system (e.g. tetrahydrofuran or toluene), followed by treatment with, for example,
(a) acetic anhydride, optionally in the presence of a base (e.g. triethylamine or sodium acetate),
(b) acetyl chloride, oxalyl chloride and the like, followed by treatment with a suitable base (e.g. triethylamine), or
(c) hexamethyldisilane and ZnBr₂,
under conditions known to those skilled in the art; or
(b) reaction of a compound of formula IIK

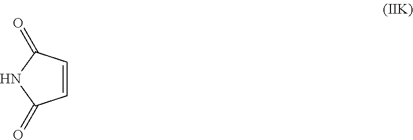

with a compound of formula IIL

X-LG³ (IIL)

wherein X is as defined herein in formula I (or any particular feature or embodiments thereof) and LG³ is a suitable leaving group (for example, when X is alkyl, a chloro or bromo, or X is aromatic, an iodo or a boronic acid or ester), in the presence of a suitable solvent (such as an organic solvent, e.g. tetrahydrofuran or dichloromethane) and a suitable base, and optionally (e.g. when X is aromatic) in the presence of a suitable catalyst (such as Cu(OAc)₂), under conditions known to those skilled in the art.

Further, compounds of formula IIB may be prepared by reaction of a compound of formula IIM

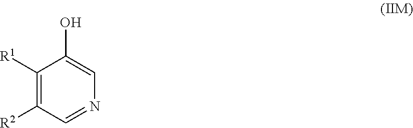

wherein R¹ and R² are as defined herein in formula II (or any particular feature or embodiments thereof), with a compound of formula IIN

Y-LG³ (IIN)

wherein Y is as defined herein in formula II (or any particular feature or embodiments thereof) and LG³ is a suitable leaving group (such as chloro or bromo) in the presence of a suitable solvent (e.g. trifluoroacetic acid, acetic acid, toluene, tetrahydrofuran, or mixtures thereof), under conditions known to those skilled in the art.

Similarly, compounds of formula IIC may be prepared by reaction of a compound of formula IIM as defined herein with a compound of formula IIN as defined herein, in the presence of a suitable solvent (such as acetonitrile, propanol, toluene or tetrahydrofuran) followed by treatment with a suitable base (such as triethylamine or NaOH) or an anion exchange resin (such as IRA-401 (OH)), under conditions known to those skilled in the art.

Further, compounds of formula IID (for example, when LM in formula IID is molybdenum(hydridotris(1-pyrazolyl)borate)(CO)₂)) may be prepared by reaction of a compound of formula IIO

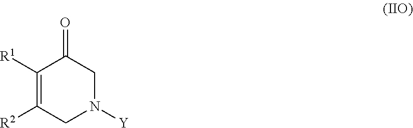

sequentially with: (a) Mo(CO)₃(DMF)₃ and tert-butyldimethylsilylchloride; (b) potassium hydridotris(1-pyrazolyl)borate)(CO)₂); (c) tetrabutylammonium fluoride; (d) methyl iodide; (e) triphenylcarbenium hexafluorophosphate; and (f) triethylamine, for example, according to the consitions described in Malinakova, H. C. and Liebeskind, L. S., Org Letters, 2, 3909 (2000), the contents of which are incorporated herein by reference, or under other conditions known to those skilled in the art, in which the skilled person will also understand that intermediates formed in the sequential reactions (a) to (e) may need to be isolated and purified.

Similarly, compounds of formulae IIH, IIK, IIJ, IIL, IIM, IIN and IIO are either commercially available, are known in the literature, or may be obtained either by analogy with the processes described herein, or by conventional synthetic procedures, in accordance with standard techniques, from available starting materials using appropriate reagents and reaction conditions.

The substituents R¹, R², W, X, and Y as hereinbefore defined, may be modified one or more times, after or during the processes described above for preparation of compounds of formula II byway of methods that are well known to those skilled in the art. Examples of such methods include substitutions, reductions, oxidations, dehydrogenations, alkylations, dealkylations, acylations, hydrolyses, esterifications, etherifications, halogenations and nitrations. The precursor groups can be changed to a different such group, or to the groups defined in formula II, at any time during the reaction sequence. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.

Compounds of the invention may be isolated from their reaction mixtures and, if necessary, purified using conventional techniques as known to those skilled in the art. Thus, processes for preparation of compounds of the invention as described herein may include, as a final step, isolation and optionally purification of the compound of the invention (e.g. isolation and optionally purification of the compound of formula II).

It will be appreciated by those skilled in the art that, in the processes described above and hereinafter, the functional groups of intermediate compounds may need to be protected by protecting groups. The protection and deprotection of functional groups may take place before or after a reaction in the above-mentioned schemes.

Protecting groups may be applied and removed in accordance with techniques that are well known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3rd edition, T. W. Greene & P. G. M. Wutz, Wiley-Interscience (1999).

### Preparation of Compounds of Formula III

A suitable process for the preparation of a compound of formula III as hereinbefore defined may comprise:

(i) reaction of a compound of formula IIIA

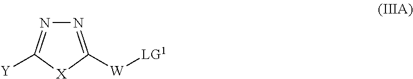

wherein W, X and Y are as defined herein in formula I (or any particular feature or embodiments thereof) and LG¹ represents a suitable leaving group (such as a halo, e.g. Cl), with a compound of formula IIIB

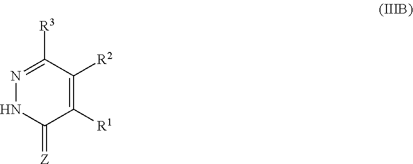

wherein R¹ to R³ and Z are as defined herein in formula III (or any particular feature or embodiments thereof), in the presence of a suitable base (such as a metal carbonate, e.g. sodium carbonate) and in the presence of a suitable solvent (such as a polar organic solvent, e.g. N,N′-dimethylformamide); or
(ii) reaction of a compound of formula IIIC

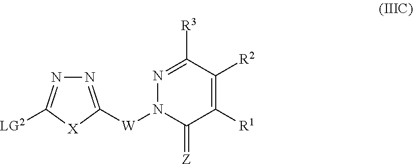

wherein W, X, Z and R¹ to R³ are as defined herein in formula I (or any particular feature or embodiments thereof) and LG² represents a suitable leaving group (such as a halo, e.g. Cl), with a compound of formula IIID

Y—B¹ (IIID)

wherein Y is as defined herein in formula III (or any particular feature or embodiments thereof) and B¹ represents a group suitable for participating in a coupling (e.g. a Pd-catalysed coupling) reaction (such as a boronic acid, e.g. forming a —B(OH)₂ group) in the presence of (e.g. in the presence of a catalytic amount of) a suitable catalyst (such as a Pd catalyst, for example a Pd(0) catalyst, e.g. tetrakis(triphenylphosphine)palladium(0)) and in the presence of a suitable solvent (such as a polar organic solvent, e.g. tetrahydrofuran).

Compounds of formulae IIIA, IIIB, IIIC and IIID are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.

For example, compounds of formula IIIA where X represents O may be prepared by reaction of a compound of formula IIIE

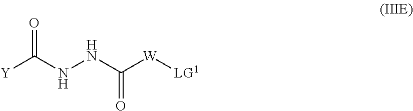

wherein W, and Y are as defined herein in formula III (or any particular feature or embodiments thereof) and LG¹ is as defined herein in formula IMA in the presence of a reagent suitable for performing the ring closure, such as phosphoryl chloride (POCl₃; e.g. in neat POCl₃) and in the presence of a suitable solvent (such as a polar organic solvent, e.g. N,N′-dimethylformamide or tetrahydrofuran), under conditions known to those skilled in the art.

Similarly, compounds of formula IMA where X represents S may be prepared by reaction of a compound of formula IIIE as defined herein in the presence of phosphorous pentasulphide or Lawesson's reagent, under conditions known to those skilled in the art.

Further, compounds of formula IIIB where Z represents O and R³ represents H may be prepared by reaction of compounds of formula INF

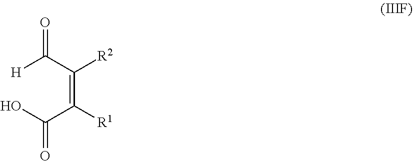

wherein R¹ and R² are as defined herein in formula III (or any particular feature or embodiments thereof), with hydrazine or a salt thereof (e.g. hydrazine sulphate or hydrazine chloride), under conditions known to those skilled in the art.

Similarly, compounds of formulae IIIE and IMF are either commercially available, are known in the literature, or may be obtained either by analogy with the processes described herein, or by conventional synthetic procedures, in accordance with standard techniques, from available starting materials using appropriate reagents and reaction conditions.

The substituents R¹ to R³, W, X, Y, Z, B¹, LG¹ and LG² as hereinbefore defined, may be modified one or more times, after or during the processes described above for preparation of compounds of formula III by way of methods that are well known to those skilled in the art. Examples of such methods include substitutions, reductions, oxidations, dehydrogenations, alkylations, dealkylations, acylations, hydrolyses, esterifications, etherifications, halogenations and nitrations. The precursor groups can be changed to a different such group, or to the groups defined in formula III, at any time during the reaction sequence. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. La rock, Wiley-VCH, 1999.

Compounds for use in the invention may be isolated from their reaction mixtures and, if necessary, purified using conventional techniques as known to those skilled in the art. Thus, processes for preparation of compounds of the invention as described herein may include, as a final step, isolation and optionally purification of the compound of the invention (e.g. isolation and optionally purification of the compound of formula III).

It will be appreciated by those skilled in the art that, in the processes described above and hereinafter, the functional groups of intermediate compounds may need to be protected by protecting groups. The protection and deprotection of functional groups may take place before or after a reaction in the above-mentioned schemes.

Protecting groups may be applied and removed in accordance with techniques that are well known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3rd edition, T. W. Greene & P. G. M. Wutz, Wiley-Interscience (1999).

### Preparation of Compounds of Formula IV

A suitable process for the preparation of a compound of formula IV as hereinbefore defined may comprise:

(i) reaction of a compound of formula IVA

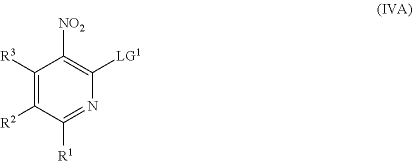

wherein R¹, R² and R³ are as defined herein (i.e. for compounds of the invention, or any particular feature or embodiment thereof) and LG¹ represents a suitable leaving group (such as halo, e.g. chloro), with a compound of formula IVB

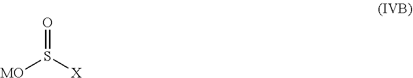

wherein X is as defined herein (i.e. for compounds of the invention, or any particular feature or embodiments thereof) and M represents an alkali metal ion (such as a Na ion), in the presence of a suitable acid (such as a concentrated acid, e.g. a concentrated mineral acid, for example concentrated HCl, e.g. concentrated aqueous HCl) and in the presence of a suitable solvent (such as a polar organic solvent, e.g. N,N′-dimethylacetamide, N,N′-dimethylformamide or tetrahydrofuran), and optionally in the presence of a suitable phase transfer catalyst (such as a quaternary ammonium salt, e.g. tetra-butyl ammonium chloride);
(ii) particularly where at least one Y is present and represents an electron-withdrawing group (such as —NO₂), reaction of a compound of formula IVC

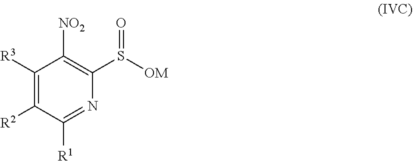

wherein R¹, R² and R³ are as defined herein (i.e. for compounds of the invention, or any particular feature or embodiments thereof) and M represents an alkali metal ion (such as a Na ion), with a compound of formula IVD

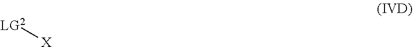

wherein X is as defined herein in formula IV (i.e. for compounds of the invention, or any particular feature or embodiments thereof) and LG² represents a suitable leaving group (such as halo, e.g. chloro), in the presence of a suitable acid (such as a concentrated acid, e.g. a concentrated mineral acid, for example concentrated HCl, e.g. concentrated aqueous HCl) and in the presence of a suitable solvent (such as a polar organic solvent, e.g. N,N′-dimethylacetamide, N,N′-dimethylformamide or tetrahydrofuran), and optionally in the presence of a suitable phase transfer catalyst (such as a quaternary ammonium salt, e.g. tetra-butyl ammonium chloride);
(iii) reaction of a compound of formula IVA as hereinbefore defined with a compound of formula IVB as hereinbefore defined, in the presence of a suitable metal halide (such as a suitable metal iodide, e.g. CuI, or a suitable metal bromide, e.g. CuBr; which metal halide may be present in excess, such as in amount corresponding to at least 2 molar equivalents of the compound of formula IVA and/or the compound of formula IVB) and in the presence of a suitable solvent (such as a polar organic solvent, e.g. N,N′-dimethylacetamide, N,N′-dimethylformamide, tetrahydrofuran or 3-dimethyl-2-imidazolidinone), under conditions known to those skilled in the art;
(iv) reaction of a compound of formula IVC as hereinbefore defined (particularly where at least one R⁴ is present and represents an electron-withdrawing group, such as —NO₂) with a compound of formula IVD as hereinbefore defined, in the presence of a suitable metal halide (such as a suitable metal iodide, e.g. CuI, or a suitable metal bromide, e.g. CuBr; which metal halide may be present in excess, such as in amount corresponding to at least 2 molar equivalents of the compound of formula IVC and/or the compound of formula IVD) and in the presence of a suitable solvent (such as a polar organic solvent, e.g. N,N′-dimethylacetamide, N,N′-dimethylformamide, tetrahydrofuran or 3-dimethyl-2-imidazolidinone), under conditions known to those skilled in the art;
(v) reaction of a compound of formula IVE

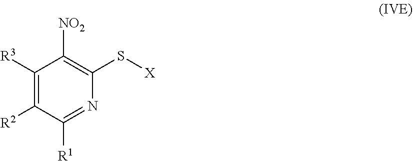

wherein R¹ to R³ and X are as defined herein (i.e. for compounds of the invention, or any particular feature or embodiments thereof), with a suitable oxidising agent (such as a hypochlorite salt, e.g. sodium hypochlorite, a peroxymonosulfate salt, e.g. potassium peroxymonosulfate (Oxone), a percarboxylic acid, e.g. meta-chloroperoxybenzoic acid (mCPBA), or potassium permanganate) in the presence of a suitable solvent (such as a polar organic solvent, e.g. N,N′-dimethylacetamide, N,N′-dimethylformamide or terahydrofuran), and optionally in the presence of water, under conditions known to those skilled in the art;
(vi) particularly where one or more Y is present and represents an electron donating group (such as an alkyl group), reaction of a compound of formula IVF

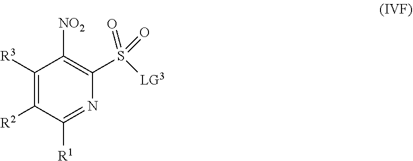

wherein R¹, R² and R³ are as defined herein (i.e. for compounds of the invention, or any particular feature or embodiments thereof) and LG³ represents a suitable leaving group (such as halo, e.g. chloro) with a compound of formula IVG

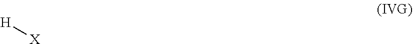

wherein X is as defined (i.e. for compounds of the invention, or any particular feature or embodiments thereof), in the presence of a suitable Lewis acid (such as AlCl₃) and in the presence of a suitable solvent (such as an organic solvent, e.g. dichloromethane or dichloroethane);
(vii) reaction of a compound of formula IVF as defined herein with a compound of formula IVG as defined herein (for example, where one or more Y group is present in the alpha position relative to the point of attachment to the sulfonyl group and represents a suitable directing group), in the presence of a suitable catalyst (such as palladium(II) acetate) and a suitable base (such as a alkali metal carbonate, e.g. potassium carbonate), and in the presence of a suitable solvent (such as an organic solvent, e.g. dichloromethane);
(viii) reaction of a compound of formula V as defined herein with a compound of formula IVH

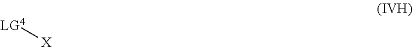

wherein X is as defined herein (i.e. for compounds of the invention, or any particular feature or embodiments thereof) and LG⁴ represents a suitable leaving group (such as a boronic acid), in the presence of a suitable catalyst (such as a suitable metal halide, e.g. CuBr, or phenanthroline) and in the presence of a suitable solvent (such as an organic solvent, e.g. dichloromethane or dichloroethane); or
(ix) reaction of a compound of formula IVC as defined herein with (a) a compound of formula IVG as defined herein having at least one Y group, or (b) a compound of formula IVG as defined herein but having a group that may be converted to a Y group, wherein the Y group or group that may be converted to a Y group is present in the alpha position relative to the essential H substituent and represents a suitable directing group (such as a suitable amide, e.g. —C(O)N(H)C(CH₃)₂-2-pyridinyl), in the presence of a suitable catalyst and/or oxidant (such as copper(II) acetate and/or silver carbonate), and in the presence of a suitable solvent (such as an organic solvent, e.g. dichloroethane), which step may further comprise conversion of the group that may be converted to a Y group to the required Y group, under conditions known to those skilled in the art.

Compounds of formulae IVA, IVC, IVB, IVD, IVE, IVF, IVG and IVH are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.

In particular, compounds of formula IVE may be prepared by reaction of a compound of formula IVJ

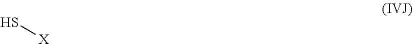

wherein X is as defined herein (i.e. for compounds of the invention, or any particular feature or embodiments thereof), with a compound of formula IVA as herein before defined, under conditions known to those skilled in the art, such as in the presence of a suitable base (such as a metal carbonate, e.g. potassium carbonate, a metal hydroxide, e.g. sodium hydroxide, or an amine base, e.g. triethyl amine), and in the presence of a suitable solvent (such as a polar organic solvent, e.g. N,N′-dimethylacetamide, N,N′-dimethylformamide or tetrahydrofuran, or a mixture of a polar organic solvent and water), under conditions known to those skilled in the art.

Similarly, compounds of formula IVE (particularly where at least one Y is present and represents an electron-withdrawing group, such as —NO₂) may be prepared by reaction of a compound of formula IVK

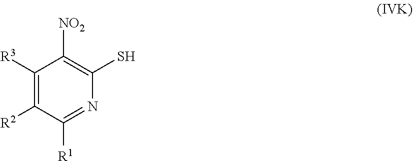

wherein R¹, R² and R³ are as defined herein (i.e. for compounds of the invention, or any particular feature or embodiments thereof), with a compound of formula IVD as described herein, under conditions known to those skilled in the art (for example, where the R⁴ groups present in the compound of formula IVD are not sufficiently electron withdrawing, the reaction may be performed in the presence of a suitable catalyst, such as palladium(II) acetate or copper oxide, in which case the suitable base may be an alkali metal tert-butoxide, such as Kt-OBu).

Similarly, compounds of formulae IVJ and IVK are either commercially available, are known in the literature, or may be obtained either by analogy with the processes described herein, or by conventional synthetic procedures, in accordance with standard techniques, from available starting materials using appropriate reagents and reaction conditions.

The substituents R¹ to R³ and Y, as hereinbefore defined, may be modified one or more times, after or during the processes described above for preparation of compounds of formula IV by way of methods that are well known to those skilled in the art. Examples of such methods include substitutions, reductions, oxidations, dehydrogenations, alkylations, dealkylations, acylations, hydrolyses, esterifications, etherifications, halogenations and nitrations. The precursor groups can be changed to a different such group, or to the groups defined in formula I, at any time during the reaction sequence. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.

Compounds of the invention may be isolated from their reaction mixtures and, if necessary, purified using conventional techniques as known to those skilled in the art. Thus, processes for preparation of compounds of the invention as described herein may include, as a final step, isolation and optionally purification of the compound of the invention.

It will be appreciated by those skilled in the art that, in the processes described above and hereinafter, the functional groups of intermediate compounds may need to be protected by protecting groups. The protection and deprotection of functional groups may take place before or after a reaction in the above-mentioned schemes.

Protecting groups may be applied and removed in accordance with techniques that are well known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3rd edition, T. W. Greene & P. G. M. Wutz, Wiley-Interscience (1999).

### Preparation of Compounds of Formula V

A suitable process for the preparation of a compound of formula V as hereinbefore defined may comprise:

(i) reaction of a compound of formula VA

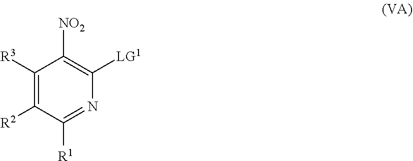

wherein R¹, R² and R³ are as defined herein (i.e. for compounds of the invention, or any particular feature or embodiment thereof) and LG¹ represents a suitable leaving group (such as halo, e.g. chloro), with a compound of formula VB

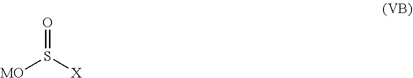

wherein X is as defined herein (i.e. for compounds of the invention, or any particular feature or embodiments thereof) and M represents an alkali metal ion (such as a Na ion), in the presence of a suitable acid (such as a concentrated acid, e.g. a concentrated mineral acid, for example concentrated HCl, e.g. concentrated aqueous HCl) and in the presence of a suitable solvent (such as a polar organic solvent, e.g. N,N′-dimethylacetamide, N,N′-dimethylformamide or tetrahydrofuran), and optionally in the presence of a suitable phase transfer catalyst (such as a quaternary ammonium salt, e.g. tetra-butyl ammonium chloride);
(ii) reaction of a compound of formula VC

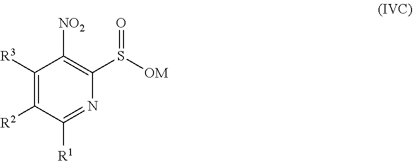

wherein R¹, R² and R³ are as defined herein (i.e. for compounds of the invention, or any particular feature or embodiments thereof) and M represents an alkali metal ion (such as a Na ion), with a compound of formula VD

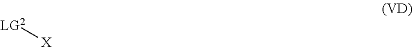

wherein X is as defined herein in formula V (i.e. for compounds of the invention, or any particular feature or embodiments thereof) and LG² represents a suitable leaving group (such as halo, e.g. chloro), in the presence of a suitable acid (such as a concentrated acid, e.g. a concentrated mineral acid, for example concentrated HCl, e.g. concentrated aqueous HCl) and in the presence of a suitable solvent (such as a polar organic solvent, e.g. N,N′-dimethylacetamide, N,N′-dimethylformamide or tetrahydrofuran), and optionally in the presence of a suitable phase transfer catalyst (such as a quaternary ammonium salt, e.g. tetra-butyl ammonium chloride);
(iii) reaction of a compound of formula VA as hereinbefore defined with a compound of formula VB as hereinbefore defined, in the presence of a suitable metal halide (such as a suitable metal iodide, e.g. CuI, or a suitable metal bromide, e.g. CuBr; which metal halide may be present in excess, such as in amount corresponding to at least 2 molar equivalents of the compound of formula VA and/or the compound of formula VB) and in the presence of a suitable solvent (such as a polar organic solvent, e.g. N,N′-dimethylacetamide, N,N′-dimethylformamide, tetrahydrofuran or 3-dimethyl-2-imidazolidinone), under conditions known to those skilled in the art;
(iv) reaction of a compound of formula VC as hereinbefore defined with a compound of formula VD as hereinbefore defined, in the presence of a suitable metal halide (such as a suitable metal iodide, e.g. CuI, or a suitable metal bromide, e.g. CuBr; which metal halide may be present in excess, such as in amount corresponding to at least 2 molar equivalents of the compound of formula VC and/or the compound of formula VD) and in the presence of a suitable solvent (such as a polar organic solvent, e.g. N,N′-dimethylacetamide, N,N′-dimethylformamide, tetrahydrofuran or 3-dimethyl-2-imidazolidinone), under conditions known to those skilled in the art;
(v) reaction of a compound of formula VE

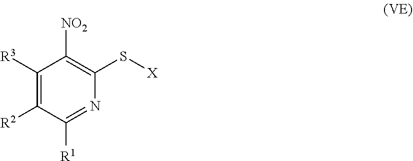

wherein R¹ to R³ and X are as defined herein (i.e. for compounds of the invention, or any particular feature or embodiments thereof), with a suitable oxidising agent (such as a hypochlorite salt, e.g. sodium hypochlorite, a peroxymonosulfate salt, e.g. potassium peroxymonosulfate (Oxone), a percarboxylic acid, e.g. meta-chloroperoxybenzoic acid (mCPBA), or potassium permanganate) in the presence of a suitable solvent (such as a polar organic solvent, e.g. N,N′-dimethylacetamide, N,N′-dimethylformamide or terahydrofuran), and optionally in the presence of water, under conditions known to those skilled in the art;
(vi) reaction of a compound of formula VF

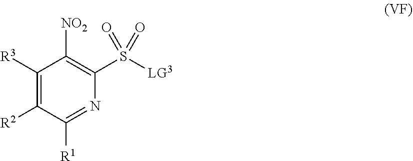

wherein R¹, R² and R³ are as defined herein (i.e. for compounds of the invention, or any particular feature or embodiments thereof) and LG³ represents a suitable leaving group (such as halo, e.g. chloro) with a compound of formula VG

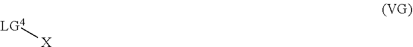

wherein X is as defined herein (i.e. for compounds of the invention, or any particular feature or embodiments thereof) and LG⁴ represents a suitable leaving group (such as a boronic acid), in the presence of a suitable catalyst (such as a suitable metal halide, e.g. CuBr, or phenanthroline) and in the presence of a suitable solvent (such as an organic solvent, e.g. dichloromethane or dichloroethane).

Compounds of formulae VA, VC, VB, VD, VE, VF and VG are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.

In particular, compounds of formula VE may be prepared by reaction of a compound of formula VH

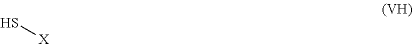

wherein X is as defined herein (i.e. for compounds of the invention, or any particular feature or embodiments thereof), with a compound of formula VA as herein before defined, under conditions known to those skilled in the art, such as in the presence of a suitable base (such as a metal carbonate, e.g. potassium carbonate, a metal hydroxide, e.g. sodium hydroxide, or an amine base, e.g. triethyl amine), and in the presence of a suitable solvent (such as a polar organic solvent, e.g. N,N′-dimethylacetamide, N,N′-dimethylformamide or tetrahydrofuran, or a mixture of a polar organic solvent and water), under conditions known to those skilled in the art.

Similarly, compounds of formula VE may be prepared by reaction of a compound of formula VJ

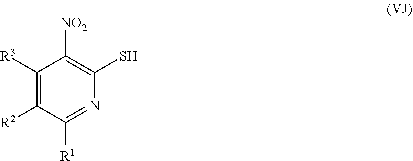

wherein R¹, R² and R³ are as defined herein (i.e. for compounds of the invention, or any particular feature or embodiments thereof), with a compound of formula VD as described herein, under conditions known to those skilled in the art (for example, where the R⁴ groups present in the compound of formula VD are not sufficiently electron withdrawing, the reaction may be performed in the presence of a suitable catalyst, such as palladium(II) acetate or copper oxide, in which case the suitable base may be an alkali metal tert-butoxide, such as Kt-OBu).

Similarly, compounds of formulae VH and VJ are either commercially available, are known in the literature, or may be obtained either by analogy with the processes described herein, or by conventional synthetic procedures, in accordance with standard techniques, from available starting materials using appropriate reagents and reaction conditions.

The substituents R¹ to R³ and Y, as hereinbefore defined, may be modified one or more times, after or during the processes described above for preparation of compounds of formula V by way of methods that are well known to those skilled in the art. Examples of such methods include substitutions, reductions, oxidations, dehydrogenations, alkylations, dealkylations, acylations, hydrolyses, esterifications, etherifications, halogenations and nitrations. The precursor groups can be changed to a different such group, or to the groups defined in formula V, at any time during the reaction sequence. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.

Compounds for use in the invention may be isolated from their reaction mixtures and, if necessary, purified using conventional techniques as known to those skilled in the art. Thus, processes for preparation of compounds of the invention as described herein may include, as a final step, isolation and optionally purification of the compound of the invention (e.g. isolation and optionally purification of the compound of formula V).

It will be appreciated by those skilled in the art that, in the processes described above and hereinafter, the functional groups of intermediate compounds may need to be protected by protecting groups. The protection and deprotection of functional groups may take place before or after a reaction in the above-mentioned schemes.

Protecting groups may be applied and removed in accordance with techniques that are well known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3rd edition, T. W. Greene & P. G. M. Wutz, Wiley-Interscience (1999).

### Preparation of Compounds of Formula VI

A suitable process for the preparation of a compound of formula VI as hereinbefore defined may comprise:

(i) reaction of a compound of formula VIA

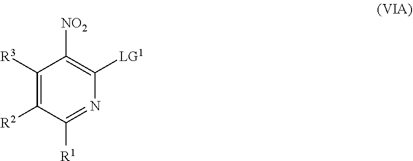

wherein R¹, R² and R³ are as defined herein (i.e. for compounds of the invention, or any particular feature or embodiment thereof) and LG¹ represents a suitable leaving group (such as halo, e.g. chloro), with a compound of formula VIB

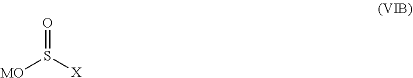

wherein X is as defined herein (i.e. for compounds of the invention, or any particular feature or embodiments thereof) and M represents an alkali metal ion (such as a Na ion),
in the presence of a suitable acid (such as a concentrated acid, e.g. a concentrated mineral acid, for example concentrated HCl, e.g. concentrated aqueous HCl) and in the presence of a suitable solvent (such as a polar organic solvent, e.g. N,N′-dimethylacetamide, N,N′-dimethylformamide or tetrahydrofuran), and optionally in the presence of a suitable phase transfer catalyst (such as a quaternary ammonium salt, e.g. tetra-butyl ammonium chloride);
(ii) reaction of a compound of formula VIC

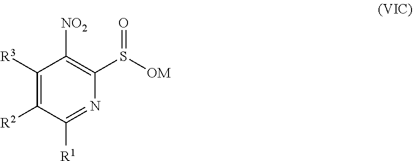

wherein R¹, R² and R³ are as defined herein (i.e. for compounds of the invention, or any particular feature or embodiments thereof) and M represents an alkali metal ion (such as a Na ion), with a compound of formula VID

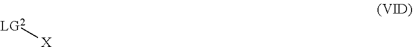

wherein X is as defined herein in formula VI (i.e. for compounds of the invention, or any particular feature or embodiments thereof) and LG² represents a suitable leaving group (such as halo, e.g. chloro), in the presence of a suitable acid (such as a concentrated acid, e.g. a concentrated mineral acid, for example concentrated HCl, e.g. concentrated aqueous HCl) and in the presence of a suitable solvent (such as a polar organic solvent, e.g. N,N′-dimethylacetamide, N,N′-dimethylformamide or tetrahydrofuran), and optionally in the presence of a suitable phase transfer catalyst (such as a quaternary ammonium salt, e.g. tetra-butyl ammonium chloride);
(iii) reaction of a compound of formula VIA as hereinbefore defined with a compound of formula VIB as hereinbefore defined, in the presence of a suitable metal halide (such as a suitable metal iodide, e.g. CuI, or a suitable metal bromide, e.g. CuBr; which metal halide may be present in excess, such as in amount corresponding to at least 2 molar equivalents of the compound of formula VIA and/or the compound of formula VIB) and in the presence of a suitable solvent (such as a polar organic solvent, e.g. N,N′-dimethylacetamide, N,N′-dimethylformamide, tetrahydrofuran or 3-dimethyl-2-imidazolidinone), under conditions known to those skilled in the art;
(iv) reaction of a compound of formula VIC as hereinbefore defined with a compound of formula VID as hereinbefore defined, in the presence of a suitable metal halide (such as a suitable metal iodide, e.g. CuI, or a suitable metal bromide, e.g. CuBr; which metal halide may be present in excess, such as in amount corresponding to at least 2 molar equivalents of the compound of formula VICE and/or the compound of formula VID) and in the presence of a suitable solvent (such as a polar organic solvent, e.g. N,N′-dimethylacetamide, N,N′-dimethylformamide, tetrahydrofuran or 3-dimethyl-2-imidazolidinone), under conditions known to those skilled in the art;
(v) reaction of a compound of formula VIE

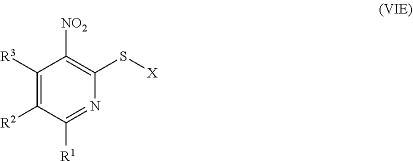

wherein R¹ to R³ and X are as defined herein (i.e. for compounds of the invention, or any particular feature or embodiments thereof), with a suitable oxidising agent (such as a hypochlorite salt, e.g. sodium hypochlorite, a peroxymonosulfate salt, e.g. potassium peroxymonosulfate (Oxone), a percarboxylic acid, e.g. meta-chloroperoxybenzoic acid (mCPBA), or potassium permanganate) in the presence of a suitable solvent (such as a polar organic solvent, e.g. N,N′-dimethylacetamide, N,N′-dimethylformamide or terahydrofuran), and optionally in the presence of water, under conditions known to those skilled in the art;
(vi) reaction of a compound of formula VIF

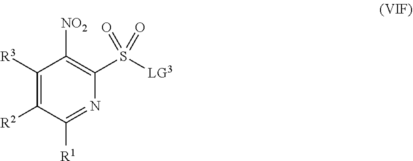

wherein R¹, R² and R³ are as defined herein (i.e. for compounds of the invention, or any particular feature or embodiments thereof) and LG³ represents a suitable leaving group (such as halo, e.g. chloro) with a compound of formula VIG

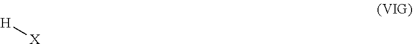

wherein X is as defined (i.e. for compounds of the invention, or any particular feature or embodiments thereof), in the presence of a suitable Lewis acid (such as AlCl₃) and in the presence of a suitable solvent (such as an organic solvent, e.g. dichloromethane or dichloroethane).

Compounds of formulae VIA, VIC, VIB, VID, VIE, VIF and VIG are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.

In particular, compounds of formula VIE may be prepared by reaction of a compound of formula VIH

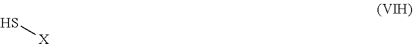

wherein X is as defined herein (i.e. for compounds of the invention, or any particular feature or embodiments thereof), with a compound of formula VIA as herein before defined, under conditions known to those skilled in the art, such as in the presence of a suitable base (such as a metal carbonate, e.g. potassium carbonate, a metal hydroxide, e.g. sodium hydroxide, or an amine base, e.g. triethyl amine), and in the presence of a suitable solvent (such as a polar organic solvent, e.g. N,N′-dimethylacetamide, N,N′-dimethylformamide or tetrahydrofuran, or a mixture of a polar organic solvent and water), under conditions known to those skilled in the art.

Similarly, compounds of formula VIE may be prepared by reaction of a compound of formula VIJ

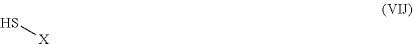

wherein X is as defined herein (i.e. for compounds of the invention, or any particular feature or embodiments thereof), with a compound of formula VIA as herein before defined, under conditions known to those skilled in the art, such as in the presence of a suitable base (such as a metal carbonate, e.g. potassium carbonate, a metal hydroxide, e.g. sodium hydroxide, or an amine base, e.g. triethyl amine), and in the presence of a suitable solvent (such as a polar organic solvent, e.g. N,N′-dimethylacetamide, N,N′-dimethylformamide or tetrahydrofuran, or a mixture of a polar organic solvent and water), under conditions known to those skilled in the art.

Similarly, compounds of formula VIE (particularly where at least one Y is present and represents an electron-withdrawing group, such as —NO₂) may be prepared by reaction of a compound of formula VIK

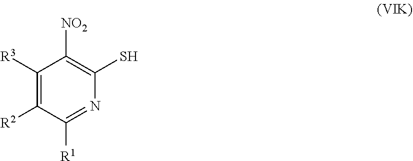

wherein R¹, R² and R³ are as defined herein (i.e. for compounds of the invention, or any particular feature or embodiments thereof), with a compound of formula VID as described herein, under conditions known to those skilled in the art (for example, where the R⁴ groups present in the compound of formula VID are not sufficiently electron withdrawing, the reaction may be performed in the presence of a suitable catalyst, such as palladium(II) acetate or copper oxide, in which case the suitable base may be an alkali metal tert-butoxide, such as Kt-OBu).

Similarly, compounds of formulae VIJ and VIK are either commercially available, are known in the literature, or may be obtained either by analogy with the processes described herein, or by conventional synthetic procedures, in accordance with standard techniques, from available starting materials using appropriate reagents and reaction conditions.

The substituents R¹ to R³ and Y, as hereinbefore defined, may be modified one or more times, after or during the processes described above for preparation of compounds of formula VI by way of methods that are well known to those skilled in the art. Examples of such methods include substitutions, reductions, oxidations, dehydrogenations, alkylations, dealkylations, acylations, hydrolyses, esterifications, etherifications, halogenations and nitrations. The precursor groups can be changed to a different such group, or to the groups defined in formula VI, at any time during the reaction sequence. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.

Compounds of the invention may be isolated from their reaction mixtures and, if necessary, purified using conventional techniques as known to those skilled in the art. Thus, processes for preparation of compounds of the invention as described herein may include, as a final step, isolation and optionally purification of the compound of the invention (e.g. isolation and optionally purification of the compound of formula VI).

It will be appreciated by those skilled in the art that, in the processes described above and hereinafter, the functional groups of intermediate compounds may need to be protected by protecting groups. The protection and deprotection of functional groups may take place before or after a reaction in the above-mentioned schemes.

Protecting groups may be applied and removed in accordance with techniques that are well known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3rd edition, T. W. Greene & P. G. M. Wutz, Wiley-Interscience (1999).

### Preparation of Compounds of Formula VII

A suitable process for the preparation of a compound of formula VII as hereinbefore defined may comprise:

(i) reaction of a compound of formula VIIA

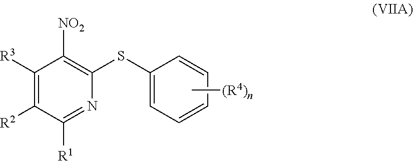

wherein R¹ to R⁴ and n are as defined herein in formula VII (or any particular feature or embodiments thereof), with a suitable oxidising agent (such as meta-chloroperoxybenzoic acid (mCPBA)) in the presence of a suitable solvent (such as dichloromethane (DCM)), under conditions known to those skilled in the art.

Compounds of formulae VIIA may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.

In particular, compounds of formula VIIA may be prepared by reaction of a compound of formula VIIB

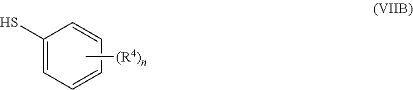

wherein R⁴ and n are as defined herein in formula VII (or any particular feature or embodiments thereof), with a compound of formula VIIC

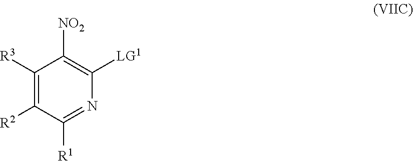

wherein R¹, R² and R³ are as defined herein in formula VII (or any particular feature or embodiment thereof) and LG¹ represents a suitable leaving group (such as halo, e.g. chloro), under conditions known to those skilled in the art, such as in the presence of a suitable base (such as a metal carbonate, e.g. potassium carbonate, a metal hydroxide, e.g. sodium hydroxide, or an amine base, e.g. triethyl amine), and in the presence of a suitable solvent (such as a polar organic solvent, e.g. N,N′-dimethylacetamide, N,N′-dimethylformamide or tetrahydrofuran, or a mixture of a polar organic solvent and water), under conditions known to those skilled in the art.

Compounds of formulae VIIB and VIIC are either commercially available, are known in the literature, or may be obtained either by analogy with the processes described herein, or by conventional synthetic procedures, in accordance with standard techniques, from available starting materials using appropriate reagents and reaction conditions.

The substituents R¹ to R⁴, as hereinbefore defined, may be modified one or more times, after or during the processes described above for preparation of compounds of formula I by way of methods that are well known to those skilled in the art. Examples of such methods include substitutions, reductions, oxidations, dehydrogenations, alkylations, dealkylations, acylations, hydrolyses, esterifications, etherifications, halogenations and nitrations. The precursor groups can be changed to a different such group, or to the groups defined in formula I, at any time during the reaction sequence. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.

Compounds of the invention may be isolated from their reaction mixtures and, if necessary, purified using conventional techniques as known to those skilled in the art. Thus, processes for preparation of compounds of the invention as described herein may include, as a final step, isolation and optionally purification of the compound of the invention (e.g. isolation and optionally purification of the compound of formula VII).

It will be appreciated by those skilled in the art that, in the processes described above and hereinafter, the functional groups of intermediate compounds may need to be protected by protecting groups. The protection and deprotection of functional groups may take place before or after a reaction in the above-mentioned schemes.

Protecting groups may be applied and removed in accordance with techniques that are well known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3rd edition, T. W. Greene & P. G. M. Wutz, Wiley-Interscience (1999).

### Preparation of Compounds of Formula VIII

A suitable process for the preparation of a compound of formula VIII as hereinbefore defined may comprise:

(i) where n represents 2, reaction of a compound of formula VIIIA

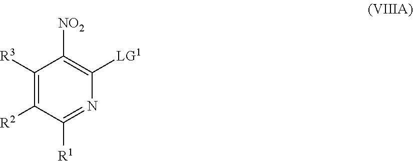

wherein R¹, R² and R³ are as defined herein (i.e. for compounds of the invention, or any particular feature or embodiment thereof) and LG¹ represents a suitable leaving group (such as halo, e.g. chloro), with a compound of formula VIIIB

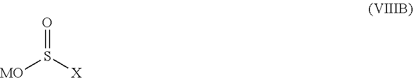

wherein X is as defined herein (i.e. for compounds of the invention, or any particular feature or embodiments thereof) and M represents an alkali metal ion (such as a Na ion),
in the presence of a suitable acid (such as a concentrated acid, e.g. a concentrated mineral acid, for example concentrated HCl, e.g. concentrated aqueous HCl) and in the presence of a suitable solvent (such as a polar organic solvent, e.g. N,N′-dimethylacetamide, N,N′-dimethylformamide or tetrahydrofuran), and optionally in the presence of a suitable phase transfer catalyst (such as a quaternary ammonium salt, e.g. tetra-butyl ammonium chloride);
(ii) where n represents 2, particularly where at least one Y is present and represents an electron-withdrawing group (such as —NO₂), reaction of a compound of formula VIIIC

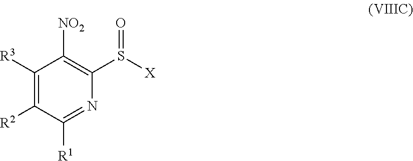

wherein R¹, R² and R³ are as defined herein (i.e. for compounds of the invention, or any particular feature or embodiments thereof) and M represents an alkali metal ion (such as a Na ion), with a compound of formula VIIID

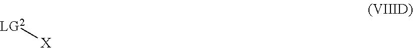

wherein X is as defined herein in formula VIII (i.e. for compounds of the invention, or any particular feature or embodiments thereof) and LG² represents a suitable leaving group (such as halo, e.g. chloro), in the presence of a suitable acid (such as a concentrated acid, e.g. a concentrated mineral acid, for example concentrated HCl, e.g. concentrated aqueous HCl) and in the presence of a suitable solvent (such as a polar organic solvent, e.g. N,N′-dimethylacetamide, N,N′-dimethylformamide or tetrahydrofuran), and optionally in the presence of a suitable phase transfer catalyst (such as a quaternary ammonium salt, e.g. tetra-butyl ammonium chloride);
(iii) where n represents 2, reaction of a compound of formula VIIIA as hereinbefore defined with a compound of formula VIIIB as hereinbefore defined, in the presence of a suitable metal halide (such as a suitable metal iodide, e.g. CuI, or a suitable metal bromide, e.g. CuBr; which metal halide may be present in excess, such as in amount corresponding to at least 2 molar equivalents of the compound of formula VIIIA and/or the compound of formula VIIIB) and in the presence of a suitable solvent (such as a polar organic solvent, e.g. N,N′-dimethylacetamide, N,N′-dirnethylforrnamide, tetrahydrofuran or 3-dimethyl-2-imidazolidinone), under conditions known to those skilled in the art;
(iv) where n represents 2, reaction of a compound of formula VIIIC as hereinbefore defined (particularly where at least one R⁴ is present and represents an electron-withdrawing group, such as —NO₂) with a compound of formula VIIID as hereinbefore defined, in the presence of a suitable metal halide (such as a suitable metal iodide, e.g. CuI, or a suitable metal bromide, e.g. CuBr; which metal halide may be present in excess, such as in amount corresponding to at least 2 molar equivalents of the compound of formula VIIIC and/or the compound of formula VIIID) and in the presence of a suitable solvent (such as a polar organic solvent, e.g. N,N′-dimethylacetamide, N,N′-dimethylformamide, tetrahydrofuran or 3-dimethyl-2-imidazolidinone), under conditions known to those skilled in the art;
(v) reaction of a compound of formula VIIIE

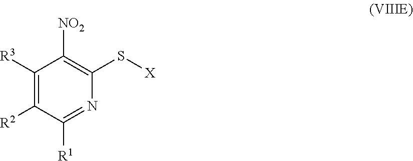

wherein R¹ to R³ and X are as defined herein (i.e. for compounds of the invention, or any particular feature or embodiments thereof), with a suitable oxidising agent (i.e. an oxidising agent chosen and used in a manner as required to achieved the desired degree of oxidation; such as a hypochlorite salt, e.g. sodium hypochlorite, a peroxymonosulfate salt, e.g. potassium peroxymonosulfate (Oxone), a percarboxylic acid, e.g. mete-chloroperoxybenzoic acid (mCPBA), or potassium permanganate) in the presence of a suitable solvent (such as a polar organic solvent, e.g. N,N′-dimethylacetamide, N,N′-dimethylformamide or terahydrofuran), and optionally in the presence of water, under conditions known to those skilled in the art;
(vi) where n represents 2, particularly where one or more Y is present and represents an electron donating group (such as an alkyl group), reaction of a compound of formula VIIIF

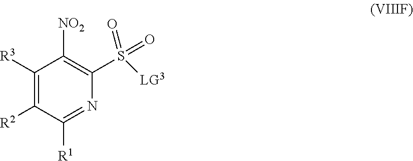

wherein R¹, R² and R³ are as defined herein (i.e. for compounds of the invention, or any particular feature or embodiments thereof) and LG³ represents a suitable leaving group (such as halo, e.g. chloro) with a compound of formula VIIIG

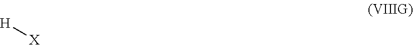

wherein X is as defined (i.e. for compounds of the invention, or any particular feature or embodiments thereof), in the presence of a suitable Lewis acid (such as AlCl₃) and in the presence of a suitable solvent (such as an organic solvent, e.g. dichloromethane or dichloroethane);
(vii) where n represents 2, reaction of a compound of formula VIIIF as defined herein with a compound of formula VIIIIG as defined herein (for example, where one or more Y group is present in the alpha position relative to the point of attachment to the sulfonyl group and represents a suitable directing group), in the presence of a suitable catalyst (such as palladium(II) acetate) and a suitable base (such as a alkali metal carbonate, e.g. potassium carbonate), and in the presence of a suitable solvent (such as an organic solvent, e.g. dichloromethane);
(viii) where n represents 2, reaction of a compound of formula V as defined herein with a compound of formula VIIIH

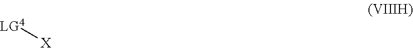

wherein X is as defined herein (i.e. for compounds of the invention, or any particular feature or embodiments thereof) and LG⁴ represents a suitable leaving group (such as a boronic acid), in the presence of a suitable catalyst (such as a suitable metal halide, e.g. CuBr, or phenanthroline) and in the presence of a suitable solvent (such as an organic solvent, e.g. dichloromethane or dichloroethane); or
(ix) where n represents 2, reaction of a compound of formula VIIIC as defined herein with (a) a compound of formula VIIIG as defined herein having at least one Y group, or (b) a compound of formula VIIIG as defined herein but having a group that may be converted to a Y group, wherein the Y group or group that may be converted to a Y group is present in the alpha position relative to the essential H substituent and represents a suitable directing group (such as a suitable amide, e.g. —C(O)N(H)C(CH₃)₂-2-pyridinyl), in the presence of a suitable catalyst and/or oxidant (such as copper(II) acetate and/or silver carbonate), and in the presence of a suitable solvent (such as an organic solvent, e.g. dichloroethane), which step may further comprise conversion of the group that may be converted to a Y group to the required Y group, under conditions known to those skilled in the art.
Compounds of formulae VIIIA, VIIB, VIIIC, VIIID, VIIIE, VIIIF, VIIIG and VIIIH are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.
In particular, compounds of formula VIIIE may be prepared by reaction of a compound of formula VIIIJ

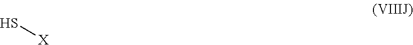

wherein X is as defined herein (i.e. for compounds of the invention, or any particular feature or embodiments thereof), with a compound of formula VIIIA as herein before defined, under conditions known to those skilled in the art, such as in the presence of a suitable base (such as a metal carbonate, e.g. potassium carbonate, a metal hydroxide, e.g. sodium hydroxide, or an amine base, e.g. triethyl amine), and in the presence of a suitable solvent (such as a polar organic solvent, e.g. N,N′-dimethylacetamide, N,N′-dimethylformamide or tetrahydrofuran, or a mixture of a polar organic solvent and water), under conditions known to those skilled in the art.
Similarly, compounds of formula VIIIE (particularly where at least one Y is present and represents an electron-withdrawing group, such as —NO₂) may be prepared by reaction of a compound of formula VIIIK

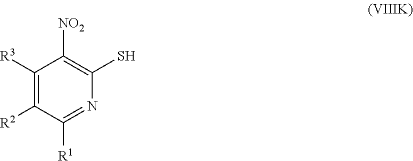

wherein R¹, R² and R³ are as defined herein (i.e. for compounds of the invention, or any particular feature or embodiments thereof), with a compound of formula VIIID as described herein, under conditions known to those skilled in the art (for example, where the R⁴ groups present in the compound of formula VIIID are not sufficiently electron withdrawing, the reaction may be performed in the presence of a suitable catalyst, such as palladium(II) acetate or copper oxide, in which case the suitable base may be an alkali metal tert-butoxide, such as Kt-OBu).
Similarly, compounds of formulae VIIIJ and VIIIK are either commercially available, are known in the literature, or may be obtained either by analogy with the processes described herein, or by conventional synthetic procedures, in accordance with standard techniques, from available starting materials using appropriate reagents and reaction conditions.
The substituents R¹ to R³ and Y, as hereinbefore defined, may be modified one or more times, after or during the processes described above for preparation of compounds of formula VIII by way of methods that are well known to those skilled in the art. Examples of such methods include substitutions, reductions, oxidations, dehydrogenations, alkylations, dealkylations, acylations, hydrolyses, esterifications, etherifications, halogenations and nitrations. The precursor groups can be changed to a different such group, or to the groups defined in formula VIII, at any time during the reaction sequence. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.
Compounds of the invention may be isolated from their reaction mixtures and, if necessary, purified using conventional techniques as known to those skilled in the art. Thus, processes for preparation of compounds of the invention as described herein may include, as a final step, isolation and optionally purification of the compound of the invention.
It will be appreciated by those skilled in the art that, in the processes described above and hereinafter, the functional groups of intermediate compounds may need to be protected by protecting groups. The protection and deprotection of functional groups may take place before or after a reaction in the above-mentioned schemes.
Protecting groups may be applied and removed in accordance with techniques that are well known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3rd edition, T. W. Greene & P. G. M. Wutz, Wiley-Interscience (1999).

### Preparation of Compounds of Formula IX

A suitable process for the preparation of a compound of formula IX as hereinbefore defined may comprise:

(i) where n represents 2, reaction of a compound of formula IXA

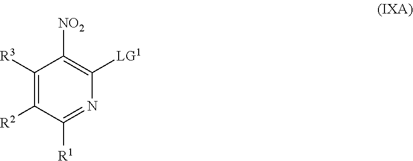

wherein R¹, R² and R³ are as defined herein (i.e. for compounds of the invention, or any particular feature or embodiment thereof) and LG¹ represents a suitable leaving group (such as halo, e.g. chloro), with a compound of formula IXB

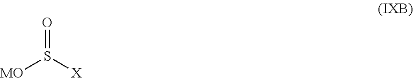

wherein X is as defined herein (i.e. for compounds of the invention, or any particular feature or embodiments thereof) and M represents an alkali metal ion (such as a Na ion), in the presence of a suitable acid (such as a concentrated acid, e.g. a concentrated mineral acid, for example concentrated HCl, e.g. concentrated aqueous HCl) and in the presence of a suitable solvent (such as a polar organic solvent, e.g. N,N′-dimethylacetamide, N,N′-dimethylformamide or tetrahydrofuran), and optionally in the presence of a suitable phase transfer catalyst (such as a quaternary ammonium salt, e.g. tetra-butyl ammonium chloride);
(ii) where n represents 2, reaction of a compound of formula IXC

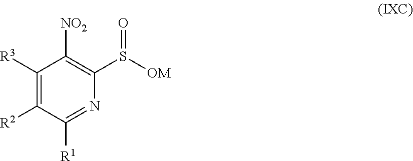

wherein R¹, R² and R³ are as defined herein (i.e. for compounds of the invention, or any particular feature or embodiments thereof) and M represents an alkali metal ion (such as a Na ion), with a compound of formula IXD

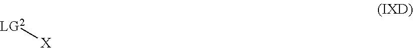

wherein X is as defined herein in formula IX (i.e. for compounds of the invention, or any particular feature or embodiments thereof) and LG² represents a suitable leaving group (such as halo, e.g. chloro), in the presence of a suitable acid (such as a concentrated acid, e.g. a concentrated mineral acid, for example concentrated HCl, e.g. concentrated aqueous HCl) and in the presence of a suitable solvent (such as a polar organic solvent, e.g. N,N′-dimethylacetamide, N,N′-dimethylformamide or tetrahydrofuran), and optionally in the presence of a suitable phase transfer catalyst (such as a quaternary ammonium salt, e.g. tetra-butyl ammonium chloride);
(iii) where n represents 2, reaction of a compound of formula IXA as hereinbefore defined with a compound of formula IXB as hereinbefore defined, in the presence of a suitable metal halide (such as a suitable metal iodide, e.g. CuI, or a suitable metal bromide, e.g. CuBr; which metal halide may be present in excess, such as in amount corresponding to at least 2 molar equivalents of the compound of formula IXA and/or the compound of formula IXB) and in the presence of a suitable solvent (such as a polar organic solvent, e.g. N,N′-dimethylacetamide, N,N′-dimethylformamide, tetrahydrofuran or 3-dimethyl-2-imidazolidinone), under conditions known to those skilled in the art;
(iv) where n represents 2, reaction of a compound of formula IXC as hereinbefore defined with a compound of formula IXD as hereinbefore defined, in the presence of a suitable metal halide (such as a suitable metal iodide, e.g. CuI, or a suitable metal bromide, e.g. CuBr; which metal halide may be present in excess, such as in amount corresponding to at least 2 molar equivalents of the compound of formula IXC and/or the compound of formula IXD) and in the presence of a suitable solvent (such as a polar organic solvent, e.g. N,N′-dimethylacetamide, N,N′-dimethylformamide, tetrahydrofuran or 3-dimethyl-2-imidazolidinone), under conditions known to those skilled in the art;
(v) reaction of a compound of formula IXE

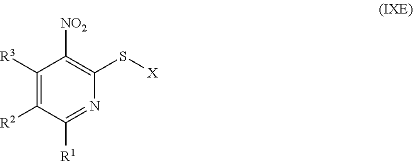

wherein R¹ to R³ and X are as defined herein (i.e. for compounds of the invention, or any particular feature or embodiments thereof), with a suitable oxidising agent (i.e. an oxidising agent chosen and used in a manner as required to achieved the desired degree of oxidation; such as a hypochlorite salt, e.g. sodium hypochlorite, a peroxymonosulfate salt, e.g. potassium peroxymonosulfate (Oxone), a percarboxylic acid, e.g. meta-chloroperoxybenzoic acid (mCPBA), or potassium permanganate) in the presence of a suitable solvent (such as a polar organic solvent, e.g. N,N′-dimethylacetamide, N,N′-dimethylformamide or terahydrofuran), and optionally in the presence of water, under conditions known to those skilled in the art;
(vi) where n represents 2, reaction of a compound of formula IXF

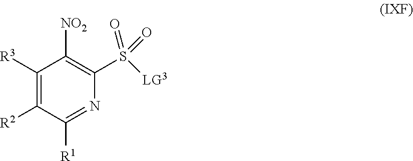

wherein R¹, R² and R³ are as defined herein (i.e. for compounds of the invention, or any particular feature or embodiments thereof) and LG³ represents a suitable leaving group (such as halo, e.g. chloro) with a compound of formula IXG

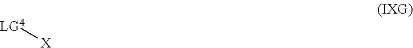

wherein X is as defined herein (i.e. for compounds of the invention, or any particular feature or embodiments thereof) and LG⁴ represents a suitable leaving group (such as a boronic acid), in the presence of a suitable catalyst (such as a suitable metal halide, e.g. CuBr, or phenanthroline) and in the presence of a suitable solvent (such as an organic solvent, e.g. dichloromethane or dichloroethane).
Compounds of formulae IXA, IIB, IXC, IXD, IXE, IXF and IXG are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J.
A. Joule, K. Mills and G. F. Smith, 3^{rd} edition, published by Chapman & Hall, “Comprehensive Heterocyclic Chemistry II” by A. R. Katritzky, C. W. Rees and E. F. V.

### Scriven, Pergamon Press, 1996 and “Science of Synthesis”, Volumes 9-17 (Hetarenes and Related Ring Systems), Georg Thieme Verlag, 2006.

In particular, compounds of formula IXE may be prepared by reaction of a compound of formula IXH

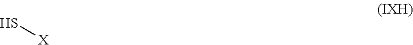

wherein X is as defined herein (i.e. for compounds of the invention, or any particular feature or embodiments thereof), with a compound of formula IXA as herein before defined, under conditions known to those skilled in the art, such as in the presence of a suitable base (such as a metal carbonate, e.g. potassium carbonate, a metal hydroxide, e.g. sodium hydroxide, or an amine base, e.g. triethyl amine), and in the presence of a suitable solvent (such as a polar organic solvent, e.g. N,N′-dimethylacetamide, N,N′-dimethylformamide or tetrahydrofuran, or a mixture of a polar organic solvent and water), under conditions known to those skilled in the art.
Similarly, compounds of formula IXE may be prepared by reaction of a compound of formula IXJ

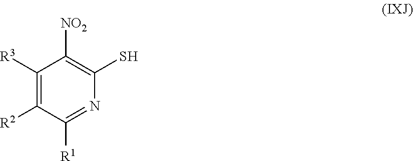

wherein R¹, R² and R³ are as defined herein (i.e. for compounds of the invention, or any particular feature or embodiments thereof), with a compound of formula IXD as described herein, under conditions known to those skilled in the art (for example, where the R⁴ groups present in the compound of formula IXD are not sufficiently electron withdrawing, the reaction may be performed in the presence of a suitable catalyst, such as palladium(II) acetate or copper oxide, in which case the suitable base may be an alkali metal tert-butoxide, such as Kt-OBu).
Similarly, compounds of formulae IXH and IXJ are either commercially available, are known in the literature, or may be obtained either by analogy with the processes described herein, or by conventional synthetic procedures, in accordance with standard techniques, from available starting materials using appropriate reagents and reaction conditions.
The substituents R¹ to R³ and Y, as hereinbefore defined, may be modified one or more times, after or during the processes described above for preparation of compounds of formula IX by way of methods that are well known to those skilled in the art. Examples of such methods include substitutions, reductions, oxidations, dehydrogenations, alkylations, dealkylations, acylations, hydrolyses, esterifications, etherifications, halogenations and nitrations. The precursor groups can be changed to a different such group, or to the groups defined in formula IX, at any time during the reaction sequence. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.
Compounds of the invention may be isolated from their reaction mixtures and, if necessary, purified using conventional techniques as known to those skilled in the art. Thus, processes for preparation of compounds of the invention as described herein may include, as a final step, isolation and optionally purification of the compound of the invention (e.g. isolation and optionally purification of the compound of formula IX).

It will be appreciated by those skilled in the art that, in the processes described above and hereinafter, the functional groups of intermediate compounds may need to be protected by protecting groups. The protection and deprotection of functional groups may take place before or after a reaction in the above-mentioned schemes.

Protecting groups may be applied and removed in accordance with techniques that are well known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3rd edition, T. W. Greene & P. G. M. Wutz, Wiley-Interscience (1999).

### Preparation of Compounds of Formula X

A suitable process for the preparation of a compound of formula X as hereinbefore defined may comprise:

(i) where n represents 2, reaction of a compound of formula XA

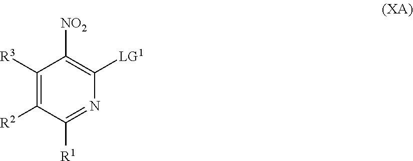

wherein R¹, R² and R³ are as defined herein (i.e. for compounds of the invention, or any particular feature or embodiment thereof) and LG¹ represents a suitable leaving group (such as halo, e.g. chloro), with a compound of formula XB

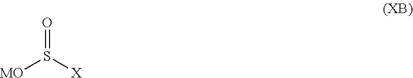

wherein X is as defined herein (i.e. for compounds of the invention, or any particular feature or embodiments thereof) and M represents an alkali metal ion (such as a Na ion),
in the presence of a suitable acid (such as a concentrated acid, e.g. a concentrated mineral acid, for example concentrated HCl, e.g. concentrated aqueous HCl) and in the presence of a suitable solvent (such as a polar organic solvent, e.g. N,N′-dimethylacetamide, N,N′-dimethylformamide or tetrahydrofuran), and optionally in the presence of a suitable phase transfer catalyst (such as a quaternary ammonium salt, e.g. tetra-butyl ammonium chloride);
(ii) where n represents 2, reaction of a compound of formula XC

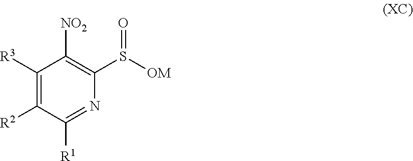

wherein R¹, R² and R³ are as defined herein (i.e. for compounds of the invention, or any particular feature or embodiments thereof) and M represents an alkali metal ion (such as a Na ion), with a compound of formula XD

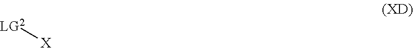

wherein X is as defined herein in formula X (i.e. for compounds of the invention, or any particular feature or embodiments thereof) and LG² represents a suitable leaving group (such as halo, e.g. chloro), in the presence of a suitable acid (such as a concentrated acid, e.g. a concentrated mineral acid, for example concentrated HCl, e.g. concentrated aqueous HCl) and in the presence of a suitable solvent (such as a polar organic solvent, e.g. N,N′-dimethylacetamide, N,N′-dimethylformamide or tetrahydrofuran), and optionally in the presence of a suitable phase transfer catalyst (such as a quaternary ammonium salt, e.g. tetra-butyl ammonium chloride);
(iii) where n represents 2, reaction of a compound of formula XA as hereinbefore defined with a compound of formula XB as hereinbefore defined, in the presence of a suitable metal halide (such as a suitable metal iodide, e.g. CuI, or a suitable metal bromide, e.g. CuBr; which metal halide may be present in excess, such as in amount corresponding to at least 2 molar equivalents of the compound of formula XA and/or the compound of formula XB) and in the presence of a suitable solvent (such as a polar organic solvent, e.g. N,N′-dimethylacetamide, N,N′-dimethylformamide, tetrahydrofuran or 3-dimethyl-2-imidazolidinone), under conditions known to those skilled in the art;
(iv) where n represents 2, reaction of a compound of formula XC as hereinbefore defined with a compound of formula XD as hereinbefore defined, in the presence of a suitable metal halide (such as a suitable metal iodide, e.g. CuI, or a suitable metal bromide, e.g. CuBr; which metal halide may be present in excess, such as in amount corresponding to at least 2 molar equivalents of the compound of formula XC and/or the compound of formula XD) and in the presence of a suitable solvent (such as a polar organic solvent, e.g. N,N′-dimethylacetamide, N,N′-dimethylformamide, tetrahydrofuran or 3-dimethyl-2-imidazolidinone), under conditions known to those skilled in the art;
(v) reaction of a compound of formula XE

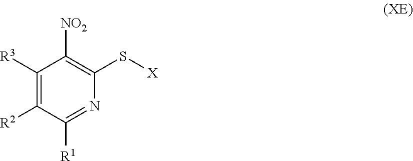

wherein R¹ to R³ and X are as defined herein (i.e. for compounds of the invention, or any particular feature or embodiments thereof), with a suitable oxidising agent (i.e. an oxidising agent chosen and used in a manner as required to achieved the desired degree of oxidation; such as a hypochlorite salt, e.g. sodium hypochlorite, a peroxymonosulfate salt, e.g. potassium peroxymonosulfate (Oxone), a percarboxylic acid, e.g. meta-chloroperoxybenzoic acid (mCPBA), or potassium permanganate) in the presence of a suitable solvent (such as a polar organic solvent, e.g. N,N′-dimethylacetamide, N,N′-dimethylformamide or terahydrofuran), and optionally in the presence of water, under conditions known to those skilled in the art;
(vi) where n represents 2, reaction of a compound of formula XF

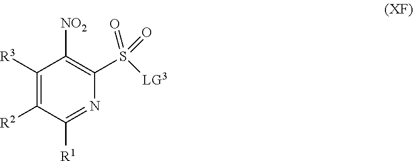

wherein R¹, R² and R³ are as defined herein (i.e. for compounds of the invention, or any particular feature or embodiments thereof) and LG³ represents a suitable leaving group (such as halo, e.g. chloro) with a compound of formula XG

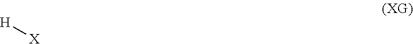

wherein X is as defined (i.e. for compounds of the invention, or any particular feature or embodiments thereof), in the presence of a suitable Lewis acid (such as AlCl₃) and in the presence of a suitable solvent (such as an organic solvent, e.g. dichloromethane or dichloroethane).
Compounds of formulae XA, XB, XC, XD, XE, XF and XG are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.
In particular, compounds of formula IV may be prepared by reaction of a compound of formula XH

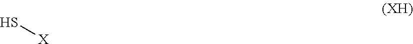

wherein X is as defined herein (i.e. for compounds of the invention, or any particular feature or embodiments thereof), with a compound of formula XA as herein before defined, under conditions known to those skilled in the art, such as in the presence of a suitable base (such as a metal carbonate, e.g. potassium carbonate, a metal hydroxide, e.g. sodium hydroxide, or an amine base, e.g. triethyl amine), and in the presence of a suitable solvent (such as a polar organic solvent, e.g. N,N′-dimethylacetamide, N,N′-dimethylformamide or tetrahydrofuran, or a mixture of a polar organic solvent and water), under conditions known to those skilled in the art.
Similarly, compounds of formula XE may be prepared by reaction of a compound of formula XJ

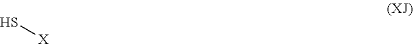

wherein X is as defined herein (i.e. for compounds of the invention, or any particular feature or embodiments thereof), with a compound of formula XA as herein before defined, under conditions known to those skilled in the art, such as in the presence of a suitable base (such as a metal carbonate, e.g. potassium carbonate, a metal hydroxide, e.g. sodium hydroxide, or an amine base, e.g. triethyl amine), and in the presence of a suitable solvent (such as a polar organic solvent, e.g. N,N′-dimethylacetamide, N,N′-dimethylformamide or tetrahydrofuran, or a mixture of a polar organic solvent and water), under conditions known to those skilled in the art.
Similarly, compounds of formula XE (particularly where at least one Y is present and represents an electron-withdrawing group, such as —NO₂) may be prepared by reaction of a compound of formula XK

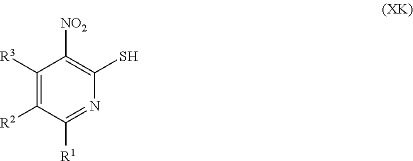

wherein R¹, R² and R³ are as defined herein (i.e. for compounds of the invention, or any particular feature or embodiments thereof), with a compound of formula XD as described herein, under conditions known to those skilled in the art (for example, where the R⁴ groups present in the compound of formula XD are not sufficiently electron withdrawing, the reaction may be performed in the presence of a suitable catalyst, such as palladium(II) acetate or copper oxide, in which case the suitable base may be an alkali metal tert-butoxide, such as Kt-OBu).
Similarly, compounds of formulae XJ and XK are either commercially available, are known in the literature, or may be obtained either by analogy with the processes described herein, or by conventional synthetic procedures, in accordance with standard techniques, from available starting materials using appropriate reagents and reaction conditions.
The substituents R¹ to R³ and Y, as hereinbefore defined, may be modified one or more times, after or during the processes described above for preparation of compounds of formula X by way of methods that are well known to those skilled in the art. Examples of such methods include substitutions, reductions, oxidations, dehydrogenations, alkylations, dealkylations, acylations, hydrolyses, esterifications, etherifications, halogenations and nitrations. The precursor groups can be changed to a different such group, or to the groups defined in formula X, at any time during the reaction sequence. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.
Compounds of the invention may be isolated from their reaction mixtures and, if necessary, purified using conventional techniques as known to those skilled in the art. Thus, processes for preparation of compounds of the invention as described herein may include, as a final step, isolation and optionally purification of the compound of the invention (e.g. isolation and optionally purification of the compound of formula X).
It will be appreciated by those skilled in the art that, in the processes described above and hereinafter, the functional groups of intermediate compounds may need to be protected by protecting groups. The protection and deprotection of functional groups may take place before or after a reaction in the above-mentioned schemes.
Protecting groups may be applied and removed in accordance with techniques that are well known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3rd edition, T. W. Greene & P. G. M. Wutz, Wiley-Interscience (1999).

As discussed above, in one aspect, the present invention provides a selenium compromised thioredoxin reductase-derived apoptotic protein (SecTRAP) forming agent for use in treating a T-cell infiltrated cancer in a subject, wherein said agent has immunostimulatory activity thereby causing said subject to raise (or stimulate or enhance or elicit) an immune response against said cancer (in said subject).

Without wishing to be bound by theory, it is believed that SecTRAP forming agents for use in the present invention inhibit the C-terminal active site of the enzyme TrxR (but do not abolish or do not significantly inhibit the activity at the N-terminal active site of TrxR), thereby causing an increase in the level of reactive oxygen species, a lowering of thioredoxin (Trx) production and release (e.g. lowering the concentration of reduced Trx) (and possibly lowering the concentration of PDI and/or lowering the concentration of reduced redox-active proteins and systems), and thereby a reduction in the size of the Treg cell (regulatory T cell) population (e.g. in the tumour microenvironment), which causes (or stimulates or enhances) an anti-cancer immune response. Further, and wishing not to be bound by theory, cytotoxic T-cells can be attracted to infiltrate the tumor microenvironment after TrxR/Trx-rich cells have lyzed in the tumor tissue as a consequence of SecTRAP action on said TrxR/T rx-rich cells.

T-cell infiltrated cancers (or T-cell infiltrated tumors) are preferred cancers to be treated in accordance with the present invention. Without wishing to be bound by theory, given the inventors' surprising finding that SecTRAP forming agents can stimulate an anti-cancer immune response, T-cell infiltrated cancers may be particularly attractive for treatment in accordance with the present invention as such cancers may already have a tumour microenvironment that is “set-up” or “poised” or “primed” (e.g. in terms of the T-cell populations present) to react to the immunostimulatory activity conferred by the SecTRAP forming agents.

T-cell infiltrated cancers are cancers (or tumours) that have a population (or a significant or physiologically relevant population) of T-cells (immune cells) in the tumour microenvironment (or intratumoural space). Typically, T-cell infiltrated cancers are characterized by the presence of CD8+ cytotoxic T-cells, CD4+ helper cells and CD4+ regulatory T cells (Tregs). CD8+ T cells are the key effector cell population that mediate effective anti-cancer activity. Tregs have an immunosuppressive role in the tumour microenvironment. A person skilled in the art would be readily able to determine whether or not (and the degree to which) a given cancer (or tumour) is T-cell infiltrated. For example, a biopsy of tumour tissue could be done and analysed for the presence of (or absence of) T-cells or the degree of T-cell infiltration, e.g. based on the cell surface marker profile of the T-cells. For example, Treg cells can be identified on the basis of the marker profile CD4+CD25+FOXP3+.

T-cell infiltrated tumours may also comprise B-cells, macrophages, myeloid-derived suppressor cells, NK cells, neutrophils and/or mast cells.

Within the tumor microenvironment, immune cells generally considered to be antitumoral are cytotoxic T lymphocytes (CD8+) and natural killer cells (NK). Immune cells considered to promote tumor growth are tumor-associated macrophages (TAMs), neutrophils, and mast cells. Some immune cell types, e.g. regulatory T cells (Tregs) and myeloid-derived suppressor cells (MDSCs) can inhibit immune reactions against tumor cells. Generally, the tumor cells together with the associated stroma will direct which immune cells dominate within the TME.

As mentioned above, a person skilled in the art would be readily able to determine whether or not (and the degree to which) a given cancer (or tumour) is T-cell infiltrated. There are various methods known in the art for determining levels and types of various immune cells within a tumor tissue (and thus whether or not, or the degree to which a given cancer is T-cell infiltrated). A number of tumor tissue biomarker assay methods and recommended guidelines for clinical use are reviewed in (Masucci et al. 2016).

Samples (e.g. tumour samples) can be taken by core-needle biopsy. One method of assessing T-cell infiltration involves hematoxylin- and eosin-staining (H&E) of tumor tissue samples. Using H&E, the immune infiltrate can be normally scored based on density and distribution of lymphocytes. Another method uses immunohistochemical (IHC) techniques where certain immune-cell expressed markers are evaluated. IHC stains can be done using CD3, CD4, CD8+, FOXP3+, individually or in combinations. A combination of CD3 and CD4 may be used to determine the extent of T cell infiltration. Flow cytometric-based immunophenotyping assays can be used on live cells from biopsies. Another method is to use gene expression of immune markers. Tumor T-cell markers include granzyme A, granzyme B, perforin, Eomesodermin, IFN-gamma, TNF, CXCL9, CXCL10, CD8A, CD4, FOXP3, ICOS, and CTLA4. Other markers and marker panels can be used to assess T-cell infiltration include one or more of the markers selected from the group consisting of CD45, CD3, CD4, CD8, Ki67, CTLA-4, PD-1, PD-L1, LAG-3, TIM-3, ICOS, CD25, OX40, ICOS, T-bet, CD25, FOXP3, CD127, Ki67, CD45RA, CTLA-4, GITR, CD103, Neuropilin-1, Helios, CD45RA, CD45RO, CD16, CD56, CD69, CD19, CD20, and CD27.

One marker of all leukocytes is CD45. CD45 is expressed on almost all hematopoietic cells except for mature erythrocytes.

One marker for all T cells is CD3.

Markers of T cells include CD3, CD4, CD8, Ki67, CTLA-4, PD-1, PD-L1, LAG-3, TIM-3, and ICOS.

Markers of T cell activation include CD25, OX40, ICOS and CTLA4.

Markers of CD4+Th1 cells include T-bet.

Markers of Treg cells include CD3, CD4, CD25, FOXP3, CD127, Ki67, CD45RA, CTLA-4, GITR, CD103, Neuropilin-1, and Helios.

One marker panel for Treg cells is CD4+CD25+FOXP3+. CD25 is a gene that is expressed largely by lymphocytes and to a particularly strong extent by Tregs. Thus, Tregs (Treg cells) may be characterized by the expression of CD4, CD25 and FoxP3. Another marker panel for Treg cells is CD3+, CD4+, CD25+, FOXP3+ and CD45+. Thus, Tregs (Treg cells) may be characterized by the expression of CD3, CD4, CD25, FoxP3 and CD45.

Markers of naïve T cells include CD45RA.

Markers of memory T cells include CD45RO.

Naïve versus memory CD4+ cells can be expressed as the ratio CD4+CD45RO+/CD4+.

Naïve versus memory CD8+ cells can be expressed as the ratio CD8+CD45RO+/CD8+.

Markers of activated natural killer (NK) cells include CD16, CD56 and CD69.

Markers of B cells include CD19, CD20, and CD27.

One marker for total B cells in tissue is CD20, and CD19 using flow cytometry.

Naïve versus memory B cells can be expressed as the ratio CD19+CD27+/CD19+.

In some embodiments, uses and methods of the present invention may comprise a step of determining (or assessing) whether or not (and optionally the degree or level to which) a cancer (or tumour) is immune cell infiltrated (e.g. T-cell infiltrated). In some embodiments, such a step is performed prior to the start of treatment with a SecTRAP forming agent. As discussed elsewhere herein, such a step may permit the selection of subjects to be treated in accordance with the invention. For example, a subject having a cancer that has been determined (or categorized) as immune cell infiltrated (e.g. T-cell infiltrated) would, in some embodiments, be a preferred subject for treatment in accordance with the invention.

In some embodiments, an assessment of whether or not a cancer is T-cell infiltrated (or the degree of T-cell infiltration) may be done by tumour biopsy followed by examination or analysis of the cells present based on the cell surface marker profile (or other marker profiling e.g. based on gene expression). Suitable marker profiles are known to the skilled person and are discussed herein. One suitable method for the analysis of a cell marker profile (e.g a cell surface marker profile) is flow cytometry. The skilled person is familiar with flow cytometric methods and could readily select suitable reagents for use in such methods (e.g. antibodies against given markers), e.g. as described in the Example section herein.

In some embodiments, an assessment of whether or not a cancer is T-cell infiltrated (or the degree of T-cell infiltration) may be done by analysing (e.g. the presence or absence or level of) one or more cytokines (e.g. a cytokine profile) in a sample (e.g. a tumour sample).

In T-cell infiltrated tumors there will typically exist a signature cytokine profile depending on which types cells are present and which are dominant. For example, CD4+T-helper cell subsets have been defined based on their signature cytokine profiles (Golubovskaya & Wu 2016). CD4+ T cells, upon activation by antigen-presenting cells (APCs), differentiate into cytokine-expressing effector helper T (Th) cells, which are classified as Th1, Th2, Th17, and T follicular helper (Tfh) cell subsets on the basis of their cytokine secretion and immune regulatory function. Another example is T effector cells, where central memory T-cells, effector memory T-cells and effector memory RA T-cells have distinguishable cytokine signaling signatures (Willinger et al. 2005).

As described elsewhere herein, T-cell infiltration (e.g. presence, absence or degree of) may be assessed in cancer (or tumour) samples (or specimens). Tumor samples may be in the form of core biopsies or tumor sections. Tumor sections are wider than core biopsies, and are likely to provide a more accurate picture of infiltration. TIL levels are normally scored (e.g. by pathologists and/or biologists) on hematoxylin- and eosin-stained (H&E) samples. Immunohistochemistry and/or analysis of immune gene signatures may also be used. The samples are typically contained on (or presented on) a slide. Using H&E, the immune infiltrate is normally scored based on density and distribution of lymphocytes. Counting is performed visually using high-powered fields (HPFs). IHC stains can be done using CD3, CD4, CD8+, FOXP3+ individually or in combinations. A combination of CD3 and CD4 has been used to determine extent of T cell infiltrate. Tumor infiltrating lymphocytes are usually defined by their location, either as intratumoral lymphocytes or stromal lymphocytes. The % of intratumoral lymphocytes is typically based on the total area or tumor nests occupied by intraepithelial mononuclear cells or mononuclear cells in direct contact with individual tumor cells. The % of stromal lymphocytes is typically based on the total area of stroma occupied by mononuclear cells. For an individual patient, the percentage of infiltration is typically based on the mean of all samples assessed, due to variation in infiltration levels between different sections of the same tumor.

Immunological changes in peripheral blood and tumor can potentially reflect tumor response to treatment in patients. Immune biomarkers can be tumor-derived and/or immune cell-derived. The parameters measured at the tumor site can include specific tumor and immune changes before and after treatment. Pre-treatment and post-treatment biopsies may be analyzed for lymphocyte infiltration by IHC and flow cytometric-based immunophenotyping assays. Tumor specimens can be analysed by protein assays, genomics, e.g. next generation sequencing, transcriptomics, and protein function. Tumor-infiltration by lymphocytes is primarily measured by IHC, using marker panels. Multiplex staining, with up to seven fluorescent dyes, can be used. A number of methods and recommended guidelines for clinical use are reviewed in (Masucci et al. 2016). For example, using IHC on tumor specimens showed that anti-PD-1 blockade in responding patients resulted in increased levels of CD8+ T-cells in the invasive tumour margin and inside the tumour center. Next generation sequencing, or whole-genome sequencing (WES), can be used to assess tumor mutational load and tumour antigen-specific T-cell accumulation at the tumour site (by so-called T-cell receptor sequence usage). Baseline expression of PD-L1 on immune cells or tumor cells can be qualitatively scored using formalin-fixed, paraffin-embedded (FFPE) tissue, using commercially available kits.

A panel of tumor T-cell markers that can indicate a response to anti-PD-L1 can include granzyme A, granzyme B, perforin, Eomesodermin, IFN-γ, TNF, CXCL9, CXCL10, CD8A, CD4, FOXP3, ICOS, and CTLA4. Reference genes can be SP2, GUSB, TMEM55B and VPS33B (Herbst et al. 2014).

There are commercially available systems that can be used for tumour tissue gene expression profiling. For example, the nCounter GX PanCancer Immune Profiling Panel is a comprehensive set of 770 genes combining markers for 24 different immune cell types and populations, 30 common cancer antigens, and genes that represent all categories of immune response including key checkpoint blockade genes. The nCounter PanCancer Progression Panel is a panel to aid assessment of cancer progression. The panel has 770 genes from four major biological processes that contribute to increased tumor growth and invasiveness, including angiogenesis, the epithelial-to-mesenchymal transition, extracellular matrix remodelling, and metastasis (nanoString Technologies).

In some preferred embodiments, there is a high grade (or high level) of cancer (tumour) T-cell infiltration. H&E staining or any other appropriate method may be used to determine this, e.g. other methods described herein. A skilled person is able to determine what represents high grade infiltration. In some embodiments, high grade infiltration is a situation where tumour infiltrating lymphocytes (TILs) represent ≥40% or ≥50% (or more) of the total cells within a tumour. For example, in LPBC (lymphocyte predominant breast cancer), one measure of high grade infiltration has been defined for the situation where TILs comprise more than half of the cells within a tumor (Pruned et al. 2016).

For reporting the extent of tumor infiltration by lymphocytes (e.g. T-cells), certain thresholds may be used to define minimal, moderate and extensive infiltration. Purely by way of Example, minimal infiltration may represent the case where intratumoral lymphocytes are less than 5%, and, stromal lymphocytes are less than 10% (of the total number of cells in the tumour). Moderate infiltration may represent the case where intratumoral lymphocytes are more than 5%, or, stromal lymphocytes are more than 10% (of the total number of cells in the tumour or the stroma). However, cut-off values and extent of infiltration may be assessed in different ways.

In some embodiments, T-cell infiltrated cancers (or tumours) are tumours in which infiltrating lymphocytes (TILs) (e.g. intratumoural lymphocytes) represent ≥5%, or ≥10%, ≥15%, ≥20%, ≥25%, ≥30%, ≥40% or, ≥50% (or more) of the total cells within a tumour. In some embodiments, T-cell infiltrated cancer (or tumours) are tumours in which infiltrating lymphocytes (TILs) (e.g. stromal lymphocytes) represent ≥10%, ≥15%, ≥20%, ≥25%, ≥30%, ≥40% or, ≥50% (or more) of the total cells within the tumour or the stroma.

The tumor microenvironment (TME) can be infiltrated by immune cells to a certain extent depending on the tumor type and stage.

When discussing grade of infiltration, this typically relates to comparisons between normal and tumor tissue, where high indicates that the frequency of immune cells in the tumor microenvironment is significantly higher than that in tumor-surrounding tissue (tumor-enriched). High grade infiltration can also refer to comparisons between different tumor types, between subtypes within a tumor type, and between tumor stages for a specific tumor type. For example, in the case of breast cancer, infiltration is reported to be higher in some breast cancer subtypes than others, and varies from patient to patient (Pruned et al. 2016). Infiltration (e.g. in breast cancer) can be higher in the stromal region than in the tumor bed. Triple-negative breast cancer is one subtype associated with a high degree of infiltration compared to other breast cancer subtypes.

Tumor infiltrating immune cells possess the functional capacity to promote both anti- and pro-tumorigenic effects, where the directionality and extent of effect is governed, in part, by cellular and molecular constituents of the tumor microenvironment that vary within and across tumor types. Purely by way of example, in breast cancer, melanoma, head and neck cancer, colorectal cancer (and other cancers), the TME can be infiltrated by immune cells. Infiltration is higher in the stromal region than in the tumor bed. TNBC (triple negative breast cancer) is one subtype associated with a high degree of infiltration. The presence of TILs can indicate the presence of a tumor-specific immune response, but it seems that the role of the immune system for primary tumor control is relatively ineffective as these antigenic primary cancers still can successfully grow. Immune cell infiltration into the tumor, and recognition and killing of tumor cells can be aided by various antibodies which enhance the function of immune cells. In view of the present invention, SecTRAP forming agents can added to the clinician's toolkit for treating cancer by stimulating an anti-cancer immune response.

The composition of the tumor microenvironment (TME) influences the growth of the tumor. The TME comprises the tumor bed, the stroma, extracellular matrix, and vessels (lymphatic and blood). The stroma consists of endothelial cells, mesenchymal stem cells, cancer-associated fibroblasts, pericytes, and can interact both with tumor cells and immune cells. Cells of the stroma can promote tumor progression, metastasis and chemoresistance. Tumor cells can re-programme infiltrating immune cells and stromal elements to a pro-tumorigenic mode of action via cell-to-cell contacts and by secreted factors

Tumors (e.g. tumours experiencing an anti-cancer immune response in accordance with the invention) can exhibit a T cell-inflamed phenotype. This phenotype is characterised by high (or higher) TIL (tumour-infiltrating lymphocytes) levels, e.g. in comparison with normal or control levels (e.g. levels in normal tissue or tumour-surrounding tissue), increased expression of T-cell activation markers, chemokines that recruit T cells, and may also be characterised by negative immune regulators including FoxP3+ Treg cells and IDO (indoleamine-pyrrole 2,3-dioxygenase). A type I IFN signature may also be present. In contrast, a non-T cell-inflamed phenotype, is characterised by a lack of (or lower number of) TILs, e.g. in comparison with normal or control levels (e.g. in normal tissue or tumour-surrounding tissue), but is rich in suppressive Th2 cytokines and chemokines, TAMs and MSDCs. The latter phenotype is also known as chronic inflammation.

Within the TME, immune cells generally considered to be antitumoral are cytotoxic T lymphocytes (CD8+) and natural killer cells (NK). Thus, in some embodiments of the present invention an anti-cancer immune response may be characterised by the presence of (or increased level of, e.g. in comparison to non-cancerous or normal tissue or tumour-surrounding tissue) cytotoxic T lymphocytes (CD8+) and/or natural killer cells (NK). Immune cells considered to promote tumor growth are tumor-associated macrophages (TAMs), neutrophils, and mast cells. Some immune cell types, e.g. regulatory T cells (Tregs) and myeloid-derived suppressor cells (MDSCs) can inhibit immune reactions against tumor cells. Generally, the tumor cells together with the associated stroma will direct which immune cells dominate within the TME.

The three major types of lymphocytes are T-cells, B-cells and natural killer (NK) cells. T-cells function in cell-mediated, cytotoxic adaptive immunity. NK cells function in cell-mediated, cytotoxic innate immunity, and B cells act in antibody-driven adaptive immunity. T-cells are divided into CD8+ and CD4+ groups, where CD8+ are have cytotoxic activity whereas CD4+ are helper cells. Memory T-cells are circulating antigen-experienced T-cells, which can rapidly expand after encountering the presented antigen in the target tissue.

Effector T-cell is a broad category that includes various T-cell types that actively respond to a stimulus, such as co-stimulation. This includes helper, cytotoxic (killer), regulatory, and potentially other T-cell types. Cytotoxic CD8+ effector cells are the group of cells that perform the active elimination of tumor cells.

T helper cells (TH cells) assist other white blood cells in immunologic processes, including maturation of B-cells into plasma cells and memory B-cells, and activation of cytotoxic T-cells and macrophages. These cells are also known as CD4+ T-cells because they express the CD4 glycoprotein on their surfaces. Helper T-cells become activated when they are presented with peptide antigens by MHC class II molecules, which are expressed on the surface of antigen-presenting cells (APCs). Once activated, they divide rapidly and secrete cytokines that regulate or assist in the active immune response. TH cells can differentiate into one of several subtypes, including TH1, TH2, TH3, TH17, TH9, or TFH, which secrete different cytokines to facilitate different types of immune responses. Signalling from the ARC directs T cells into particular subtypes.

Regulatory T cells (Tregs) segregate into two primary categories: thymus-derived natural Tregs (nTregs) that develop from the interaction between immature T cells and thymic epithelial stromal cells, and inducible Tregs (iTregs) that arise from the conversion of CD4+FoxP3− T cells into FoxP3 expressing cells. Several publications have shown that CpG demethylation of regulatory elements in the FOXP3 locus support stable expression of FOXP3 and a suppressive phenotype. Normally, these Treg subsets complement one another's actions by maintaining tolerance of self-antigens, thereby suppressing autoimmunity. Tregs normally account for only 5-10% of all circulating CD4+ T cells. Tregs can be identified by expression of the FoxP3, and are known to supress the suppression of any type of effector T cell, by secretion of specific inhibitory cytokines such as IL-10, IL-35, and TGF-β, or by direct cell-cell contact. Suppression is seen by down-regulation of IL-2 and/or interferon-gamma (IFN-γ) production in effector T cells. Treg deregulation can lead to autoimmune disease, whilst gain of function can lead to carcinogenesis. In most cases, CD4+CD25+ Treg cells suppress the anti-tumor immune response in 2 aspects: one mode is via cells in the tumor draining regional lymph node; the other mode is through the tumor tissue. In the tumor draining regional lymph node cells, many proliferative CD4+CD25+ Treg cells inhibit the proliferation of effector cells within the same lymph node. In the tumor tissue, CD4+CD25+ Treg cells prevent effector T cells from killing tumor cells.

MDSCs have a role in tumour growth and metastasis via promotion of immune privilege (ability to tolerate the introduction of antigens without eliciting an inflammatory immune response), tumour microenvironment remodelling, establishment of a pre-metastatic niche (a scenario where non-cancer cells promote future metastasis) and interaction with tumour to promote differentiation, invasion and angiogenesis. Myeloid-derived suppressor cells accumulate in the blood, lymph nodes, bone marrow, and at tumor sites in many human cancers and animal tumor models, and inhibit both adaptive and innate immunity. They notably have the capacity to inhibit CD8+ T-cell antigen-specific reactivity by different mechanisms, mainly through their capacities to produce nitric oxide and radical oxygen species, and their presence within a tumor favours tumor progression. MDSCs have been implicated in promoting angiogenesis, tumor cell invasion, and metastases. The presence of MDSCs correlates with reduced survival in human cancers, including breast cancer and colorectal cancer. Human MDSCs express markers such as CD11b+ and CD33+ but are mostly negative for HLA-DR and lineage-specific antigens (Lin), including CD3, CD19, and CD57. Monocytic MDSCs are HLA-DR, CD11b+, CD33+ and CD14+, and granulocytic MDSCs are HLA-DR−, CD11b+, CD33+, CD15+. Mature MSDCs express HLA-DR.

High levels of infiltration by tumor-associated macrophages (TAMs) in TNBC tumors generally associates with poor prognosis. In vivo TAM phenotype depends on the location, tumor type and stromal interactions. The phenotype is determined during differentiation from monocyte. The M1-type TAMs are generally considered pro-inflammatory and promote anti-tumor immune responses, whereas the M2-type is anti-inflammatory and known to have immunosuppressive properties including low antigen presenting capability and low cytotoxic functions. Clinical data from human invasive breast cancer samples show that the abundance of TAMs correlates with high tumor grade, low hormone receptor status and reduced relapse-free and overall-survival. M1 and M2 macrophages can be distinguished based on the differential expression of transcription factors and surface molecules and the disparities in their cytokine profile and metabolism. Reports suggest that macrophages can directly suppress T-cell responses through PD-L1. PD-L1 is notably expressed on macrophages.

According to in vitro co-culture studies, the TNBC cell line MDA-MB-231 can promote monocyte differentiation into M2-type macrophages. In M1-type macrophages, the thioredoxin activation pathway is significantly downregulated. It has been shown that extracellular Trx1 can bind to the surface of macrophages and be internalized. Trx1 promotes differentiation to the M2-phenotype (Hadri et al. 2012), which is a phenotype associated with tumor promotion.

Neutrophils are the most abundant type of granulocytes and the most abundant type of leukocytes. Neutrophils are a type of phagocyte and are normally found in the bloodstream. During the beginning (acute) phase of inflammation, particularly as a result of bacterial infection, environmental exposure, and some cancers, neutrophils are one of the first-responders of inflammatory cells to migrate towards the site of inflammation. They migrate, via chemotaxis, through the blood vessels, then through interstitial tissue, following chemical signals such as Interleukin-8 (IL-8), C5a, fMLP, Leukotriene B4 and H₂O₂.

In preferred embodiments, T-cell infiltrated cancers to be treated in accordance with the invention have high levels of Tregs (CD4+CD25+FOXP3+) in the tumour microenviroment (high levels of intratumoural Tregs).

High levels of Tregs typically means that that the frequency (or number or prevalence) of Tregs in the tumor microenvironment is significantly higher than that in tumor-surrounding tissue (tumor-enriched). The discussion of high level (or high grade) T-cell infiltration may be applied, mutatis mutandis, to Tregs in particular.

One important ratio within a tumor is the ratio of CD8+ cells to Treg cells. Apart from a general high level of infiltration, a higher level of Treg will mean mean a lower CD8+/Treg ratio (e.g. as compared to normal tissue or tumour-surrounding tissue) which has been suggested to be detrimental in various tumor types. Thus, in some embodiments, the treatment of cancers having a low CD8+/Treg ratio is preferred.

In some embodiments, T-cell infiltrated cancers have high levels of Th2 CD4+ T cells, myeloid derived suppressor cells, M2 macrophages and/or neutrophils in the tumour microenviroment (high levels of intratumoural Tregs).

In some embodiments, T-cell infiltrated cancers to be treated have a high (or higher) ratio of Tregs to CD8+ T cells.

High levels of Tregs in the tumour microenvironment have been associated with worsened disease outcomes in many cancer types. Thus depleting Treg populations or inhibiting Treg activity in particular within the tumour microenvironment is desirable. Without wishing to be bound by theory, it is believed that treatments in accordance with the invention reduce the Treg cell population size (and/or activity), and for example lead to a decrease in the ratio of Tregs to CD8+ T cells. It is believed this would be therapeutically beneficial as there would be more anti-cancer effector T-cell activity in the tumour microenvironment.

Thus, in some embodiments, the immune response against cancer (or anti-cancer response or anti-cancer immune response) is characterized by a reduction in the level of Tregs. The level of Tregs may be the relative level of Tregs or the absolute level (or number) of Tregs, as discussed below. Typically, the reduction in the level of Tregs is a reduction within the tumour (or cancer) or in the tumour microenvironment (or cancer microenvironment), or in a sample such as a biopsy or cell suspension that has been obtained from the tumour (or cancer) or tumour microenvironment (or cancer microenvironment). In some embodiments, such a sample may be processed (e.g. prior to analysis), for example to obtain a cell suspension from the tumour (or cancer) or tumour microenvironment (or cancer microenvironment).

Cells may be categorized (or identified or designated) as Tregs based on their marker profile (e.g. cell surface marker profile). Treg cell markers and marker profiles are described elsewhere herein. Cells may be categorized (or identified or designated) as Tregs if they express (i.e. are positive for) CD4, CD25 and FoxP3. Put another way, cells may be categorized (or identified or designated) as Tregs if they are CD4+, CD25+ and FoxP3+. Cells may be categorized (or identified or designated) as Tregs if they express (i.e. are positive for) CD45, CD3, CD4, CD25 and FoxP3. Put another way, cells may be categorized (or identified or designated) as Tregs if they are CD45+, CD3+, CD4+, CD25+ and FoxP3+. Marker profiles may be assessed by a flow cytometry method employing appropriate antibodies, such as the method described in Example 2 herein.

A reduction in the level of Tregs may be any measurable reduction or decrease, e.g. when compared with a control level. Preferably, the level is significantly reduced, compared to the level found in an appropriate control sample or subject or population (e.g a healthy or normal subject or population, or sample therefrom, or a treated subject's “baseline” level). More preferably, the significantly reduced levels are statistically significant, preferably with a probability value of <0.05 or <0.01. Appropriate control levels (or control samples or values) could be readily chosen by a person skilled in the art. Appropriate control “values” could also be readily determined without running a control “sample” in every test, e.g. by reference to the range for normal subjects and/or by reference to the “baseline” level in a subject being treated.

The control level may correspond to the level of Tregs in the same individual subject, or a sample from said subject (e.g. a tumour biopsy), measured at an earlier time point (e.g. comparison with a “baseline” level in that subject, e.g. the level in the subject prior to the start of treatment in accordance with the invention, or the level at an earlier time point during the treatment regime). This type of control level (i.e. a control level from an individual subject) is particularly useful for embodiments of the invention where serial or periodic measurements of Tregs in individuals, either healthy or ill, are taken looking for changes in the levels of Tregs. In this regard, an appropriate control level will be the individual's own baseline, stable, nil, previous or dry value (as appropriate) as opposed to a control or cutoff level found in the general population Control levels may also be referred to as “normal” levels or “reference” levels. The control level may be a discrete figure or a range.

In some embodiments, the reduction in the level of Tregs is a reduction of at least 1%, at least 2%, at least 3%, at least 4%, at least 5%, at least 10%, at least 20%, at least 30%, at least 40%, at least 50%, at least 60%, at least 70%, at least 80%, at least 90% or even 100%, e.g. in comparison with a control level.

The level of Tregs (e.g. within a tumour or cancer or tumour microenvironment or cancer microenvironment) may be expressed as the percentage of Tregs (e.g. as characterised by expression CD45, CD3, CD4, CD25 and FoxP3) in the CD45+ cell population (e.g. in a sample such as a tumour biopsy). Alternatively viewed, the level of Tregs (e.g. within a tumour or cancer or tumour microenvironment or cancer microenvironment) may be considered as the size of the sub-population of Tregs (e.g. as characterised by expression CD45, CD3, CD4, CD25 and FoxP3) from within the total population of CD45 positive (CD45+) cells (e.g. in a sample such as a tumour biopsy), which may be expressed as a percentage of the total CD45+ cells. Alternatively viewed, the level of Tregs (e.g. within a tumour or cancer or tumour microenvironment or cancer microenvironment) may be considered the proportion of the total CD45+ cell population (e.g. in a sample such as a tumour biopsy) that is Tregs (e.g. as characterised by expression CD45, CD3, CD4, CD25 and FoxP3), which may be expressed as a percentage of the total CD45+ cells.

Thus, in some embodiments, the level of Tregs is the relative level of Tregs or the relative population size of Tregs or the relative proportion of Tregs (e.g. relative to the total CD45+ cell level or the total CD45+ population size). Thus, in some embodiments, there is a reduction in the relative level of Tregs (or in the relative population size of Tregs), e.g. relative to the total CD45+ cell level or the total CD45+ population size. In some embodiments, the absolute number of Tregs (e.g. within a tumour or tumour microenvironment) is reduced.

In some embodiments, the immune response against cancer (or anti-cancer response or anti-cancer immune response) is characterized by an increase in the level of CD8+ T-cells (or CD8+ effector T-cells or CD8+ cytotoxic T-cells) and/or other cytotoxic immune cells (preferably an increase in the level of CD8+ T-cells). The level of CD8+ T-cells and/or other cytotoxic immune cells may be the relative level of CD8+ T-cells and/or other cytotoxic immune cells or the absolute level (or number) of CD8+ T-cells and/or other cytotoxic immune cells, as discussed below. Typically, the increase in the level of CD8+ T-cells and/or other cytotoxic immune cells is an increase within the tumour or cancer or in the tumour microenvironment or cancer microenvironment, or in a sample such as a biopsy or cell suspension that has been obtained from the tumour (or cancer) or tumour microenvironment (or cancer microenvironment). In some embodiments, such a sample may be processed (e.g. prior to analysis), for example to obtain a cell suspension from the tumour or tumour microenvironment.

Cells may be categorized (or identified or designated) as CD8+ T-cells based on their marker profile (e.g. cell surface marker profile), typically of course based on the expression of CD8. CD8+ T-cell marker profiles are known in the art (e.g. a panel of markers comprising CD8+ and CD45+, or a panel of markers comprising CD8+ and CD45+ and CD3+). Additional markers for CD8+ effector T cells are CD27, CD28, CD45RA and CCR7. Thus, CD8+ T-cells (CD8+ effector T-cells) may be characterized by the expression of one or more (or all) of CD27, CD28, CD45RA and CCR7, typically of course in addition to CD8 (and optionally also CD3). Marker profiles, e.g. CD8 expression status, may be assessed by a flow cytometry method employing appropriate antibodies, such as the method described in Example 4 herein. Marker profiles of other cytotoxic immune cells are known in the art.

An increase in the level of CD8+ T-cells and/or other cytotoxic immune cells (preferably an increase in the level of CD8+ T-cells) may be any measurable increase or elevation, e.g. when compared with a control level. Preferably, the level is significantly increased, compared to the level found in an appropriate control sample or subject or population (e.g a healthy or normal subject or population, or sample therefrom, or a treated subject's “baseline” level). More preferably, the significantly increased levels are statistically significant, preferably with a probability value of <0.05 or <0.01. Appropriate control levels (or control samples or values) could be readily chosen by a person skilled in the art. Appropriate control “values” could also be readily determined without running a control “sample” in every test, e.g. by reference to the range for normal subjects and/or by reference to the “baseline” level in a subject being treated.

The control level may correspond to the level of CD8+ T-cells and/or other cytotoxic immune cells in the same individual subject, or a sample from said subject (e.g. a tumour biopsy), measured at an earlier time point (e.g. comparison with a “baseline” level in that subject, e.g. the level in the subject prior to the start of treatment in accordance with the invention, or the level at an earlier time point during the treatment regime). This type of control level (i.e. a control level from an individual subject) is particularly useful for embodiments of the invention where serial or periodic measurements of CD8+ T-cells and/or other cytotoxic immune cells in individuals, either healthy or ill, are taken looking for changes in the levels of CD8+ T-cells and/or other cytotoxic immune cells. In this regard, an appropriate control level will be the individual's own baseline, stable, nil, previous or dry value (as appropriate) as opposed to a control or cutoff level found in the general population Control levels may also be referred to as “normal” levels or “reference” levels. The control level may be a discrete figure or a range.

In some embodiments, the increase in the level of CD8+ T-cells and/or other cytotoxic immune cells (preferably an increase in the level of CD8+ T-cells) is an increase of at least 1%, at least 2%, at least 3%, at least 4%, at least 5%, at least 10%, at least 20%, at least 30%, at least 40%, at least 50%, at least 60%, at least 70%, at least 80%, at least 90%, at least 100%, at least 200%, at least 300%, at least 400% or at least 500% (e.g. up to 10%, up to 50%, up to 100%, up to 200%, up to 300%, up to 400% or up to 500%), e.g. in comparison with a control level.

The level of CD8+ T-cells and/or other cytotoxic immune cells (e.g. within a tumour or cancer or tumour microenvironment or cancer microenvironment) may be expressed as the percentage of CD8+ T-cells and/or other cytotoxic immune cells in the CD45+ cell population (e.g. in a sample such as a tumour biopsy). Alternatively viewed, the level of CD8+ T-cells and/or other cytotoxic immune cells (e.g. within a tumour or cancer or tumour microenvironment or cancer microenvironment) may be considered as the size of the sub-population of CD8+ T-cells and/or other cytotoxic immune cells from within the total population of CD45 positive (CD45+) cells (e.g. in a sample such as a tumour biopsy), which may be expressed as a percentage of the total CD45+ cells. Alternatively viewed, the level of CD8+ T-cells and/or other cytotoxic immune cells (e.g. within a tumour or cancer or tumour microenvironment or cancer microenvironment) may be considered the proportion of the total CD45+ cell population (e.g. in a sample such as a tumour biopsy) that is CD8+ T-cells and/or other cytotoxic immune cells, which may be expressed as a percentage of the total CD45+ cells.

Thus, in some embodiments, the level of CD8+ T-cells and/or other cytotoxic immune cells is the relative level of CD8+ T-cells and/or other cytotoxic immune cells or the relative population size of CD8+ T-cells and/or other cytotoxic immune cells or the relative proportion of CD8+ T-cells and/or other cytotoxic immune cells (e.g. relative to the total CD45+ cell level or the total CD45+ population size). Thus, in some embodiments, there is an increase in the relative level of CD8+ T-cells and/or other cytotoxic immune cells (or in the relative population size of CD8+ T-cells and/or other cytotoxic immune cells), e.g. relative to the total CD45+ cell level or the total CD45+ population size. In some embodiments, the absolute number of CD8+ T-cells and/or other cytotoxic immune cells (e.g. within a tumour or cancer or tumour microenvironment or cancer microenvironment) is increased.

In some embodiments, the immune response against cancer (or anti-cancer response or anti-cancer immune response) is characterized by a reduction in the ratio of Tregs to CD8+ T-cells (or CD8+ effector T-cells or CD8+ cytotoxic T-cells) and/or other cytotoxic immune cells.

Alternatively viewed, in some embodiments, the immune response against cancer (or anti-cancer response or anti-cancer immune response) is characterized by a reduction in the ratio of Tregs/CD8+ T-cells and/or other cytotoxic immune cells.

Thus, in some embodiments, the immune response against cancer is characterized by a reduction in the level of Tregs relative to the level of CD8+ T-cells and/or other cytotoxic immune cells.

Preferably, the immune response against cancer is characterized by a reduction in the ratio of Tregs to CD8+ T-cells. Thus, preferably, the immune response against cancer is characterized by a reduction in the ratio of Tregs/CD8+ T-cells. Thus, preferably, the immune response against cancer is characterized by a reduction in level of Tregs relative to the level of CD8+ T-cells.

Conversely, in some embodiments, the immune response against cancer (or anti-cancer response or anti-cancer immune response) is characterized by an increase in the ratio of CD8+ T-cells (or CD8+ effector T-cells or CD8+ cytotoxic T-cells) and/or other cytotoxic immune cells to Tregs. Alternatively viewed, in some embodiments, the immune response against cancer is characterized by an increase in the ratio of CD8+ T-cells and/or other cytotoxic immune cells/Tregs. Thus, in some embodiments, the immune response against cancer is characterized by an increase in the level of CD8+ T-cells and/or other cytotoxic immune cells relative to the level of Tregs.

Preferably, the immune response against cancer is characterized by an increase in the ratio of CD8+ T-cells to Tregs. Thus, preferably, the immune response against cancer is characterized by an increase in the ratio of CD8+ T-cells/Tregs. Thus, preferably the immune response against cancer is characterized by an increase in the level of CD8+ T-cells relative to the level of Tregs.

Typically, the reduction in the ratio of Tregs to CD8+ T-cells and/or other cytotoxic immune cells (Tregs/CD8+ T-cells and/or other cytotoxic immune cells) is a reduction in this ratio within the tumour or cancer or in the tumour microenvironment or cancer microenvironment, or in a sample such as a biopsy or cell suspension that has been obtained from the tumour or cancer or tumour microenvironment or cancer microenvironment. In some embodiments, such a sample may be processed (e.g. prior to analysis), for example to obtain a cell suspension from the tumour or cancer or tumour microenvironment or cancer microenvironment.

A reduction in the ratio of Tregs to CD8+ T-cells and/or other cytotoxic immune cells may be any measurable reduction or decrease, e.g. when compared with a control ratio. Preferably, the ratio is significantly reduced, compared to the ratio found in an appropriate control sample or subject or population (e.g a healthy or normal subject or population, or sample therefrom, or a treated subject's “baseline” ratio). More preferably, the significantly reduced ratios are statistically significant, preferably with a probability value of <0.05 or <0.01. Appropriate control ratios (or control samples or values) could be readily chosen by a person skilled in the art. Appropriate control “values” could also be readily determined without running a control “sample” in every test, e.g. by reference to the range for normal subjects and/or by reference to the “baseline” ratio in a subject being treated.

The control ratio may correspond to the ratio of Tregs to CD8+ T-cells and/or other cytotoxic immune cells in the same individual subject, or a sample from said subject (e.g. a tumour biopsy), measured at an earlier time point (e.g. comparison with a “baseline” ratio in that subject, e.g. the ratio in the subject prior to the start of treatment in accordance with the invention, or the ratio at an earlier time point during the treatment regime). This type of control ratio (i.e. a control ratio from an individual subject) is particularly useful for embodiments of the invention where serial or periodic measurements of this ratio in individuals, either healthy or ill, are taken looking for changes in the ratio of Tregs to CD8+ T-cells and/or other cytotoxic immune cells. In this regard, an appropriate control ratio will be the individual's own baseline, stable, nil, previous or dry value (as appropriate) as opposed to a control or cutoff ratio found in the general population Control ratios may also be referred to as “normal” ratios or “reference” ratios. The control ratio may be a discrete figure or a range.

In some embodiments, a reduction in the ratio of Tregs to CD8+ T-cells and/or other cytotoxic immune cells may be a reduction by a factor of at least 1.5, at least 2, at least 3, at least 4, at least 5 or at least 10 (e.g. a factor of 1.5 to 5 or a factor of 1.5 to 10 or a factor of 2 to 5 or a factor of 2 to 10), for example in comparison to a control ratio. Purely by way of example, if a control ratio of Tregs to CD8+ T-cells and/or other cytotoxic immune cells is 1 to 1 (1:1), then a reduction in the ratio of Tregs to CD8+ T-cells and/or other cytotoxic immune cells by a factor of 2 would result in a ratio of Tregs to CD8+ T-cells and/or other cytotoxic immune cells of 1 to 2 (1:2).

The discussion above in relation to a “reduction” or “decrease” in the ratio of Tregs to CD8+ T-cells and/or other cytotoxic immune cells can be applied conversely (mutatis mutandis) to “increases” in the ratio of CD8+ T-cells and/or other cytotoxic immune cells to Tregs.

Thus, in some embodiments, an increase in the ratio of CD8+ T-cells and/or other cytotoxic immune cells to Tregs may be an increase by a factor of at least 1.5, at least 2, at least 3, at least 4, at least 5 or at least 10 (e.g. a factor of 1.5 to 5 or a factor of 1.5 to 10 or a factor of 2 to 5 or a factor of 2 to 10), for example in comparison to a control ratio. Purely by way of example, if a control ratio of CD8+ T-cells and/or other cytotoxic immune cells to Tregs is 1 to 1 (1:1), then an increase in the ratio of CD8+ T-cells and/or other cytotoxic immune cells to Tregs by a factor of 2 would result in a ratio of CD8+ T-cells and/or other cytotoxic immune cells to Tregs of 2 to 1 (2:1).

In some embodiments, the immune response against cancer (or anti-cancer response or anti-cancer immune response) is characterized by an increase in the level of CD8+ immune cells (e.g. CD45+CD8+ immune cells). The discussion above in relation to increases in the level of CD8+ T-cells may be applied, mutatis mutandis, to increases in the level of CD8+ immune cells.

In some embodiments, the immune response against cancer (or anti-cancer response or anti-cancer immune response) is characterized by a reduction in the ratio of Tregs to CD8+ immune cells and/or other cytotoxic immune cells. Conversely, in some embodiments, the immune response against cancer (or anti-cancer response or anti-cancer immune response) is characterized by an increase in the ratio of CD8+ immune cells and/or other cytotoxic immune cells to Tregs. The discussion above in relation ratios that involve CD8+ T-cells may be applied, mutatis mutandis, to ratios that involve CD8+ immune cells.

Tregs have been implicated in mediating immunosuppression in patients with a number of different malignancies, including ovarian, pancreatic, breast, colorectal, lung, and esophageal cancer. Many tumors types are infiltrated by Tregs, and depletion of Treg cells from the tumor microenvironment can enhance or restore anti-tumor immunity. One of the reasons for Treg accumulation at tumor site can be due to the production of chemokines by tumor cells and stroma in the tumor microenvironment, which mediates Treg influx within the tumor tissue. Furthermore, the conversion of FoxP3− cells into FoxP3+ cells in presence of TGF-3 or increased proliferation of Tregs can lead to their expansion. The presence of FOXP3+ in the tumor-infiltrating lymphocyte (TIL) population has been reported to be associated with poor clinical outcome in a variety of cancer types, including prostatic, lung, hepatocellular and renal cell carcinomas. The suppressive functions of FoxP3+ Tregs can be regulated by the presence of different factors, such as Helios, CTLA-4, and PD-1.

A meta-analysis of 25 published studies comprising over 22 000 patients, showed that immune infiltrates are associated with overall survival (OS) in TNBC. FOXP3-expressing lymphocytes were associated with worse disease-free survival and overall survival (Mao et al. 2016).

In the glioma microenvironment, the anti-tumor effector T cells can be critically suppressed and/or overwhelmed by Tregs. In glioma patient tissues, tumor-infiltrating CD8+ T cells were phenotypically CD8+ and CD25−, indicating that these effector cells were not activated or proliferating. The CD4+ T cells were more numerous than CD8+ T cells within the gliomas, and the majority of CD4+ T cells were Tregs as evidenced by positive intracellular staining for Foxp3. In another study, the CD4+CD25+Foxp3+ T cells were found only in gliomas, whereas Tregs were absent from control brain tissue specimens (Humphries et al. 2010). Glioma patients have an increased fraction of systemic Tregs, which corresponds to decreased T cell effector activity and a shift from pro-inflammatory Th1 cytokines to an anti-inflammatory Th2 milieu. In addition to an increase in systemic TReg, intratumoral Treg numbers increase profoundly in low and high grade astrocytomas. Expression of the immune checkpoint protein CTLA-4 increases on Tregs in glioma-bearing mice. Treatment of mice with anti-CTLA-4 mAb induces an intratumoral shift from a high population of FoxP3+ Treg cells to a high population of pro-inflammatory IFN-γ-producing CD4+ cells (Gedeon et al. 2014).

Some ovarian tumours evoke an immune response, which can be assessed by tumour infiltrating lymphocytes (TILs). Increased levels of both intratumoral and stromal TILs are associated with a better prognosis. Immune infiltration has also been shown to be enriched in some molecular subtypes of ovarian cancer, and molecular subtypes show distinct survival characteristics. Low numbers of intratumoral CD3+ T-cell numbers were identified in high-risk subtypes with lower overall survival (Tothill et al. 2008). Tregs are present in ovarian tumor tissues and may have an immunopathological role. An increase in the number of tumor Treg cells is a significant predictor of increased risk for death and for reduced survival in ovarian cancer. According to one study, human tumor Treg cells suppressed tumor-specific T cell immunity and contributed to growth of human tumors in vivo. Human Treg cells preferentially move to and accumulate in tumors and ascites, but rarely enter draining lymph nodes in later cancer stages. CD4+CD25+ T cells accumulated in malignant ascites and tumor tissue in individuals with ovarian cancers, whereas CD4+CD25+ T cells were undetectable in normal ovarian tissues from five control subjects without cancer. All (100%) of the tumor infiltrating FOXP3+ cells were CD25+ T cells, and 90% of CD25+ T cells were FOXP3+ cells in the tumor mass. 80% of FOXP3+CD25+ cells were in close contact with CD8+ T cells. Trafficking of Treg cells to tumors in vivo was mediated by CCL22 signaling originating from tumor tissue, from tumor cells and macrophages. Tregs inhibited the function of tumor-infiltrating T cells by inhibiting production of IL-12 and IFN-γ (Curiel et al. 2004).

CRC (colorectal cancer) is mainly an inflammation-associated cancer, and Tregs are expanded in tumor microenvironment (TME) and play important roles in the pathogenesis of CRC. There is significant accumulation of CD45+CD3+ cells within colorectal tumor tissue, of which the majority are CD4⁺. This suggests a pro-tumorigenic function of infiltrating CD4+ T cells in CRC patients. These tumor-infiltrating CD4+ T cells expressed high CD25, which indicated the presence of Tregs. Several studies demonstrated that CD4⁺CD25⁺FOXP3⁺ Treg in CRC patients were capable of inhibiting tumor associated antigen-specific immune responses, and that Treg are enriched in patients with CRC (Clarke et al. 2006). A study showed that CD4+FoxP3+Helios+ Tregs are expanded in the TME of CRC patients compared with normal tissue and peripheral blood, and that the CD4⁺FoxP3⁺Helios⁺ Treg subset expressed higher levels of immune checkpoint receptors indicating a potent immunosuppressive potential. In CRC tumor tissue, frequencies of FoxP3+Helios+ Tregs were elevated compared with FoxP3-Helios+ and FoxP3+Helios− Tregs. Helios was shown to be a marker for T cell activation and proliferation, and its expression is essential for the stable Treg inhibitory activity. Helios is also a marker of activated Tregs expressing immunosuppressive molecules GARP/latency-associated peptide (LAP).

Gastric cancer patients have higher numbers of Treg not only in tissue but also in the blood compared to healthy controls. Gastric cancer patients from whom tumors have been removed have significantly lower levels of CD4+CD25high T cells. Gastric tumor mucosa has an increased Treg to CD8 ratio. It was shown that 90% of the CD4+CD25high cells in gastric tumor mucosa express high levels of FOXP3 and that this cell population is suppressive (Kindlund et al. 2017). CD4+CD25+CD127low/− regulatory T cells express Foxp3 and suppress effector T cell proliferation and contribute to gastric cancers progression (Shen et al. 2009).

Inflammation appears to play a role in the pathogenesis of PDAC (pancreatic ductal adenocarcinoma), and the evolving immune response in the context of chronic inflammation may facilitate subsequent invasion and metastasis. Treg cell infiltration is a prominent feature of pancreatic ductal adenocarcinoma. PDAC Intratumoral CD8+ T cells express high levels of the immune checkpoint molecule programmed cell death-1, providing an additional mechanism through which T-cell activation may be regulated by tumor cells or immunosuppressive myeloid cells (Pillarisetty 2014). In PDAC, the accumulation of Treg cells in the tumor microenvironment occurs during the preinvasive stage of the disease, and is associated with poor prognosis and reduced survival. Early accumulation of suppressive immune cells precedes and outweighs antitumor cellular immunity. In murine models, Treg cell ablation is sufficient to induce effective anti-tumor immune response in both early and advanced pancreatic development. Treg cells engage in extended interactions with tumor-associated CD11c+ dendritic cells (DCs) and restrain their immunogenic function, by suppressing the expression of costimulatory ligands necessary for CD8⁺ T cell activation. Treg cells restrain the expansion of tumor-associated CD11c+ DCs and their capacity to provide co-stimulation to T cells. Neuropilin-1 is abundantly expressed in intratumoral Treg cells at all stages of pancreatic tumor progression. Immune tolerance in the pancreatic TME may in part be driven by a feed-forward mechanism involving the reciprocal interaction between DCs and Treg cells that mutually reinforces their immunosuppressive activities (Jang et al. 2017).

A comprehensive meta-analysis found that the frequency of Tregs in the HCC (hepatocellular cancer) tumor microenvironment was significantly higher than that in tumor-surrounding tissue and biopsy specimens from healthy livers, and that there is an obvious association between Tregs and pathogenesis of HCC (Zhao et al. 2014). Peripheral blood levels of CD4+CD25+FOXP3+ Treg cells in HCC patients were significantly higher than those of the control group, and levels of CD4⁺CD25⁺FOXP3⁺ Treg cells in the peripheral blood of advanced (stage III-IV) HCC patients were higher than those of early (stage I-II) HCC patients, which implies that the presence of CD4⁺CD25⁺ Treg cells was closely related to tumor progression (Lan et al. 2017).

In a meta-analysis of 29 trials with over 86 000 patients, high levels of CD8-expressing cells infiltrating the tumour or in the tumour stroma of non-small cell lung cancer (NSCLC) specimens were associated with better OS, whereas FOXP3-expressing Treg cells in the tumour stroma had association with worse progression-free and overall survival (Geng et al. 2015).

The prognostic impact of infiltrating immune cells in melanoma in mostly positive. High TIL levels are generally associated with improved overall survival. Higher densities of CD8+ T cells correlated best with survival, a higher density of CD45+ leukocytes, T cells, and B cells also correlated with increased survival. High Foxp3 expression in lymph nodes predicts for worse progression free survival in stage III melanoma patients, but did not impact overall survival (Knol et al. 2011). Gyorki et al. reported an increase in CD4+Foxp3+T-regulatory cell proportion in progressive tumors together with a 2.8-fold lower CD8+/CD4+Foxp3+ ratio in the tumor compared with the blood, suggesting a possible mechanism of immune escape (Gyorki et al. 2013). A high percentage of intratumoral Neuropilin-1-positive (Nrp1) Tregs correlates with poor prognosis in melanoma. Using a mouse model of melanoma where Nrp1-deficient (Nrp1^{-/-}) and wild-type (Nrp1⁺) Tregs can be assessed in a competitive environment, it was found that a high proportion of intratumoral Nrp1^{-/-} Tregs produce interferon-γ (IFN-γ), which drives the fragility of surrounding wild-type Tregs, boosts anti-tumor immunity, and facilitates tumor clearance (Overacre-Delgoffe et al. 2017).

A high percentage of intratumoral Neuropilin-1-positive Tregs correlates with poor prognosis in head and neck squamous cell carcinoma. Neuropilin-1 (Nrp1), a receptor for TGF-β1, is required to maintain intratumoral Treg stability and function (Overacre-Delgoffe et al. 2017).

Cancers for treatment in accordance with the invention express TrxR (e.g. TrxR1) and Trx (e.g. Trx1), and preferably express high (or increased) levels of TrxR and Trx. TrxR and Trx are expressed in many cancer types and indeed may be overexpressed in many cancer types (e.g. in brain cancer and breast cancer such as metastatic breast cancer). Cancers for treatment in accordance with the invention may additionally (or alternatively) overexpress (or have higher levels of) other redox-active proteins such as PDI (Protein Disulphide Isomerase), and peroxiredoxins (as non-limiting examples). Thus, in some embodiments, a cancer for treatment in accordance with the present invention may overexpress thioredoxin reductase (TrxR) and/or thioredoxin and/or PDI. In some embodiments, a cancer for treatment in accordance with the present invention may overexpress thioredoxin reductase (TrxR) and thioredoxin and PDI. The level of TrxR and/or Trx (and/or other redox-active proteins) can be assessed by a skilled person by any appropriate means.

High levels (or overexpression or increased level) of TrxR and Trx typically means high (or overexpressed or increased) in relation to (i.e. higher than, preferably significantly higher than) the level of TrxR and Trx in tissue surrounding a cancer (or tumour), or in normal tissue (e.g. if the cancer in question is breast cancer a high level (or overexpressed level) of TrxR or Trx can be a higher level than in normal or healthy breast tissue).

Preferred cancers to be treated in accordance with the present invention are (i) T-cell infiltrated (preferably highly T-cell infiltrated), e.g. as discussed above, and (ii) express high levels (or overexpress or have increased levels) of TrxR and Trx (and may additionally express high levels of, or overexpress or have higher levels of, one or more downstream ROS-active proteins), e.g. as discussed above.

Alternatively, cancers to be treated in accordance with the present invention have a high capacity for T-cell infiltration to the tumor tissue and may display high CD8+/Treg ratios in plasma. Subjects with high TrxR/Trx levels in the tumor can be identified from taking a biopsy specimen from the tumor tissue, or/and alternatively by measuring plasma levels of TrxR and Trx. Sometimes, it could be advantageous to use a SecTRAP inhibitor in combination with an immunostimulatory agent, and sometimes it could be advantageous to use a SecTRAP inhibitor in combination with a Trx antibody that additionally lowers the extracellular Trx levels in the tumor microenvironment.

The inventors have found that metastatic and advanced cancers often overexpress TrxR and Trx and are typically heavily immune-cell infiltrated. Thus, in some embodiments, the treatment of metatstatic or advanced cancers is preferred.

Preferred cancers for treatment in accordance with the invention include breast cancer (e.g triple negative breast cancer), pancreatic cancer, colon cancer, head and neck cancer (e.g. head and neck squamous cell carcinoma), prostate cancer, brain tumours (e.g. astrocytic brain tumours), melanoma, ovarian cancer, urothelial cancer, colorectal cancer, lung cancer, gastric cancer, renal cancer, hepatocellular cancer, oesophageal cancer, acute promyelocytic leukaemia (APL) and brain cancer (e.g. glioma such as malignant glioma).

Preferably, the cancer is a solid (or bulky) tumour. Thus, in some embodiments the cancer is not a haematological cancer (e.g. not a leukaemia).

In a preferred embodiment, the cancer to be treated is breast cancer (preferably triple negative breast cancer) or brain cancer (preferably malignant glioma).

In a particularly preferred embodiment, the cancer to be treated is breast cancer, particularly triple negative breast cancer. In some embodiments, the triple negative breast cancer is an invasive ductal carcinoma.

In a preferred embodiment, the cancer to be treated is brain cancer.

In some embodiments, the cancer to be treated is a metastatic cancer. In some embodiments the cancer is a progressive cancer or an advanced cancer. In particular, metastatic, progressive or advanced cancers that highly express (or overexpress) TrxR and Trx and that are T-cell-infiltrated (preferably highly T-cell infiltrated) are preferred.

In some embodiments, the cancer to be treated is selected from the group consisting of (or comprising):

soft tissue cancers, such as sarcoma (e.g. angiosarcoma, fibrosarcoma, rhabdomyosarcoma, liposarcoma), myxoma, rhabdomyoma, fibroma, lipoma and teratoma;
lung cancers, such as bronchogenic carcinoma (e.g. squamous cell, undifferentiated small cell, undifferentiated large cell, adenocarcinoma), alveolar (or bronchiolar) carcinoma, bronchial adenoma, sarcoma, lymphoma, chondromatous hamartoma, mesothelioma, including non-small cell lung cancer;
gastrointestinal cancers: such as esophageal cancers (e.g. squamous cell carcinoma, adenocarcinoma, leiomyosarcoma, lymphoma), stomach cancers (e.g. carcinoma, lymphoma, leiomyosarcoma), pancreatic cancers (e.g. ductal adenocarcinoma, insulinoma, glucagonoma, gastrinoma, carcinoid tumors, vipoma), small bowel cancers (e.g. adenocarcinoma, lymphoma, carcinoid tumors, Kaposi's sarcoma, leiomyoma, hemangioma, lipoma, neurofibroma, fibroma), large bowel cancers (e.g. adenocarcinoma, tubular adenoma, villous adenoma, hamartoma, leiomyoma);
genitourinary tract cancers, such as cancer of the kidney (e.g. adenocarcinoma, Wilm's tumor (nephroblastoma), lymphoma, leukemia), bladder and urethra (e.g. squamous cell carcinoma, transitional cell carcinoma, adenocarcinoma), prostate (e.g. adenocarcinoma, sarcoma), testis (e.g. seminoma, teratoma, embryonal carcinoma, teratocarcinoma, choriocarcinoma, sarcoma, interstitial cell carcinoma, fibroma, fibroadenoma, adenomatoid tumors, lipoma);
liver cancers, such as hepatoma (e.g. hepatocellular carcinoma), cholangiocarcinoma, hepatoblastoma, angiosarcoma, hepatocellular adenoma, hemangioma;
bone cancers, such as osteogenic sarcoma (e.g. osteosarcoma), fibrosarcoma, malignant fibrous histiocytoma, chondrosarcoma, Ewing's sarcoma, malignant lymphoma (e.g. reticulum cell sarcoma), multiple myeloma, malignant giant cell tumor chordoma, osteochronfroma (e.g osteocartilaginous exostoses), benign chondroma, chondroblastoma, chondromyxofibroma, osteoid osteoma and giant cell tumors;
cancers of the head and/or nervous system, such as cancer of the skull (e.g. osteoma, hemangioma, granuloma, xanthoma, osteitis deformans), meninges (e.g. meningioma, meningiosarcoma, gliomatosis), brain (e.g. astrocytoma, medulloblastoma, glioma, ependymoma, germinoma (pinealoma), glioblastoma multiform, oligodendroglioma, schwannoma, retinoblastoma, congenital tumors), spinal cord (e.g. neurofibroma, meningioma, glioma, sarcoma);
gynecological cancers, such as cancers of the uterus (e.g. endometrial carcinoma), cervix (cervical carcinoma, pre-tumor cervical dysplasia), ovaries (e.g. ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), cancers of the vulva (e.g. squamous cell carcinoma, intraepithelial carcinoma, adenocarcinoma, fibrosarcoma, melanoma), vagina (e.g. clear cell carcinoma, squamous cell carcinoma, botryoid sarcoma (embryonal rhabdomyosarcoma)), fallopian tubes (e.g. carcinoma);
haematologic cancers, such as cancers of the blood and bone marrow (e.g. myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma);
skin cancers, such as malignant melanoma, basal cell carcinoma, squamous cell carcinoma, Kaposi's sarcoma, moles dysplastic nevi, lipoma, angioma, dermatofibroma, keloids; neurofibromatosis and Adrenal glands; and
neuroblastomas.
More particular cancers that may be mentioned include those corresponding to the cell lines used in the examples provided herein.
More particular cancers that may be mentioned include:
head and neck cancer (such as throat cancer, e.g. pharyngeal squamous cell carcinoma);
colon cancer (such as colorectal carcinoma);
skin cancer (such as epidermoid (skin) carcinoma);
gastrointestinal cancers (such as pancreatic cancer, e.g. pancreatic ductal carcinoma); breast cancer (such as mammary adenocarcinoma, e.g. metastatic mammary adenocarcinoma);
lung cancer (such as carcinoma); and
haematologic cancers (such as leukemia, e.g. acute monocytic leukemia).
In some embodiments, the cancer is selected from pancreatic cancer, ovarian cancer and colorectal cancer.
For example, in certain embodiments, the cancer is selected from colorectal cancer (including those processing Ras mutations), small cell lung cancer, non-small cell lung cancer (NSCLC), and glioma.
In other embodiments, the cancer is selected from non-small cell lung cancer, ovarian cancer, metastatic breast cancer, pancreatic cancer, hepatobiliary cancer (including hepatocellular cancer, bile duct cancer and cholangiocarcinoma), and gastric cancer.
In further embodiments, the cancer is selected from colorectal cancer (including Ras mutations), small cell lung cancer, non-small cell lung cancer, ovarian cancer, hepatobiliary cancer (including hepatocellular cancer, bile duct cancer and cholangiocarcinoma), gastric cancer, testicular cancer, and head and neck squamous cell carcinoma.
In certain embodiments of the present invention, the cancer is selected from leukemia (including acute myeloid leukemia, acute lymphoblastic leukemia, chronic myeloid leukemia, and chronic lymphoid leukemia), lymphoma (including mantle cell lymphoma, Hodgkin's lymphoma and non-Hodgkin's lymphoma), and prostate cancer.

Subjects treated in accordance with the present invention are preferably humans. Veterinary treatments (e.g. for cows, sheep, pigs, dogs, cats, horses) are also contemplated. Typically of course, subjects in accordance with the present invention are subjects having cancer. Preferred cancer types are described elsewhere herein.

In preferred embodiments, subjects treated in accordance with the present invention have an active (or functioning) immune system, more preferably a fully active (or fully functioning) immune system. Accordingly, preferably subjects being treated in accordance with the present invention are capable of raising an effective immune response. Thus, preferably, subjects are typically and preferably not exposed to (and preferably have not been exposed to) any immunosuppressive agent as such subjects may not be capable of raising an effective immune response. Thus, preferably, subjects are typically and preferably not taking (and preferably have not taken) an immunosuppressive agent. Preferably subjects have not been otherwise diagnosed as having an impaired immune system. Preferred subjects thus do not have an impaired immune system (or are not immunodeficient) as it is believed that such subjects will not be capable of raising an effective immune response.

In some embodiments, the subject is a subject for which there is no alternative therapeutic option (e.g. no effective alternative therapeutic option).

The term “treatment” or “therapy” includes any treatment or therapy which results in an improvement in the health or condition of a patient, or of a symptom of the cancer they are suffering. “Treatment” is not limited to curative therapies (e.g. those which result in the elimination of cancer cells or tumours or metastases from the patient), but includes any therapy which has a beneficial effect on the cancer or the patient, for example, tumour regression or reduction, reduction of metastatic potential, increased overall survival, extension or prolongation of life or remission, induction of remission, a slow-down or reduction of disease progression or the rate of disease progression, or of tumour development, subjective improvement in quality of life, reduced pain or other symptoms related to the disease, improved appetite, reduced nausea, or an alleviation of any symptom of the cancer.

In preferred subjects to be treated in accordance with the invention, the subject's serum level of Trx is typically increased (e.g. in comparison to a control level). In normal individuals the serum level of Trx is typically in the range of 10 ng to 80 ng/ml (0.8-6.6 nM). In preferred embodiments, subjects to be treated in accordance with the present invention have an increased serum Trx level, e.g. higher than 80 ng/ml (e.g. ≥100 ng/ml or ≥120 ng/ml). The “increased” in Trx level may be any measurable increase or elevation, e.g. when compared with a control level (e.g. an increased level may be a serum Trx level above 80 ng/ml or ≥100 ng/ml or ≥120 ng/ml). Preferably, the level is significantly increased, compared to the level found in an appropriate control sample or subject (e.g a healthy or normal subject or sample therefrom). More preferably, the significantly increased levels are statistically significant, preferably with a probability value of <0.05 or <0.01. Appropriate control levels (or control samples or values) could be readily chosen by a person skilled in the art. Appropriate control “values” could also be readily determined without running a control “sample” in every test, e.g. by reference to the range for normal subjects.

As discussed elsewhere herein, the present inventors have found that SecTRAP forming agents can harness the subject's own immune system to target the cancer. This finding has opened up the useful possibility of prospectively identifying subjects (patients) that may benefit from treatment with a SecTRAP forming agent in accordance with the invention. The clinician considering treatment options for a cancer patient, now armed with the knowledge that SecTRAP forming agents elicit an anti-cancer immune response, can now prospectively and specifically select those patients most likely to benefit from the treatment. In this regard, in some embodiments, the clinician may choose to treat subjects known to have an active (e.g. fully active) immune system.

The clinician could also elect to treat those patients that are known (or suspected) to have a T-cell infiltrated tumour and known to express TrxR/Trx (e.g. high levels of TrxR and/or Trx or overexpressed levels of TrxR and/or Trx, or e.g. high levels of TrxR and/or Trx and other redox proteins, or overexpressed levels of TrxR and/or Trx and other redox proteins). The clinician could also elect to treat those patients that are known (or suspected) to have a T-cell infiltrated tumour and known to express (or overexpress) other redox proteins and/or genes (e.g. PDI, protein disulfide isomerase, which is a substrate of TrxR and/or peroxiredoxins). Thus, in some embodiments, the patient (or subject) having cancer (e.g. a T-cell infiltrated tumour) to be treated may overexpress TrxR and/or Trx and/or PDI. In some embodiments the patient having a cancer (e.g. a T-cell infiltrated tumour) to be treated overexpresses TrxR and Trx and PDI. The levels of TrxR and/or Trx (or other redox proteins and/or genes) could be assessed in a blood-based or biopsy-based assay (or diagnostic), e.g. in a blood sample or a serum sample. In some embodiments the patient (subject) having a cancer (e.g. a T-cell infiltrated tumour) to be treated has overexpressed levels of TrxR and/or Trx and/or PDI, said levels being as determined in a blood or serum sample. In some embodiments the patient (or subject) having cancer (e.g. a T-cell infiltrated tumour) to be treated has overexpressed levels of TrxR and Trx and PDI, said levels being as determined in a blood or serum sample.

In some embodiments the patient (subject) having cancer (e.g. a T-cell infiltrated tumour) to be treated has overexpressed levels of TrxR and/or Trx and/or PDI in blood or serum. In some embodiments the patient (or subject) having cancer (e.g. a T-cell infiltrated tumour) to be treated has overexpressed levels of TrxR and Trx and PDI in blood or serum.

Overexpression (or increased or higher levels) may be as determined in comparison to any appropriate control (e.g. control level or control sample or biopsy). For example, the control level may be the level in a sample (e.g. blood or serum sample or tissue sample or biopsy) from a healthy subject (e.g. a subject not having cancer). Appropriate control levels (or control samples or values) could be readily chosen by a person skilled in the art. Appropriate control “values” could also be readily determined without running a control “sample” in every test, e.g. by reference to the range for normal subjects.

Preferably, the overexpression (or overexpressed level), higher level, or increased level are significant, preferably statistically significant, preferably with a probability value of <0.05 or <0.01. In some embodiments, increased levels may be an increase of at least 5%, at least 10%, at least 20%, at least 30%, at least 40%, at least 50%, at least 60%, at least 70%, at least 75%, at least 80%, at least 90% or at least 100% (e.g. as compared to a control level).

Thus, given the finding underpinning the present invention a clinician can make a much more considered choice when deciding whether or not to treat a given cancer subject (patient) with a SecTRAP forming agent. In some embodiments, the methods and uses of the present invention may comprise an initial step of selecting a cancer patient to be treated with a SecTRAP forming agent, wherein said step comprises determining whether or not (or the degree to which) the cancer is immune cell infiltrated (e.g. T-cell infiltrated). A subject selected for treatment may be a subject having an immune cell infiltrated cancer (e.g. T-cell infiltrated cancer) in accordance with the invention (e.g. a highly immune cell infiltrated or highly T-cell infiltrated cancer). In some embodiments, the methods and uses of the present invention may comprise an initial step of selecting a cancer patient to be treated with a SecTRAP forming agent, wherein said step comprises determining whether or not (or the degree to which) the cancer is T-cell infiltrated and the TrxR/Trx expression level in the cancer (and/or the level of redox proteins and/or genes e.g. as described elsewhere herein). A subject selected for treatment may be a subject having a T-cell infiltrated cancer in accordance with the invention (e.g. a highly T-cell infiltrated cancer) and expressing (preferably highly expressing or overexpressing) TrxR and/or Trx.

In some embodiments, treatments in accordance with the present invention may be used in subjects at risk of cancer relapse or recurrence or metastasis. Thus, alternatively viewed, in some embodiments, SecTRAP forming agents are used in the prevention of cancer relapse or recurrence or metastasis. Further alternatively viewed, in some embodiments the SecTRAP forming agent protects (e.g. provides long-term protection) against cancers that recur and/or metastasize. Thus, the SecTRAP forming agent may provide anti-cancer or anti-tumour immunity, e.g. provide long-term protection against cancer relapse, recurrence and/or metastasis.

In one aspect, the invention provides a method of screening for (e.g. diagnosis of or prognosis of) cancer, including prognostic and predictive outcome biomarkers, which comprises determining the level (and/or activity) of TrxR and/or Trx (preferably TrxR and Trx e.g. a reduced form of Trx, or total Trx), and optionally determining the level of downstream redox active proteins, in a sample (e.g. a blood sample or a biopsy) that has been obtained from a subject. Typically, an increased level (and/or activity) in said sample of TrxR and/or Trx (preferably TrxR and Trx) in comparison to a control level or activity (e.g. the level or activity in normal or healthy tissue) is indicative of cancer in said subject.

In some embodiments, screening is prognostic screening or predictive outcome biomarker screening.

Such a method may be used for determining the clinical severity of cancer or disease progression in a subject. In such methods the level (and/or activity) of TrxR and/or Trx (preferably TrxR and Trx) in the sample shows an association with the severity of the cancer (or progression of the disease or prognosis). Thus, the level (and/or activity) of TrxR and/or Trx (preferably TrxR and Trx e.g. a reduced form of Trx) may be indicative of the severity of the cancer (or prognosis) with high (or higher) levels or activities (e.g. in comparison to an appropriate control sample) typically being indicative of more severe disease (or poor or worse prognosis). In some embodiments, the more increased the level (or activity) of TrxR and/or Trx (preferably TrxR and Trx) in comparison to a control, the greater the likelihood of a more severe form of cancer (e.g. the greater the likelihood of a more aggressive form of cancer). Any appropriate control (or control sample) cane be used and the skilled person will be able to select an appropriate control (e.g. a control level or activity in a cancer of known severity prognosis). In some embodiments, the methods of the invention can thus be used in the selection of patients for therapy. For example, patients identified as having a severe form of cancer (or poor prognosis), e.g. based on the level of TrxR and/or Trx (and/or additional redox proteins including PDI), may be selected for treatment with a SecTRAP forming agent in accordance with the invention. Thus, methods used for screening (e.g for diagnosis or prognosis or for determining the clinical severity of cancer or disease progression) may further comprise a step of treating the subject with a SecTRAP forming agent.

TrxR/Trx may thus be used as a biomarker of disease progression, and is typically elevated in tumors and blood in various cancer forms. TrxR and Trx can be determined in tumor tissue and blood using, for example, mRNA expression analysis, IHC, ELISA and enzymatic assays

Protein-disulfide isomerase (PDI), a thiol-containing protein that can be reduced both by TrxR and reduced Trx is highly up-regulated in various cancer types, including kidney, lung, brain, ovarian, melanoma, prostrate, and male germ cell tumors. PDI can be useful as a biomarker together with TrxR/Trx. PDI can be used as a biomarker for cancers including breast cancer, glial cell cancer and colorectal cancer.

Serial (periodical) measuring of the level of TrxR and/or Trx (preferably TrxR and Trx) (and/or the level or activity of PDI and/or additional redox proteins) may also be used to monitor the severity of cancer looking for altering levels over time. Observation of altered levels (increase or decrease as the case may be) may also be used to guide and monitor therapy, both in the setting of subclinical disease, i.e. in the situation of “watchful waiting” (also known as “active surveillance”) before treatment or surgery, e.g. before initiation of pharmaceutical therapy, or during or after treatment to evaluate the effect of treatment and to look for signs of therapy failure.

As mentioned above, in accordance with the present invention, in addition to cytotoxic activity, SecTRAP forming agents have immunostimulatory activity thereby causing the subject being treated to raise (or stimulate or enhance or elicit) an immune response against said cancer. Put another way, they can elicit an anti-cancer immune response in subjects (via the formation of a SecTRAP).

The presence or occurrence of an anti-cancer immune response (or the ability of a compound to elicit an anti-cancer immune response) can be determined by any appropriate means and the skilled person is familiar with these. For example, a given compound can be tested for whether or not it elicits (or causes) an anti-cancer immune response by administering the compound to an immunodeficient animal (e.g. mouse) cancer model and an immunocompetent animal (e.g. mouse) cancer model, wherein improved (or increased) anti-cancer activity in the immunocompetent model relative to the immunodeficient model is indicative of an anti-cancer immune response (or immunostimulatory activity) of the compound. In some embodiments, anti-cancer activity may be expressed as % TGI (tumour growth inhibition). % TGI is calculated as 1-(median tumor volume of treated animals/median tumor volume of vehicle control)×100. In some embodiments, a compound gives a % TGI that is at least 10%, 20%, 30%, 40%, 50%, 60%, 70%, 80%, 90%, 100%, 110%, 120%, 130%, 140%, 150%, 160%, 170%, 180%, 190%, 200%, 220%, 240%, 260%, 280%, 300%, 350%, 400%, 450%, or 500% higher, e.g. up to 200%, 300%, 400%, 500%, or 1000% higher, or more, in an immunocompetent animal (e.g. mouse) cancer model than in an immunodeficient animal (e.g. mouse) cancer model.

It is widely acknowledged that the immune system can recognize and respond to tumour cells either naturally or following therapeutic intervention. The immune response towards tumors (anti-cancer immune response) is well studied, and is usually illustrated by a stepwise procedure named as the cancer-immunity cycle (Chen & Mellman 2013, Immunity, 39 (1), 1-10). It has been proposed that the cancer-immunity cycle must occur for productive antitumor immunity to develop. The cycle comprises the following steps: 1) release of tumor antigens by tumor cells; 2) antigen presentation to the immune system; 3) T-cell priming and activation; 4) T-cell trafficking to the tumor site; 5) tumor infiltration by T-cells; 6) tumor cell recognition by T-cells 7) tumor cell killing by T-cells. Normally the cycle is an ongoing process at the tumor site, and continues until either the tumor is destroyed or the tumor succeeds in evading the immune system. Evasion mechanisms include escaping T-cell recognition, interference with T-cell trafficking, metabolism and functions, induction of resistance to T-cell killing, and apoptosis of T-cells. The effect of therapeutic agents harnessing (or stimulating) the immune system to fight tumor tissue can depend on several factors. These include the level of immune infiltration in the tumor microenvironment (TME) relative to tumor cells, the ratio of pro-tumorigenic and antitumoral immune subsets and the expression levels of therapeutic targets over the various subtypes of cells present in the tumor.

An anti-cancer immune response may be characterised by, or occur in, a cancer (or tumour) that is T-cell infiltrated (e.g. as described elsewhere herein).

In another aspect (or in certain embodiments), the present invention provides a selenium compromised thioredoxin reductase-derived apoptotic protein (SecTRAP) forming agent for use in treating cancer in a subject, wherein said agent causes said subject to raise (or increase) the number of tumor-infiltrating T-cells and an immune response against said cancer. This thereby provides a therapeutic benefit. In yet another aspect (or in certain embodiments), the present invention provides a selenium compromised thioredoxin reductase-derived apoptotic protein (SecTRAP) forming agent for use in treating cancer in a subject, having e.g. high CD8+(cytotoxic T-cell)-to-regulatory T-cell ratio in plasma, wherein said agent has immunostimulatory activity thereby causing said subject to raise an immune response against said cancer.

In another aspect, the present invention provides a selenium compromised thioredoxin reductase-derived apoptotic protein (SecTRAP) forming agent for use in treating a T-cell infiltrated cancer in a subject, wherein said agent works with or works synergistically with the immune system of the subject being treated, in order for there to be, or to enhance, an anti-cancer response, in particular a clinically beneficial anti-cancer response.

In some preferred embodiments of the present invention the SecTRAP forming agent is used as the sole active agent (sole active agent in the treatment regimen). Thus, in some preferred embodiments the treatment is a monotherapy. Monotherapy refers to the use of a single drug to treat a disease or condition, in this case a T-cell infiltrated cancer. Thus, in some preferred embodiments the SecTRAP forming agent is used alone. By “sole active agent” (or sole active ingredient) is meant the sole agent or ingredient that is therapeutically active (or biologically active). Thus, components such as preservatives or excipients or agents that are not relevant to the disease being treated are not considered to be active agents.

Particularly preferably, cancer treatments in accordance with the present invention are done in the absence of an immunosuppressor (absence of an agent that suppresses or inhibits the ability of the subject being treated to raise an immune response). Put another way, preferably the treatment regimen does not include an immunosuppressor.

As mentioned above, in some embodiments SecTRAP forming agents may be used as the sole active agent in a cancer treatment regimen (as discussed elsewhere herein). However, in some embodiments, the SecTRAP forming agent is combined with one or more further (additional) active agents. For example, in some embodiments, a cancer therapy in accordance with the present invention may be characterised by the administration of an immunostimulant (an agent that stimulates or enhances a subject's immune system or immune response).

Thus, in one aspect, the present invention provides:

A combination of
(i) a selenium compromised thioredoxin reductase-derived apoptotic protein (SecTRAP) forming agent; and
(ii) an immunostimulatory agent

for use in treating cancer in a subject.

For such combination treatments, the cancer to be treated is not necessarily a T-cell infiltrated cancer, although in some embodiments the treatment of T-cell infiltrated cancers is preferred. The immunostimulatory agent (or immunostimulant) may stimulate and/or recruit therapeutically useful T-cells (e.g cytotoxic T-cells) to the cancer tissue and thus it is not necessary for the cancer being treated to already be T-cell infiltrated prior to the combination treatment.

An immunostimulant may be, speaking generally, administered to a subject substantially simultaneously with the SecTRAP forming agent, such as from a single pharmaceutical composition or from two pharmaceutical compositions administered closely together (at the same or a similar time). Alternatively, an immunostimulant may be administered to a subject at a time prior to or sequential to the administration of the SecTRAP forming agent. “At a time prior to or sequential to”, as used herein, means “staggered”, such that an immunostimulant is administered to a subject at a time distinct to the administration of the SecTRAP forming agent. Generally, the two agents may be administered at times effectively spaced apart to allow the two agents to exert their respective therapeutic effects, i.e., they are administered at “biologically effective time intervals”.

In some preferred embodiments, the immunostimulant is an agent that targets T-cells, either directly or indirectly, and preferably stimulates (or contributes to) a T-cell immune response.

In some embodiments the immunostimulant is a T-cell agonist, for example OX-40.

In some preferred embodiments, the immunostimulant is a checkpoint inhibitor (or an immune checkpoint inhibitor). Checkpoint inhibitors are a well-known class of anti-cancer agents which can promote immune activity, e.g. in a tumour microenvironment. A checkpoint inhibitor is a type of drug that blocks (or inhibits) certain proteins on some types of immune system cells, such as T-cells, or some cancer cells. These proteins usually help suppress immune responses and can help prevent T-cells from killing cancer cells. When these proteins are blocked (or inhibited) by checkpoint inhibitors, the immune system can be released from an inhibited state and T-cells are better able to kill cancer cells. Given that SecTRAP forming agents used in accordance with the invention can induce an anti-cancer immune response, for this effect to be maximised, it is important that subjects being treated are able to raise an immune response (have an active immune system). Thus, combination treatments with immunostimulants (e.g. checkpoint inhibitors) would be beneficial as they promote immune activity.

In some preferred embodiments, the checkpoint inhibitor is (or comprises) an antibody, for example an antibody (e.g. a monoclonal antibody), or antigen binding fragment thereof, that binds to PD-L1, PD-1, CTLA-4, TIM-3 or LAG-3. In a particularly preferred embodiment, the checkpoint inhibitor is (or comprises) an antibody (e.g. a monoclonal antibody) that binds to PD-L1. In another preferred embodiment, the checkpoint inhibitor is (or comprises) an antibody (e.g. a monoclonal antibody) that binds to PD-1. Thus, in some preferred embodiments the checkpoint inhibitor is an anti-PD-L1 antibody or an anti-PD-1 antibody. In some preferred embodiments, the anti-PD-1 antibody is Pembrolizumab. Thus, in some preferred embodiments, the invention provides a combination of (i) a selenium compromised thioredoxin reductase-derived apoptotic protein (SecTRAP) forming agent and (ii) an anti-PD-1 antibody (preferably Pembrolizumab) for use in treating cancer in a subject. In some preferred embodiments, the invention provides a combination of (i) the compound OT-1096 and (ii) an anti-PD-1 antibody (preferably Pembrolizumab) for use in treating cancer in a subject.

In some embodiments, the checkpoint inhibitor may be a non-antibody based molecule, e.g. a small molecule inhibitor.

In some embodiments, the immunostimulant is an IDO (Indoleamine-pyrrole 2,3-dioxygenase) inhibitor.

In other embodiments, the immunostimulant is a chemotherapeutic drug.

In some embodiments, the immunostimulant (immunostimulatory agent) is not a chemotherapeutic drug (e.g. is not gemcitabine or carboplatin or cyclophosphamide).

Embodiments of the uses of the invention described above apply, mutatis mutandis, to this combination therapy aspect of the invention.

In another aspect, the present invention provides:

A combination of
(i) a selenium compromised thioredoxin reductase-derived apoptotic protein (SecTRAP) forming agent; and
(ii) a Thioredoxin antibody

for use in treating cancer in a subject.

By thioredoxin antibody is meant an antibody (e.g. a monoclonal antibody), or an antigen binding fragment thereof, that binds to thioredoxin (or an anti-thioredoxin antibody). Embodiments of the uses of the invention described above apply, mutatis mutandis, to this combination therapy aspect of the invention.

Without wishing to be bound by theory, it is believed that Trx (thioredoxin) is secreted at higher than normal levels from tumours and that secreted Trx attracts (or steers) Treg cells towards the tumour leading to a worse outcome. Again, without wishing to be bound by theory, it is believed that an antibody directly targeting Trx could be administered (e.g. systemically) to bind to tumor-secreted Trx, thus helping to reduce or prevent Treg cell migration to the tumour.

In some embodiments, the subject to be treated with a SecTRAP forming agent in accordance with the present invention may also be undergoing treatment with, or may have undergone treatment with, a chemotherapeutic drug and/or radiotherapy. In some embodiments, the subject may have undergone multiple lines of therapy (e.g. 2 or 3 lines of therapy) with a chemotherapeutic drug and/or radiotherapy prior to treatment with a SecTRAP forming agent in accordance with the present invention. Without wishing to be bound by theory, the chemotherapy and/or radiotherapy (e.g. prior treatment with chemotherapy and/or radiotherapy) may stimulate and/or recruit cytotoxic T-cells to the cancer tissue and meaning that cancer is already T-cell infiltrated (e.g. highly T-cell infiltrated) or is in the process of being T-cell infiltrated. It is established in the art that chemotherapy and radiotherapy can lead to T-cell infiltration into cancer. If a subject has undergone chemotherapy and/or radiotherapy prior to treatment with a SecTRAP forming agent, they can be considered as having being induced (having had induction treatment to stimulate T-cell infiltration) prior to treatment with a SecTRAP forming agent. If desired, T-cell infiltration in cancer after (or during) chemotherapy and/or radiotherapy can be assessed (e.g. the degree) of T-cell infiltration assessed prior to the commencement of the SecTRAP forming agent treatment. In some embodiments, SecTRAP forming agent treatment would be commenced in those subjects identified as having T-cell infiltrated tumours (e.g. highly T-cell infiltrated tumours).

Chemotherapy and/or radiotherapy can be used to enhance an anti-cancer immune response. Chemotherapy can increase TrxR expression (e.g. via nrf2 activation) and T-cell infiltration in tumours.

The skilled person will be familiar with appropriate chemotherapeutic drugs (and regimens) and radiotherapies. In some embodiments, the chemotherapeutic drug is an anthracycline (e.g. Doxorubicin), cyclophosphamide or a platinum agent (e.g. cisplatin).

As the SecTRAP forming agents used in accordance with the invention can elicit an anti-cancer immune response in subjects (via the formation of a SecTRAP), it is believed that the a lower dose and/or less less frequent doses may be administered to a subject, i.e. a lower dose and/or less less frequent doses than may have been given if the sole mode of action of these agents was via a direct cytotoxic effect. The clinician can thus make a more considered approach when considering the dosing regimen and may opt to administer lower and/or less frequent doses in the knowledge that the SecTRAP forming agents will harness the patient's immune system to confer an anti-cancer effect (and that a direct cytotoxic effect is not the only mode of cancer cell killing).

SecTRAP forming agents for use in the present invention may be included in formulations (or compositions). Such formulations may be for pharmaceutical or veterinary use. Suitable diluents, excipients and carriers for use in such formulations are known to the skilled person.

SecTRAP forming agents (or formulations) for use in accordance with the present invention may be administered to a subject via any appropriate route.

The SecTRAP forming agents (or formulations) may be presented, for example, in a form suitable for oral, nasal, parenteral, intravenal, topical, rectal or intrathecal administration. Preferably, the compositions are presented in a form suitable for systemic (e.g. intravenous) administration.

The pharmaceutical compositions (formulations) may be administered parenterally. Parenteral administration may be performed by subcutaneous, intramuscular or intravenous injection, e.g. by means of a syringe. Alternatively, parenteral administration (e.g. i.v. infusion) can be performed by means of an infusion pump. Intraperitoneal (i.p.) administration or intratumoral administration (e.g. injection) may be used in some embodiments. Intrathecal administration (e.g. by injection into the spinal column), may be used in some embodiments.

In preferred embodiments, SecTRAP forming agents are systemically administered. In particularly preferred embodiments the SecTRAP forming agents (or formulations) are administered intravenously (i.v.) or intraperitoneally (i.p.).

In some preferred embodiments of the present invention, the subject is a human subject (preferably with an active immune system), the administration is systemic administration (e.g. i.v. or i.p. administration), and the cancer is a T-cell infiltrated cancer.

In some embodiments, the SecTRAP forming agents (or formulations) are administered intratumourally (e.g. by direct injection into the tumour).

In some embodiments, the SecTRAP forming agents (or formulations) are administered intrathecally (e.g. by direct injection into the spinal column).

The active compounds defined herein may be presented in the conventional pharmacological forms of administration, such as tablets, coated tablets, nasal sprays, solutions, emulsions, liposomes, powders, capsules or sustained release forms. Conventional pharmaceutical excipients as well as the usual methods of production may be employed for the preparation of these forms.

Injection solutions may, for example, be produced in the conventional manner, such as by the addition of preservation agents, such as p-hydroxybenzoates, or stabilizers, such as EDTA. The solutions are then filled into injection vials or ampoules.

Dosages, and dosage regimens, may vary based on parameters such as the age, weight and sex of the subject. Appropriate dosages can be readily established. Appropriate dosage units can readily be prepared.

The pharmaceutical compositions for use in the present invention may additionally comprise further therapeutically active ingredients as described above in the context of co-administration (or combination) regimens. However, as discussed elsewhere herein, in some preferred embodiments, in compositions for use in the present invention the SecTRAP forming agent is the sole active agent present.

The present invention also provides a method of treating a T-cell infiltrated cancer in a subject, said method comprising administering to a subject in need thereof a therapeutically effective amount of selenium compromised thioredoxin reductase-derived apoptotic protein (SecTRAP) forming agent, wherein said agent has immunostimulatory activity thereby causing the said subject to raise (or stimulate or enhance or elicit) an immune response against said cancer. Embodiments of the uses of the invention described above apply, mutatis mutandis, to this aspect of the invention.

A therapeutically effective amount can be determined based on the clinical assessment and can be readily monitored.

The present invention also provides the use of selenium compromised thioredoxin reductase-derived apoptotic protein (SecTRAP) forming agent in the manufacture of a medicament for treating a T-cell infiltrated cancer wherein said agent has immunostimulatory activity thereby causing the said subject to raise (or stimulate or enhance or elicit) an immune response against said cancer. Embodiments of the uses of the invention described above apply, mutatis mutandis, to this aspect of the invention.

The present invention also provides a method of treating cancer in a subject, said method comprising administering to a subject in need thereof a combination of a therapeutically effective amount of a selenium compromised thioredoxin reductase-derived apoptotic protein (SecTRAP) forming agent and an immunostimulant (or immunostimulatory agent). Embodiments of the uses of the invention described above apply, mutatis mutandis, to this aspect of the invention.

The present invention also provides the use of selenium compromised thioredoxin reductase-derived apoptotic protein (SecTRAP) forming agent in the manufacture of a medicament for treating cancer wherein said treatment further comprises the administration of an immunostimulant. The present invention also provides the use of a combination of a selenium compromised thioredoxin reductase-derived apoptotic protein (SecTRAP) forming agent and an immunostimulant (or immunostimulatory agent) in the manufacture of a medicament for treating cancer. Embodiments of the uses of the invention described above apply, mutatis mutandis, to this aspect of the invention.

The present invention also provides a method of treating cancer in a subject, said method comprising administering to a subject in need thereof a combination of a therapeutically effective amount of a selenium compromised thioredoxin reductase-derived apoptotic protein (SecTRAP) forming agent and a thioredoxin antibody. Embodiments of the uses of the invention described above apply, mutatis mutandis, to this aspect of the invention.

The present invention also provides the use of selenium compromised thioredoxin reductase-derived apoptotic protein (SecTRAP) forming agent in the manufacture of a medicament for treating cancer wherein said treatment further comprises the administration of a thioredoxin antibody. The present invention also provides the use of a combination of a selenium compromised thioredoxin reductase-derived apoptotic protein (SecTRAP) forming agent and a thioredoxin antibody in the manufacture of a medicament for treating cancer. Embodiments of the uses of the invention described above apply, mutatis mutandis, to this aspect of the invention.

In another aspect, the present invention provides a SecTRAP forming agent for use in stimulating (or enhancing or activating) the immune system in a subject (preferably in a subject having cancer). Alternatively viewed, the present invention provides a SecTRAP forming agent for use in stimulating (or eliciting or increasing) an immune response in a subject. Alternatively viewed, the present invention provides a SecTRAP forming agent for use in stimulating (or eliciting or increasing) an anti-cancer immune response in a subject. Alternatively viewed, the present invention provides a method for enhancing the immune system (or stimulating an immune response, in particular an anti-cancer immune response) in a subject, said method comprising administering a SecTRAP forming agent to said subject. The present invention also provides a use of a SecTRAP forming agent in the manufacture of a medicament for enhancing the immune system in a subject (or stimulating an immune response in a subject, e.g. an anti-cancer immune response). Embodiments of the uses of the invention described elsewhere herein apply, mutatis mutandis, to these aspects of the invention.

Alternatively viewed, the present invention provides a method for reducing the number (or level) of Tregs in a tumour (e.g. in the tumour microenvironment) and/or to increase the number of CD8+ T-cells in a tumour (e.g. in the tumour microenvironment), or to decrease the ratio of Tregs to CD8+ T-cells e.g. in the tumour microenvironment), said method comprising administering to a subject in need thereof a therapeutically effective amount of a selenium compromised thioredoxin reductase-derived apoptotic protein (SecTRAP) forming agent.

Alternatively viewed, the present invention provides a SecTRAP forming agent for use in reducing the number (or level) of Tregs in a tumour (e.g. in the tumour microenvironment) and/or to increase the number of CD8+ T-cells and/or other cytotoxic immune cells in a tumour (e.g. in the tumour microenvironment), or to decrease the ratio of Tregs to CD8+ T-cells and/or other cytotoxic immune cells, e.g. in the tumour microenvironment).

In one aspect, the present invention provides a selenium compromised thioredoxin reductase-derived apoptotic protein (SecTRAP) forming agent for use in reducing the level of regulatory T cells (Tregs) in a subject (e.g. in a tumour or tumour microenvironment in a subject). Embodiments of the uses of the invention described above apply, mutatis mutandis, to this aspect of the invention. For example, what is meant by “level of regulatory T cells (Tregs)” is discussed elsewhere herein.

In one aspect, the present invention provides a selenium compromised thioredoxin reductase-derived apoptotic protein (SecTRAP) forming agent for use in increasing the level of CD8+ T-cells (or CD8+ effector T-cells or CD8+ cytotoxic T-cells) and/or other cytotoxic immune cells in a subject (e.g. in a tumour or tumour microenvironment in a subject). Embodiments of the uses of the invention described above apply, mutatis mutandis, to this aspect of the invention.

In one aspect, the present invention provides a selenium compromised thioredoxin reductase-derived apoptotic protein (SecTRAP) forming agent for use in reducing the ratio of Tregs to CD8+ T-cells and/or other cytotoxic immune cells in a subject (e.g. in a tumour or tumour microenvironment in a subject). Embodiments of the uses of the invention described above apply, mutatis mutandis, to this aspect of the invention.

Conversely, in another aspect, the present invention provides a selenium compromised thioredoxin reductase-derived apoptotic protein (SecTRAP) forming agent for use in increasing the ratio of CD8+ T-cells and/or other cytotoxic immune cells to Tregs in a subject (e.g. in a tumour or tumour microenvironment in a subject). Embodiments of the uses of the invention described above apply, mutatis mutandis, to this aspect of the invention.

In another aspect, the present invention also provides the combination of a selenium compromised thioredoxin reductase-derived apoptotic protein (SecTRAP) forming agent and an immunostimulant (or immunostimulatory agent) for use in reducing the level of regulatory T cells (Tregs) in a subject (e.g. in a tumour or tumour microenvironment in a subject). Embodiments of the uses of the invention described above apply, mutatis mutandis, to this aspect of the invention. For example, what is meant by “level of regulatory T cells (Tregs)” is discussed elsewhere herein.

Alternatively viewed the present invention provides a method of reducing the level of regulatory T cells (Tregs) in a subject (e.g. in a tumour or tumour microenvironment in a subject), said method comprising administration to said subject of an effective amount of selenium compromised thioredoxin reductase-derived apoptotic protein (SecTRAP) forming agent. Embodiments of the uses of the invention described above apply, mutatis mutandis, to this aspect of the invention. For example, what is meant by “level of regulatory T cells (Tregs)” is discussed elsewhere herein.

The present invention also provides a method of reducing the level of regulatory T cells (Tregs) in a subject (e.g. in a tumour or tumour microenvironment in a subject), said method comprising administering to a subject in need thereof a combination of a therapeutically effective amount of a selenium compromised thioredoxin reductase-derived apoptotic protein (SecTRAP) forming agent and an immunostimulant (or immunostimulatory agent). Embodiments of the uses of the invention described above apply, mutatis mutandis, to this aspect of the invention. For example, what is meant by “level of regulatory T cells (Tregs)” is discussed elsewhere herein.

The present invention also provides the use of selenium compromised thioredoxin reductase-derived apoptotic protein (SecTRAP) forming agent in the manufacture of a medicament for reducing the level of regulatory T cells (Tregs) in a subject (e.g. in a tumour or tumour microenvironment in a subject). Such a treatment may further comprise the administration of an immunostimulant. The present invention also provides the use of a combination of a selenium compromised thioredoxin reductase-derived apoptotic protein (SecTRAP) forming agent and an immunostimulant (or immunostimulatory agent) in the manufacture of a medicament for reducing the level of regulatory T cells (Tregs) in a subject (e.g. in a tumour or tumour microenvironment in a subject). Embodiments of the uses of the invention described above apply, mutatis mutandis, to this aspect of the invention. For example, what is meant by “level of regulatory T cells (Tregs)” is discussed elsewhere herein.

Any aspects or embodiments of the invention that are not discussed explicitly herein in connection with combination therapies, e.g with immunostimulatory agents or thioredoxin antibodies, may in some embodiments, further comprise the use (or administration) of a further therapeutic agent such as an immunostimulatory agent or a thioredoxin antibody, and any features of aspects or embodiments of the invention may be applied, mutatis mutandis to such combination therapies.

In another aspect, the present invention provides a combination of a selenium compromised thioredoxin reductase-derived apoptotic protein (SecTRAP) forming agent and a targeted therapeutic agent or a cytotoxic therapeutic agent for use in treating cancer in a subject. Embodiments of the uses of the invention described above apply, mutatis mutandis, to this aspect of the invention. Targeted therapeutic agents are those agents that block (or inhibit) the growth of cancer cells by interfering with (or inhibiting) specific molecules (e.g. proteins such as enzymes or receptors) that are required for (or implicated in) cancer growth or proliferation (as opposed to simply interfering with all rapidly dividing cells, e.g. as per traditional chemotherapies). Targeted therapeutic agents include, but are not limited to, Gleevec (Imatinib), Avastin (Bevacizumab) and Everolimus. Cytotoxic therapeutic agents include, but are not limited to, carboplatin, a taxol or a vinca alkaloid.

In another aspect, the present invention provides a method of treating cancer in a subject, said method comprising administering to a subject in need thereof a combination of a therapeutically effective amount of a selenium compromised thioredoxin reductase-derived apoptotic protein (SecTRAP) forming agent and a targeted therapeutic agent or a cytotoxic therapeutic agent. Embodiments of the uses of the invention described above apply, mutatis mutandis, to this aspect of the invention.

In another aspect, the present invention provides the use of a selenium compromised thioredoxin reductase-derived apoptotic protein (SecTRAP) forming agent in the manufacture of a medicament for treating cancer wherein said treatment further comprises the administration of a targeted therapeutic agent or a cytotoxic therapeutic agent. Embodiments of the uses of the invention described above apply, mutatis mutandis, to this aspect of the invention.

In another aspect, the present invention provides a selenium compromised thioredoxin reductase-derived apoptotic protein (SecTRAP) forming agent for use in treating an immune cell infiltrated cancer in a subject, wherein said agent has immunostimulatory activity thereby causing said subject to raise (or stimulate or cause or enhance or elicit) an immune response against said cancer. Embodiments of the uses of the invention described above apply, mutatis mutandis, to this aspect of the invention. Also, types of immune cells that may infiltrate a cancer in accordance with this aspect of the invention are also evident from the discussion elsewhere herein. Such immune cells include, for example, T-cells (e.g. CD8+ T-cells and Tregs), natural killer (NK) cells, tumour-associated macrophages (TAMs), neutrophils, mast cells and myeloid-derived suppressor cells (MDSCs). Discussion elsewhere herein in relation to T-cell infiltration (and T-cell infiltrated cancers), for example degrees of infiltration and methods of determining or selecting T-cell-infiltrated cancers, may be applied, mutatis mutandis, to this aspect of the invention (which relates to treating an immune cell infiltrated cancer).

The present invention also provides a method of treating an immune cell infiltrated cancer in a subject, said method comprising administering to a subject in need thereof a therapeutically effective amount of selenium compromised thioredoxin reductase-derived apoptotic protein (SecTRAP) forming agent, wherein said agent has immunostimulatory activity thereby causing the said subject to raise (or stimulate or cause or enhance or elicit) an immune response against said cancer. Embodiments of the uses of the invention described above apply, mutatis mutandis, to this aspect of the invention.

The present invention also provides the use of selenium compromised thioredoxin reductase-derived apoptotic protein (SecTRAP) forming agent in the manufacture of a medicament for treating an immune cell infiltrated cancer wherein said agent has immunostimulatory activity thereby causing the said subject to raise (or stimulate or enhance or elicit) an immune response against said cancer. Embodiments of the uses of the invention described above apply, mutatis mutandis, to this aspect of the invention.

In a further aspect, the present invention provides kits comprising one or more of the SecTRAP forming agents or formulations as defined above for use according to the invention. Preferred SecTRAP forming agents are described elsewhere herein. The kits may comprise further components (e.g. a further immunostimulatory agent and/or a chemotherapeutic agent). Each component may be provided in a separate compartment or vessel. Where convenient and practical, mixtures of components could be provided. The components may be provided in dry, e.g. crystallised, freeze dried or lyophilised, form or in solution, typically such liquid compositions will be aqueous and buffered with a standard buffer such as Tris, HERBS, etc.

Such kits may comprise further components (e.g. as described above).

Preferably the kits are for use in treating cancer (such as T-cell infiltrated cancers), e.g. are for use in the methods or uses of the present invention as described herein.

In one aspect, the present invention provides a kit comprising a SecTRAP forming agent and an immunostimulatory agent (e.g. a checkpoint inhibitor such as an anti-PD-L1 antibody or an anti-PD-1 antibody e.g. Pembrolizumab).

In one aspect, the present invention provides a kit comprising a SecTRAP forming agent and an anti-thioredoxin antibody.

The kits may also be provided with instructions for using the kit in accordance with the invention or with directions for how such instructions may be obtained.

The invention will be further described with reference to the following non-limiting Example with reference to the following drawings in which:

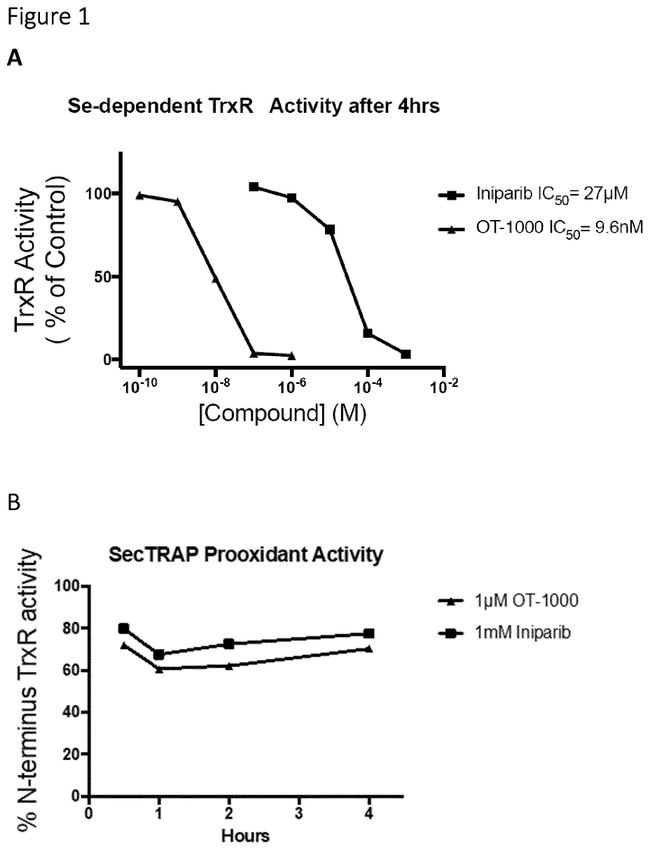

FIG. 1: (A) Compounds were incubated in the presence of NADPH-reduced TrxR for 4 hours. After incubation, Sec-dependent, C-terminal TrxR activity was determined with the addition of DTNB. Activity was normalized to DMSO only Vehicle control and TrxR lacking blank controls. (B) OT-1000 and Iniparib were incubated in the presence of NADPH-reduced TrxR for various time points at concentrations aimed to completely inhibit the Sec-dependent, C-terminal, activity of TrxR. N-terminal substrate, SecTRAP, activity was determined with the addition of Juglone and the following of NADPH consumption.

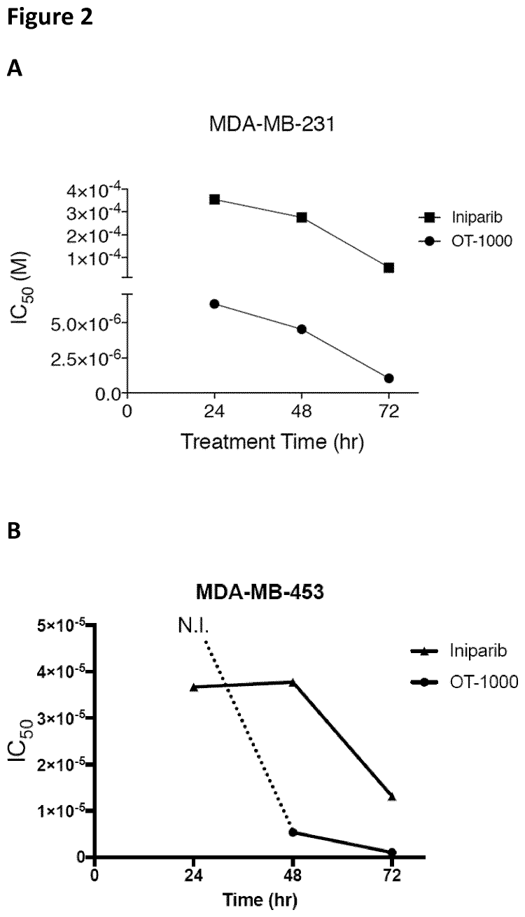

FIG. 2: Sensitivity of breast cancer cell lines MDA-MB-231 (A) and MDA-MB-453 (B) to OT-1000. Cells were incubated in the presence of multiple concentrations of Iniparib or OT-1000 for 24, 48, or 72 hours. Cell viability was then assessed using the CellQuanti assay. Relative cell viability was determined using DMSO only and blank controls. Linear regression analysis was applied to determine the inhibitory concentration to 50% of control (IC50).

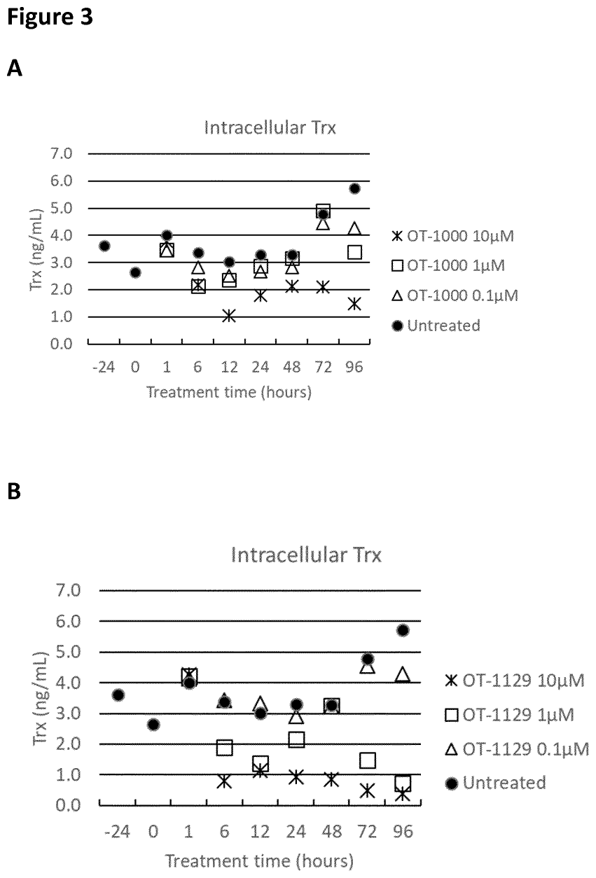

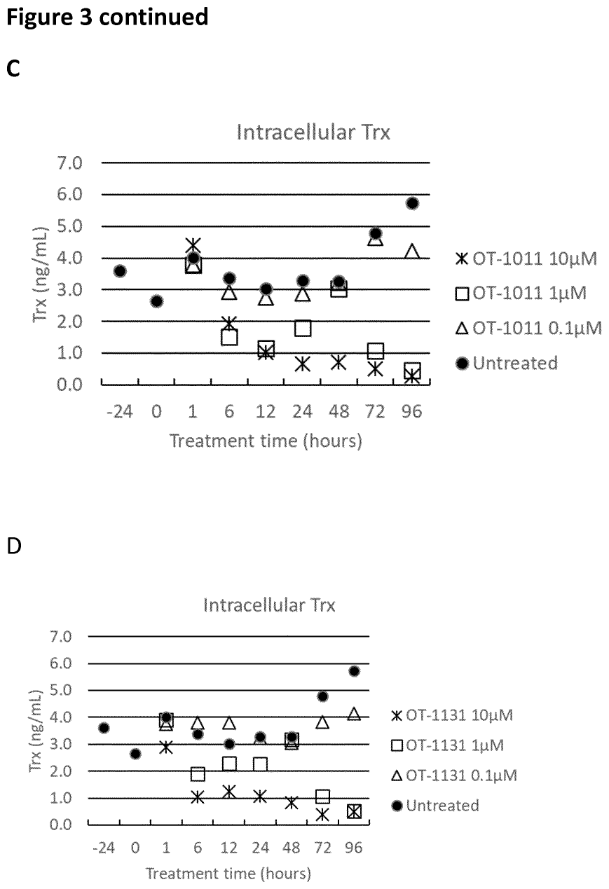

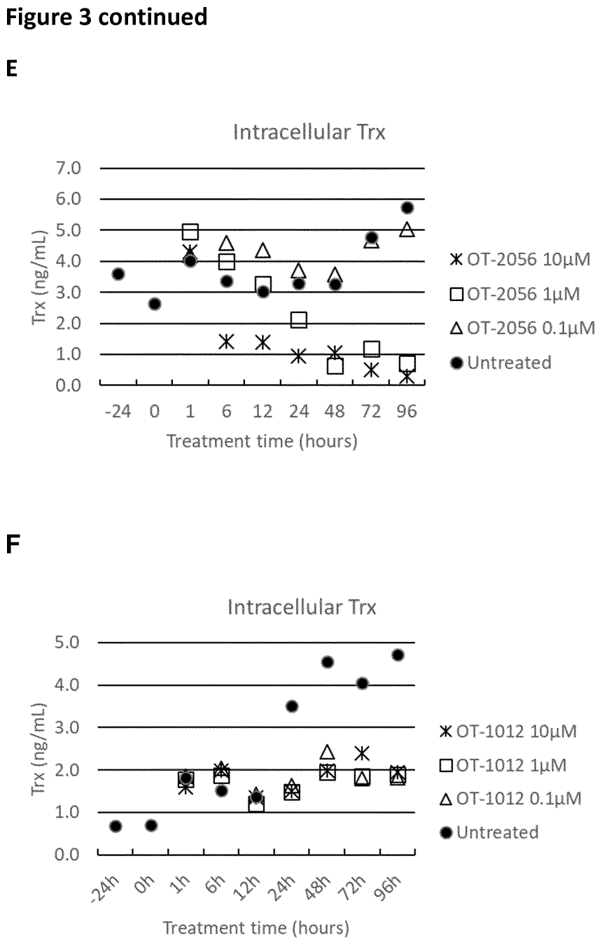

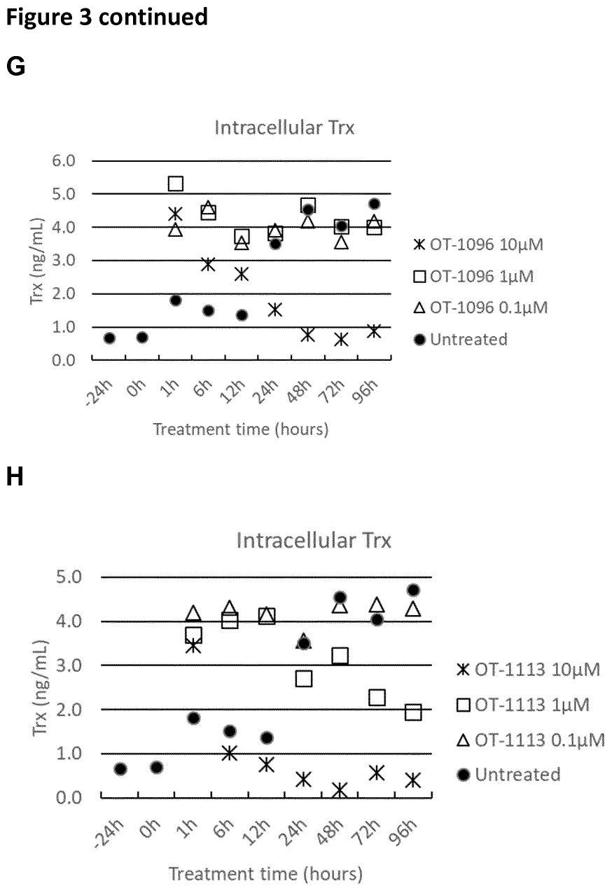

FIG. 3: (A) 07-1000; (B) OT-1129; (C) OT-1011; (D) OT-1131; (E) OT-2056; (F) OT-1012; (G) OT-1096; (H) OT-1113. Intracellular Trx levels in MDA-MB-231 tumor cells during treatment with various concentrations of the stated compound, compared with untreated cells, over 96 hours of treatment. At each sampling time, the cell supernatant was removed from the cells, the cells were washed and lysed, and the total amount of Trx from all cells in the cell lysates was determined using ELISA.

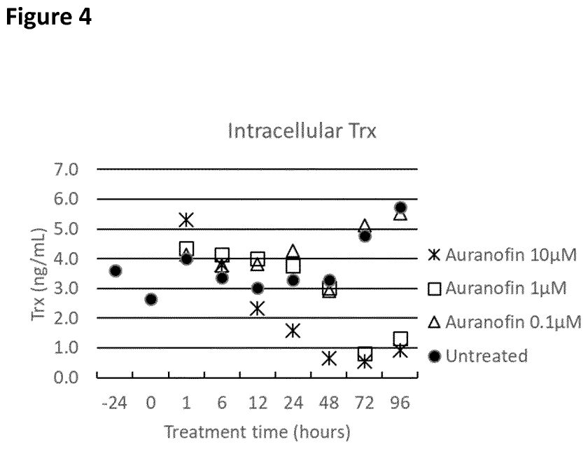

FIG. 4: Intracellular Trx levels in MDA-MB-231 tumor cells during treatment with various concentrations of auranofin, compared with untreated cells, over 96 hours of treatment. At each sampling time, the cell supernatant was removed from the cells, the cells were washed and lysed, and the amount of Trx in the cell lysates was determined using ELISA.

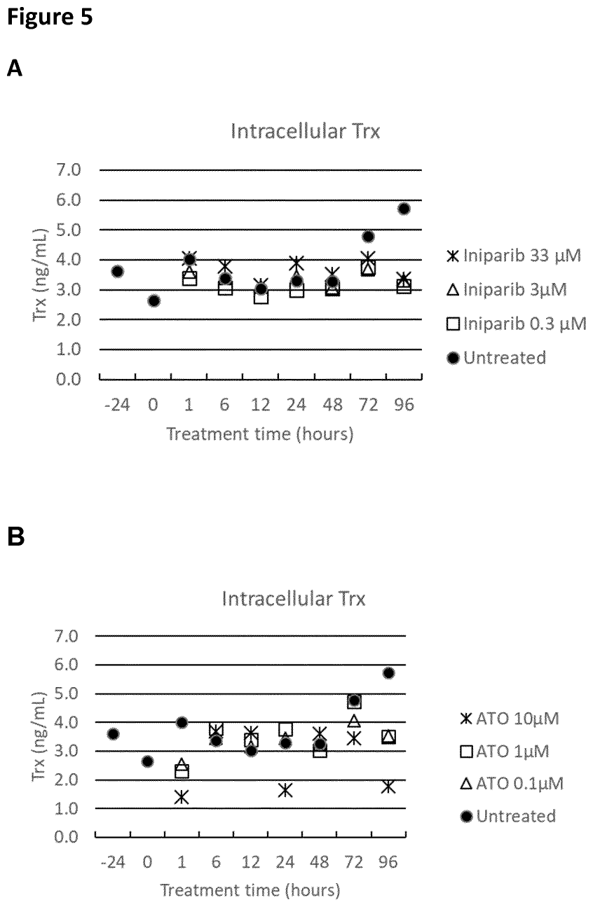

FIG. 5: (A) Intracellular Trx levels in MDA-MB-231 tumor cells during treatment with various concentrations of Iniparib, compared with untreated cells, over 96 hours of treatment. At each sampling time, the cell supernatant was removed from the cells, the cells were washed and lysed, and the amount of Trx in the cell lysates was determined using ELISA. (B) Intracellular Trx levels in MDA-MB-231 tumor cells during treatment with various concentrations of ATO, compared with untreated cells, over 96 hours of treatment. At each sampling time, the cell supernatant was removed from the cells, the cells were washed and lysed, and the amount of Trx in the cell lysates was determined using ELISA.

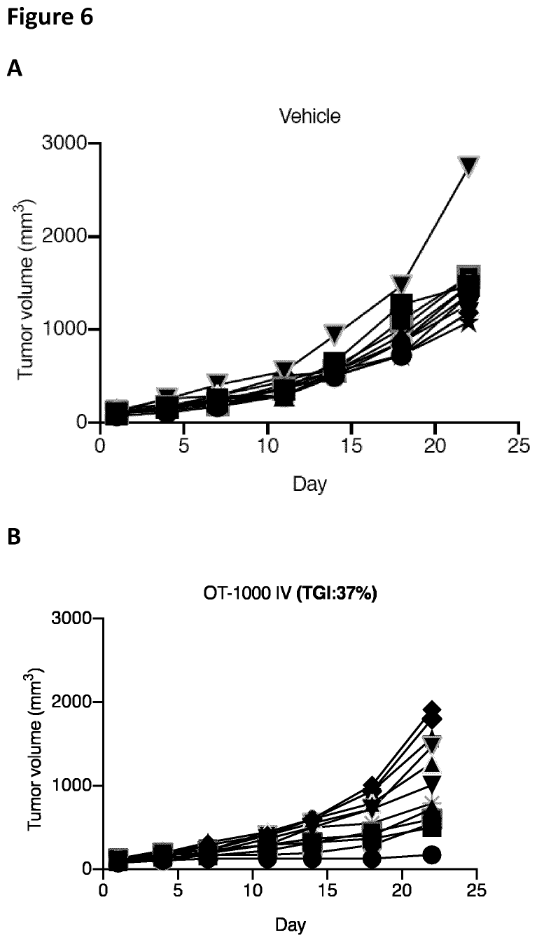

FIG. 6: (A) Control. Individual tumor growth of MDA-MB-231 xenografts in immunodeficient athymic mice treated with vehicle control (I.V.) 5/2 (5 days on, 2 days off), then three times per week for two weeks. (B) OT-1000. Individual tumor growth of MDA-MB-231 xenografts in immunodeficient athymic nude mice treated with 10 mg/kg OT-1000 I.V. 5/2, then three times per week for two weeks. Total growth inhibition (TGI) represents the percentage of the median tumor volume of OT-1000 treated group compared to the median tumor volume in the vehicle control group.

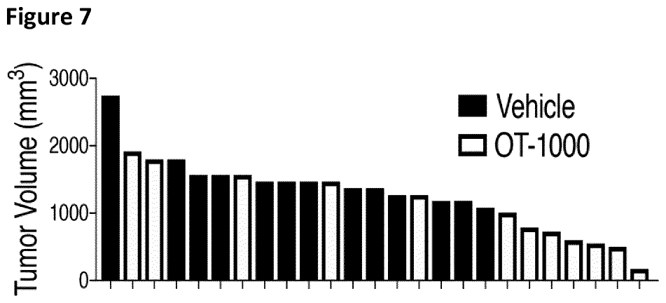

FIG. 7: Waterfall plot of final xenograft tumor volumes of MDA-MB-231 cancer cells in immunodeficient athymic nude mice after 22 days of treatment treated with OT-1000 or vehicle control. The waterfall plot presents individual measured tumor sizes, to visualize distribution.

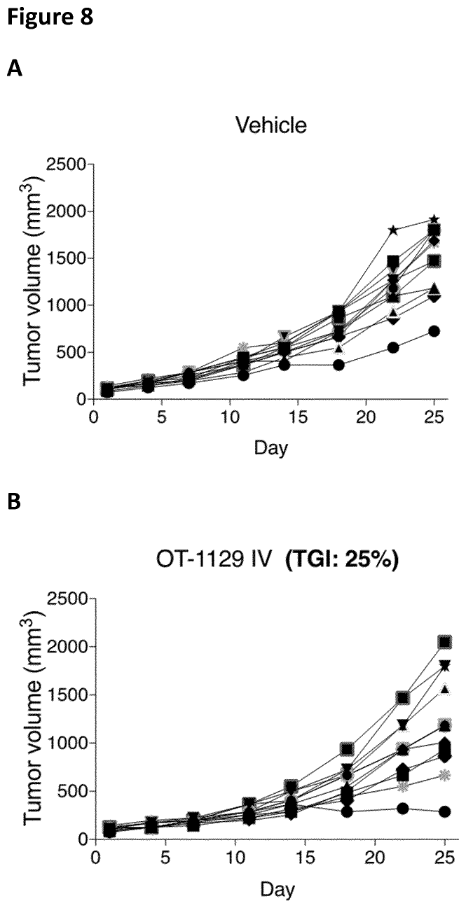

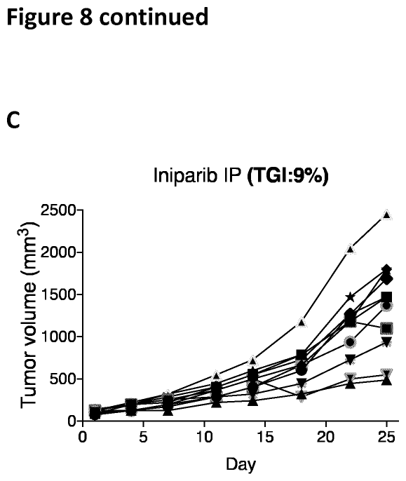

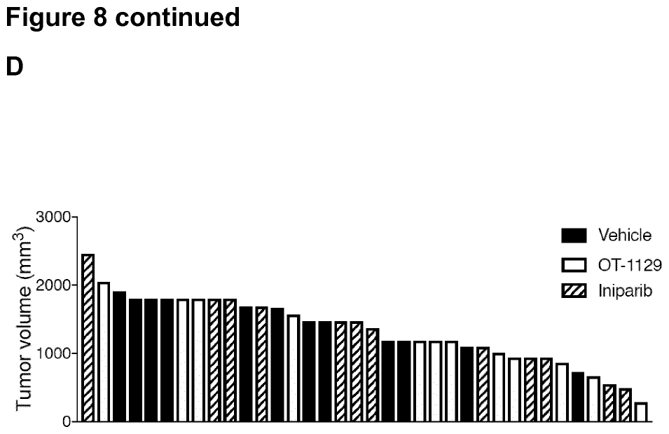

FIG. 8: (A) Control. Individual tumor growth of MDA-MB-231 xenografts in immunodeficient athymic mice treated with vehicle control. (B) OT-1129. Individual tumor growth of MDA-MB-231 xenografts in immunodeficient athymic nude mice treated with OT-1129. Total growth inhibition (TGI) represents the percentage of the median tumor volume of OT-1000 treated group compared to the median tumor volume in the vehicle control group. (C) Iniparib. Individual tumor growth of MDA-MB-231 xenografts in immunodeficient athymic nude mice treated with Iniparib. Total growth inhibition (TGI) represents the percentage of the median tumor volume of Iniparib treated group compared to the median tumor volume in the vehicle control group. (D) Waterfall plot of final xenograft tumor volumes of MDA-MB-231 cancer cells in immunodeficient athymic nude mice after 25 days of treatment treated with OT-1129, Iniparib, or vehicle control.

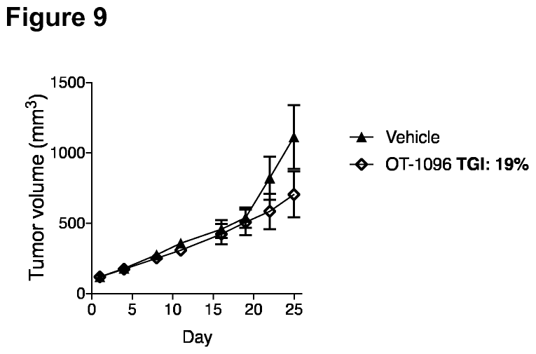

FIG. 9: MDA-MB-231 xenograft tumor growth in immunodeficient athymic nude mice treated with OT-1096 or vehicle control.

Athymic nude mice were inoculated orthotopically with 5×10⁶ MDA-MB-231 breast cancer cells into the mammary fat pad and randomized for treatment when tumors reached an average volume of 80-120 mm³ (N=12 in each group). Mice were treated with 10 mg/kg OT-1096 via i.v. injection or with vehicle i.v., once a day using a 5 day on two day off (5/2) dosing regimen for the duration of the experiment. Xenograft tumor volume was assessed using caliper measurements for 25 days. Data is represented as mean tumor volume±SEM. Statistical significance (p<0.05) was determined using a Two-way repeated measures ANOVA with Sidak's multiple comparison test. Mean tumor volume for mice treated with OT-1096 was statistically significant compared to vehicle at day 25.

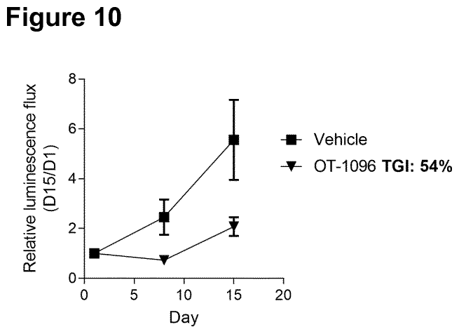

FIG. 10: Relative luminescence flux in primary 4T1-luc2 tumors implanted into the mammary fat pad of immunocompetent BALB/C mice and treated with OT-1096 or vehicle control. Luminescent flux in each mouse was normalized to baseline values determined at day 1 imaging.

Female BALB/c immunocompetent mice were implanted with 1×10⁵ 4T1-luc2 murine tumor cells into the mammary fat pad. Upon growth of the tumors between 60-90 mm³ the animals were selected for imaging. Mice were randomized and enrolled for treatment based on imaging flux values. Mice were treated once daily with 5 mg/kg of either OT-1096 or vehicle control using a 5/2 (five days on, two days off) dosing protocol. Upon days 1, 8, and 15 whole body imaging was performed on the mice to follow tumor cell luminescence. Analysis consists of primary tumor luminescence in mice. Each mouse was normalized to its own baseline luminescence from day 1. Mice that did not have metastasis present at the first day of imaging were included in the study (N=9). Data is represented as mean±SEM. Statistical significance (p<0.05) was determined using a Mann-Whitney test. The relative luminescence flux for mice treated with OT-1096 was significant compared to vehicle at day 15.

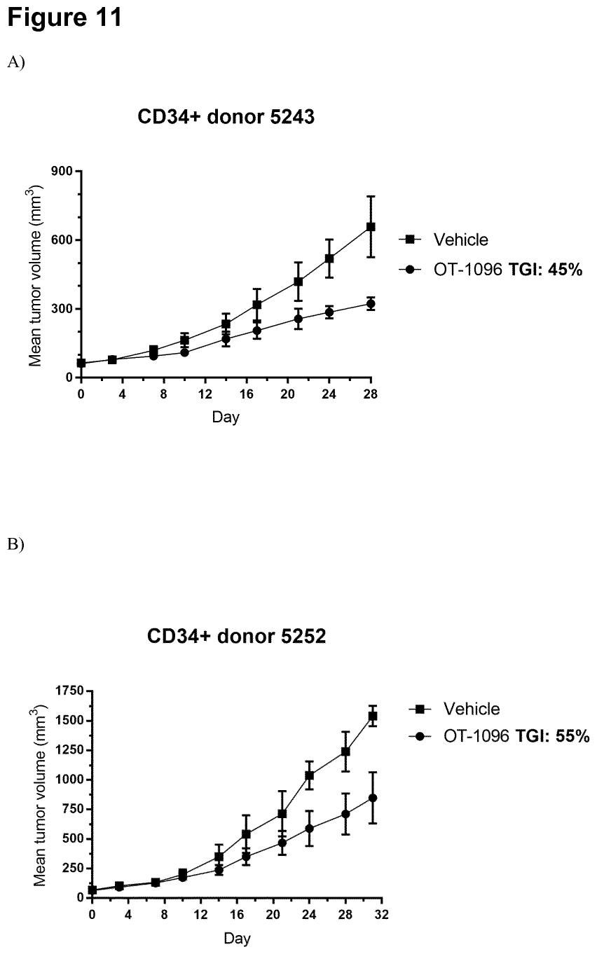

FIG. 11: Primary tumor growth of TM00098 patient derived triple-negative breast cancer xenografts in humanized immunocompetent NSG mice (Hu-CD34-NSG) treated with OT-1096 or vehicle control. A) Humanized NSG mice (Hu-CD34-NSG) engrafted with CD34+ cells from donor 5243 and subsequently implanted with TNBC PDX TM00098. B) Humanized NSG mice (Hu-CD34-NSG) engrafted with CD34+ cells from donor 5252 and subsequently implanted with TNBC PDX TM00098. Female NSG mice were implanted with human CD34+ hematopoietic stem cells from multiple donors and the level of human CD45+ cells were measured in the peripheral blood 12 weeks post engraftment. Mice with >25% human CD45+ cells in the peripheral blood were determined to have a humanized immune system (Hu-CD34-NSG™) mice and were enrolled into the study. The Hu-CD34-NSG mice were implanted with TM00098 patient-derived xenografts (PDX) subcutaneously on the right flank. The TM00098 PDX cancer cells originate from a primary tumor of a patient suffering from a grade 3 TNBC invasive ductal carcinoma. When the tumors reached a volume between 60-120 mm³ mice were treated with either 10 mg/kg OT-1096 three times a week intravenously (donor 5243 n=5, donor 5252 n=7) or with a vehicle three times a week intravenously (donor 5243 n=3, donor 5252 n=2). In case of tail vein swelling when the test substance or vehicle could not be administered intravenously, Intraperitoneal injection was applied. Tumor volume was measured using a digital caliper two times a week for the duration of the study. Animals that reached a body condition score of ≤2, a body weight loss of ≥20% or a tumor volume >2000 mm³ were euthanized before study terminus. Animals with ulcerated tumors were also euthanized before study terminus. Data is represented as mean tumor volume±SEM. Statistical significance (p<0.05) was determined using a Two-way repeated measures ANOVA with Sidak's multiple comparison test. Mean tumor volume for mice treated with OT-1096 was statistically significant compared to vehicle at day 24 and 28 for CD34+ donor 5243 and at day 31 for CD34+ donor 5252.

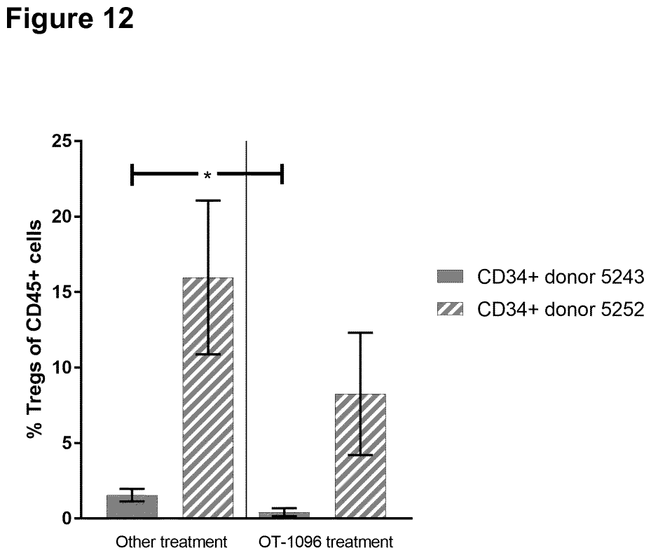

FIG. 12: Treg levels of TM00098 patient derived triple-negative breast cancer xenografts in humanized immunocompetent NSG mice (Hu-CD34-NSG) comparing OT-1096 treatment to other treatment. All animals treated with OT-1096 alone or OT-1096 in combination with Pembrolizumab have been grouped (OT-1096 treatment). All animals treated with PBS, OT-1096's vehicle or Pembrolizumab alone have been grouped (other treatment). Data is presented as mean % Tregs of CD45+ cells±SEM. OT-1096 treatment (donor 5243 n=5, donor 5252 n=11) other treatment (donor 5243 n=7, donor 5252 n=6). Statistical significance (p<0.05, *) was determined using a Mann-Whitney test.

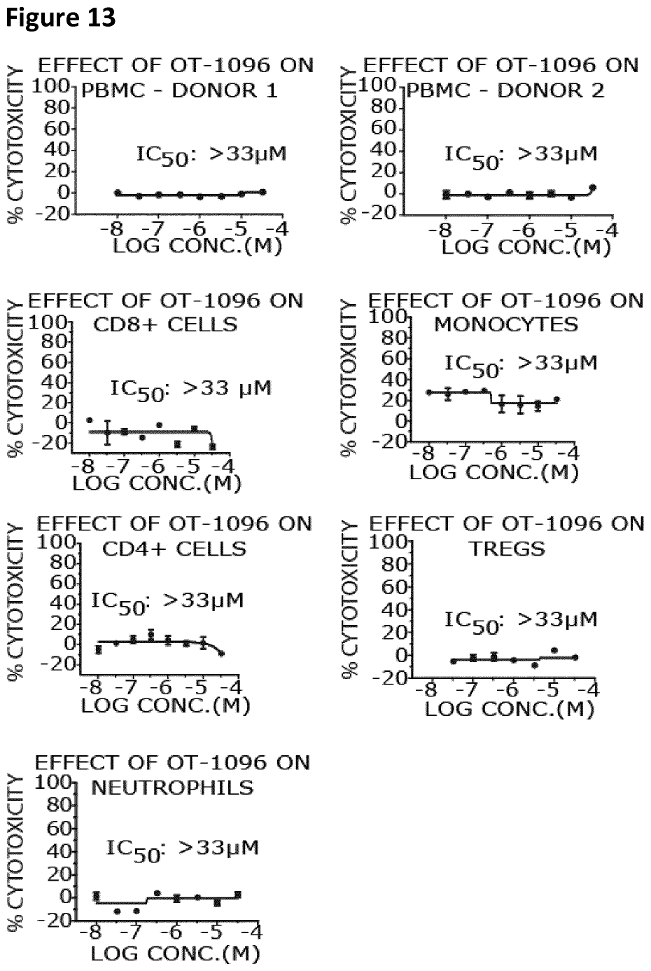

FIG. 13: Viability of immune cells isolated from human blood donors after treatment with OT-1096 for 24 h. Data is presented as mean cytotoxicity±SEM. N=3.

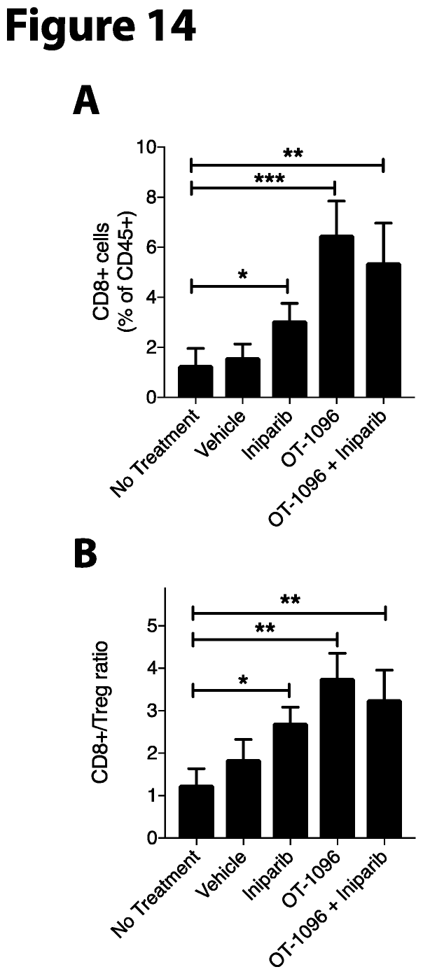

FIG. 14: Intratumoural immune cell populations of engrafted tumors after treatment with OT-1096, Iniparib, or the combination of OT-1096 and Iniparib. Panel (A) shows CD8+ cell levels. Panel (B) shows the ratio of CD8+ cells to Treg cells.

### EXAMPLE 1

### General Methods for Chemistry

All reagents were obtained commercially and used as received. All reactions were run under a nitrogen atmosphere. Reactions were monitored by thin layer chromatography (TLC) with detection using the appropriate staining reagent or by ESI-LCMS (positive ion mode with UV detection at 254 nm). All ¹H NMRs were recorded on Bruker Advance 400 MHz spectrometer with multinuclear probe in the appropriate solvent. ¹H NMR and ¹³C NMR spectra were recorded on Bruker Avance 400 (¹H NMR: 400 MHz; ¹³C NMR: 100 MHz) using tetramethylsilane as internal standard for ¹H NMR spectra in CDCl₃. Residual solvent peak for DMSO-d₆ (39.43 ppm), or CDCl₃ (77.00 ppm) for ¹³C NMR spectra. The residual solvent peak of DMSO-d₆ in ¹H NMR is 2.5 ppm. Abbreviations used are: s, singlet; d, doublet; t, triplet; m, multiplet; br, broad singlet. Coupling constants are expressed in Hz.
For all reactions, analytical grade solvents were used. All moisture sensitive reactions were carried out in oven-dried glassware (70° C.).
For crude LCMS monitoring, mass spectra were obtained with API 2000 mass spectrophotometer from Applied Biosystems. Samples were infused at 2 ul/min, and spectra were obtained in positive or negative ionization mode.
Precoated aluminum sheets (Merck, 254 nm) were used for TLC. Column chromatography was performed on Swambe silica gel 100-200 mesh.

### Materials and Methods

### Determination of Sec-Dependent TrxR Activity (DTNB Assay)

Sec-dependent TrxR activity was assessed using a 5′-5-dithiobis-(2-nitrobenzoic acid) (DTNB) assay. Multiple concentrations of compounds were incubated for various time points in reaction buffer, consisting of 50 mM Tris pH 7.5 with 2 mM EDTA and 0.1 mg/mL bovine serum albumin, and containing recombinant rat TrxR and 250 μM NADPH. 2.5 mM DTNB was added to each well and TNB⁻ production was followed at OD₄₁₂. Activity was determined following the change in TNB⁻ over time and normalized to DMSO only (Vehicle) and TrxR lacking (blank) controls. The amino acid sequence of rat TrxR1 is available in Gen Bank, with accession number AAF32362.1.

### Glutathione Reductase (GR) Activity Assay

GR activity was determined incubating compounds at various concentrations with GR from baker's yeast and 250 μM NADPH for 15 minutes, whereupon 10 mM oxidized glutathione (GSSG) was added to each well and NADPH consumption was followed at OD₃₄₀. Activity was determined following the consumption of NADPH over time and normalized to DMSO only (Vehicle) and GR lacking (blank) controls.

### Determination of Sec-Independent SecTRAP Activity (Juglone Assay)

SecTRAP forming capabilities of compounds with TrxR was determined using the Juglone Assay. Compounds were incubated in the presence of recombinant TrxR and 250 μM NADPH for 15 minutes at concentrations fully inhibiting enzyme activity in the DTNB assay. 100 μM juglone was then added to each well and NADPH consumption was followed at OD₃₄₀ to determine sustained reductive capacity at the N-terminus of TrxR (SecTRAP Activity). Activity was determined following the consumption of NADPH over time and normalized to DMSO only (Vehicle) and TrxR lacking (blank) controls.

### NADPH-Dependent TrxR Activity

Irreversible inhibition of TrxR was determined by incubating compounds in the presence of recombinant TrxR, with or without 250 μM NADPH, for 90 minutes. Aliquots were used in a DTNB assay to determine inhibition of enzyme activity. Compounds were incubated in the presence of TrxR with 250 μM NADPH at concentrations used to fully inhibit Sec-dependent TrxR activity, as confirmed using the DTNB assay. Incubation samples were then added to a reaction buffer containing 250 μM NADPH and 100 μM juglone, whereupon NADPH consumption was followed at OD₃₄₀ to determine sustained SecTRAP activity.

### Cellular Assays

### Cell Culture

Cell cultures were maintained at 37° C. in 5% CO₂ in medium containing 20 mg/mL penicillin/streptomycin, 2 mM L-glutamine, and 10% fetal bovine serum (FBS). Experiments were performed in triplicate in medium containing 10% FBS and 25 nM sodium selenite. All compounds were diluted in DMSO, 0.01% final concentration.

### Cell Culture Media

MDA-MB-453 (ATCC HTB-131) cells were grown in media with sodium pyruvate. MDA-MB-231 cells (ATCC HTB-26) were grown in DMEM media or L-15 supplemented with Glutamax (1×). U87-MG (ATCC HTB-14) and MDA-MB-468 (ATCC HTB-132) cells were cultured in DMEM supplemented with Glutamax (1×). NB-4 (DSMZ ACC-207) cells were cultured in RPMI 1640.

### CellQuanti-Blue Cell Viability Assay

Cells were plated at 2000 cells/well into 96-well plates in media containing 10% FBS. The following day, compounds were added to each well and incubated for 72 hrs. CellQuanti-Blue cell viability reagent was added to each well and the plates were subsequently incubated at 37° C. for 3 hours. Viability was determined fluorometrically using an Enspire plate reader (G.E. Healthcare, USA) excitation: 530 nm, emission: 590 nm. Viability was normalized to DMSO controls and blank wells with media.

### Alamar-Blue Cell Viability Assay

MDA-MB-231 cells were plated 2000 cells/well in 96-well black optical plates in the presence of 10% FBS media containing 25 nM selenite. The following day cells were treated with various concentrations of compounds (0.1% DMSO final) and incubated for 72 hrs. After the incubation Alamar Blue reagent was added to each well and incubated for additional 3 hrs. Fluorescence was read ex:530 nm/em:590 nm, and percent of viability was determined using DMSO vehicle and no cell (blank) controls.

### MTT Cell Viability Assay

Breast cancer and glioblastoma cell lines were plated 4000 cells/well in 96-well plates in the presence of 10% FBS media. The following day cells were treated with various concentrations of the example compounds (0.1% DMSO final) and incubated for 72 hrs. After the incubation an MTT assay was performed to access cell viability. Percent of viability was determined using DMSO vehicle and no cell (blank) controls.

### Determination of Trx Intracellular and Extracellular Levels

The human mammary carcinoma cell line MDA-MB-231 was obtained from the American Type Culture Collection (ATCC; Manassas, Va.) and cultured in Leibovitz's L-15 medium (Thermo Fisher Scientific, USA) supplemented with 10% (v/v) heat-inactivated fetal bovine serum (Thermo Fisher Scientific, USA), 10 mM HERBS (Sigma), 25 mM sodium bicarbonate (Sigma), 1× Glutamax (Sigma), 100 IU/mL penicillin and 100 μg/ml streptomycin (Sigma) in a 5% CO₂ atmosphere at 37° C. Briefly, cells were seeded at 7500 cells/well in 96-well plates. After 24 h, the cells were treated either with different concentrations of test compounds or DMSO (0.5%; vehicle control) respectively and further incubated at 37° C. in a CO₂ incubator. Culture supernatants were collected at different time points, like, immediately after cell plating (−24 h), 0 h (at the time of compound addition), and 1 h, 6 h, 12 h, 24 h, 48 h, 72 h and 96 h following compound addition. Subsequently, cells were lysed from corresponding wells with 0.1% Triton X-100 and the lysates were collected. Trx levels were measured in both the culture supernatants and cell lysates by ELISA using TXN (Human) ELISA Kit [Abnova; Cat no: KA0535] following manufacturer instruction. A standard curve was prepared using different concentrations of the Trx standard (provided in the kit) with the help of GraphPad Prism software (version 5.0; La Jolla, Calif., USA). Trx concentrations for the unknown samples were calculated from the standard curve. The data were expressed as the means of three replicates.

### In Vivo Assays

### Efficacy of OT-1000 Towards Inhibition of MDA-MB-231 Xenograft Tumor Growth in Athymic Nude Mice

Athymic nude mice were inoculated orthotopically with 5×10⁶ MDA-MB-231 breast cancer cells into the mammary fat pad, and randomized for treatment when tumors reached an average volume of 80-120 mm³ (N=12 in each group). Mice were treated with 10 mg/kg OT-1000 via i.v. injection or with vehicle i.v., once a day for the first five days, followed by two days of no treatment, and then three times per week for two weeks. Xenograft tumor volume was assessed using caliper measurements for 22 days. Athymic nude mouse model is described by Richmond A, and Su Y., Disease Models & Mechanisms. 2008; 1(2-3):78-82.

### Efficacy of OT-1129 and Iniparib Towards Inhibition of MDA-MB-231 Xenograft Tumor Growth in Athymic Nude Mice

Athymic nude mice were inoculated orthotopically with 5×10⁶ MDA-MB-231 breast cancer cells into the mammary fat pad, and randomized for treatment when tumors reached an average volume of 80-120 mm³ (N=12 in each group). Mice were treated with 25 mg/kg OT-1129 via i.v. injection, 25 mg/kg Iniparib via i.p. injection, or with vehicle i.v., once a day for the first five days, followed by two days of no treatment, then three times per week for two weeks and four days totaling 12 doses. Xenograft tumor volume was assessed using caliper measurements for 25 days.
MDA-MB-231 Xenograft Tumor Growth in Immunodeficient Athymic Nude Mice Treated with OT-1096 or Vehicle Control.
Athymic nude mice were inoculated orthotopically with 5×10⁶ MDA-MB-231 breast cancer cells into the mammary fat pad and randomized for treatment when tumors reached an average volume of 80-120 mm³ (N=12 in each group). Mice were treated with 10 mg/kg OT-1096 via i.v. injection or with vehicle i.v., once a day using a 5 day on two day off (5/2) dosing regimen for the duration of the experiment. Xenograft tumor volume was assessed using caliper measurements for 25 days. Data is represented as mean tumor volume±SEM. Statistical significance (p<0.05) was determined using a Two-way repeated measures ANOVA with Sidak's multiple comparison test. Mean tumor volume for mice treated with OT-1096 was statistically significant compared to vehicle at day 25.
Relative Luminescence Flux in Primary 4T1-Luc2 Immunocompetent Tumors Implanted into the Mammary Fat Pad of BALB/C Mice and Treated with OT-1096 or Vehicle Control.
Female BALB/c immunocompetent mice were implanted with 1×10⁵ 4T1-luc2 murine tumor cells into the mammary fat pad. Upon growth of the tumors between 60-90 mm³ the animals were selected for imaging. Mice were randomized and enrolled for treatment based on imaging flux values. Mice were treated once daily with 5 mg/kg of either OT-1096 or vehicle control using a 5/2 (five days on, two days off) dosing protocol. Upon days 1, 8, and 15 whole body imaging was performed on the mice to follow tumor cell luminescence. Analysis consists of primary tumor luminescence in mice. Each mouse was normalized to its own baseline luminescence from day 1. Mice that did not have metastasis present at the first day of imaging were included in the study (N=9). Data is represented as mean±SEM. Statistical significance (p<0.05) was determined using a Mann-Whitney test. The relative luminescence flux for mice treated with OT-1096 was significant compared to vehicle at day 15.
Primary Tumor Growth of TM00098 Patient Derived Xenografts-Triple Negative Breast Cancer in Immunocompetent Humanized NSG Mice (Hu-CD34-NSG) Treated with OT-1096 or Vehicle Control.
Female NSG mice were implanted with human CD34+ hematopoietic stem cells from multiple donors and the level of human CD45+ cells were measured in the peripheral blood 12 weeks post engraftment. Mice with >25% human CD45+ cells in the peripheral blood were determined to have a humanized immune system (Hu-CD34-NSG™) mice and were enrolled into the study. The Hu-CD34-NSG mice were implanted with TM00098 patient-derived xenografts (PDX) subcutaneously on the right flank. The TM00098 PDX cancer cells originate from a primary tumor of a patient suffering from a grade 3 TNBC invasive ductal carcinoma. When the tumors reached a volume between 60-120 mm³ mice were treated with either 10 mg/kg OT-1096 three times a week intravenously (donor 5243 n=5, donor 5252 n=7) or with a vehicle three times a week intravenously (donor 5243 n=3, donor 5252 n=2). In case of tail vein swelling when the test substance or vehicle could not be administered intravenously, Intraperitoneal injection was applied. Tumor volume was measured using a digital caliper two times a week for the duration of the study. Animals that reached a body condition score of ≤2, a body weight loss of ≥20% or a tumor volume >2000 mm³ were euthanized before study terminus. Animals with ulcerated tumors were also euthanized before study terminus. Data is represented as mean tumor volume±SEM. Statistical significance (p<0.05) was determined using a Two-way repeated measures ANOVA with Sidak's multiple comparison test. Mean tumor volume for mice treated with OT-1096 was statistically significant compared to vehicle at day 24 and 28 for CD34+ donor 5243 and at day 31 for CD34+ donor 5252.

### Synthesis of Compounds

### Synthesis of OT-1000 (2-((4-Chlorophenyl)sulfonyl)-6-methoxy-3-nitropyridine)

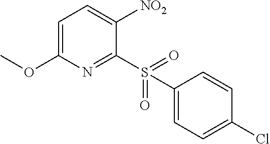 [SMILES: COc1ccc([N+](=O)[O-])c(S(=O)(=O)c2ccc(Cl)cc2)n1]

To a stirred solution of 6-methoxy-2-chloro-3-nitro pyridine(1; 5.0 g, 26.596 mmol) in dimethyl acetamide (75 mL) was added sodium 4-chlorobenzene-sulphinate (2; 7.92 g, 39.894 mmol) and tetra-N-butylammonium chloride (2.22 g, 7.979 mmol) and Cone HCl 0.75 mL at room temperature. The reaction mixture was stirred at 80° C. for 1 hour. Progress of reaction was monitored by LCMS. The whole reaction mixture was poured on crushed ice to get solid. This solid compound was filtered through sintered funnel and thoroughly dried under vacuum to isolate 2-((4-Chlorophenyl)sulfonyl)-6-methoxy-3-nitropyridine as desired product (7.1 g, 81.21%). ¹H NMR (400 MHz, CDCl₃) δ 8.10 (d, J=8.6 Hz, 1H), 8.0 (d, J=7.9 Hz, 2H), 7.56 (d, J=7.9 Hz, 2H), 6.95 (d, J=8.7 Hz, 1H), 3.69 (s, 3H). LCMS [m/z (M+H)+=(Calculated for C₁₂H₉N₂O₅SCI+H: 329) found: 329], Purity at λ=220 nm: 98.73%

### Synthesis of OT-1011 (2-Benzylsulfonyl-6-methoxy-3-nitropyridine)

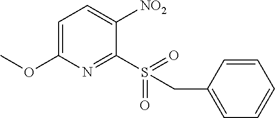 [SMILES: COc1ccc([N+](=O)[O-])c(S(=O)(=O)Cc2ccccc2)n1]

### Step-1

To a solution of 6-methoxy-2-chloro-3-nitro pyridine (1; 5.0 g, 26.596 mmol) in dimethylformamide (20 mL) was added potassium carbonate (4.441 g, 32.181 mmol) and benzyl mercaptan (3.595 g, 28.989 mmol) at room temperature. The reaction mixture was stirred for overnight at room temperature. Progress of reaction was monitored by LCMS. The reaction mixture was quenched with ice cold water (30 ml) and was extracted with ethyl acetate (300 mL). The organic layer was washed with water (3×50 mL) followed by brine (1×50 mL). The organic layer was dried over anhydrous sodium sulphate and was evaporated under reduced pressure to give the crude product which was purified by column chromatography eluting with 2% ethyl acetate in hexane affording the step-1 compound (3.2 g, 43.55%) as yellow solid.

### Step-2

To a solution of step-1 compound (Step-1; 15.0 g, 54.348 mmol) in dichloromethane (200 mL) was added m-chloro per benzoic acid (32.71 g, 190.17 mmol) at room temperature. The reaction mixture was stirred at room temperature for overnight.
Progress of reaction was monitored by LCMS. The reaction mixture was diluted with dichloromethane (500 mL) and washed with saturated sodium sulphite solution (2×100 mL) followed by brine (1×200 mL). The organic layer was dried over anhydrous sodium sulphate and was evaporated under reduced pressure to give the crude product which was purified by column chromatography eluting with 10% ethyl acetate in hexane affording the title compound OT-1011 (8.0 g, 92.15%) as off white solid. ¹H NMR (400 MHz, CDCl₃) δ 8.06 (d, J=8.6 Hz, 1H), 7.38 (m, 5H), 6.99 (d, J=8.7 Hz, 1H), 4.82 (s, 2H), 3.96 (s, 3H). LCMS [m/z (M+H)+=(Calculated for C₁₃H₁₂N₂O₅S+H: 309) found: 309], Purity at λ=220 nm: 100%

### Synthesis of OT-1012 (6-Methoxy-3-nitro-2-(pyridin-2-ylsulfonyl)pyridine)

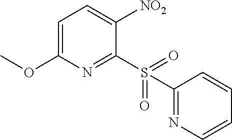 [SMILES: COc1ccc([N+](=O)[O-])c(S(=O)(=O)c2ccccn2)n1]

### Step-1

To a solution of 6-methoxy-2-chloro-3-nitro pyridine (1; 5.0 g, 26.596 mmol) in dimethylformamide (20 mL) was added potassium carbonate (4.441 g, 32.181 mmol) and 2-mercapto pyridine (3.22 g, 28.989 mmol) at room temperature. The reaction mixture was stirred for overnight at room temperature. Progress of reaction was monitored by LCMS. The reaction mixture was quenched with ice cold water (30 ml) and was extracted with ethyl acetate (300 mL). The organic layer was washed with water (3×50 mL) followed by brine (1×50 mL). The organic layer was dried over anhydrous sodium sulphate and was evaporated under reduced pressure to give the crude product which was purified by column chromatography eluting with 5% ethyl acetate in hexane affording the step-1 compound (2.5 g, 35.7%) as yellow solid.

### Step-2

To a solution of step-1 compound (6.36 g, 24.183 mmol) in dichloromethane (150 mL) was added m-chloro per benzoic acid (14.55 g, 84.639 mmol) at room temperature. The reaction mixture was stirred at room temperature for overnight. Progress of reaction was monitored by LCMS. The reaction mixture was diluted with dichloromethane (300 mL) and washed with saturated sodium sulphite solution (2×50 mL) followed by brine (1×50 mL). The organic layer was dried over anhydrous sodium sulphate and was evaporated under reduced pressure to give the crude product which was purified by column chromatography eluting with 5% ethyl acetate in hexane affording the title compound OT-1012 (2.5 g, 36%) as off white solid. ¹H NMR (400 MHz, CDCl₃) δ 8.70-8.69 (m, 1H), 8.29-8.26 (m, 2H), 8.05-8.01 (m, 1H), 7.57-7.54 (m, 1H), 6.98 (d, J=8.8 Hz, 1H), 3.66 (s, 3H). LCMS [m/z (M+H)+=(Calculated for C₁₁H₉N₃O₅S+H: 296) found: 296], Purity at λ=220 nm: 98.76%

### Synthesis of QT-1096 (6-methoxy-3-nitro-2-(octylsulfonyl)pyridine)

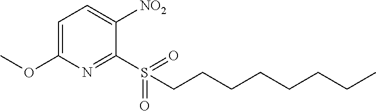 [SMILES: CCCCCCCCS(=O)(=O)c1nc(OC)ccc1[N+](=O)[O-]]

### Step-1

To a solution of 6-methoxy-2-chloro-3-nitro pyridine(1; 250 mg, 0.1.33 mmol) in dimethylformamide (5 mL) was added potassium carbonate (220 mg, 1.596 mmol) and octane-1-thiol (213 mg, 1.463 mmol) at room temperature. The reaction mixture was stirred overnight at room temperature. Progress of reaction was monitored by LCMS. The reaction mixture was quenched with ice cold water (10 ml) and was extracted with ethyl acetate (20 mL). The organic layer was washed with water (3×10 mL) followed by brine (1×10 mL). The organic layer was dried over anhydrous sodium sulphate and was evaporated under reduced pressure to give the crude product which was purified by column chromatography eluting with 4% ethyl acetate in hexane affording the step-1 compound (150 mg, 37.8%) as yellow solid.

### Step-2

To a solution of step-1 compound (150 mg, 0.798 mmol) in dichloromethane (10 mL) was added m-chloro per benzoic acid (494 mg, 2.872 mmol) at room temperature. The reaction mixture was stirred at room temperature overnight. Progress of reaction was monitored by LCMS. The reaction mixture was diluted with dichloromethane (20 mL) and washed with saturated sodium sulphite solution (2×20 mL) followed by brine (1×20 mL). The organic layer was dried over anhydrous sodium sulphate and was evaporated under reduced pressure to give the crude product which was purified by column chromatography eluting with 20% ethyl acetate in hexane affording the title compound OT-1096 (46 mg, 27.69%) as off white solid. ¹H-NMR [CDCl3, δ 8.13 (d, J=9 Hz, 1H), 7.04 (d, J=8 Hz, 1H), 4.07 (s, 3H), 3.56 (t, J=8 Hz, 2H), 1.89-1.86 (m, 2H), 1.46-1.44 (m, 2H), 1.27-1.25 (m, 8H), 0.86 (m, 3H)]. LCMS [m/z (M+H)+=(Calculated for C₁₄H₂₂N₂O₅S+H: 331) found: 331], Purity at λ=220 nm: 100%

### Synthesis of OT-1113 (methyl 3-((6-methoxy-3-nitropyridin-2-yl)sulfonyl)propanoate)

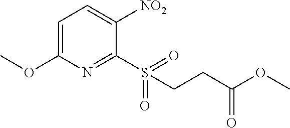

### Step-1

To a solution of 6-methoxy-2-chloro-3-nitro pyridine (250 mg, 1.33 mmol) in dimethylformamide (5 mL) was added potassium carbonate (220 mg, 1.59 mmol) and 3-Mercapto-propionic acid methyl ester (175 mg, 1.46 mmol) at room temperature. The reaction mixture was stirred overnight at room temperature. Progress of reaction was monitored by LCMS. The reaction mixture was quenched with ice cold water (10 ml) and was extracted with ethyl acetate (20 mL). The organic layer was washed with water (3×10 mL) followed by brine (1×10 mL). The organic layer was dried over anhydrous sodium sulphate and was evaporated under reduced pressure to give the crude product which was purified by column chromatography eluting with 5% ethyl acetate in hexane affording the step-1 compound (148 mg, 43.42%) as yellow solid.

### Step-2

To a solution of step-1 compound (148 mg, 0.54 mmol) in dichloromethane (10 mL) was added m-chloro per benzoic acid (477.65 mg, 2.72 mmol) at room temperature. The reaction mixture was stirred at room temperature overnight. Progress of reaction was monitored by LCMS. The reaction mixture was diluted with dichloromethane (20 mL) and washed with saturated sodium sulfite solution (2×20 mL) followed by brine (1×20 mL). The organic layer was dried over anhydrous sodium sulphate and was evaporated under reduced pressure to give the crude product which was purified by column chromatography eluting with 8% ethyl acetate in hexane affording the title compound OT-1113 (104 mg, 62.87%) as off white solid. ¹H-NMR [DMSO-d6, δ 8.49 (d, J=9 Hz, 1H), 7.37 (d, J=9 Hz, 1H), 4.01-3.97 (m, 5H), 3.59 (s, 3H), 2.84 (t, J=7 Hz, 2H)]. LCMS [m/z (M+H)+=(Calculated for C₁₀H₁₂N₂O₇S+H: 305) found: 305], Purity at λ=220 nm: 98.40%

### Synthesis of OT-1129 (2-(ethylsulfonyl)-6-methoxy-3-nitropyridine)

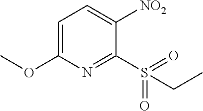 [SMILES: CCS(=O)(=O)c1nc(OC)ccc1[N+](=O)[O-]]

### Step-1

To a solution of 6-methoxy-2-chloro-3-nitro pyridine (250 mg, 1.33 mmol) in dimethylformamide (5 mL) was added potassium carbonate (220 mg, 1.59 mmol) and ethane thiol (1.33 mg, 2.66 mmol) at room temperature. The reaction mixture was stirred overnight at room temperature. Progress of reaction was monitored by LCMS. The reaction mixture was quenched with ice cold water (10 ml) and was extracted with ethyl acetate (20 mL). The organic layer was washed with water (3×10 mL) followed by brine (1×10 mL). The organic layer was dried over anhydrous sodium sulphate and was evaporated under reduced pressure to give the crude product which was purified by column chromatography eluting with 2% ethyl acetate in hexane affording the step-1 compound (187 mg, 69.73%) as yellow solid

### Step-2

To a solution of step-1 compound 3 (187 mg, 1.16 mmol) in dichloromethane (10 mL) was added m-chloro per benzoic acid (602 mg, 3.5 mmol) at room temperature. The reaction mixture was stirred at room temperature overnight. Progress of reaction was monitored by LCMS. The reaction mixture was diluted with dichloromethane (20 mL) and washed with saturated sodium sulfite solution (2×20 mL) followed by brine (1×20 mL). The organic layer was dried over anhydrous sodium sulphate and was evaporated under reduced pressure to give the crude product which was purified by column chromatography eluting with 12% ethyl acetate in hexane affording the title compound OT-1129 (108 mg, 50.18%) as off white solid. ¹H-NMR [DMSO-d6, δ 8.48 (d, J=9 Hz, 1H), 7.36 (d, J=9 Hz, 1H), 4.02 (s, 3H), 3.72-3.67 (m, 2H), 1.26 (t, J=7 Hz, 3H)]. LCMS [m/z (M+H)+=(Calculated for C₈H₁₀N2O₅S+H: 247) found: 247], Purity at λ=220 nm: 100%

### Synthesis of OT-1131 ((2-(ethylsulfinyl)-6-methoxy-3-nitropyridine)

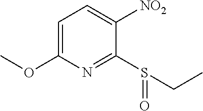

### Step-1

To a solution of 6-methoxy-2-chloro-3-nitro pyridine(1; 5 g, 26.59 mmol) in dimethylformamide (50 mL) was added potassium carbonate (4.44 g, 31.95 mmol) and ethane thiol (2,1.81 g, 29.25 mmol) at room temperature. The reaction mixture was stirred overnight at room temperature. Progress of reaction was monitored by LCMS. The reaction mixture was quenched with ice cold water (35 ml) where in solid precipitated from the reaction mixture. The solid were filtered and washed with ice cold water (3×30 mL) and was dried under reduced pressure affording the compound-3 (4.8 g, 84.24%) as yellow solid.

### Step-2

To a solution of compound-3 (4.8 g, 22.42 mmol) in dichloromethane (100 mL) was added m-chloro per benzoic acid (8.84 g, 51.40 mmol) at room temperature. The reaction mixture was stirred at room temperature overnight. Progress of reaction was monitored by LCMS. The reaction mixture was diluted with dichloromethane (20 mL) and washed with saturated sodium sulphite solution (2×80 mL) followed by brine (1×80 mL). The organic layer was dried over anhydrous sodium sulphate and was evaporated under reduced pressure to give the crude product which was purified by column chromatography eluting with 80% ethyl acetate in hexane affording the title compound OT-1131 (2.6 g, 60.61%) as yellow solid. ¹H NMR (DMSO-d6, 400 MHz) δ 8.55 (d, J=8.9 Hz, 1H), 7.16 (d, J=8.9 Hz, 1H), 4.08 (s, 3H), 3.26-3.18 (m, 1H), 2.97-2.88 (m, 1H), 1.23 (t, J=7.3 Hz, 3H). LCMS [m/z (M+H)+=(Calculated for C₈H₁₀N₂O₄S+H: 231) found: 231], Purity at λ=220 nm: 99.38%

### Synthesis of OT-2056 (exo-4,11-Dibenzyl-4,11-diazatricyclo[5.3.1.0^{2,6}]undec-9-ene-3,5,8-trione)

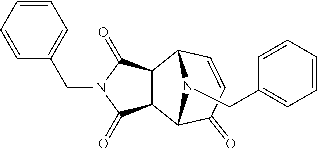 [SMILES: O=C1C=C[C@H]2[C@@H]3C(=O)N(Cc4ccccc4)C(=O)[C@@H]3[C@@H]1N2Cc1ccccc1]

is as described in the published PCT patent application WO 2017027358 A1.
Iniparib may be synthesised, for example, as described in WO 1994026730A2.
Auranofin may be synthesised, for example, as described in U.S. Pat. No. 4,200,738A

### Results

### SecTRAP Forming Activity of Compounds

A compound (agent) may be classified as a SecTRAP forming agent if C-terminal activity of TrxR as assessed by a DTNB assay is inhibited but N-terminal activity of TrxR as a assessed by a juglone assay is not significantly inhibited (or not fully inhibited or not abolished).

The minimal concentration of compound at which 100% inhibition is observed in the DTNB assay was established and then, using that concentration of the given compound, the effect on juglone reduction in the juglone assay was assessed. Higher % values for juglone activity (juglone reduction activity) in this assay are indicative of stronger prooxidant activity.

The SecTRAP forming activity of various compounds is summarised in Table A below. TrxR IC50 is the concentration at which 50% of Thioredoxin Reductase activity is inhibited (Molar concentration), as assessed in the DTNB assay. GR IC50 Concentration at which 50% of Glutathione Reductase activity is inhibited (Molar concentration), as assessed by the GR activity assay. Juglone Act (% @ 100% DTNB inhibition) means the % activity observed in the juglone assay when the compound is used at the concentration that achieves 100% inhibition of TrxR in the DTNB assay.

TABLE A

| | | | Juglone Act | Compound |
|---|---|---|---|---|
| | | | (% @ 100% | is an |
| | | | DTNB | example |
| Compound | TrxR IC50 | GR IC50 | inhibition) | of formula # |
| OT-1000 | 1.45E−08 | >30E−06 | 84.35 | I |
| OT-1099 | 1.04E−08 | >30E−06 | 42.86 | I |
| OT-1104 | 1.849E−07 | >30E−06 | 43.48 | I |
| OT-1109 | 1.829E−08 | >30E−06 | 49.28 | I |
| OT-1098 | 1.266E−08 | >30E−06 | 53.62 | I |
| OT-1083 | 1.145E−08 | >30E−06 | 60.87 | I |
| OT-1084 | 2.576E−10 | >30E−06 | 69.57 | I |
| OT-1094 | 1.139E−08 | >30E−06 | 75.36 | I |
| OT-2056 | 8.335E−08 | >30E−06 | 107.692 | II |
| OT-1218 | 1.848E−06 | >30E−06 | 58.6931 | III |
| OT-1012 | 2.684E−08 | >30E−06 | 89.8 | IV |
| OT-1118 | 1.08E−08 | >30E−06 | 42.86 | IV |
| OT-1119 | 8.549E−10 | 0.000011 | 44.64 | IV |
| OT-1122 | 3.991E−09 | >30E−06 | 53.57 | IV |
| OT-1108 | 6.901E−09 | >30E−06 | 57.97 | IV |
| OT-1128 | 1.595E−07 | >30E−06 | 65.31 | V |
| OT-1087 | 9.69E−08 | >30E−06 | 65.99 | V |
| OT-1124 | 1.032E−07 | >30E−06 | 73.47 | V |
| OT-1129 | 1.241E−07 | >30E−06 | 74.83 | V |
| OT-1114 | 1.217E−07 | >30E−06 | 75.51 | V |
| OT-1011 | 1.82E−08 | >30E−06 | 83.67 | V |
| OT-1127 | 2.041E−07 | >30E−06 | 84.35 | V |
| OT-1113 | 6.057E−08 | >30E−06 | 91.16 | V |
| OT-1088 | 7.599E−09 | >30E−06 | 33.33 | V |
| OT-1117 | 6.78E−09 | >30E−06 | 35.71 | V |
| OT-1101 | 6.686E−08 | >30E−06 | 39.29 | V |
| OT-1103 | 1.882E−08 | >30E−06 | 39.29 | V |
| OT-1090 | 4.35E−09 | >30E−06 | 46.38 | V |
| OT-1089 | 7.161E−09 | >30E−06 | 47.83 | V |
| OT-1092 | 2.03E−08 | >30E−06 | 52.17 | V |
| OT-1100 | 5.051E−09 | >30E−06 | 58.93 | V |
| OT-1081 | 1.03E−09 | >30E−06 | 65.22 | V |
| OT-1095 | 7.33E−09 | >30E−06 | 71.01 | V |
| OT-1091 | 2.532E−08 | >30E−06 | 71.01 | V |
| OT-1096 | 1.231E−08 | >30E−06 | 86.96 | V |
| OT-1115 | 1.622E−07 | >30E−06 | 70.07 | VI |
| OT-1116 | 9.304E−08 | >30E−06 | 70.07 | VI |
| OT-1086 | 3.906E−07 | >30E−06 | 79.59 | VI |
| OT-1025 | 3.157E−09 | >30E−06 | 89.12 | VII |
| Auranofin | 7.345E−09 | >30E−06 | 72.11 | |
| ATO | 3.100E−06 | >30E−06 | 82.75 | |
| Iniparib | 1.770E−04 | >30E−06 | 90.48 | |

Example SecTRAP data are shown for various compounds tested (Table A—above). The data shows TrxR IC50 values for each compound, and retained juglone activities (N-terminal dithiol motif activity) of TrxR in the situation where TrxR is 100% inhibited at the C-terminal active site by compound, and where the concentration of the compound was equal to that required to obtain 100% inhibition.

The compounds are also specific towards TrxR over GR, as shown by the IC50 values, which is a proof of target specificity (Table A). GR is relevant in the sense that GR represents a main off-target candidate for the compounds in question. The IC50 values for TrxR are generally significantly lower than for GR, meaning that much lower amounts of compound are required to inhibit TrxR than GR. GR inhibition could cause damage to normal cells.

We have discovered that Iniparib is a SecTRAP forming compound. We believe that in clinical trials Iniparib performed well in cancers where TrxR/Trx is overexpressed and where there is intratumoural immune-cell infiltration, e.g. Triple Negative Breast Cancer. Iniparib has shown clinical benefit, as compared to control, in the 2nd and 3rd line setting, i.e. in patients who have received induction treatment with chemotherapy prior to treatment with Iniparib.

As mentioned above, we have discovered that Iniparib is a SecTRAP forming compound. As also described herein it has been surprisingly found that SecTRAP forming agents have, in addition to a direct cytotoxic effect, a an ability to confer an anti-cancer immune response. The fact that we have found that Iniparib is a SecTRAP forming agent and that SecTRAP forming agents can confer an anti-cancer immune response is consistent with the positive results in clinical trials in infiltrated tumours.

ATO (arsenic trioxide, As₂O₃) can increase cellular levels of ROS via several targets and cause apoptosis. Here we have discovered that, and provide definite evidence that, ATO also is a TrxR SecTRAP forming compound.

Auranofin is related to production of reactive oxygen species as well as the intracellular levels of TrxR. Here we have shown that auranofin is a SecTRAP-forming agent.

Compounds that inhibit the C-terminal active site of TrxR will lower or prevent reduction of the substrate Trx, which the is normal cellular reducing activity of TrxR. This will lead to a buildup of oxidized Trx. Reduced Trx is required to maintain low levels of reactive oxygen species in the cell. The thioredoxin system directly acts as a reactive oxygen species scavenger (Das, K. C. & Das, C. K. (2000) Biochem. Biophys. Res. Commun. 277, 443-447) and also maintains other intracellular pathways also performing this action. For example, reduced Trx is a direct electron donor to peroxiredoxins or thioredoxin peroxidases, which are major hydrogen peroxide-scavenging enzymes that normally keep the level of reactive oxygen species in the cell under control (Fang J, et al. J Biol Chem. 2005 Jul. 1; 280(26):25284-90). If, in addition, the N-terminal dithiol motif is still active after inhibition of the C-terminal active site, toxicity in the form of reactive oxygen species is produced in the cell via the N-terminal active site.

We compared OT-1000 to Iniparib with regard to inhibition of C-terminal activity and retained N-terminal activity. In one example, OT-1000 and Iniparib were assayed at various concentrations, with a binding time of 4 hours. Both compounds inhibited TrxR C-terminal activity, and in this assay, OT-1000 (IC50=27 μM) was 1000-fold more potent than Iniparib (IC50=9.6 nM) (FIG. 1A).

Compared head to head within the same experiment, we also show that for Iniparib at 1 mM, N-terminal juglone reduction activity is retained at a similar level to that obtained for OT-1000 at 1 μM (FIG. 1B).

We determined the cytotoxic, or cell-killing effect of various SecTRAP-forming compounds on the cancer cell lines MDA-MB-231 (a breast cancer cell line), MDA-MB-468 (a breast cancer cell line), NB-4 (a leukaemia cell line), and U-87 MG (a glioblastoma cell line) (Table X and Y). The data in Table X was obtained using the Alamar Blue cell viability assay. The data in Table Y was obtained using the MTT cell viability assay.

TABLE X

| Name | MDA-MB-231 (IC50, M) |
|---|---|
| Auranofin | 1.40E−06 |
| Iniparib | 5.44E−05 |
| OT-1000 | 3.53E−06 |
| OT-1011 | 3.81E−06 |
| OT-1012 | 3.61E−06 |
| OT-1025 | 2.92E−06 |
| OT-1081 | 3.70E−05 |
| OT-1083 | 4.20E−06 |
| OT-1084 | 3.44E−06 |
| OT-1086 | 1.23E−05 |
| OT-1087 | 5.60E−06 |
| OT-1088 | 1.80E−06 |
| OT-1089 | 7.90E−07 |
| OT-1090 | 1.30E−06 |
| OT-1091 | 9.45E−07 |
| OT-1092 | 5.24E−06 |
| OT-1094 | 4.21E−06 |
| OT-1095 | 7.13E−06 |
| OT-1096 | 4.51E−06 |
| OT-1098 | 3.52E−06 |
| OT-1099 | 1.73E−06 |
| OT-1100 | 1.85E−06 |
| OT-1101 | 2.66E−06 |
| OT-1103 | 5.22E−06 |
| OT-1104 | 4.17E−06 |
| OT-1108 | 3.09E−06 |
| OT-1109 | 3.82E−06 |
| OT-1113 | 8.09E−06 |
| OT-1114 | 1.14E−05 |
| OT-1115 | 5.08E−06 |
| OT-1116 | 4.53E−06 |
| OT-1117 | 4.36E−06 |
| OT-1118 | 4.54E−06 |
| OT-1122 | 1.09E−05 |
| OT-1124 | 1.25E−05 |
| OT-1127 | 5.13E−06 |
| OT-1128 | 3.31E−06 |
| OT-1129 | 2.95E−06 |

TABLE Y

| | NB-4 | MDA-MB-468 | U-87 MG | MDA-MB-231 |
|---|---|---|---|---|
| Name | (IC50, M) | (IC50, M) | (IC50, M) | (IC50, M) |
| Auranofin | | 3.10E−07 | 1.72E−06 | 1.66E−06 |
| Iniparib | 5.76E−05 | >33 | >33E−06 | >33E−06 |
| OT-1000 | 1.20E−06 | 9.21E−06 | 1.27E−05 | 3.14E−06 |
| OT-1011 | 1.21E−06 | 2.89E−06 | 6.11E−06 | 1.80E−06 |
| OT-1012 | | 4.34E−06 | 8.20E−06 | 2.97E−06 |
| OT-1025 | 8.30E−07 | 3.86E−06 | 6.76E−06 | 5.26E−06 |
| OT-1084 | 9.40E−07 | | | 3.44E−06 |
| OT-1086 | | 4.00E−06 | 1.06E−05 | 9.97E−06 |
| OT-1087 | | 3.88E−06 | 7.52E−06 | 7.73E−06 |
| OT-1096 | 3.22E−06 | | | |
| OT-1113 | 1.17E−06 | 3.90E−06 | 5.23E−06 | 4.90E−06 |
| OT-1114 | | 5.47E−06 | 1.63E−05 | 1.84E−05 |
| OT-1115 | | 4.34E−06 | 6.11E−06 | 7.53E−06 |
| OT-1116 | | 8.94E−06 | 8.89E−06 | 8.94E−06 |
| OT-1117 | 1.20E−06 | | | 4.36E−06 |
| OT-1124 | | 9.77E−06 | 1.95E−05 | >33E−06 |
| OT-1127 | | 4.90E−06 | 6.00E−06 | 7.10E−06 |
| OT-1128 | | 4.68E−06 | 6.92E−06 | 5.91E−06 |
| OT-1129 | 1.12E−06 | 3.16E−06 | 5.95E−06 | 5.12E−06 > NL > |
| | | | | 4.88E−06 |
| OT-1132 | | >10E−06 | >33E−06 | >33E−06 |
| OT-1133 | | 6.40E−07 | 2.48E−06 | 1.42E−06 |
| OT-1134 | | >33E−06 | >33E−06 | >33E−06 |
| OT-1135 | | 9.60E−07 | 2.53E−06 | 1.02E−06 |
| OT-1244 | | >33E−06 | >33E−06 | >33E−06 |
| OT-1245 | | 1.85E−06 | 9.25E−06 | 3.84E−06 |
| OT-1246 | | >0.000033 | >0.000033 | >0.000033 |
| OT-1247 | | 2.10E−06 | 9.27E−06 | 3.54E−06 |
| OT-1248 | | >0.000033 | >0.000033 | >0.000033 |
| OT-1249 | | 2.40E−06 | | 3.57E−06 |
| OT-1250 | | >0.000033 | >0.000033 | >0.000033 |
| OT-1251 | | 5.58E−06 | | 1.16E−05 |
| OT-1252 | | | | 3.58E−06 |
| OT-1253 | | | | >0.000033 |
| OT-2056 | 4.20E−07 | | | |

It is also shown that MDA-MB-231 and MDA-MB-453 cultured breast cancer cells are increasingly sensitive to OT-1000 exposure overtime. MDA-MB-231 and MDA-MB-453 are model cell lines for triple-negative breast cancer, and are characterized by basal-like properties, which in turn are associated with aggressiveness in the clinical setting. This time-dependent efficacy means that the mechanistic induction of oxidative stress is not due to a promiscuous reactivity of the compounds, but is an active function resulting from SecTRAP formation (FIGS. 2A and 2B). Cells were incubated in the presence of multiple concentrations of Iniparib or OT-1000 for 24, 48, or 72 hours. Cell viability was then assessed using the CellQuanti-Blue assay. Relative cell viability was determined using DMSO only and blank controls. Linear regression analysis was applied to determine the inhibitory concentration to 50% of control (IC50). In these conditions, OT-1000 was 100-fold more potent than Iniparib in MDA-MB-231 cells, and OT-1000 was more potent in MDA-MB-453 cells than Iniparib.

During the tumor cell killing process with the compounds described herein, we have shown in vitro, using MDA-MB-231 tumor cells, that the intracellular amount of Trx is decreased. This is shown for OT-1000 and OT-1129 as examples. For OT-1000 and OT-1129 treatments (0.1 μM, 1 μM and 10 μM), intracellular Trx appears to decline during treatment of MBA-MD-231 tumor cells. The effects were greater with increasing concentration of compound (FIGS. 3A and B).

Total intracellular Trx at end of experiment was lower in OT-1000 and OT-1129-treated cell cultures than for untreated cell cultures. In these experiments, OT-1129 had a more rapid effect than OT-1000.

The intracellular Trx level was also assessed during treatment with OT-1011, OT-1131, OT-2056, OT-1012, OT-1096 and OT-1013 (FIG. 3C, D, E, F, G and H). At 1 μM and 10 μM OT-1011, intracellular levels of Trx are reduced compared with control cells within 6-12 hours, and then remain lower than control. At 1 μM and 10 μM OT-1131, intracellular levels of Trx are reduced compared with control cells within 6 hours, and then remain lower than control. At 1 μM and 10 μM OT-2056, intracellular levels of Trx are reduced compared with control cells after 24 hours and 6 hours respectively, and then remain lower than control. At all doses of OT-1012, intracellular levels of Trx are reduced compared with control cells after 12 hours, and then remain lower than control. At 10 μM of OT-1096, intracellular levels of Trx are reduced compared with control cells after 24 hours, and then remain lower than control. At 1 μM and 10 μM of OT-1113, intracellular levels of Trx are reduced compared with control cells after 48 hours and 65 hours respectively, and then remain lower than control.

Auranofin had a similar effect to the OT compounds (0.1 μM, 1 μM and 10 μM) (FIGS. 4A and 4B).

Iniparib also reduced the intracellular Trx levels in comparison to the level seen with untreated cells, e.g at the 96 h time point (FIG. 5A).

ATO also reduced the intracellular Trx levels in comparison to the level seen with untreated cells, e.g at the 96 h time point (FIG. 5B).

Eventually, the tumor cells die and stop producing Trx. During cell death there will likely be a transient local increase of extracellular Trx during tumor treatment, after which Trx levels will decline to zero. Without wishing to be bound by theory, in the situation where we treat tumors in vivo with the compounds herein, after a certain amount of time tumor cell-derived Trx will be decreased with the consequence that Treg suppressive activity in the tumor will be diminished. With diminished Treg activity, antitumoral T cell activity should increase. During tumor cell destruction, tumor cell-specific material will be processed by the immune system at the priming stage to further boost an adaptive anti-tumoral response. Further, without being bound by theory, it is possible that Trx, released during the cytolytic burst will attract a new Tcell population migrating into the tumor microenvironment with favorable composition, eg having high CD8+/Treg ratio that will trigger an anti-tumoral response.

We show herein the anti-cancer effect of compounds including OT-1000 in immunoincompetent (or in other words immunodeficient or immunocompromised) mice. In one example, OT-1000 demonstrated a tumor growth inhibition rate (TGI) of 37% in MDA-MB-231 xenograft-bearing athymic nude mice treated intravenously (IV) with 10 mg/kg OT-1000, with administration of compound occurring 11 times over the course of 22 days (FIGS. 6A and 6B, vehicle and OT-1000 IV). The graphs depict individual tumor volumes. Total growth inhibition (TGI) represents the percentage of the median tumor volume of the OT-1000 treated group compared to the median tumor volume in the vehicle.

The data is also visualized in a waterfall plot which presents individual measured tumor sizes at end of experiment, to visualize distribution of tumor size over treatment arms. More tumors of smaller size are observed for OT-1000 treated animals than vehicle-treated animals, indicating anti-tumor efficacy (FIG. 7). That is, OT-1000-treated tumors are more frequently of lower size than vehicle-treated tumors.

In the same model system, MDA-MB-231 xenograft-bearing immunodeficient or immunocompromised athymic nude mice, OT-1129 achieved a TGI of 25% when given intravenously and Iniparib achieved a TGI of 9% when given intraperitoneally. The figures show plots of individual tumor growth (FIGS. 8A, B and C).

Final tumor volumes after treatment with OT-1129, Iniparib or vehicle (MDA-MB-231 xenografts in immunodeficient or immunocompromised athymic nude mice) are also visualized in a waterfall plot (FIG. 8D). Vehicle-treated tumor volumes are skewed towards larger size. In the OT-1129 arm, tumor volumes are skewed towards smaller volumes. That is, OT-1129-treated tumors are more frequently of lower size than vehicle-treated tumors.

The SecTRAP forming compound OT-1096 has an % TGI of 19% when treating immunodeficient or immunocompromised athymic nude mice implanted with MDA-MB-231 Xenografts. OT-1096 was administered IV at 10 mg/kg using a 5 day on two day off (5/2) dosing regimen. The % TGI equals 1-(median of tumor volume of treated animals/median tumor volume of vehicle control)×100. (FIG. 9)

Surprisingly, the SecTRAP forming compound OT-1096 displays a pronounced increased efficacy in immunocompetent mice in comparison with immunoincompetent mice (i.e. immunodeficient or immunocompromised). In BALB/c mice possessing an intact immune system and 4T1-luc2 mammary tumors, representing a TNBC murine tumor, OT-1096 achieved a % TGI of 54% when treated with only 5 mg/kg OT-1096 5/2 for 15 days. The % TGI equals 1-(median of tumor volume of treated animals/median tumor volume of vehicle control)×100. The tumor volume was measured by lucipherase bioluminescence). (FIG. 10).
Furthermore, in Hu-CD34-NSG mice, possessing a humanized immune system and patient derived TNBC tumor xenografts, OT-1096 elicited a % TGI of 45% and 55% in mice bearing human immune cells from two separate donors. This is again a pronounced increased efficacy demonstrated in immunocompetent mice in comparison with immunoincompetent mice (i.e. immunodeficient or immunocompromised). The % TGI equals 1-(median of tumor volume of treated animals/median tumor volume of vehicle control)×100. The tumor volume was measured with caliper (FIGS. 11A and B).
One important conclusion we draw when comparing treatments with OT-1096 performed in immunoincompetent mice and immunocompetent mice, is that the % TGI is severalfold higher in the immunocompetent models (exemplified by BALB/c mice possessing an intact immune system and 4T1-luc2 mammary tumors as well as in Hu-CD34-NSG mice, possessing a humanized immune system and patient derived TNBC tumor xenografts) compared to the immunodeficient or immunocompromised model (MDA-MB-231 xenografts in immunodeficient or immunocompromised athymic nude mice). Further surprisingly, this was achieved with either a lower dose of OT-1096 (a lower dose was used in the study with immunocompetent BALB/c mice with 4T1-luc2 tumors (FIG. 10) compared to MDA-MB-231 xenografts in immunodeficient or immunocompromised athymic nude mice (FIG. 9) or with a lower dosing frequency (a lower dosing frequency was used in the study with Hu-CD34-NSG mice, possessing a humanized immune system and patient derived TNBC tumor xenografts (FIG. 11) compared to MDA-MB-231 xenografts in immunodeficient or immunocompromised athymic nude mice (FIG. 9). Both dosing differences in the two immunocompetent models (BALB/c mice possessing an intact immune system and 4T1-luc2 mammary tumors and Hu-CD34-NSG mice, possessing a humanized immune system and patient derived TNBC tumor xenografts) resulted in a net lower exposure of drug to the mice relative to the immunodeficient or immunocompromised treated mice (MDA-MB-231 xenografts in immunodeficient or immunocompromised athymic nude mice). These lower doses or lower dosing frequencies resulting in a pronounced increased potency of OT-1096 with decreased amounts of compound in two different immunocompetent models show there is an integral interaction with the immune system, where SecTRAP forming compounds work in concert with the immune system to increase anticancer efficacy. Therefore, a new effect of SecTRAP forming compounds exists, working in concert with (or stimulating) the immune system to combat cancer cell growth. The finding that SecTRAP forming agents are able to stimulate (or enhance) an anti-cancer immune response, in addition to having a direct cytotoxic effect, is surprising and it is believed this finding should translate into benefits in the clinic. Purely by way of example, as a result of the finding that SecTRAP forming agents are able to stimulate (or enhance) an anti-cancer immune response, new clinical opportunities and considerations have been opened up, e.g. the possibility of less frequent and/or lower doses and/or the ability to select cancer types sensitive to treatment as well as those subjects that might benefit particularly from treatment (patient stratification) with SecTRAP forming agents (e.g. those with T-cell cell infiltrated tumours).

### EXAMPLE 2

TM00098 Patient Derived Xenografts-Triple Negative Breast Cancer in Immunocompetent Humanized NSG Mice (Hu-CD34-NSG) Treated with OT-1096 Alone or in Combination with Pembrolizumab—Analysis of Treg Levels.
The study with immunocompetent humanized NSG mice (Hu-CD34-NSG) described in Example 1 herein was expanded and the effect on Treg levels of OT-1096 alone or OT-1096 in combination with Pembrolizumab (an anti-PD1 antibody) was assessed.

### Materials and Methods

Female NSG mice were implanted with human CD34+ hematopoietic stem cells from multiple donors and the level of human CD45+ cells were measured in the peripheral blood 12 weeks post engraftment. Mice with >25% human CD45+ cells in the peripheral blood were determined to have a humanized immune system (Hu-CD34-NSG™) mice and were enrolled into the study. The Hu-CD34-NSG mice were implanted with TM00098 patient-derived xenografts (PDX) subcutaneously on the right flank. The TM00098 PDX cancer cells originate from a primary tumor of a patient suffering from a grade 3 TNBC invasive ductal carcinoma. When the tumors reached a volume between 60-120 mm³ mice were treated with either 10 mg/kg OT-1096 three times a week intravenously (donor 5243 n=5, donor 5252 n=7) or with OT-1096's vehicle three times a week intravenously (donor 5243 n=3, donor 5252 n=2) or with 10 mg/kg initial dose, thereafter 5 mg/kg Pembrolizumab two times a week intraperitoneally (donor 5243 n=3, donor 5252 n=3) or with PBS (Pembrolizumab vehicle) two times a week intraperitoneally (donor 5243 n=1, donor 5252 n=3) or with a combination of OT-1096 and Pembrolizumab using their respective treatment schedule (donor 5243 n=3, donor 5252 n=6). In case of tail vein swelling when the test substance or vehicle could not be administered intravenously, Intraperitoneal injection was applied. Animals that reached a body condition score of ≤2, a body weight loss of ≥20% or a tumor volume >2000 mm³ were euthanized before study terminus. Animals with ulcerated tumors were also euthanized before study terminus. Tumor volume was measured using a digital caliper two times a week for the duration of the study. Treatment occurred until sacrifice at day 41. Tumors from the remaining animals were collected and subjected to flow cytometry measurements of infiltrated Treg levels. Tumors were processed into single cell suspensions and resuspended at a concentration of 10×10⁶ cells/mL. Tumor suspension (50 μL) was incubated for 15-20 minutes in the dark at ART (ambient room temperature) with the following antibodies, Human (hu) CD45 FITC clone HI30, BioLegend, huCD4 PECy7 clone SK3, BioLegend, FoxP3 PE clone 259D, BioLegend, huCD25 APC clone M-A251, BioLegend, huCD3 V605 clone OKT3, BioLegend, 7-AAD, BioLegend. Flow cytometric data acquisition was performed using the FACSCantoll flow cytometer. Data was acquired using BD FACSDiva software. Cell populations was determined by electronic gating (P1=total leukocytes) on the basis of forward versus side scatter. The flow cytometer was set to collect 100,000 P1 events. The percentage of Tregs of CD45+ cells were calculated. Tregs were characterised by being viable CD45+, CD4+, FoxP3+, CD25+, CD3+ cells. The 7-AAD reagent was used to exclude non-viable cells in the flow cytometry analysis. Animals that were sacrificed prior to day 38 and animals that received more than 2 IP doses were excluded from analysis. Data is presented as mean % Tregs of CD45+ cells±SEM. Statistical significance (p<0.05) was determined using a Mann-Whitney test.

### Results and Discussion

The levels of tumor infiltrated Tregs at day 41 decreased for OT-1096 treated tumors for both donors (FIG. 12). This is shown by the flow cytometry measurements of infiltrating Tregs within the tumors, which revealed that the Treg levels were decreased in tumors that had been treated with OT-1096 (FIG. 12). Treg levels with both the OT-1096 alone treatment and the OT-1096+ Pembrolizumab combination treatment were decreased in comparison with vehicle controls (data not shown).

The data in this Example thus provides a further demonstration that a new effect of SecTRAP forming compounds exists, working in concert with (or stimulating) the immune system to combat cancer cell growth. As discussed elsewhere herein, Tregs have an immunosuppressive role in the tumour microenvironment and thus can inhibit an anti-cancer immune response. Thus, depleting Treg populations or inhibiting Treg activity in particular within the tumour microenvironment is desirable.

### EXAMPLE 3

Viability of Isolated Immune Cells after Treatment with OT-1096

### Materials and Methods

Viability of neutrophils after treatment with OT-1096 for 24 h was assessed using donated blood from a healthy male volunteer (HBsAg, HIV I&II negative), Age: 27 yrs (Blood collection date: 1 Jun. 2017) where neutrophils were isolated using dextran sedimentation followed by hypotonic lysis and final isolation by Histopaque 1077 (Sigma-Aldrich). Viability was assessed using an MTT assay.

Viability of PBMC (peripheral blood mononuclear cells) after treatment with OT-1096 for 24 h was assessed using donated blood from two different donors. Donor-1, a healthy male volunteer (HBsAg, HIV I&II negative), Age: 23 yrs (Blood collection date: 1 Jun. 2017). Donor-2, a healthy male volunteer (HBsAg, HIV I&II negative), Age: 40 yrs (Blood collection date: 8 Jun. 2017). The PBMC was isolated using Histopaque 1077 (Sigma-Aldrich). Viability was assessed using an MTT assay.

Viability of monocytes after treatment with OT-1096 for 24 h was assessed using donated blood from a healthy male volunteer (HBsAg, HIV I&II negative), Age: 38 yrs (Blood collection date: 8 Jun. 2017) where monocytes was isolated by Histopaque 1077 (Sigma-Aldrich) followed by purification with MiniMACS system of ‘Miltenyi Biotec’ using CD14 microbeads (Cat No. 130-050-201). Viability was assessed using an MTT assay.

Viability of CD8+ cells after treatment with OT-1096 for 24 h was assessed using donated blood from a healthy male volunteer (HBsAg, HIV I&II negative), Age: 38 yrs (Blood collection date: 21 Jun. 2017) where CD8+ cells were isolated by Histopaque 1077 (Sigma-Aldrich) followed by purification with MiniMACS system of ‘Miltenyi Biotec’ using CD8+ T Cell Isolation Reagent (Cat No. 130-096-495). Viability was assessed using an MTT assay.

Viability of CD4+ cells after treatment with OT-1096 for 24 h was assessed using donated blood from a healthy male volunteer (HBsAg, HIV I&II negative) where CD4+ cells were isolated by Histopaque 1077 (Sigma-Aldrich) followed by purification with MiniMACS system of ‘Miltenyi Biotec’ using CD4+ T Cell Isolation Reagent (Cat No. 130-096-533). Viability was assessed using an MTT assay.

Viability of Tregs (CD4+CD25+FOXP3) after treatment with OT-1096 for 24 h was assessed using donated blood from healthy human volunteer (HBsAg, HIV I&II negative) where Tregs were isolated by Histopaque 1077 (Sigma-Aldrich) followed by purification with MiniMACS system of ‘Miltenyi Biotec’ using CD4+CD25+ Regulatory T Cell Isolation Reagent (130-091-301). Viability was assessed using an MTT assay.

### Results

The viability of the various isolated immune cells populations after treatment with OT-1096 for 24 h was assessed (FIG. 13). No (or low) cytotoxicity in any of the isolated immune cell populations was observed for treatment with OT-1096 up to 33 μM.

### EXAMPLE 4

Intratumoral Immune Cell Populations of Engrafted Tumor Cells after OT-1096, Iniparib, or the Combination of OT-1096 and Iniparib

### Materials and Methods

Intratumoral immune cell populations within engrafted 4T1 cells in BALB/c mice. 4T1 cells are a murine mammary carcinoma cell line. BALB/c mice are immunocompetent. 1×10⁶ 4T1 tumor cells in 0% Matrigel were implanted orthotopically into the mammary fat pad. Enrollment of the mice into the treatment arms commenced when the tumors reached an average volume between 175-200 mm³. Upon enrollment mice were treated bid, twice per day, intratumorally with vehicle, 1 mg/kg Iniparib, 1 mg/kg OT-1096, or the combination of OT-1096 and Iniparib for 5 days (N=10 per group). Tumor volumes were measured using a digital caliper on day 1, 3, and 6 of the study. On day 6 mice were euthanized and tumors were resected for FACs analysis. Populations of murine immune cells including CD45+, CD4+, CD8+, and Tregs were analyzed. CD45+ positive cells were analyzed as the percentage of live cells. CD4+, CD8+, and Tregs cells were analyzed as percentage of CD45+ positive cells. Statistically significant differences between no treatment and treatment groups was determined using a Mann-Whitney test (*p<0.05, **p<0.01, *** p<0.001).

### Results

Direct injection of Iniparib, OT-1096, or the combination of Iniparib and OT-1096 significantly increased CD8+ levels relative to no treatment controls (FIG. 14A). This shows that there is increased infiltration of CD8+ cells in tumors after OT-1096 treatment (or after treatment with the combination of Iniparib and OT-1096), compared to the no treatment or vehicle controls. The ratio of CD8+ to Treg cells was also statistically significantly increased relative to no treatment controls (FIG. 14B). This shows that there is an increase in the ratio of CD8+ cells to Treg cells in tumours after OT-1096 treatment (or treatment with the combination of Iniparib and OT-1096), compared to the no treatment or vehicle controls.
The data also indicates that treatment with Iniparib alone increases CD8+ levels in tumours as compared to the no treatment or vehicle controls (FIG. 14A) and that treatment with Iniparib alone increases the ratio of CD8+ cells to Treg cells in tumours as compared to the no treatment or vehicle controls (FIG. 14B).
It is known in the art that an increase in the ratio of CD8+/Treg in cancer is correlated to better survival probability and thus the finding that treatment with OT-1096, Iniparib or the combination of Iniparib and OT-1096, increases the ratio of CD8+ cells to Treg cells in tumours indicates that such treatments (and treatments with other SecTRAP forming agents) are useful cancer therapies and that such compounds elicit anti-cancer immune activity and so may be particularly useful in the treatment of cancers (e.g. immune cell infiltrated cancers such as T-cell infiltrated cancers).

### REFERENCES

Anestål, K. et al., 2008. Cell death by SecTRAPs: thioredoxin reductase as a prooxidant killer of cells. PloS one, 3(4), p. e1846.
Anestål, K. & Arnér, E. S. J., 2003. Rapid induction of cell death by selenium-compromised thioredoxin reductase 1 but not by the fully active enzyme containing selenocysteine. Journal of Biological Chemistry, 278(18), pp. 15966-15972.
Arnér, E. S., Björnstedt, M. & Holmgren, A., 1995. 1-Chloro-2,4-dinitrobenzene is an irreversible inhibitor of human thioredoxin reductase. Loss of thioredoxin disulfide reductase activity is accompanied by a large increase in NADPH oxidase activity. The Journal of biological chemistry, 270(8), pp. 3479-82.
Arnér, E. S. J., 2009. Focus on mammalian thioredoxin reductases—Important selenoproteins with versatile functions. Biochimica et Biophysica Acta—General Subjects, 1790(6), pp. 495-526.
Cenas, N. et al., 2004. Interactions of quinones with thioredoxin reductase: a challenge to the antioxidant role of the mammalian selenoprotein. The Journal of biological chemistry, 279(4), pp. 2583-92.
Cheng, Q. et al., 2010. The selenium-independent inherent pro-oxidant NADPH oxidase activity of mammalian thioredoxin reductase and its selenium-dependent direct peroxidase activities. The Journal of biological chemistry, 285(28), pp. 21708-23.
Chew, E.-H. et al., 2008. Thioredoxin reductase inhibition by antitumor quinols: a quinol pharmacophore effect correlating to antiproliferative activity. FASEB journal: official publication of the Federation of American Societies for Experimental Biology, 22(6), pp. 2072-83.
Duan, D. et al., 2014. Shikonin targets cytosolic thioredoxin reductase to induce ROS-mediated apoptosis in human promyelocytic leukemia HL-60 cells. Free radical biology & medicine, 70, pp. 182-93.
Duan, D. et al., 2016. Targeting thioredoxin reductase by parthenolide contributes to inducing apoptosis of HeLa cells. Journal of Biological Chemistry, 291(19), pp. 10021-10031.
Eriksson, S. E., 2011. THIOREDOXIN REDUCTASE AS A TARGET ENZYME FOR ELECTROPHILIC ANTICANCER DRUGS. Karolinska.
Fang, J., Lu, J. & Holmgren, A., 2005. Thioredoxin reductase is irreversibly modified by curcumin: A novel molecular mechanism for its anticancer activity. Journal of Biological Chemistry, 280(26), pp. 25284-25290.
Golubovskaya, V. & Wu, L., 2016. Different subsets of T cells, memory, effector functions, and CAR-T immunotherapy. Cancers, 8(3).
Hedström, E. et al., 2009. p53-dependent inhibition of TrxR contributes to the tumor-specific induction of apoptosis by RITA. Cell cycle (Georgetown, TexJ, 8(21), pp. 3576-3583.
Herbst, R. S. et al., 2014. Predictive correlates of response to the anti-PD-L1 antibody MPDL3280A in cancer patients. Nature, 515(7528), pp. 563-567.
Jan, Y.-H. et al., 2014. Cross-linking of thioredoxin reductase by the sulfur mustard analogue mechlorethamine (methylbis(2-chloroethyl)amine) in human lung epithelial cells and rat lung: selective inhibition of disulfide reduction but not redox cycling. Chemical research in toxicology, 27(1), pp. 61-75.
Kaminska, K. K. et al., 2016. Indolin-2-one compounds targeting thioredoxin reductase as potential anticancer drug leads. Oncotarget, 7(26), pp. 3-6.
Lu, J., Chew, E.-H. & Holmgren, A., 2007. Targeting thioredoxin reductase is a basis for cancer therapy by arsenic trioxide. Proceedings of the National Academy of Sciences of the United States of America, 104(30), pp. 12288-93.
Masucci, G. V. et al., 2016. Validation of biomarkers to predict response to immunotherapy in cancer: Volume I—pre-analytical and analytical validation. Journal for immunotherapy of cancer, 4(1), p. 76.
Nordberg, J. et al., 1998. Mammalian thioredoxin reductase is irreversibly inhibited by dinitrohalobenzenes by alkylation of both the redox active selenocysteine and its neighboring cysteine residue. Journal of Biological Chemistry, 273(18), pp. 10835-10842.
Paz, M. M. et al., 2012. A new mechanism of action for the anticancer drug Mitomycin C: Mechanism-based inhibition of thioredoxin reductase. Chemical Research in Toxicology, 25(7), pp. 1502-1511.
Peng, X. et al., 2013. APR-246/PRIMA-1^{MET} inhibits thioredoxin reductase 1 and converts the enzyme to a dedicated NADPH oxidase. Cell Death and Disease, 4, e881.
Pruneri, G. et al., 2016. Clinical validity of tumor-infiltrating lymphocytes analysis in patients with triple-negative breast cancer. Annals of Oncology, 27(2), pp. 249-256.
Rackham, O. et al., 2011. Substrate and inhibitor specificities differ between human cytosolic and mitochondrial thioredoxin reductases: Implications for development of specific inhibitors. Free radical biology & medicine, 50(6), pp. 689-99.
Saccoccia, F. et al., 2014. Thioredoxin reductase and its inhibitors. Current protein & peptide science, 15(6), pp. 621-46.
Sachweh, M. C. C. et al., 2015. Redox effects and cytotoxic profiles of MJ25 and auranofin towards malignant melanoma cells. Oncotarget, 6(18), pp. 16488-16506.
Stafford, W. C., 2015. ELUCIDATION OF THIOREDOXIN REDUCTASE 1 AS AN ANTICANCER DRUG TARGET.
Wang, X., Zhang, J. & Xu, T., 2007. Cyclophosphamide as a potent inhibitor of tumor thioredoxin reductase in vivo. Toxicology and applied pharmacology, 218(1), pp. 88-95.
Willinger, T. et al., 2005. Molecular signatures distinguish human central memory from effector memory CD8 T cell subsets. Journal of immunology (Baltimore, Md.: 1950), 175(9), pp. 5895-903.
Witte, A. B. et al., 2005. Inhibition of thioredoxin reductase but not of glutathione reductase by the major classes of alkylating and platinum-containing anticancer compounds. Free Radical Biology and Medicine, 39(5), pp. 696-703.
Zhang, B. et al., 2017. Thioredoxin reductase inhibitors: a patent review. Expert opinion on therapeutic patents, 27(5), pp. 547-556.
Clarke, S. L. et al., 2006. CD4+CD25+FOXP3+ regulatory T cells suppress anti-tumor immune responses in patients with colorectal cancer. PLoS ONE, 1(1), pp. 2-7.
Curiel, T. J. et al., 2004. Specific recruitment of regulatory T cells in ovarian carcinoma fosters immune privilege and predicts reduced survival. Nature medicine, 10(9), pp. 942-9.
Gedeon, P. C. et al., 2014. Antibody-Based Immunotherapy for Malignant Glioma. Seminars in Oncology, 41(4), pp. 496-510.
Geng, Y. et al., 2015. Prognostic Role of Tumor-Infiltrating Lymphocytes in Lung Cancer: a Meta-Analysis. Cellular physiology and biochemistry: international journal of experimental cellular physiology, biochemistry, and pharmacology, 37(4), pp. 1560-71.
Gyorki, D. E. et al., 2013. Immunological insights from patients undergoing surgery on ipilimumab for metastatic melanoma. Annals of surgical oncology, 20(9), pp. 3106-11.
Humphries, W. et al., 2010. The Role of Tregs in Glioma-Mediated Immunosuppression: Potential Target for Intervention. Neurosurgery Clinics of North America, 21(1), pp. 125-137.
Jang, J. E. et al., 2017. Crosstalk between Regulatory T Cells and Tumor-Associated Dendritic Cells Negates Anti-tumor Immunity in Pancreatic Cancer. Cell Reports, 20(3), pp. 558-571.
Kindlund, B. et al., 2017. CD4(+) regulatory T cells in gastric cancer mucosa are proliferating and express high levels of IL-10 but little TGF-3. Gastric cancer: official journal of the International Gastric Cancer Association and the Japanese Gastric Cancer Association, 20(1), pp. 116-125.
Knol, A. C. et al., 2011. Prognostic value of tumor-infiltrating Foxp3+ T-cell subpopulations in metastatic melanoma. Experimental dermatology, 20(5), pp. 430-4.
Lan, Y. et al., 2017. Change in the Treg/Th17 cell imbalance in hepatocellular carcinoma patients and its clinical value. Medicine, 96(32), p. e7704. A
Mao, Y. et al., 2016. The prognostic value of tumor-infiltrating lymphocytes in breast cancer: A systematic review and meta-analysis. PLoS ONE, 11(4), pp. 1-13.
Overacre-Delgoffe, A. E. et al., 2017. Interferon-γ Drives Treg Fragility to Promote Anti-tumor Immunity. Cell, 169(6), p. 1130-1141.ell.
Pillarisetty, V. G., 2014. The pancreatic cancer microenvironment: an immunologic battleground. Oncolmmunology, 3(8), p. e950171.
Pruneri, G. et al., 2016. Clinical validity of tumor-infiltrating lymphocytes analysis in patients with triple-negative breast cancer. Annals of Oncology, 27(2), pp. 249-256.
Shen, L.-S. et al., 2009. CD4(+)CD25(+)CD127(low/−) regulatory T cells express Foxp3 and suppress effector T cell proliferation and contribute to gastric cancers progression. Clinical immunology (Orlando, Fla.), 131(1), pp. 109-18.
Tothill, R. W. et al., 2008. Novel molecular subtypes of serous and endometrioid ovarian cancer linked to clinical outcome. Clinical Cancer Research, 14(16), pp. 5198-5208.
Zhao, H.-Q. et al., 2014. Roles of Tregs in development of hepatocellular carcinoma: a meta-analysis. World journal of gastroenterology, 20(24), pp. 7971-8.

1. A selenium compromised thioredoxin reductase-derived apoptotic protein (SecTRAP) forming agent for use in treating an immune cell infiltrated cancer in a subject, wherein said agent stimulates an anti-cancer immune response.

2. The selenium compromised thioredoxin reductase-derived apoptotic protein (SecTRAP) forming agent for use according to claim 1, wherein said immune cell infiltrated cancer is a T-cell infiltrated cancer.

3. The selenium compromised thioredoxin reductase-derived apoptotic protein (SecTRAP) forming agent for use according to claim 1 or claim 2, wherein said anti-cancer immune response is characterized by
(i) a reduction in the level of Tregs; and/or
(ii) an increase in the level of CD8+ T-cells and/or other cytotoxic immune cells; and/or
(iii) a reduction in the ratio of Tregs to CD8+ T-cells and/or other cytotoxic immune cells.

4. The selenium compromised thioredoxin reductase-derived apoptotic protein (SecTRAP) forming agent for use according to any one of claims 1 to 3, wherein said SecTRAP forming agent is is a compound of formula XI 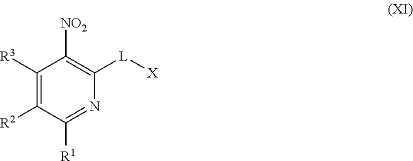
or a pharmaceutically acceptable salt thereof, wherein:
L represents —S(O)₂— or —S(O)—
X represents a heteroaryl group or heterocyclyl, connected to L via a carbon atom, or C₁₋₁₂ alkyl, C₂₋₁₂ alkenyl, C₂₋₁₂ alkynyl, or phenyl, each optionally substituted by one or more groups independently selected from Y;
R¹, R² and R³ each independently represent H, halo, R^{a1}, —CN, -A^{a1}-C(Q^{a1})R^{b1}, -A^{b1}-C(Q^{b1})N(R^{c1})R^{d1}, -A^{c1}-C(Q^{c1})OR^{e1}, -A^{d1}-S(O)ₚR^{f1}, -A^{e1}-S(O)ₚN(R^{g1})R^{h1}, -A^{f1}-S(O)ₚORⁱ¹, —N₃, —N(R^{j1})R^{k1}, —N(H)CN, —NO₂, —ONO₂, —OR^{l1} or —SR^{m1};
each A^{a1} to A^{f1} independently represents a single bond, —N(R^{p1})— or —O—;
each Q^{a1} to Q^{c1} independently represents ═O, ═S, ═NRⁿ¹ or ═N(OR^{o1});
each R^{a1} and R^{f1} independently represents C₁₋₆ alkyl, C₂₋₆ alkenyl or C₂₋₆ alkynyl each optionally substituted by one or more groups independently selected from G^{1a}, or heterocyclyl optionally substituted by one or more groups independently selected from G^{1b};
each R^{b1}, R^{c1}, R^{d1}, R^{e1}, R^{g1}, R^{h1}, Rⁱ¹, R^{j1}, R^{k1}, R^{l1}, R^{m1}, Rⁿ¹, R^{o1} and R^{p1} independently represents H, C₁₋₆ alkyl, C₂₋₆ alkenyl or C₂₋₆ alkynyl each optionally substituted by one or more groups independently selected from G^{1a}, or heterocyclyl optionally substituted by one or more groups independently selected from G^{1b}; or
any of R^{c1} and R^{d1}, R^{g1} and R^{h1} and/or R^{j1} and R^{k1} are linked together to form, together with the nitrogen atom to which they are attached, a 3- to 6-membered ring, which ring optionally contains one further heteroatom and which ring optionally is substituted by one or more groups independently selected from G^{1b}, C₁₋₃ alkyl, C₂₋₃ alkenyl or C₂₋₃ alkynyl each optionally substituted by one or more G^{1a}, and ═O;
each G^{1a} and G^{1b} independently represents halo, —CN, —N(R^{a2})R^{b2}, —OR^{c2}, —SR^{d2} or ═O;
each R^{a2}, R^{b2}, R^{c2} and R^{d2} independently represents H or C₁₋₆ alkyl, C₂₋₆ alkenyl or C₂₋₆ alkynyl each optionally substituted by one or more fluoro; or
R^{a2} and R^{b2} are linked together to form, along with the nitrogen atom to which they are attached, a 3- to 6-membered ring, which ring optionally contains one further heteroatom and which ring optionally is substituted by one or more groups independently selected from fluoro and C₁₋₃ alkyl, C₂₋₃ alkenyl or C₂₋₃ alkynyl each optionally substituted by one or more fluoro;
each Y independently represents halo, R^{a3}, —CN, -A^{a2}-C(Q^{a2})R^{b3}, -A^{b2}-C(Q^{b2})N(R^{c3})R^{d3}, -A^{c2}-C(Q^{c2})OR^{e3}, -A^{d2}-S(O)_{q}R^{f3}, -A^{e2}-S(O)_{q}N(R^{g3})R^{h3}, -A^{f2}-S(O)_{q}ORⁱ³, —N₃, —N(R^{j3})R^{k3}, —N(H)CN, —NO₂, —ONO₂, —OR^{l3}, —SR^{m3} or ═O
each Q^{a2} to Q^{c2} independently represents ═O, ═S, ═NRⁿ³ or ═N(OR^{o3});
each A^{a2} to A^{f2} independently represents a single bond, —N(R^{p3})— or —O—;
each R^{a3} and R^{f3} independently represents C₁₋₆ alkyl, C₂₋₆ alkenyl or C₂₋₆ alkynyl each optionally substituted by one or more groups independently selected from G^{2a}, heterocyclyl optionally substituted by one or more groups independently selected from G^{2b}, aryl optionally substituted by one or more groups independently selected from G^{2c}, or heteroaryl optionally substituted by one or more groups independently selected from G^{2d};
each R^{b3}, R^{c3}, R^{d3}, R^{e3}, R^{g3}, R^{h3}, Rⁱ³, R^{j3}, R^{k3}, R^{l3}, R^{m3}, Rⁿ³, R^{o3} and R^{p3} independently represents H, C₁₋₆ alkyl, C₂₋₆ alkenyl or C₂₋₆ alkynyl each optionally substituted by one or more groups independently selected from G^{2a}, heterocyclyl optionally substituted by one or more groups independently selected from G^{2b}, aryl optionally substituted by one or more groups independently selected from G^{2c}, or heteroaryl optionally substituted by one or more groups independently selected from G^{2d}; or
any two R^{c3} and R^{d3}, R^{g3} and R^{h3} and/or R^{j3} and R^{k3} are linked together to form, along with the nitrogen atom to which they are attached, a 3- to 6-membered ring, which ring optionally contains one further heteroatom and which ring optionally is substituted by one or more groups independently selected from halogen, C₁₋₃alkyl optionally substituted by one or more halogens, ═O, heterocyclyl optionally substituted by one or more groups independently selected from G^{2b}, aryl optionally substituted by one or more groups independently selected from G^{2c}, or heteroaryl optionally substituted by one or more groups independently selected from G^{2d};
each G^{2a} independently represents halo, —CN, —N(R^{j4})R^{k4}, —OR^{l4}, —SR^{m4} or ═O;
each G^{2b} independently represents halo, R^{a4}, —CN, —N(R^{j4})R^{k4}, —OR^{l4}, —SR^{m4} or ═O;
each G^{2c} and G^{2d} independently represents halo, R^{a4}, —CN, -A^{a3}-C(Q^{a4})R^{b4}, -A^{b3}-C(Q^{b3})N(R^{c4})R^{d4}, -A^{c3}-C(Q^{c3})OR^{e4}, -A^{d3}-S(O)_{q}R^{f4}, -A^{e3}-S(O)_{q}N(R^{g4})R^{h4}, -A^{f3}-S(O)_{q}ORⁱ⁴, —N₃, —N(R^{j4})R^{k4}, —N(H)CN, —NO₂, —ONO₂, —OR^{l4} or —SR^{m4};
each Q^{a3} to Q^{c3} independently represents ═O, ═S, ═NRⁿ⁴ or ═N(OR^{o4});
each A^{a3} to A^{f3} independently represents a single bond, —N(R^{p4})— or —O—;
each R^{a4} and R^{f4} independently represents C₁₋₆ alkyl, C₂₋₆ alkenyl or C₂₋₆ alkynyl each optionally substituted by one or more groups independently selected from G^{3a}, heterocyclyl optionally substituted by one or more groups independently selected from G^{3b}, aryl optionally substituted by one or more groups independently selected from G^{3c}, or heteroaryl optionally substituted by one or more groups independently selected from G^{3d};
each R^{b4}, R^{c4}, R^{d4}, R^{e4}, R^{g4}, R^{h4}, Rⁱ⁴, R^{j4}, R^{k4}, R^{l4}, R^{m4}, Rⁿ⁴, R^{o4} and R^{p4} independently represents H, C₁₋₆ alkyl, C₂₋₆ alkenyl or C₂₋₆ alkynyl each optionally substituted by one or more groups independently selected from G^{3a} or heterocyclyl optionally substituted by one or more groups independently selected from G^{3b}, aryl optionally substituted by one or more groups independently selected from G^{3c}, or heteroaryl optionally substituted by one or more groups independently selected from G^{3d}; or
any of R^{c4} and R^{d4}, R^{g4} and R^{h4} and/or R^{j4} and R^{k4} are linked together to form, together with the nitrogen atom to which they are attached, a 3- to 6-membered ring, which ring optionally contains one further heteroatom and which ring optionally is substituted by one or more groups independently selected G^{3b};
each G^{3a} and G^{3b} independently represents halo, R^{a5}, —CN, —N(R^{b5})R^{c5}, —OR^{d5}, —SR^{e5} or ═O;
G^{3c} and G^{3d} independently representing halo, R^{a5}, —CN, -A^{a4}-C(Q^{a4})R^{b5}, -A^{b4}-C(Q^{b4})N(R^{c5})R^{d5}, -A^{c4}-C(Q^{c4})OR^{e5}, -A^{d5}-S(O)_{q}R^{f5}, -A^{e4}-S(O)_{q}N(R^{g5})R^{h5}, -A^{f4}-S(O)_{q}ORⁱ⁵, —N₃, —N(R^{j5})R^{k5}, —N(H)CN, —NO₂, —ONO₂, —OR^{l5} or —SR^{m5},
each Q^{a4} to Q^{c4} independently represents ═O, ═S, ═NRⁿ⁵ or ═N(OR^{o5});
each A^{a4} to A^{f4} independently represents a single bond, —N(R^{p5})— or —O—;
with each R^{f5} to R^{p5} independently representing H, or C₁₋₆ alkyl, C₂₋₆ alkenyl or C₂₋₆ alkynyl each optionally substituted by one or more groups independently selected from G⁴, or with each R^{g5} and R^{h5}, and R^{j5} and R^{k5} being linked together to form, together with the nitrogen atom to which they are attached, a 3- to 6-membered ring, which ring optionally contains one further heteroatom and which ring optionally is substituted by one or more groups independently selected from G⁴;
each R^{a5} independently represents C₁₋₆ alkyl, C₂₋₆ alkenyl or C₂₋₆ alkynyl each optionally substituted by one or more groups independently selected from G⁴;
each R^{b5}, R^{c5}, R^{d5} and R^{e5} independently represents H, or C₁₋₆ alkyl, C₂₋₆ alkenyl or C₂₋₆ alkynyl each optionally substituted by one or more groups independently selected from G⁴; or
each R^{b5} and R^{c5} are linked together to form, together with the nitrogen atom to which they are attached, a 3- to 6-membered ring, which ring optionally contains one further heteroatom and which ring optionally is substituted by one or more groups independently selected from G⁴;
each G⁴ independently represents halo, R^{a6}, —CN, —N(R^{b6})R^{c6}, —OR^{d6} or ═O;
each R^{a6} independently represents C₁₋₆ alkyl, C₂₋₆ alkenyl or C₂₋₆ alkynyl each optionally substituted by one or more fluoro;
each R^{b6}, R^{c6} and R^{d6} independently represents H, or C₁₋₆ alkyl, C₂₋₆ alkenyl or C₂₋₆ alkynyl each optionally substituted by one or more fluoro; and
each p and q independently represents 1 or 2.

5. The selenium compromised thioredoxin reductase-derived apoptotic protein (SecTRAP) forming agent for use according to any one of claims 1 to 4, wherein said SecTRAP forming agent is selected from the group consisting of:

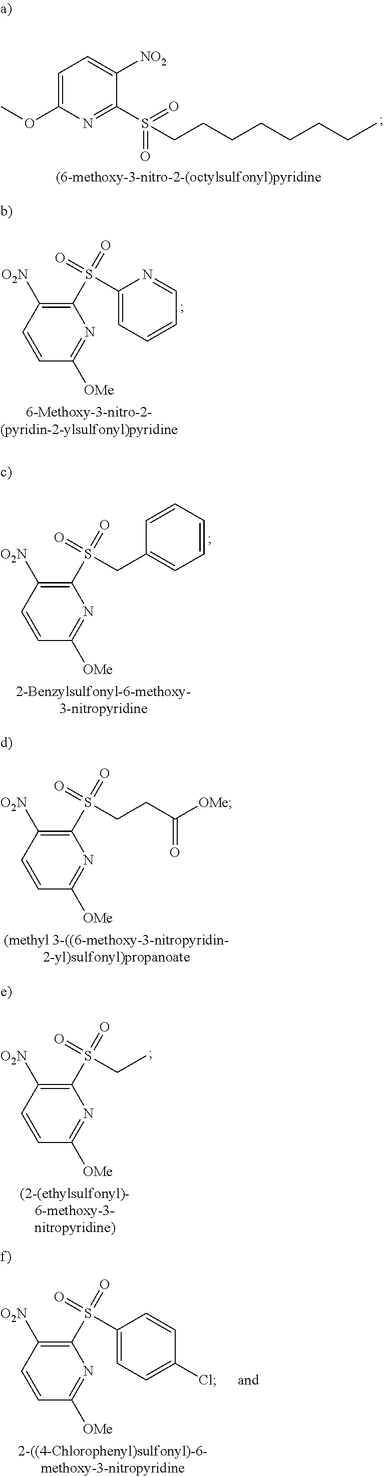

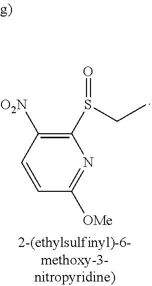

6. The selenium compromised thioredoxin reductase-derived apoptotic protein (SecTRAP) forming agent for use according to any one of claims 1 to 5, wherein said SecTRAP forming agent is selected from the group consisting of:

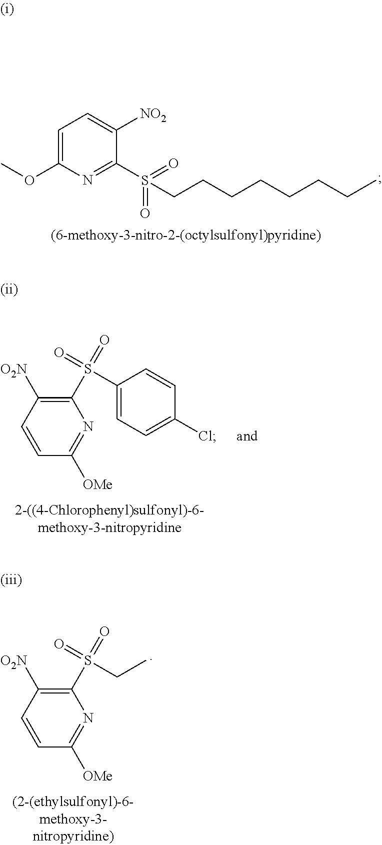 [SMILES: CCCCCCCCS(=O)(=O)c1nc(OC)ccc1[N+](=O)[O-] ; COc1ccc([N+](=O)[O-])c(S(=O)(=O)c2ccc(Cl)cc2)n1 ; "and" ; CCS(=O)(=O)c1nc(OC)ccc1[N+](=O)[O-]]

7. A selenium compromised thioredoxin reductase-derived apoptotic protein (SecTRAP) forming agent for use according to any one of claims 1 to 3, wherein said SecTRAP forming agent is a compound of formula II 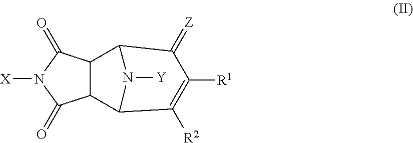
or a pharmaceutically acceptable salt thereof, wherein:
X represents C₁₋₁₂ alkyl optionally substituted by one or more groups independently selected from G^{1a}, heterocycloalkyl optionally substituted by one or more groups independently selected from G^{1b}, aryl optionally substituted by one or more groups independently selected from G^{1c}, or heteroaryl optionally substituted by one or more groups independently selected from G^{1d};
Y represents C₁₋₁₂ alkyl optionally substituted by one or more groups independently selected from G^{2a}; heterocycloalkyl optionally substituted by one or more groups independently selected from G^{2b}, aryl optionally substituted by one or more groups independently selected from G^{2c}, or heteroaryl optionally substituted by one or more groups independently selected from G^{2d};
Z represents O, S, NR^{a} or N(OR^{b});
R¹ and R² independently represents H or C₁₋₆ alkyl, the latter group being optionally substituted by one or more groups independently selected from halo and —OC₁₋₆ alkyl optionally substituted by one or more halo;
each G^{1a}, G^{1b}, G^{1c} and G^{1d} independently represents halo, R^{a1}, —CN, -A^{a1}-C(Q^{a1})R^{b1}, -A^{b1}-C(Q^{b1})N(R^{c1})R^{d1}, -A^{c1}-C(Q^{c1})OR^{e1}, -A^{d1}-S(O)ₙR^{f1}, -A^{e1}-S(O)ₙC(O)R^{g1}, -A^{f1}-S(O)ₙN(R^{h1})Rⁱ¹, -A^{g1}-S(O)ₙOR^{j1}, —N₃, —N(R^{k1})R^{l1}, —N(H)CN, —NO₂, —OR^{m1}, —SRⁿ¹ or =Q^{d1};
each A^{a1} to A^{g1} independently represents a single bond, —N(R^{o1})—, —C(Q^{e1})N(R^{p1})— or —O—;
each Q^{a1} to Q^{e1} independently represents ═O, ═S, ═NR^{q1} or ═N(OR^{r1});
R^{a} and R^{b} each independently represent H or C₁₋₆ alkyl, the latter group being optionally substituted by one or more groups independently selected from halo and —OC₁₋₆ alkyl optionally substituted by one or more halo;
each R^{a1} and R^{f1} independently represents C₁₋₆ alkyl optionally substituted by one or more groups independently selected from G^{3a}, heterocycloalkyl optionally substituted by one or more groups independently selected from G^{3b}, aryl optionally substituted by one or more groups independently selected from G^{3c}, or heteroaryl optionally substituted by one or more groups independently selected from G^{3d};
each R^{b1}, R^{c1}, R^{d1}, R^{e1}, R^{g1}, R^{h1}, Rⁱ¹, R^{j1}, R^{k1}, R^{l1}, R^{m1}, Rⁿ¹, R^{q1} and R^{r1} independently represents H, C₁₋₆ alkyl optionally substituted by one or more groups independently selected from G^{3a}, heterocycloalkyl optionally substituted by one or more groups independently selected from G^{3b}, aryl optionally substituted by one or more groups independently selected from G^{3c}, or heteroaryl optionally substituted by one or more groups independently selected from G^{3d};
or any two R^{c1} and R^{d1}, R^{h1} and Rⁱ¹ and/or R^{k1} and R^{l1} are linked together to form, along with the nitrogen atom to which they are attached, a 3- to 6-membered ring, which ring optionally contains one further heteroatom and which ring optionally is substituted by one or more groups independently selected from halo, C₁₋₃ alkyl optionally substituted by one or more halo, and ═O;
each R^{o1} and R^{p1} independently represents H or C₁₋₆ alkyl optionally substituted by one or more halo;
each G^{2a}, G^{2b}, G^{2c} and G^{2d} independently represents halo, R^{a2}, —CN, -A^{a2}-C(Q^{a2})R^{b2}, -A^{b2}-C(Q^{b2})N(R^{c2})R^{d2}, -A^{c2}-C(Q^{c2})OR^{e}, -A^{d2}-S(O)ₚR^{f2}, -A^{e2}-S(O)ₚC(O)R^{g2}, -A^{f2}-S(O)ₚN(R^{h2})Rⁱ², -A^{g2}-S(O)ₚOR^{j2}, —N₃, —N(R^{k2})R^{l2}, —N(H)CN, —NO₂, —OR^{m2}, —SRⁿ² or =Q^{d2};
each A^{a2} to A^{g2} independently represents a single bond, —N(R^{o2})—, —C(Q^{e2})N(R^{p2})— or —O—;
each Q^{a2} to Q^{e3} independently represents ═O, ═S, ═NR^{q2} or ═N(OR^{r2});
each R^{a2} independently represents heterocycloalkyl optionally substituted by one or more groups independently selected from G^{4b}, aryl optionally substituted by one or more groups independently selected from G^{4c}, or heteroaryl optionally substituted by one or more groups independently selected from G^{4d};
each R^{f2} independently represents C₁₋₆ alkyl optionally substituted by one or more groups independently selected from G^{4a}, heterocycloalkyl optionally substituted by one or more groups independently selected from G^{4b}, aryl optionally substituted by one or more groups independently selected from G^{4c}, or heteroaryl optionally substituted by one or more groups independently selected from G^{4d};
each R^{b2}, R^{c2}, R^{d2}, R^{e2}, R^{g2}, R^{h2}, Rⁱ², R^{j2}, R^{k2}, R^{l2}, R^{m2}, Rⁿ², R^{q2} and R^{r2} independently represents H, C₁₋₆ alkyl optionally substituted by one or more groups independently selected from G^{4a}, heterocycloalkyl optionally substituted by one or more groups independently selected from G^{4b}, aryl optionally substituted by one or more groups independently selected from G^{4c}, or heteroaryl optionally substituted by one or more groups independently selected from G^{4d};
or any two R^{c2} and R^{d2}, R^{h2} and Rⁱ² and/or R^{k2} and R^{l2} are linked together to form, along with the nitrogen atom to which they are attached, a 3- to 6-membered ring, which ring optionally contains one further heteroatom and which ring optionally is substituted by one or more groups independently selected from halo, C₁₋₃ alkyl optionally substituted by one or more halo, and ═O;
each R^{o2} and R^{p2} independently represents H or C₁₋₆ alkyl optionally substituted by one or more halo;
each G^{3a} independently represents halo, —CN, -A^{a3}-C(Q^{a3})R^{b3}, -A^{b3}-C(Q^{b3})N(R^{c3})R^{d3}, -A^{c3}-C(Q^{c3})OR^{e3}, -A^{d3}-S(O)_{q}R^{f3}, -A^{e3}-S(O)_{q}C(O)R^{g3}, -A^{f3}-S(O)_{q}N(R^{h3})Rⁱ³, -A^{g3}-S(O)_{q}OR^{j3}, —N₃, —N(R^{k3})Rⁱ³, —N(H)CN, —NO₂, —OR^{m3}, —SRⁿ³ or =Q^{d3};
each G^{3b}, G^{3c} and G^{3d} independently represents halo, R^{a3}, —CN, -A³-C(Q^{a3})R^{b3}, -A^{b3}-C(Q^{b3})N(R^{c3})R^{d3}, -A^{c3}-C(Q^{c3})OR^{e3}, -A^{d3}-S(O)_{q}R^{f3}, -A^{e3}-S(O)_{q}C(O)R^{g3}, -A^{f3}-S(O)_{q}N(R^{h3})Rⁱ³, -A^{g3}-S(O)_{q}OR^{j3}, —N₃, —N(R^{k3})R^{l3}, —N(H)CN, —NO₂, —OR^{m3}, —SRⁿ³ or =Q^{d3};
each A^{a3} to A^{g3} independently represents a single bond, —N(R^{o3})—, —C(Q^{e3})N(R^{p3})— or —O—;
each Q^{a3} to Q^{e3} independently represents ═O, ═S, ═NR^{q3} or ═N(OR^{r3});
each R^{a3} and R^{f3} independently represents C₁₋₆ alkyl optionally substituted by one or more groups independently selected from G^{5a}, or heterocycloalkyl optionally substituted by one or more groups independently selected from G^{5b};
each R^{b3}, R^{c3}, R^{d3}, R^{e3}, R^{g3}, R^{h3}, Rⁱ³, R^{j3}, R^{k3}, R^{l3}, R^{m3}, Rⁿ³, R^{q3} and R^{r3} independently represents H, C₁₋₆ alkyl optionally substituted by one or more groups independently selected from G^{5a}, or heterocycloalkyl optionally substituted by one or more groups independently selected from G^{5b};
or any two R^{c3} and R^{d3}, R^{h3} and Rⁱ³ and/or R^{k3} and R^{l3} are linked together to form, along with the nitrogen atom to which they are attached, a 3- to 6-membered ring, which ring optionally contains one further heteroatom and which ring optionally is substituted by one or more groups independently selected from halo, C₁₋₃ alkyl optionally substituted by one or more halo, and ═O;
each R^{o3} and R^{p3} independently represents H or C₁₋₆ alkyl optionally substituted by one or more halo;
each G^{4a} independently represents halogen, —CN, -A^{a4}-C(Q^{a4})R^{b4}, -A^{b4}-C(Q^{b4})N(R^{c4})R^{d4}, -A^{c4}-C(Q^{c4})OR^{e4}, -A^{d4}-S(O)ᵣR^{f4}, -A^{e4}-S(O)ᵣC(O)R^{g4}, -A^{f4}-S(O)ᵣN(R^{h4})Rⁱ⁴, -A^{g4}-S(O)ᵣOR^{j4}, —N₃, —N(R^{k4})R^{l4}, —N(H)CN, —NO₂, —OR^{m4}, —SRⁿ⁴ or =Q^{d4};
each G^{4b}, G^{4c} and G^{4d} independently represents halo, R^{a4}, —CN, -A^{a4}-C(Q^{a4})R^{b4}, -A^{b4}-C(Q^{b4})N(R^{c4})R^{d4}, -A^{c4}-C(Q^{c4})OR^{e4}, -A^{d4}-S(O)ᵣR^{f4}, -A^{e4}-S(O)ᵣC(O)R^{g4}, -A^{f4}-S(O)ᵣN(R^{h4})Rⁱ⁴, -A^{g4}-S(O)ᵣOR^{j4}, —N₃, —N(R^{k4})R^{l4}, —N(H)CN, —NO₂, —OR^{m4}, —SRⁿ⁴ or =Q^{d4};
each A^{a4} to A^{g4} independently represents a single bond, —N(R^{o4})—, —C(Q^{e4})N(R^{p4})— or —O—;
each Q^{a4} to Q^{e4} independently represents ═O, ═S, ═NR^{q4} or ═N(OR^{r4});
each R^{a4} and R^{f4} independently represents C₁₋₆ alkyl optionally substituted by one or more groups independently selected from G^{6a}, heterocycloalkyl optionally substituted by one or more groups independently selected from G^{6b}, or aryl optionally substituted by one or more groups independently selected from G^{6c};
each R^{b4}, R^{c4}, R^{d4}, R^{e4}, R^{g4}, R^{h4}, Rⁱ⁴, R^{j4}, R^{k4}, R^{l4}, R^{m4}, Rⁿ⁴, R^{q4} and R^{r4} independently represents H, C₁₋₆ alkyl optionally substituted by one or more groups independently selected from G^{6a}, or heterocycloalkyl optionally substituted by one or more groups independently selected from G^{6b};
or any two R^{c4} and R^{d4}, R^{h4} and Rⁱ⁴ and/or R^{k4} and R^{l4} are linked together to form, along with the nitrogen atom to which they are attached, a 3- to 6-membered ring, which ring optionally contains one further heteroatom and which ring optionally is substituted by one or more groups independently selected from halo, C₁₋₃ alkyl optionally substituted by one or more halo, and ═O;
each R^{o4} and R^{p4} independently represents H or C₁₋₆ alkyl optionally substituted by one or more halo;
each G^{5a} and G^{6a} independently represents halo or —OC₁₋₆ alkyl optionally substituted by one or more halo;
each G^{5b}, G^{6b} and G^{6c} represents halo, C₁₋₆ alkyl optionally substituted by one or more halogens, or —OC₁₋₆ alkyl optionally substituted by one or more halo;
each n independently represents 1 or 2;
each p independently represents 1 or 2;
each q independently represents 1 or 2; and
each r independently represents 1 or 2.

8. The selenium compromised thioredoxin reductase-derived apoptotic protein (SecTRAP) forming agent for use according to any one of claims 1 to 3 or claim 7, wherein said SecTRAP forming agent is

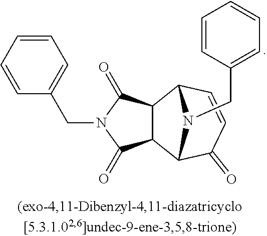 [SMILES: O=C1C=C[C@H]2[C@@H]3C(=O)N(Cc4ccccc4)C(=O)[C@@H]3[C@@H]1N2Cc1ccccc1]

9. The selenium compromised thioredoxin reductase-derived apoptotic protein (SecTRAP) forming agent for use according to any one of claims 1 to 3, wherein said SecTRAP forming agent is a compound of formula III 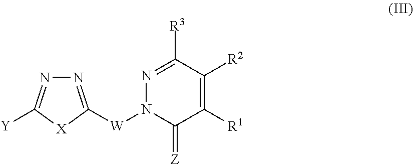
or a pharmaceutically acceptable salt thereof, wherein:
W represents C₁ alkylene optionally substituted by one or more groups independently selected from R⁴;
X represents O or S;
Y represents C₁₋₆ alkyl optionally substituted by one or more groups independently selected from G^{1a}, heterocycloalkyl optionally substituted by one or more groups independently selected from G^{1b}, aryl optionally substituted by one or more groups independently selected from G^{1c}, or heteroaryl optionally substituted by one or more groups independently selected from G^{1d};
Z represents O, S or NR⁵;
R¹ represents H, halo, R^{a1}, —CN, —C(Q^{a1})R^{b1}, —C(Q^{b1})N(R^{c1})R^{d1}, —C(Q^{c1})OR^{e1}, —S(O)ₙR^{f1}, —S(O)ₚN(R^{g1})R^{h1}, —S(O)ₚORⁱ¹ or —NO₂;
R² represents H, halo, —CN or —N₃;
R³ represents H, halo or R^{j1};
R⁴ represents halo or C₁₋₆ alkyl optionally substituted by one or more groups independently selected from G^{1e};
R⁵ represents H, R^{k1}, —OR^{l1} or —N(R^{m1})Rⁿ¹;
Q^{a1} to Q^{c1} each independently represents ═O, ═S, ═NR^{o1} or ═N(OR^{p1});
each R^{a1}, R^{f1}, R^{j1} and R^{k1} independently represents C₁₋₆ alkyl optionally substituted by one or more groups independently selected from G^{2a}, or heterocycloalkyl optionally substituted by one or more groups independently selected from G^{2b};
each R^{b1}, R^{c1}, R^{d1}, R^{e1}, R^{g1}, R^{h1}, Rⁱ¹, R^{l1}, R^{m1}, Rⁿ¹, R^{o1} and R^{p1} independently represents H, C₁₋₆ alkyl optionally substituted by one or more groups independently selected from G^{2a}, or heterocycloalkyl optionally substituted by one or more groups independently selected from G^{2b};
or any two R^{c1} and R^{d1}, R^{g1} and R^{h1} and/or R^{m1} and Rⁿ¹ are linked together to form, along with the nitrogen atom to which they are attached, a 3- to 6-membered ring, which ring optionally contains one further heteroatom and which ring optionally is substituted by one or more groups independently selected from halogen, C₁₋₃ alkyl optionally substituted by one or more halogens, and ═O;
each G^{1a}, G^{1b}, G^{1c} and G^{1d} represent halogen, R^{a2}, —CN, -A^{a1}-C(Q^{a2})R^{b2}, -A^{b1}-C(Q^{b2})N(R^{c2})R^{d2}, -A^{c1}-C(Q^{c2})OR^{e2}, -A^{d1}-S(O)_{q}R^{f2}, -A^{e1}-S(O)_{q}C(O)R^{g2}, -A^{f1}-S(O)_{q}N(R^{h2})Rⁱ², -A^{g1}-S(O)_{q}OR^{j2}, —N₃, —N(R^{k2})R^{l2}, —N(H)CN, —NO₂, —OR^{m2}, —SRⁿ² or =Q^{d2};
A^{a1} to A^{g1} each independently represents a single bond, —N(R⁶)—, —C(Q^{e2})N(R⁷)— or —O—;
Q^{a2} to Q^{e2} each independently represents ═O, ═S, ═NR^{o2} or ═N(OR^{p2});
each R⁶ and R⁷ independently represents H or C₁₋₆ alkyl optionally substituted by one or more F;
each R^{a2} and R^{f2} independently represents C₁₋₆ alkyl optionally substituted by one or more groups independently selected from G^{3a} or heterocycloalkyl optionally substituted by one or more groups independently selected from G^{3b};
each R^{b2}, R^{c2}, R^{d2}, R^{e2}, R^{g2}, R^{h2}, Rⁱ², R^{j2}, R^{k2}, R¹², R^{m2}, Rⁿ², R^{o2} and R^{p2} independently represents H, C₁₋₆ alkyl optionally substituted by one or more groups independently selected from G^{3a} or heterocycloalkyl optionally substituted by one or more groups independently selected from G^{3b}; or
any two R^{c2} and R^{d2}, R^{h2} and Rⁱ² and/or R^{k2} and R^{l2} are linked together to form, along with the nitrogen atom to which they are attached, a 3- to 6-membered ring, which ring optionally contains one further heteroatom and which ring optionally is substituted by one or more groups independently selected from halogen, C₁₋₃ alkyl optionally substituted by one or more halogens, and ═O;
each G^{1e} independently represents halo, R^{a2}, —CN, —N(R^{a3})R^{b3}, —OR^{c3} or —SR^{d3};
R^{a3}, R^{b3}, R^{c3} and R^{d3} each independently represents H or C₁₋₆ alkyl optionally substituted by one or more F;
or R^{a3} and R^{b3} are linked together to form, along with the nitrogen atom to which they are attached, a 3- to 6-membered ring, which ring optionally contains one further heteroatom and which ring optionally is substituted by one or more groups independently selected from fluoro, C₁₋₃ alkyl optionally substituted by one or more fluoro, and ═O;
each G^{2a} and G^{2b} independently represents halo, —CN, —N(R^{a4})R^{b4}, —OR^{c4}, —SR^{d4} or ═O;
each R^{a4}, R^{b4}, R^{c4} and R^{d4} independently represents H or C₁₋₆ alkyl optionally substituted by one or more F;
or R^{a4} and R^{b4} are linked together to form, along with the nitrogen atom to which they are attached, a 3- to 6-membered ring, which ring optionally contains one further heteroatom and which ring optionally is substituted by one or more groups independently selected from fluoro, C₁₋₃ alkyl optionally substituted by one or more fluoro, and ═O;
each G^{3a} and G^{3b} independently represents halo, —CN, —N(R^{a5})R^{b5}, —OR^{c5}, —SR^{d5} or ═O;
each R^{a5}, R^{b5}, R^{c5} and R^{d5} independently represents H or C₁₋₆ alkyl optionally substituted by one or more fluoro;
or R^{a5} and R^{b5} are linked together to form, along with the nitrogen atom to which they are attached, a 3- to 6-membered ring, which ring optionally contains one further heteroatom and which ring optionally is substituted by one or more groups independently selected from fluoro, C₁₋₃ alkyl optionally substituted by one or more fluoro, and ═O;
each n independently represents 0, 1 or 2,
each p independently represents 1 or 2,
each q independently represents 1 or 2.

10. The selenium compromised thioredoxin reductase-derived apoptotic protein (SecTRAP) forming agent for use according to any one of claims 1 to 3, wherein said SecTRAP forming agent is Iniparib.

11. The selenium compromised thioredoxin reductase-derived apoptotic protein (SecTRAP) forming agent for use according to any one of claims 1 to 3, wherein said SecTRAP forming agent is Auranofin.

12. The selenium compromised thioredoxin reductase-derived apoptotic protein (SecTRAP) forming agent for use according to any one of claims 1 to 11, wherein said SecTRAP forming agent is administered systemically.

13. The selenium compromised thioredoxin reductase-derived apoptotic protein (SecTRAP) forming agent for use according to any one of claims 1 to 12, wherein the SecTRAP forming agent is administered intravenously, intraperitoneally or intrathecally.

14. The selenium compromised thioredoxin reductase-derived apoptotic protein (SecTRAP) forming agent for use according to any one of claims 1 to 13, wherein said cancer overexpresses thioredoxin reductase (TrxR) or thioredoxin (Trx) or Protein Disulphide Isomerase (PDI), either individually or in any combination of the three.

15. The selenium compromised thioredoxin reductase-derived apoptotic protein (SecTRAP) forming agent for use according to any one of claims 1 to 14, wherein said cancer is breast cancer, brain cancer, advanced cancer or metastatic cancer.

16. The selenium compromised thioredoxin reductase-derived apoptotic protein (SecTRAP) forming agent for use according to any one of claims 1 to 15, wherein subjects with said cancer overexpress thioredoxin reductase (TrxR) or thioredoxin (Trx) or Protein Disulphide Isomerase (PDI), either individually or in any combination of the three in serum or blood.

17. A combination of
(i) a selenium compromised thioredoxin reductase-derived apoptotic protein (SecTRAP) forming agent; and
(ii) an immunostimulatory agent for use in treating cancer in a subject.

18. The combination for use according to claim 17, wherein said selenium compromised thioredoxin reductase-derived apoptotic protein (SecTRAP) forming agent is as defined in any one of claims 4 to 11.

19. The combination for use according to claim 17 or claim 18, wherein said selenium compromised thioredoxin reductase-derived apoptotic protein (SecTRAP) forming agent is administered as defined in claim 12 or claim 13.

20. The combination for use according to any one of claims 17 to 19, wherein said cancer or said anti-cancer immune response or said subject is as defined in any one of claims 1, 2, 3, 14, 15 or 16.

21. The combination for use according to any one of claims 17 to 20, wherein said immunostimulatory agent is an immune checkpoint inhibitor.

22. The combination for use according to claim 21, wherein said immune checkpoint inhibitor is an anti-PD-L1 antibody or an anti-PD1 antibody.

23. A combination of
(i) a selenium compromised thioredoxin reductase-derived apoptotic protein (SecTRAP) forming agent; and
(ii) a Thioredoxin antibody
for use in treating cancer in a subject.

24. The combination for use according to claim 23, wherein said use has the features as defined in any one of claims 18 to 20.

25. The combination for use according to 22, wherein said anti-PD1 antibody is Pembrolizumab.

26. A combination of
(i) a selenium compromised thioredoxin reductase-derived apoptotic protein (SecTRAP) forming agent; and
(ii) a targeted therapeutic agent or a cytotoxic therapeutic agent, for use in treating cancer in a subject.

27. The combination for use according to claim 26, wherein said targeted therapeutic agent is Imatinib, Bevacizumab or Everolimus.

28. The combination for use according to claim 26, wherein said cytotoxic therapeutic agent is carboplatin, a taxol or a vinca alkaloid.

29. The selenium compromised thioredoxin reductase-derived apoptotic protein (SecTRAP) forming agent for use according to any one of claims 1-16 or the combination for use according to any one of claims 17-28, wherein the selenium compromised thioredoxin reductase-derived apoptotic protein (SecTRAP) forming agent is

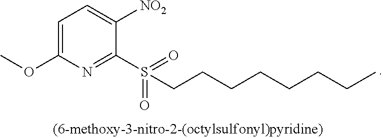 [SMILES: CCCCCCCCS(=O)(=O)c1nc(OC)ccc1[N+](=O)[O-]]

30. A method of treating an immune cell infiltrated cancer in a subject, said method comprising administering to a subject in need thereof a therapeutically effective amount of a selenium compromised thioredoxin reductase-derived apoptotic protein (SecTRAP) forming agent, wherein said agent stimulates an anti-cancer immune response.

31. A method of treating cancer in a subject, said method comprising administering to a subject in need thereof a combination of a therapeutically effective amount of a selenium compromised thioredoxin reductase-derived apoptotic protein (SecTRAP) forming agent and an immunostimulatory agent.

32. A method of treating cancer in a subject, said method comprising administering to a subject in need thereof a combination of a therapeutically effective amount of a selenium compromised thioredoxin reductase-derived apoptotic protein (SecTRAP) forming agent and a thioredoxin antibody.

33. A method of treating cancer in a subject, said method comprising administering to a subject in need thereof a combination of a therapeutically effective amount of a selenium compromised thioredoxin reductase-derived apoptotic protein (SecTRAP) forming agent and a targeted therapeutic agent or a cytotoxic therapeutic agent.

34. The method of any one of claims 30 to 33, wherein said method has features as defined in any one of claims 1 to 29.

35. Use of a selenium compromised thioredoxin reductase-derived apoptotic protein (SecTRAP) forming agent in the manufacture of a medicament for treating an immune cell infiltrated cancer wherein said agent stimulates an anti-cancer immune response.

36. Use of a selenium compromised thioredoxin reductase-derived apoptotic protein (SecTRAP) forming agent in the manufacture of a medicament for treating cancer wherein said treatment further comprises the administration of an immunostimulatory agent.

37. Use of a selenium compromised thioredoxin reductase-derived apoptotic protein (SecTRAP) forming agent in the manufacture of a medicament for treating cancer wherein said treatment further comprises the administration of a thioredoxin antibody.

38. Use of a selenium compromised thioredoxin reductase-derived apoptotic protein (SecTRAP) forming agent in the manufacture of a medicament for treating cancer wherein said treatment further comprises the administration of a targeted therapeutic agent or a cytotoxic therapeutic agent.

39. The use of any one of claims 35 to 38, wherein said use has features as defined in any one of claims 1 to 29.
